(19) United States
(12) Patent Application Publication
(10) Pub. No.: US 2020/0385371 A1
(43) Pub. Date: Dec. 10, 2020

(54) INHIBITORS OF WDR5 PROTEIN-PROTEIN BINDING

(71) Applicant: Propellon Therapeutics Inc., Toronto (CA)

(72) Inventors: Rima Al-Awar, Toronto (CA); Methvin Isaac, Brampton (CA); Joseph Babu, Oakville (CA); Yong Liu, Oakville (CA); Ahmed Mamai, Mississauga (CA); Gennady Poda, Toronto (CA); Pandiaraju Subramanian, Oakville (CA); David Uehling, Toronto (CA); Brian Wilson, Mississauga (CA); Carlos Armando Zepeda-Velazquez, Mississauga (CA)

(73) Assignee: Propellon Therapeutics Inc., Toronto, ON (CA)

(21) Appl. No.: 16/643,633

(22) PCT Filed: Sep. 6, 2018
(86) PCT No.: PCT/CA2018/051079

§ 371 Date: Mar. 2, 2020

Related U.S. Application Data
(60) Provisional application No. 62/554,812, filed on Sep. 6, 2017.

(51) Int. Cl. C07D 413/14; C07D 401/14; C07D 401/12

(52) U.S. Cl. CPC C07D 413/14; C07D 401/12; C07D 401/14

(57) ABSTRACT

The present application is directed to compounds of Formula I: (I) compositions comprising these compounds and their uses, for example as medicaments for the treatment of diseases, disorders or conditions mediated or treatable by inhibition of binding between WDR5 protein and its binding partners.

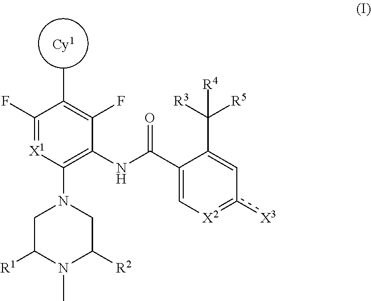

The present application claims the benefit of priority from co-pending U.S. Provisional Patent Application Ser. No. 62/554,812 filed on Sep. 6, 2017, the contents of which are incorporated herein by reference in their entirety.

### FIELD

The present application relates to compounds, to processes for their preparation, to compositions comprising them and their use for the treatment of diseases and conditions related to interactions between WDR5 and its binding partners including, but not limited to, MLL.

### BACKGROUND

Histones are the most basic units for packing DNA into nucleosomes and covalent modifications of histones, such as methylation, acetylation and phosphorylation, play a central role for regulation of gene transcription [Nat. Rev. Mol. Cell Biol. 2001, 2: 422-432; Cell 2007, 128:693-705]. Epigenetics refers to the heritable changes that control how the genome is accessed in different cell types during embryonic development and cellular differentiation [Genes. Dev. 2009; 23: 781-3]. This capability permits specialization of function between cells without altering the DNA sequence.

It is now well recognized that misregulation of histone modifications plays a key role in a wide range of human diseases, including but not limited to cancer [Cell., 2007, 10: 693-705; Nat. Rev. Cancer., 2010, 10:457-469]. Mixed Lineage Leukemia 1 (MLL1) protein is a Histone H3 Lysine 4 (H3K4) methyltransferase and is frequently misregulated in a subset of acute leukemia [Trends Mol. Med., 2004, 10: 500-507, Cell. Stem. Cell., 2007, 1:324-337]. MLL1 itself has a weak H3K4 methyltransferase activity but its enzymatic activity is dramatically enhanced when MLL1 is present in a core complex, made up of MLL1, WD repeat domain 5 protein (WDR5), Absent, Small, or Homeotic-2-Like (ASH2L) and Retinoblastoma Binding Protein 5 (RbBP5). Recent studies have clearly shown that the interaction between MLL1 and WDR5 proteins is essential for the activity of MLL1 but dispensable for the activity of other MLL family members, including MLL2, MLL3 and MLL4 [Mol. Cell., 2014, 53:247-261]. Hence, blocking the MLL1-WDR5 protein-protein interaction can specifically inhibit the activity of MLL1 H3K4 methyltransferase activity and such inhibition has the potential for the treatment of human diseases, such as, a subset of acute leukemia, whose development and progression depend upon MLL1 activity.

WDR5 is a common subunit of all six mammalian histone H3K4 methyltransferases [Dev. Biol., 2010, 339 (2):240-249]. WDR5 has 334 amino acids and contains seven typical WD40 repeat domains, each approximately 40 amino acids [Nat. Struct. Mol. Biol., 2009, 16 (7):678-680]. Structural studies suggest that the WD40 repeats form a seven-bladed propeller fold, with each blade made up of a four-stranded antiparallel sheet. This structural property suggests that WDR5 has many exposed surfaces making it a useful adaptor to interact with other proteins. Further, pulldown assays indicate that WDR5 prefers to bind dimethylated histone H3K4 peptide [Nat. Struct. Mol. Biol., 2009, 16 (7):678-680].

Because WDR5 is an essential component of the histone methylation, acetylation, and chromatin remodeling complexes, while not wishing to be limited by theory, WDR5 is believed to serve as an adaptor protein for complex assembly. However, it may also contribute to other physiological phenomena. WDR5 is an important component for assembly or stability of the virus-induced signaling adapter (VISA) associated complex, which plays a key role in virus-triggered induction of type I interferons (IFNs) and antiviral innate immune response [Proc. Nat. Acad. Sci. USA., 2010, 107(2):815-820]. Previous studies have demonstrated that VISA is located at the outer membrane of mitochondria. Interestingly, this study revealed that WDR5 was not only localized in the nucleus as believed before, but also abundantly localized in the cytoplasm. Viral infection induces translocation of WDR5 from the nucleus to the mitochondria located VISA complex, where it played a role in the assembly and stability of the VISA complex. These studies demonstrate for the first time a cytoplasmic function for WDR5, specifically in virus-triggered signaling resulting in induction of type I IFNs [Proc. Natl. Acad. Sci. U.S.A., 2010, 107(2):815-820].

### MLL1-WDR5 Complex in Leukemogenesis

Leukemia is characterized by an abnormal increase of white blood cells in the blood or bone marrow. Among all types of cancers, the morbidity of leukemia is the highest for patients below 35 years old. Over 70% of infant leukemia patients bear a translocation involving chromosome 11, resulting in the fusion of the MLL1 gene with other genes [Nat. Rev. Cancer., 2007, 7(11):823-833]. MLL1 translocations are also found in approximately 10% of adult acute myeloid leukemia (AML) patients, who were previously treated with topoisomerase II inhibitors for other types of cancers [Nat. Rev. Cancer., 2007, 7(11):823-833].

MLL1 is the human homologue of Saccharomyces cerevisiae gene Set1 and the Drosophila gene Trx. The genes encode an enzyme to catalyze the methylation of H3K4 [Nat. Rev. Cancer., 2007, 7(11):823-833]. Trimethylation of histone H3K4 is a hallmark of active gene transcription, and alteration of this process often causes changes in gene expression pattern. MLL1 translocation is also linked to altered transcription of important genes involved in stem cell maintenance and development and, thus, leads to leukemogenesis. The MLL1 gene was first discovered in leukemia patients in 1991 [Nat. Rev. Cancer., 2007, 7(11):823-833]. cDNA of the MLL1 gene contains ˜12 kb nucleotides and encodes a peptide over 4000 amino acids in length. In the cell, the premature MLL1 protein is digested by taspase, which results in two peptides: a 300 kDa N-terminal fragment and a 170 kDa C-terminal fragment. The two cleaved peptides form a heterodimer, which is complexed with other components, including WDR5, RBBP5, ASH2L and DPY30. In some leukemia patients, chromosomal translocation results in fusion of ˜4.2 kb DNA of the MLL1 N-terminal coding region with some other genes [Cancer. Cell., 2003, 4(3):197-207].

The generation of MLL1 fusion protein is sufficient to induce leukemia, which has been demonstrated in animal models [Nat. Rev. Cancer., 2007, 7(11):823-833]. The mechanisms of MLL1 fusion-mediated leukemia has been studied extensively in the past twenty years. The MLL/SET1 family members are most enzymatically active when part of the “core complex” (WRAD2), comprising the catalytic SET-domain-containing subunits bound to a sub-complex made up of the proteins WDR5, RbBP5, Ash2L and a homodimer of DPY-30. The necessity of MLL/SET1 members to bind WRAD2 for full activity is the basis of a particular drug development strategy, which seeks to disrupt the interaction between the MLL/SET subunits and WDR5. Recent efforts to pharmacologically target the MLL1 catalytic activity has centered on attempts to disrupt the MLL1-WDR5 interaction by means of Win-motif mimicking peptides and small-molecule peptidomimetics [J. Med. Chem., 2010, 53: 5179-5185; J. Am. Chem. Soc., 2013, 135: 669-682; Mol Cell., 2014; 53:247-261]. However, as with most peptide based inhibitors, MLL1-WDR5 peptidic inhibitors exhibit poor cell-based activity and lack oral bioavailability due to poor cell-permeability and their susceptibility to peptidases.

### Role of WDR5 in Other Cancers

### Bladder Cancer

WDR5 also plays a critical role in embryonic stem cell self-renewal [Cell. 2011; 145 (2):183-97] and Epithelial-Mesenchymal Transition [Mol. Cell., 2011; 43(5):811-22]. A recent study finds that H2A.Z is overexpressed in bladder cancer and activates oncogenic transcription by recruiting WDR5 and Bromodomain PHD Finger Transcription Factor (BPTF) to its target genes [Epigenetics. Chromatin., 2013; 6 (1):34.], suggesting that WDR5 may play a role in bladder cancer, but its expression pattern, role and mechanism in bladder cancer remain unclear. WDR5 is upregulated in bladder cancer tissues compared with normal tissues as determined by immunohistochemistry (IHC), and is correlated with advanced tumor stage and overall survival of bladder cancer patients. A recent study found that WDR5 is overexpressed in prostate cancer tissue compared with normal tissues [Mol. Cell., 2014 May 22; 54 (4):613-25]. Taken together, high expression levels of WDR5 may serve as a novel molecular marker for bladder cancer.

WDR5 silencing reduces cell growth in breast cancer and prostate cancer [Mol. Cell., 2014, 54 (4):613-25; Cell Rep., 2013 5 (2):302-13], but the detailed mechanism and role in vivo is still unknown. Through gain or loss of function, WDR5 was found to promote bladder cancer cell proliferation in vitro and tumor growth in vivo, and that silencing WDR5 mainly induces the G0/Ga phase cell cycle arrest. The cell cycle is regulated by cyclins and cyclin-dependent kinases. Cyclin E1 and Cyclin E2 regulate the G1 to S-phase transition, while Cyclin B1 regulates the G2 to M-phase transition. Moreover, Cyclin E is associated with high-grade, high-stage and invasive bladder cancer [Cell. Cycle., 2012; 11(7):1468-76; Am. J Pathol., 2000; 157(3):787-94]. UHMK1 (also named KIS) is overexpressed in leukemia and promotes the G1 to S-phase transition [Leuk. Res., 2008; 32 (9):1358-65]. Mechanistically, WDR5 knockdown inhibited cyclin E1, cyclin E2 and UHMK1 leading to G0/G1 phase cell cycle arrest, which might disturb the effect of cyclin B1 downregulation on G2 to M-phase transition. Additional studies showed that knockdown of MLL1, another core component of the MLL/SET1 complexes, suppressed HeLa cell proliferation by reducing the expression of cyclin B and inducing the G2/M phase cell cycle arrest [Oncogene. 2013; 32(28):3359-70]. Thus, the data reported suggests that WDR5 promotes bladder cancer cell proliferation in vitro and in vivo by regulating the cell cycle, but the role and mechanism are not the same as MLL1.

WDR5 is believed to play an essential role in cancer stem cells (CSCs). CSCs are a small subpopulation of cells in a tumor that can self-renew and differentiate into multiple lineages, and possess strong tumor-initiating capacity. CSCs have been widely identified in a number of malignancies, and the existence of CSCs in bladder cancer was found by Chan et al [Proc. Natl. Acad. Sci. USA., 2009; 106 (33):14016-21]. Several studies have found that sphere culture is an effective way to enrich cancer stem cells [Cell. 2007; 131(6):1109-23; Urol Oncol. 2012; 30(3):314-8]. It was observed that WDR5 and pluripotency transcription factors were upregulated in UM-UC-3 and T24 spheres. Through gain or loss of function, it was demonstrated that WDR5 promoted UM-UC-3 and T24 cells self-renewal in vitro and upregulated Nanog. Emerging evidence shows that Nanog is overexpressed in poorly differentiated tumors and correlated with poor survival outcome of patients with various types of cancer, including bladder cancer [Nat. Genet., 2008; 40(5):499-507; Onco. Targets. Ther., 2013; 6:1207-20]. Moreover, Nanog plays a key role in CSCs self-renewal and targeting. Nanog has shown promising therapeutic potential in several types of cancer [Cell Stem Cell. 2011; 9 (1):50-63; Oncogene. 2013; 32(37):4397-405]. WDR5 directly activates Nanog by mediating its promoter H3K4me3 level. Taken together, recent findings suggest that WDR5 plays a vital role in self-renewal of bladder cancer cells by regulating Nanog.

Further studies have demonstrated that WDR5 silencing increased cell apoptosis and decreases bladder cancer cells resistance to cisplatin. Conversely, overexpression of WDR5 enhanced chemoresistance to cisplatin. Moreover, WDR5 directly regulates important inhibitors of apoptotic proteins, MCL1 [FEBS Lett. 2010; 584(14):2981-9; Sci Rep. 2014; 4:6098] and BIRC3 [Expert Opin Ther Targets.2009; 13(11):1333-45], by H3K4me3.

In summary, WDR5 is upregulated in bladder cancer, and promotes bladder cancer cell proliferation, self-renewal and chemoresistance via activating a series of oncogenes by H3K4me3. Therefore, WDR5 is a potential biomarker for bladder cancer and a promising target for drug development [Sci Rep. 2015; 5: 8293, Genom Data. 2015; 5:27-9.].

### Acute Myeloid Leukemia (AML)

The CEBPA gene is mutated in 9% of patients with acute myeloid leukemia (AML). Selective expression of a short (30-kDa) CCAAT-enhancer binding protein-α (C/EBPα) translational isoform, termed p30, represents the most common type of CEBPA mutation in AML. The molecular mechanisms underlying p30-mediated transformation remain incompletely understood. Recent studies have shown that C/EBPα p30, but not the normal p42 isoform, preferentially interacts with WDR5, a key component of SET/MLL (SET-domain/mixed-lineage leukemia) histone-methyltransferase complexes. Accordingly, p30-bound genomic regions were enriched for MLL-dependent H3K4me3 marks. The p30-dependent increase in self-renewal and inhibition of myeloid differentiation required WDR5, as downregulation of the latter inhibited proliferation and restored differentiation in p30-dependent AML models. Small-molecule inhibitors of the WDR5-MLL interaction selectively inhibited proliferation and induced differentiation in p30-expressing human AML cells revealing the mechanism of p30-dependent transformation and establish the essential p30 cofactor WDR5 as a therapeutic target in CEBPA-mutant AML [Nat Chem Biol. 2015; 11(8):571-8].

### (c) MYCN-Amplified Neuroblastoma

MYCN gene amplification in neuroblastoma drives a gene expression program that correlates strongly with aggressive disease. Mechanistically, trimethylation of histone H3 lysine 4 (H3K4) at target gene promoters is a strict prerequisite for this transcriptional program to be enacted. WDR5 is a histone H3K4 presenter that has been found to have an essential role in H3K4 trimethylation [Cancer Res 2015; 75(23); 5143-54]. For this reason, in this study, the relationship between WDR5-mediated H3K4 trimethylation and N-Myc transcriptional programs in neuroblastoma cells were investigated. N-Myc upregulated WDR5 expression in neuroblastoma cells. Gene expression analysis revealed that WDR5 target genes included those with MYC-binding elements at promoters such as MDM2. WDR5 was demonstrated to form a protein complex at the MDM2 promoter with N-Myc, but not p53, leading to histone H3K4 trimethylation and activation of MDM2 transcription. RNAi-mediated attenuation of WDR5 upregulated expression of wild-type but not mutant p53, an effect associated with growth inhibition and apoptosis. Similarly, a small-molecule antagonist of WDR5 reduced N-Myc/WDR5 complex formation, N-Myc target gene expression, and cell growth in neuroblastoma cells. In MYCN-transgenic mice, WDR5 was overexpressed in precancerous ganglion and neuroblastoma cells compared with normal ganglion cells. Clinically, elevated levels of WDR5 in neuroblastoma specimens were an independent predictor of poor overall survival. Overall, these results identify WDR5 as a key cofactor for N-Myc-regulated transcriptional activation and tumorogenesis and as a novel therapeutic target for MYCN-amplified neuroblastomas [Cancer Res 2015; 75(23); 5143-54, Mol Cell. 2015; 58(3):440-52.].

### SUMMARY

The structural features as described above suggest that the WDR5-MLL binding is a desirable drug target. Hence, agents that bind to the WDR5 protein and compete for binding with WDR5-interacting partners can reverse the transcriptional activities of WDR5 containing complexes. Considering the challenges generally associated with inhibiting protein-protein interactions, along with the current need to treat WDR5-driven tumor types such as leukemias, bladder cancers and neuroblastomas, complementary screening approaches namely virtual screening, focused library screening and traditional structure activity relationship (SAR) studies were conducted. These studies led to the identification of compounds which inhibit the WDR5 protein-protein binding.

A novel class of compounds of Formula (I) have been prepared that show potent disruption of WDR5-MLL1 protein-protein binding and therefore have utility in the treatment of cancers and other WDR5-mediated diseases, disorders and conditions.

Therefore, in one aspect, the present application includes a compound of Formula (I) or a pharmaceutically acceptable salt and/or solvate thereof:

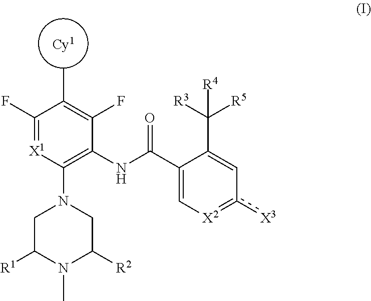

wherein:
R¹ and R² are independently selected from H and CH₃;
R³, R⁴ and R⁵ are independently selected from H and F, provided that at least two of R³, R⁴ and R⁵ are F; [symbol]
is a single or double bond, provided that one [symbol] is a single bond and the other [symbol] is a double bond;
X¹ is selected from CH and N;
X² is NH or NCH₃ when the adjacent [symbol] is a single bond or X² is CH when the adjacent [symbol] is a double bond;
X³ is F when the adjacent [symbol] is a single bond or X³ is O when [symbol] is a double bond;
Cy¹ is a substituted phenyl, substituted 5- or 6-membered heteroaromatic monocyclic ring, substituted 5- or 6-membered heterocycloalkyl monocyclic ring, an optionally substituted 9- or 10-membered aromatic bicyclic ring, optionally substituted 9- or 10-membered heteroaromatic bicyclic ring or optionally substituted 9- or 10-membered heterocycloalkyl bicyclic ring;
when Cy¹ is a monocyclic ring Cy¹ is substituted with at least one Cy² and optionally one or two F, CN or C₁₋₄alkyl; or Cy¹ is substituted with N(C₁₋₁₀alkyl)(C₁₋₁₀alkyl), OCH₂C₃₋₆cycloalkyl, OC₃₋₆cycloalkyl, OC₄₋₆hetereocycloalkyl, OC₅₋₆hetereoaryl, Ophenyl, OCH₂C₄₋₆hetereocycloalkyl, OCH₂C₅₋₆hetereoaryl, C₃₋₆cycloalkyl, phenyl, C₅₋₆hetereoaryl, C₄₋₆heterocycloalkyl, O—CH₂CH₂OC₁₋₄alkyl, OCH₂OC₁₋₄alkyl, C(O)NH₂, C(O)NHC₁₋₁₀alkyl, C(O)N(C₁₋₁₀alkyl)(C₁₋₁₀alkyl), C(O)OH, C(O)OC₁₋₁₀alkyl, C(O)OC₁₋₁₀fluoroalkyl, C(O)C₁₋₁₀alkyl, C(O)C₄₋₆cycloalkyl, C(O)C₄₋₆heterocycloalkyl, C(O)C₅₋₆heteroaryl, C(O)phenyl, C(O)OC₄₋₆cycloalkyl, C(O)OC₅₋₆heteroaryl, C(O)Ophenyl or C(O)OC₄₋₆heterocycloalkyl and optionally one or two F, CN or C₁₋₄alkyl, wherein each cycloalkyl, phenyl, heterocycloalkyl and heteroaryl in the Cy¹ substituents is optionally substituted with one to four substituents independently selected from F, C₁₋₄alkyl, C₀₋₄alkyleneNHC₁₋₄alkyl and C₀₋₄alkyleneN(C₁₋₄alkyl)(C₁₋₄alkyl);
when Cy¹ is a bicyclic ring, Cy¹ is optionally substituted with Cy² and/or one or two F, CN or C₁₋₄alkyl;
Cy² is an optionally substituted phenyl, optionally substituted 5- or 6-membered heteroaromatic monocyclic ring, optionally substituted 5- or 6-membered heterocycloalkyl monocyclic ring, an optionally substituted 9- or 10-membered aromatic bicyclic ring, optionally substituted 8-, 9- or 10-membered heteroaromatic bicyclic ring or optionally substituted 8-, 9- or 10-membered heterocycloalkyl bicyclic ring; and
the optional substituents on Cy² are independently selected from one or two of F, C₁₋₄alkyl, C₁₋₄fluoroalkyl, OC₁₋₄alkyl, OC₁₋₄fluoroalkyl and CN.

In another aspect, the present application includes a composition comprising one or more compounds of the application and a carrier.

In another aspect, the present application includes a method for inhibition of binding of WDR5 to its binding partners in a cell, either in a biological sample or in a patient, comprising administering an effective amount of one or more compounds of the application to the cell.

The present application also includes a method of treating a disease, disorder or condition that is mediated or treatable by inhibition of binding between WDR5 protein and its binding partners comprising administering a therapeutically effective amount of one or more compounds of the application to a subject in need thereof. In an embodiment of the present application, the disease, disorder or condition mediated or treatable by inhibition of binding between WDR5 protein and its binding partners is cancer.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “compound of the application” or “compound of the present application” and the like as used herein refers to a compound of Formula I, and pharmaceutically acceptable salts and/or solvates thereof.

The term “composition of the application” or “composition of the present application” and the like as used herein refers to a composition, such a pharmaceutical composition, comprising one or more compounds of the application.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and solvates, as well as a combination of, for example, a salt of a solvate of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The expression “proceed to a sufficient extent” as used herein with reference to the reactions or process steps disclosed herein means that the reactions or process steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms.

The term “fluoroalkyl” as used herein refers to an alkyl group wherein one or more, including all of the hydrogen atoms are replaced by a halogen atom. In some embodiments, the fluoroalkyl comprises at least one —CHF₂ group. In some embodiments, the fluoroalkyl comprises at least one —CF₃ group.

The term “cycloalkyl,” as used herein, whether it is used alone or as part of another group, means a saturated carbocyclic group. The number of carbon atoms that are possible in the referenced cycloalkyl group are indicated by the numerical prefix “Cₙ₁₋ₙ₂”. For example, the term C₃₋₆cycloalkyl means a cycloalkyl group having 3, 4, 5 or 6 carbon atoms.

The term “aromatic” as used herein refers to cyclic groups containing 6, 9 or 10 carbon atoms and at least one aromatic ring.

The term “heterocycloalkyl” as used herein refers to nonaromatic rings containing 5, 6, 9 or 10 atoms, and at least one non-aromatic, ring in which one or more of the atoms are a heteromoiety selected from O, S, S(O), SO₂, N, NH and NC₁₋₆alkyl. Heterocycloalkyl groups are either saturated or unsaturated (i.e. contain one or more double bonds). Heterocycloalkyl groups containing 5 or 6 atoms are monocyclic and heterocycloalkyl groups containing 9 or 10 atoms are bicyclic.

The term “heteroaryl” as used herein refers to cyclic groups containing from 5, 6, 9 or 10 atoms, at least one aromatic ring and at least one a heteromoiety selected from O, S, S(O), SO₂, N, NH and NC₁₋₆alkyl. Heteroaryl groups containing 5 or 6 atoms are monocyclic and heteroaryl groups containing 9 or 10 atoms are bicyclic.

When the prefix “Cₙ₁₋ₙ₂” appears before the terms “heterocycloalkyl” and “heteroaryl”, this indicates the number of possible carbon atoms in the ring with the remaining atoms in the ring being made up by the heteromoieties to a total of 5, 6, 9 or 10 ring atoms.

The term “bicyclic” refers to ring structures containing two rings that may be fused, bridged or spirofused.

A first ring being “fused” with a second ring means the first ring and the second ring share at least two adjacent atoms there between.

A first ring being “bridged” with a second ring means the first ring and the second ring share at least two non-adjacent atoms there between.

A first ring being “spirofused” with a second ring means the first ring and the second ring share one atom there between.

The term “adjacent” as used herein means “next to” or, with respect to an adjacent bond, it means a bond to which a referenced atom or group is attached.

The term “optionally substituted” as used herein means that a referenced group is either substituted or unsubstituted with a substituent.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “subject” as used herein includes all members of the animal kingdom including mammals, and suitably refers to humans. Thus the methods of the present application are applicable to both human therapy and veterinary applications. In an embodiment, the subject is a mammal. In another embodiment, the subject is human.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. [See, for example, S. M. Berge, et al., “Pharmaceutical Salts,” J. Pharm. Sci. 1977, 66, 1-19]. The selection of the appropriate salt may be useful, for example, so that an ester functionality, if any, elsewhere in a compound is not hydrolyzed. The selection criteria for the appropriate salt will be known to one skilled in the art.

The term “solvate” as used herein means a compound, or a salt or prodrug of a compound, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations. For example, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The “disease, disorder or condition” as used herein refers to a disease, disorder or condition mediated or treatable by inhibition of binding between WDR5 protein and its binding partners, in particular MLL1, and in particular using a WDR5 protein inhibitor, such as a compound of the application herein described.

The term “mediated or treatable by inhibition of binding between WDR5 protein and its binding partners” as used herein means that the disease, disorder or condition to be treated is affected by, modulated by and/or has some biological basis, either direct or indirect, that includes WDR5 binding, in particular, increased WDR5 binding, to its binding partners, such as MLL1. Such biological basis includes, for example, WDR5 and/or MLL1 gene overexpression or WDR5 and/or MLL1 protein over-accumulation or over-expression of proteins that are products of or precursors to WDR5-mediated and/or MLL1 gene expression. In a refined context, “mediated or treatable by inhibition of binding between WDR5 protein and its binding partners” refers to an effect mediated through inhibition of binding between WDR5 and MLL1. In a broader context, “mediated or treatable by inhibition of binding between WDR5 protein and its binding partners” can include the large number of diseases that are caused by aberrant methylation of histone 3 lysine 4 (H3K4) residues, as results from aberrant WDR5 and/or MLL1 activity. As used herein, WDR5 refers to the protein identified as GenBank Accession number NM_017588 [J. Biol. Chem. 2001, 276 (49), 46515-46522] and isoforms that include this sequence, and shorter versions. Similarly, the other WDR5 proteins are characterized and described in any of the protein databases. As used herein, MLL1 refers to the protein identified as GenBank Accession number NM_005933 [Proc. Natl. Acad. Sci. U.S.A. 1991, 88 (23), 10735-10739; DNA Cell Biol. 1995, 14 (6), 475-483] and isoforms that include this sequence, and shorter versions. Similarly, the other MLL1 proteins are characterized and described in any of the protein databases.

The term “binding” as used herein refers to any interaction between two entities, such as two proteins, that leads to a functional effect.

As used herein, the term “effective amount” or “therapeutically effective amount” means an amount of one or more compounds of the application that is effective, at dosages and for periods of time necessary to achieve the desired result. For example in the context of treating a disease, disorder or condition mediated or treatable by inhibition of binding between WDR5 protein and its binding partners, an effective amount is an amount that, for example, increases said inhibition compared to the inhibition without administration of the one or more compounds. In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

The term “administered” as used herein means administration of a therapeutically effective amount of one or more compounds or compositions of the application to a cell, tissue, organ or subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder. Neoplasms can be benign (such as uterine fibroids and melanocytic nevi), potentially malignant (such as carcinoma in situ) or malignant (i.e. cancer). Exemplary neoplastic disorders include the so-called solid tumours and liquid tumours, including but not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers.

The term “cancer” as used herein refers to cellular-proliferative disease states.

### II. Compounds and Compositions of the Application

The present application includes a compound of Formula (I) or a pharmaceutically acceptable salt and/or solvate thereof:

a compound of Formula (I) or a pharmaceutically acceptable salt and/or solvate thereof:

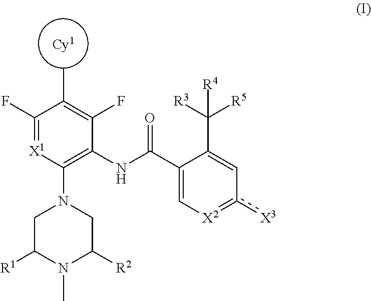

wherein:
R¹ and R² are independently selected from H and CH₃;
R³, R⁴ and R⁵ are independently selected from H and F, provided that at least two of R³, R⁴ and R⁵ are F; [symbol]
is a single or double bond, provided that one [symbol] is a single bond and the other [symbol] is a double bond;
X¹ is selected from CH and N;
X² is NH or NCH₃ when the adjacent [symbol] is a single bond or X² is CH when the adjacent [symbol]
is a double bond;
X³ is F when the adjacent [symbol] is a single bond or X³ is O when [symbol] is a double bond;
Cy¹ is a substituted phenyl, substituted 5- or 6-membered heteroaromatic monocyclic ring, substituted 5- or 6-membered heterocycloalkyl monocyclic ring, an optionally substituted 9- or 10-membered aromatic bicyclic ring, optionally substituted 9- or 10-membered heteroaromatic bicyclic ring or optionally substituted 9- or 10-membered heterocycloalkyl bicyclic ring;
when Cy¹ is a monocyclic ring Cy¹ is substituted with at least one Cy² and optionally one or two F, CN or C₁₋₄alkyl; or Cy¹ is substituted with N(C₁₋₁₀alkyl)(C₁₋₁₀alkyl), OCH₂C₃₋₆cycloalkyl, OC₃₋₆cycloalkyl, OC₄₋₆hetereocycloalkyl, OC₅₋₆hetereoaryl, Ophenyl, OCH₂C₄₋₆hetereocycloalkyl, OCH₂C₅₋₆hetereoaryl, C₃₋₆cycloalkyl, phenyl, C₅₋₆hetereoaryl, C₄₋₆heterocycloalkyl, O—CH₂CH₂OC₁₋₄alkyl, OCH₂OC₁₋₄alkyl, C(O)NH₂, C(O)NHC₁₋₁₀alkyl, C(O)N(C₁₋₁₀alkyl)(C₁₋₁₀alkyl), C(O)OH, C(O)OC₁₋₁₀alkyl, C(O)OC₁₋₁₀fluoroalkyl, C(O)C₁₋₁₀alkyl, C(O)C₄₋₆cycloalkyl, C(O)C₄₋₆heterocycloalkyl, C(O)C₅₋₆heteroaryl, C(O)phenyl, C(O)OC₄₋₆cycloalkyl, C(O)OC₅₋₆heteroaryl, C(O)Ophenyl or C(O)OC₄₋₆heterocycloalkyl and optionally one or two F, CN or C₁₋₄alkyl, wherein each cycloalkyl, phenyl, heterocycloalkyl and heteroaryl in the Cy¹ substituents is optionally substituted with one to four substituents independently selected from F, C₁₋₄alkyl, C₀₋₄alkyleneNHC₁₋₄alkyl and C₀₋₄alkyleneN(C₁₋₄alkyl)(C₁₋₄alkyl);
when Cy¹ is a bicyclic ring, Cy¹ is optionally substituted with Cy² and/or one or two F, CN or C₁₋₄alkyl;
Cy² is an optionally substituted phenyl, optionally substituted 5- or 6-membered heteroaromatic monocyclic ring, optionally substituted 5- or 6-membered heterocycloalkyl monocyclic ring, an optionally substituted 9- or 10-membered aromatic bicyclic ring, optionally substituted 8-, 9- or 10-membered heteroaromatic bicyclic ring or optionally substituted 8-, 9- or 10-membered heterocycloalkyl bicyclic ring; and
the optional substituents on Cy² are independently selected from one or two of F, C₁. 4alkyl, C₁₋₄fluoroalkyl, OC₁₋₄alkyl, OC₁₋₄fluoroalkyl and CN.

In some embodiments, the present application includes a compound of Formula I or a pharmaceutically acceptable salt and/or solvate thereof:

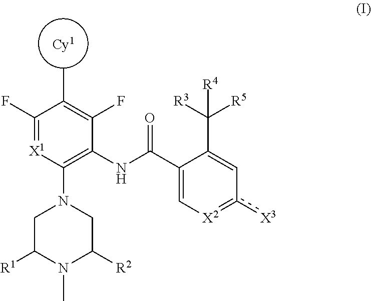

wherein:
R¹ and R² are independently selected from H and CH₃;
R³, R⁴ and R⁵ are independently selected from H and F, provided that at least two of R³, R⁴ and R⁵ are F; [symbol]
is a single or double bond, provided that one [symbol] is a single bond and the other [symbol] is a double bond;
X¹ is selected from CH and N;
X² is NH or NCH₃ when the adjacent [symbol] is a single bond and X² is CH when the adjacent [symbol] is a double bond;
X³ is F when the adjacent [symbol] is a single bond and X³ is O when [symbol] is a double bond;
Cy¹ is a substituted 5 or 6 membered aromatic, heteroaromatic, or heterocycloalkyl monocyclic ring or an optionally substituted 9 or 10 membered aromatic, heteroaromatic or heterocycloalkyl bicyclic ring;
when Cy¹ is a monocyclic ring Cy¹ is substituted with at least one Cy² and optionally one or two F; or Cy¹ is substituted with N(C₁₋₁₀alkyl)₂, OCH₂C₃₋₆cycloalkyl, OC₃₋₆cycloalkyl, OC₄₋₅hetereoccycloalkyl, OCH₂C₄₋₅hetereocycloalkyl, cycloalkyl, O—CH₂CH₂OC₁₋₄alkyl, OCH₂OC₁₋₄alkyl, C(O)NH₂, C(O)NHC₁₋₁₀alkyl, C(O)N(C₁₋₁₀alkyl)₂, C(O)OC₁₋₁₀alkyl, C(O)OC₁₋₁₀fluoroalkyl, C(O)C₄₋₆cycloalkyl, C(O)C₄₋₆heterocycloalkyl, C(O)OC₄₋₆cycloalkyl or C(O)OC₄₋₆heterocycloalkyl, wherein the cycloalkyl is optionally substituted with one to four of F and CH₃;
when Cy¹ is a bicyclic ring, Cy¹ is optionally substituted with Cy² and/or one or two F;
Cy² is an optionally substituted 5 or 6 membered aromatic, heteroaromatic, or heterocycloalkyl monocyclic ring or an optionally substituted 9 or 10 membered aromatic, heteroaromatic or heterocycloalkyl bicyclic ring; and
the optional substituents on Cy² are selected from one or two of F, C₁₋₆alkyl, C₁₋₆fluoroalkyl, OC₁₋₆alkyl, OC₁₋₆fluoroalkyl and CN.

In some embodiments, at least one of R¹ and R² is CH₃. In some embodiments, both R¹ and R² are CH₃.

In some embodiments, R¹ and R² are selected to provide one of the following groups in the compounds of Formula I:

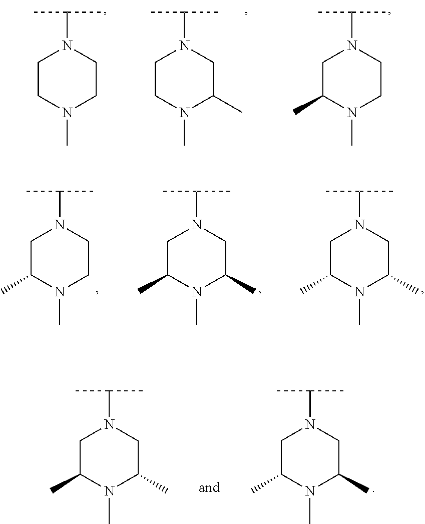

In some embodiments, R¹ and R² are selected to provide one of the following groups in the compounds of Formula I:

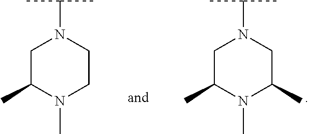

In some embodiments, R³, R⁴, R⁵, X² and X³ are selected to provide one of the following groups in the compounds of Formula I:

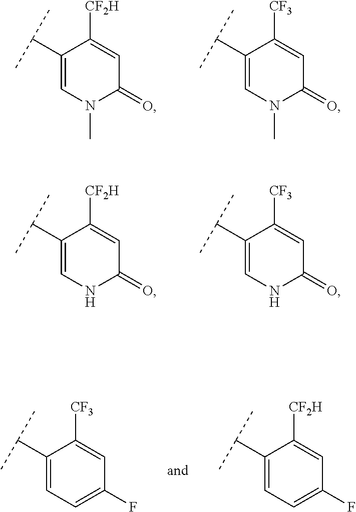

and tautomers thereof.

In some embodiments, R³, R⁴, R⁵, X² and X³ are selected to provide following groups in the compound of Formula I

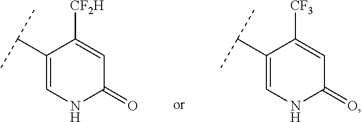

and the corresponding tautomers are

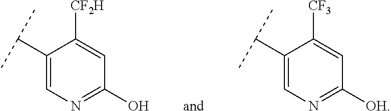

In some embodiments, when Cy¹ is a monocyclic ring Cy¹ is substituted with at least one Cy² and optionally one or two F, CN or C₁₋₄alkyl; or Cy¹ is substituted with N(CH₃)₂

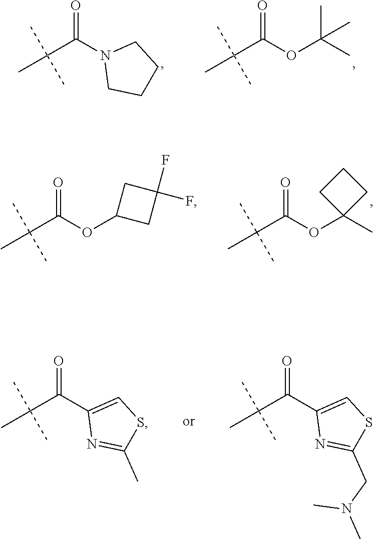

and optionally one or two F, CN or C₁₋₄alkyl.

In some embodiments, when Cy¹ is a monocyclic ring Cy¹ is substituted with at least one Cy² and optionally one or two F or CH₃; or Cy¹ is substituted with N(CH₃)₂

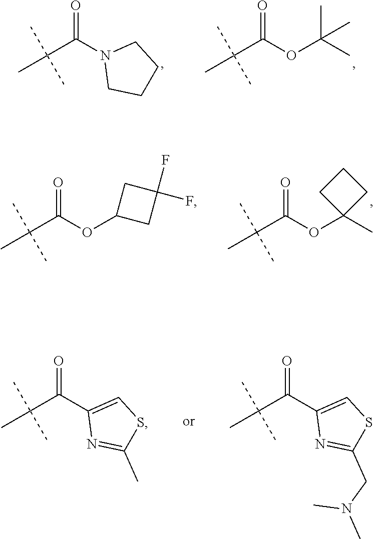

and optionally one or two or F or CH₃.

In some embodiments, when Cy¹ is a monocyclic ring Cy¹ is substituted with at least one Cy² and optionally one or two F; or Cy¹ is substituted with N(CH₃)₂

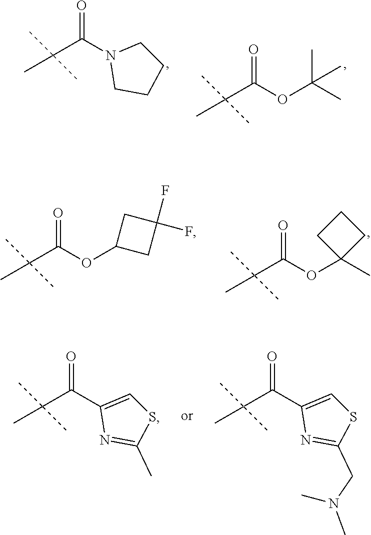

In some embodiments, when Cy¹ is a monocyclic ring Cy¹ is substituted with at least one Cy² and optionally one or two F; or Cy¹ is substituted with N(CH₃)₂,

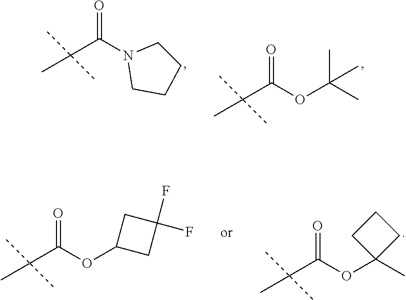

In some embodiments, Cy¹ is substituted with C(O)C₄₋₆cycloalkyl optionally substituted with one C₁₋₄alkyl, suitably methyl. In some embodiments, Cy¹ is substituted with O

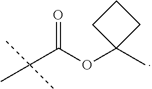

In some embodiments, Cy¹ is a monocyclic 5- or 6-membered heterocyclic ring substituted with Cy² or 5- or 6-membered heteroaromatic ring substituted with Cy². In some embodiments, Cy¹ is a 6membered heterocyclic ring substituted with Cy² at the para or meta position from the point of attachment of Cy¹ to the remainder of the compound of Formula or a 6-membered heteroaromatic ring substituted with Cy² at the para or meta position from the point of attachment of Cy¹ to the remainder of the compound of Formula I. In some embodiments, Cy¹ is a 5-membered heterocyclic ring substituted with Cy² at the beta or gamma position from the point of attachment of Cy¹ to the remainder of the compound of Formula I or 5-membered heteroaromatic ring substituted with Cy² at the beta or gamma position from the point of attachment of Cy¹ to the remainder of the compound of Formula I.

In some embodiments, Cy¹ is selected from substituted phenyl, 2,5-dihydro-1H-pyrrolyl, substituted pyrrolyl, substituted 1,2,3,6-tetrahydropyridinyl, substituted pyridinyl and substituted pyrimidinyl. In some embodiments, Cy¹ is selected from unsubstituted benzo[d][1,3]dioxolyl, unsubstituted 6-2,3-dihydrobenzo[b][1,4]dioxinyl and unsubstituted 2,3-dihydro-[1,4]dioxino[2,3-b]pyridinyl.

In some embodiments, Cy¹ is selected from:

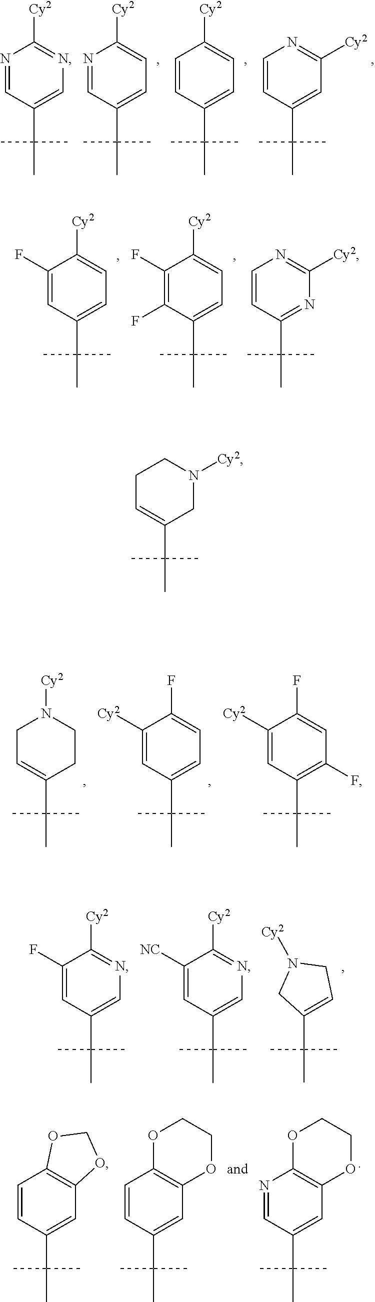

In some embodiments, Cy¹ is selected from:

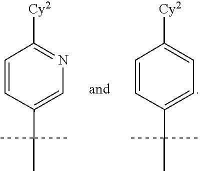

In some embodiments, Cy² is an optionally substituted 5 or 6 membered aromatic, heteroaromatic, or heterocycloalkyl monocyclic ring. In some embodiments, Cy² is an optionally substituted phenyl, optionally substituted 5- or 6-membered heteroaromatic monocyclic ring, or optionally substituted 5- or 6-membered heterocycloalkyl monocyclic ring. In some embodiments, Cy² is an optionally substituted monocyclic heterocycloalkyl ring selected from pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, piperazinyl, morpholinyl, thiomorpholinyl, pyranyl, thiopyranyl, 2,3-dihydropyranyl, tetrahydropyranyl and 1,4-dihydropyridinyl. In some embodiments, Cy² is a optionally substituted heteroaromatic ring selected from thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl, pyridyl, pyrazinyl, pyrimidinyl, triazinyl and pyridazinyl.

In some embodiments, Cy² is selected from optionally substituted morpholinyl, piperidinyl, pyrimidinyl and thiazolyl.

In some embodiments, Cy² is an optionally substituted bridged bicylic ring. In some embodiments, Cy² is an optionally substituted azabicyclo[3.2.1]octanyl.

In some embodiments, the optional substituents on Cy² are selected from one or two of F, CH₃, CF₃, OCH₃, OCF₃ and CN.

In some embodiments, Cy² is selected from:

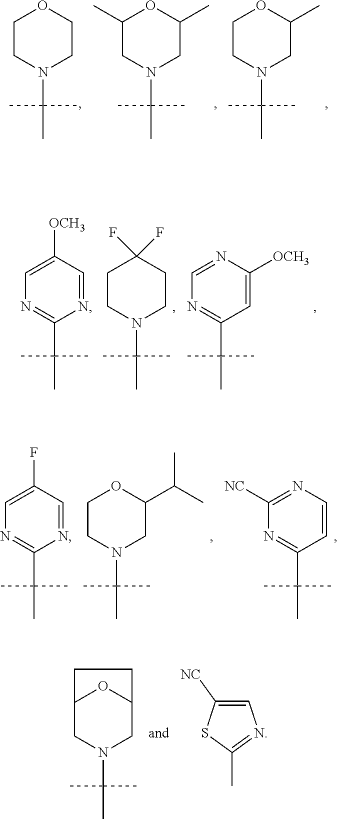

In some embodiments, Cy² is selected from:

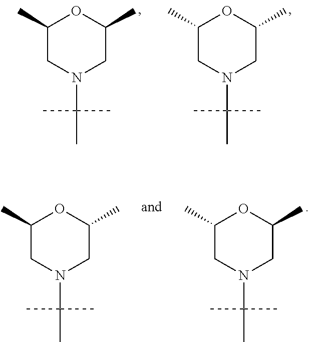

In some embodiments, Cy² is selected from:

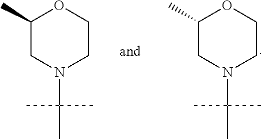

In some embodiments, the compound of Formula I has the following structure:

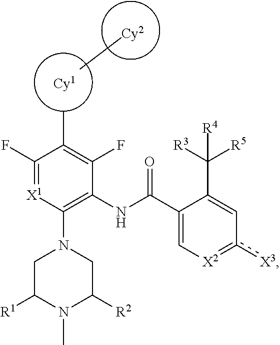

wherein:
R¹ and R² are independently selected from H and CH₃;
R³, R⁴ and R⁵ are independently selected from H and F, provided that at least two of R³, R⁴ and R⁵ are F; [symbol]
is a single or double bond, provided that one [symbol] is a single bond and the other [symbol] is a double bond;
X¹ is selected from CH and N;
X² is NH or NCH₃ when the adjacent [symbol] is a single bond and X² is CH when the adjacent [symbol] is a double bond;
X³ is F when the adjacent [symbol] is a single bond and X³ is O when [symbol] is a double bond;
Cy¹ is phenyl, 5- or 6-membered heteroaromatic monocyclic ring, or 5- or 6-membered heterocycloalkyl monocyclic ring further optionally substituted with one or two F, CN or C₁₋₄alkyl;
Cy² is an optionally substituted phenyl, optionally substituted 5- or 6-membered heteroaromatic monocyclic ring, or optionally substituted 5- or 6-membered heterocycloalkyl monocyclic ring or an optionally substituted 9- or 10-membered aromatic bicyclic ring, optionally substituted 9- or 10-membered heteroaromatic bicyclic ring or optionally substituted 9- or 10-membered heterocycloalkyl bicyclic ring; and
the optional substituents on Cy² are selected from one or two of F, C₁₋₆alkyl, C₁₋₆fluoroalkyl, OC₁₋₆alkyl, OC₁₋₆fluoroalkyl and CN, and pharmaceutically acceptable salts and/or solvates thereof.

In some embodiments, the compound of Formula I have the following structure:

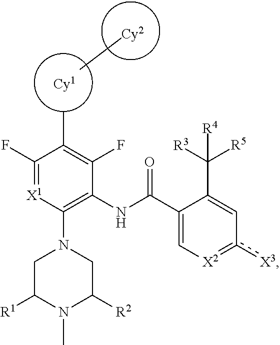

wherein:
R¹ and R² are independently selected from H and CH₃;
R³, R⁴ and R⁵ are independently selected from H and F, provided that at least two of R³, R⁴ and R⁵ are F; [symbol]
a single or double bond, provided that one [symbol] is a single bond and the other [symbol] is a double bond;
X¹ is selected from CH and N;
X² is NH or NCH₃ when the adjacent [symbol] is a single bond and X² is CH when the adjacent [symbol] is a double bond;
X³ is F when the adjacent [symbol] is a single bond and X³ is O when [symbol] is a double bond;
Cy¹ is a 5 or 6 membered aromatic, heteroaromatic, or heterocycloalkyl monocyclic ring further optionally substituted with one or two F;
Cy² is an optionally substituted 5 or 6 membered aromatic, heteroaromatic, or heterocycloalkyl monocyclic ring or an optionally substituted 9 or 10 membered aromatic, heteroaromatic or heterocycloalkyl bicyclic ring; and
the optional substituents on Cy² are selected from one or two of F, C₁₋₆alkyl, C₁₋₆fluoroalkyl, OC₁₋₆alkyl, OC₁₋₆fluoroalkyl and CN, and
pharmaceutically acceptable salts and/or solvates thereof.

In some embodiments the compounds of Formula I have one asymmetric center and the compounds exist as enantiomers. In some embodiments, the compounds of Formula I have more than one asymmetric center and they exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application. Accordingly, in some embodiments, the compounds of the present have at least one asymmetric centre and the compound is a stereoisomer.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

In some embodiments, the compound of Formula (I) is selected from:
N-(3-(6-(cyclopropylmethoxy)pyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(2-morpholinopyrimidin-5-yl)-6-((3S,5R)-3,4,5-trimethylpiperazin-1-yl)phenyl)-6-oxo-4-(trifluoromethyl)-1,6-dihydropyridine-3-carboxamide
N-(3-(2-(cyclopropylmethoxy)pyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(6-morpholinopyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,6-difluoro-4′-morpholino-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
N-(2,4-difluoro-3-(6-(2-methoxyethoxy)pyridin-3-yl)-6-((3S,5R)-3,4,5-trimethylpiperazin-1-yl)phenyl)-6-oxo-4-(trifluoromethyl)-1,6-dihydropyridine-3-carboxamide
N-(4′-(cyclopropylmethoxy)-2,6-difluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
N-(3-(2-((2S,6R)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
6-Oxo-N-(2,3′,6-trifluoro-4′-morpholino-4-((3S,5R)-3,4,5-trimethylpiperazin-1-yl)-[1,1′-biphenyl]-3-yl)-4-(trifluoromethyl)-1,6-dihydropyridine-3-carboxamide
N-[2,4-difluoro-3-(2-morpholin-4-ylpyrimidin-5-yl)-6-[(3R,5S)-3,4,5-trimethylpiperazin-1-yl]phenyl]-4-fluoro-2-(trifluoromethyl)benzamide
N-(2,4-difluoro-3-(2-morpholinopyrimidin-5-yl)-6-((3S,5R)-3,4,5-trimethylpiperazin-1-yl)phenyl)-4-fluoro-2-(trifluoromethyl)benzamide formic acid
N-(2,4-difluoro-3-(2-((S)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide formic acid
Isopropyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate
1-Methylcyclobutyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate
N-(2,4-difluoro-3-(1-(pyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(2-((S)-2-methylmorpholino)pyrimidin-5-yl)-6-((3S,5R)-3,4,5-trimethylpiperazin-1-yl)phenyl)-4-fluoro-2-(trifluoromethyl)benzamide formic acid
N-(2,4-difluoro-3-(2-((R)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide formic acid
N-(2,4-difluoro-3-(2-((R)-2-methylmorpholino)pyrimidin-5-yl)-6-((3S,5R)-3,4,5-trimethylpiperazin-1-yl)phenyl)-4-fluoro-2-(trifluoromethyl)benzamide formic acid
N-(2,4-difluoro-3-(1-pivaloyl-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
3,3-Difluorocyclobutyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate
N-(2,4-difluoro-3-(1-(pyrimidin-2-yl)-1,2,5,6-tetrahydropyridin-3-yl)-6-((3S,5R)-3,4,5-trimethylpiperazin-1-yl)phenyl)-4-(difluoromethyl)-6-oxo-1,6-dihydropyridine-3-carboxamide
1-Methylcyclobutyl 5-(3-(4-(difluoromethyl)-6-oxo-1,6-dihydropyridine-3-carboxamido)-2,6-difluoro-4-((3S,5R)-3,4,5-trimethylpiperazin-1-yl)phenyl)-3,6-dihydropyridine-1(2H)-carboxylate
Isopropyl 5-(3-(4-(difluoromethyl)-6-oxo-1,6-dihydropyridine-3-carboxamido)-2,6-difluoro-4-((3S,5R)-3,4,5-trimethylpiperazin-1-yl)phenyl)-3,6-dihydropyridine-1(2H)-carboxylate
6-Oxo-N-(2,3′,6-trifluoro-4′-(methylcarbamoyl)-4-((3S,5R)-3,4,5-trimethylpiperazin-1-yl)-[1,1′-biphenyl]-3-yl)-4-(trifluoromethyl)-1,6-dihydropyridine-3-carboxamide
N-(4′-carbamoyl-2,3′,6-trifluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
N-(4′-carbamoyl-2,2′,3′,6-tetrafluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide 6-Oxo-N-(2,2′,3′,6-tetrafluoro-4′-((2,4,4-trimethylpentan-2-yl)carbamoyl)-4-((3S,5R)-3,4,5-trimethylpiperazin-1-yl)-[1,1′-biphenyl]-3-yl)-4-(trifluoromethyl)-1,6-dihydropyridine-3-carboxamide
N-(3′-carbamoyl-2,4′,6-trifluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
6-Oxo-N-(2,4′,6-trifluoro-3′-(methylcarbamoyl)-4-((3S,5R)-3,4,5-trimethylpiperazin-1-yl)-[1,1′-biphenyl]-3-yl)-4-(trifluoromethyl)-1,6-dihydropyridine-3-carboxamide
N-(5′-carbamoyl-2,2′,4′,6-tetrafluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
N-(2,4-difluoro-3-(2-morpholinopyrimidin-5-yl)-6-((3S,5R)-3,4,5-trimethylpiperazin-1-yl)phenyl)-2-(difluoromethyl)-4-fluorobenzamide formic acid
2-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-4-fluorobenzamide formic acid
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide
Isopropyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
Isopropyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
Isopropyl (S)-3-(3-(4-(difluoromethyl)-6-oxo-1,6-dihydropyridine-3-carboxamido)-4-(3,4-dimethylpiperazin-1-yl)-2,6-difluorophenyl)-2,5-dihydro-1H-pyrrole-1-carboxylate
Isopropyl (S)-3-(4-(3,4-dimethylpiperazin-1-yl)-2,6-difluoro-3-(6-oxo-4-(trifluoromethyl)-1,6-dihydropyridine-3-carboxamido)phenyl)-2,5-dihydro-1H-pyrrole-1-carboxylate
Isopropyl (S)-5-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
Isopropyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
N-(2,4-difluoro-3-(2-morpholinopyrimidin-5-yl)-6-((3S,5R)-3,4,5-trimethylpiperazin-1-yl)phenyl)-4-(difluoromethyl)-1-methyl-6-oxo-1,6-dihydropyridine-3-carboxamide N-(2,4-difluoro-3-(2-morpholinopyrimidin-5-yl)-6-((3S,5R)-3,4,5-trimethylpiperazin-1-yl)phenyl)-4-(difluoromethyl)-6-oxo-1,6-dihydropyridine-3-carboxamide
(3,3-Difluorocyclobutyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
3,3-Difluorocyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
3,3-Difluorocyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
3,3-Difluorocyclobutyl (S)-3-(4-(3,4-dimethylpiperazin-1-yl)-2,6-difluoro-3-(6-oxo-4-(trifluoromethyl)-1,6-dihydropyridine-3-carboxamido)phenyl)-2,5-dihydro-1H-pyrrole-1-carboxylate
3,3-Difluorocyclobutyl (S)-5-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
3,3-Difluorocyclobutyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
(S)-4-(difluoromethyl)-N-(6-(3,4-dimethylpiperazin-1-yl)-2,4-difluoro-3-(1-(5-methoxypyrimidin-2-yl)-1,2,3,6-tetrahydropyridin-4-yl)phenyl)-6-oxo-1,6-dihydropyridine-3-carboxamide
(S)-4-(difluoromethyl)-N-(6-(3,4-dimethylpiperazin-1-yl)-2,4-difluoro-3-(1-(5-methoxypyrimidin-2-yl)-1,2,5,6-tetrahydropyridin-3-yl)phenyl)-6-oxo-1,6-dihydropyridine-3-carboxamide
(S)-4-(difluoromethyl)-N-(6-(3,4-dimethylpiperazin-1-yl)-2,4-difluoro-3-(1-(5-methoxypyrimidin-2-yl)-2,5-dihydro-1H-pyrrol-3-yl)phenyl)-6-oxo-1,6-dihydropyridine-3-carboxamide
(S)—N-(6-(3,4-dimethylpiperazin-1-yl)-2,4-difluoro-3-(1-(5-methoxypyrimidin-2-yl)-2,5-dihydro-1H-pyrrol-3-yl)phenyl)-6-oxo-4-(trifluoromethyl)-1,6-dihydropyridine-3-carboxamide
(S)—N-(6-(3,4-dimethylpiperazin-1-yl)-2,4-difluoro-3-(1-(5-methoxypyrimidin-2-yl)-1,2,5,6-tetrahydropyridin-3-yl)phenyl)-6-oxo-4-(trifluoromethyl)-1,6-dihydropyridine-3-carboxamide
(S)—N-(6-(3,4-dimethylpiperazin-1-yl)-2,4-difluoro-3-(1-(5-methoxypyrimidin-2-yl)-1,2,3,6-tetrahydropyridin-4-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(pyrimidin-2-yl)-1,2,5,6-tetrahydropyridin-3-yl)phenyl)-6-oxo-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
N-(3-(2-((2S,6R)-2,6-dimethylmorpholino)pyrimidin-4-yl)-6-((S)-3,4-dimethylpiperazin-1-yl)-2,4-difluorophenyl)-1-methyl-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-1-methyl-6-oxo-1,6-dihydropyridine-3-carboxamide
N-(3-(2-((2S,6R)-2,6-dimethylmorpholino)pyrimidin-4-yl)-6-((S)-3,4-dimethylpiperazin-1-yl)-2,4-difluorophenyl)-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-6-oxo-1,6-dihydropyridine-3-carboxamide
N-(6-((S)-3,4-dimethylpiperazin-1-yl)-2,4-difluoro-3-(2-((S)-2-methylmorpholino)pyrimidin-5-yl)phenyl)-6-oxo-4-(trifluoromethyl)-1,6-dihydropyridine-3-carboxamide
N-(3-(2-(4,4-difluoropiperidin-1-yl)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(6-((2S,6R)-2,6-dimethylmorpholino)pyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(6-(dimethylamino)-5-fluoropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(5-cyano-6-morpholinopyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(6-((tetrahydro-2H-pyran-4-yl)oxy)pyridin-3-yl)-6-((3S,5R)-3,4,5-trimethylpiperazin-1-yl)phenyl)-6-oxo-4-(trifluoromethyl)-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide formic acid
(S)-4-(difluoromethyl)-N-(6-(3,4-dimethylpiperazin-1-yl)-2,4-difluoro-3-(1-(6-methoxypyrimidin-4-yl)-1,2,3,6-tetrahydropyridin-4-yl)phenyl)-6-oxo-1,6-dihydropyridine-3-carboxamide
(S)—N-(6-(3,4-dimethylpiperazin-1-yl)-2,4-difluoro-3-(1-(6-methoxypyrimidin-4-yl)-1,2,5,6-tetrahydropyridin-3-yl)phenyl)-6-oxo-4-(trifluoromethyl)-1,6-dihydropyridine-3-carboxamide
N-(2,4-difluoro-3-(2-morpholinopyridin-4-yl)-6-((3S,5R)-3,4,5-trimethylpiperazin-1-yl)phenyl)-6-oxo-4-(trifluoromethyl)-1,6-dihydropyridine-3-carboxamide
N-(3-(2-(dimethylamino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(benzo[d][1,3]dioxol-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(2,3-dihydrobenzo[b][1,4]dioxin-6-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(2,3-dihydro-[1,4]dioxino[2,3-b]pyridin-7-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(6-methoxypyrimidin-4-yl)-2,5-dihydro-1H-pyrrol-3-yl)phenyl)-6-oxo-1,6-dihydropyridine-3-carboxamide
(S)—N-(6-(3,4-dimethylpiperazin-1-yl)-2,4-difluoro-3-(1-(6-methoxypyrimidin-4-yl)-2,5-dihydro-TH-pyrrol-3-yl)phenyl)-6-oxo-4-(trifluoromethyl)-1,6-dihydropyridine-3-carboxamide
(S)—N-(6-(3,4-dimethylpiperazin-1-yl)-2,4-difluoro-3-(1-(6-methoxypyrimidin-4-yl)-1,2,3,6-tetrahydropyridin-4-yl)phenyl)-6-oxo-4-(trifluoromethyl)-1,6-dihydropyridine-3-carboxamide
N-(3-(2-((2S,6R)-2,6-dimethylmorpholino)pyrimidin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-4-yl)-2,4-difluoro-6-((3S,5R)-3,4,5-trimethylpiperazin-1-yl)phenyl)-6-oxo-1,6-dihydropyridine-3-carboxamide
(S)-4-(difluoromethyl)-N-(6-(3,4-dimethylpiperazin-1-yl)-2,4-difluoro-3-(1-(6-methoxypyrimidin-4-yl)-1,2,5,6-tetrahydropyridin-3-yl)phenyl)-6-oxo-1,6-dihydropyridine-3-carboxamide
(S)—N-(6-(3,4-dimethylpiperazin-1-yl)-2,4-difluoro-3-(1-(5-fluoropyrimidin-2-yl)-2,5-dihydro-1H-pyrrol-3-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(5-fluoropyrimidin-2-yl)-1,2,3,6-tetrahydropyridin-4-yl)phenyl)-6-oxo-1,6-dihydropyridine-3-carboxamide
(S)—N-(6-(3,4-dimethylpiperazin-1-yl)-2,4-difluoro-3-(1-(5-fluoropyrimidin-2-yl)-1,2,3,6-tetrahydropyridin-4-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(5-fluoropyrimidin-2-yl)-1,2,5,6-tetrahydropyridin-3-yl)phenyl)-6-oxo-1,6-dihydropyridine-3-carboxamide
(S)—N-(6-(3,4-dimethylpiperazin-1-yl)-2,4-difluoro-3-(1-(5-fluoropyrimidin-2-yl)-1,2,5,6-tetrahydropyridin-3-yl)phenyl)-6-oxo-4-(trifluoromethyl)-1,6-dihydropyridine-3-carboxamide
N-(3-(2-((2R,6R)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
N-(3-(2-((2R,6R)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(2-((2S,6S)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
N-(3-(2-((2S,6S)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(2-((1R,5S)-8-oxa-3-azabicyclo[3.2.1]octan-3-yl)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
N-(3-(2-((1R,5S)-8-oxa-3-azabicyclo[3.2.1]octan-3-yl)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(5-fluoropyrimidin-2-yl)-2,5-dihydro-1H-pyrrol-3-yl)phenyl)-6-oxo-1,6-dihydropyridine-3-carboxamide
1-Methylcyclobutyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
1-Methylcyclobutyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
1-Methylcyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
(S)—N-(3-(1-(5-cyanothiazol-2-yl)-2,5-dihydro-1H-pyrrol-3-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(5-cyanothiazol-2-yl)-2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(1-(5-cyanothiazol-2-yl)-1,2,3,6-tetrahydropyridin-4-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(5-cyanothiazol-2-yl)-1,2,3,6-tetrahydropyridin-4-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(1-(5-cyanothiazol-2-yl)-1,2,5,6-tetrahydropyridin-3-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(5-cyanothiazol-2-yl)-1,2,5,6-tetrahydropyridin-3-yl)-6-(3,4-dimethylpiperazin-1-yl)-2,4-difluorophenyl)-6-oxo-4-(trifluoromethyl)-1,6-dihydropyridine-3-carboxamide
N-(2,4-difluoro-3-(2-((S)-2-methylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide
N-(6-((S)-3,4-dimethylpiperazin-1-yl)-2,4-difluoro-3-(2-((S)-2-isopropylmorpholino)pyrimidin-5-yl)phenyl)-6-oxo-4-(trifluoromethyl)-1,6-dihydropyridine-3-carboxamide
N-(2,4-difluoro-3-(2-((S)-2-isopropylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide
N-(6-((S)-3,4-dimethylpiperazin-1-yl)-2,4-difluoro-3-(2-((R)-2-isopropylmorpholino)pyrimidin-5-yl)phenyl)-6-oxo-4-(trifluoromethyl)-1,6-dihydropyridine-3-carboxamide
N-(2,4-difluoro-3-(2-((R)-2-isopropylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide
(S)—N-(3-(1-(2-cyanopyrimidin-4-yl)-2,5-dihydro-1H-pyrrol-3-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(2-cyanopyrimidin-4-yl)-2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(1-(2-cyanopyrimidin-4-yl)-1,2,3,6-tetrahydropyridin-4-yl)-6-(3,4-dimethylpiperazin-1-yl)-2,4-difluorophenyl)-4-(difluoromethyl)-6-oxo-1,6-dihydropyridine-3-carboxamide
2-(Difluoromethyl)-N-(3-(2-((2S,6R)-2,6-dimethylmorpholino)pyrimidin-5-yl)-2,4-difluoro-6-((3S,5R)-3,4,5-trimethylpiperazin-1-yl)phenyl)-4-fluorobenzamide formic acid
2-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-4-fluorobenzamide formic acid
4-(Difluoromethyl)-N-(3-(2-((2S,6R)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide
(S)—N-(3-(1-(2-cyanopyrimidin-4-yl)-1,2,3,6-tetrahydropyridin-4-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(1-(2-cyanopyrimidin-4-yl)-1,2,5,6-tetrahydropyridin-3-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(2-cyanopyrimidin-4-yl)-1,2,5,6-tetrahydropyridin-3-yl)-6-(3,4-dimethylpiperazin-1-yl)-2,4-difluorophenyl)-6-oxo-4-(trifluoromethyl)-1,6-dihydropyridine-3-carboxamide
4-(Difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide
4-(Difluoromethyl)-N-(3-(2-((2R,6R)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide
4-(Difluoromethyl)-N-(6-((S)-3,4-dimethylpiperazin-1-yl)-2,4-difluoro-3-(2-((R)-2-isopropylmorpholino)pyrimidin-5-yl)phenyl)-6-oxo-1,6-dihydropyridine-3-carboxamide
N-(2,4-difluoro-3-(2-((R)-2-isopropylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide
4-(Difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide
4-(Difluoromethyl)-N-(3-(2-((2S,6S)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide
4-(Difluoromethyl)-N-(6-((S)-3,4-dimethylpiperazin-1-yl)-2,4-difluoro-3-(2-((S)-2-isopropylmorpholino)pyrimidin-5-yl)phenyl)-6-oxo-1,6-dihydropyridine-3-carboxamide
N-(2,4-difluoro-3-(2-((S)-2-isopropylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide
N-(6-((S)-3,4-dimethylpiperazin-1-yl)-2,4-difluoro-3-(2-((R)-2-isopropylmorpholino)pyrimidin-4-yl)phenyl)-6-oxo-4-(trifluoromethyl)-1,6-dihydropyridine-3-carboxamide
4-(Difluoromethyl)-N-(6-((S)-3,4-dimethylpiperazin-1-yl)-2,4-difluoro-3-(2-((R)-2-isopropylmorpholino)pyrimidin-4-yl)phenyl)-6-oxo-1,6-dihydropyridine-3-carboxamide
N-(2,4-difluoro-3-(2-((S)-2-methylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide
4-(Difluoromethyl)-N-(6-((S)-3,4-dimethylpiperazin-1-yl)-2,4-difluoro-3-(2-((S)-2-methylmorpholino)pyrimidin-5-yl)phenyl)-6-oxo-1,6-dihydropyridine-3-carboxamide
N-(6-((S)-3,4-dimethylpiperazin-1-yl)-2,4-difluoro-3-(2-((R)-2-methylmorpholino)pyrimidin-5-yl)phenyl)-6-oxo-4-(trifluoromethyl)-1,6-dihydropyridine-3-carboxamide
N-(2,4-Difluoro-3-(2-((R)-2-methylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide
4-(Difluoromethyl)-N-(6-((S)-3,4-dimethylpiperazin-1-yl)-2,4-difluoro-3-(2-((R)-2-methylmorpholino)pyrimidin-5-yl)phenyl)-6-oxo-1,6-dihydropyridine-3-carboxamide
N-(2,4-difluoro-3-(2-((R)-2-methylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide
N-(3-(2-((2S,6R)-2,6-dimethylmorpholino)thiazol-4-yl)-6-((S)-3,4-dimethylpiperazin-1-yl)-2,4-difluorophenyl)-6-oxo-4-(trifluoromethyl)-1,6-dihydropyridine-3-carboxamide
4-(Difluoromethyl)-N-(3-(2-((2S,6R)-2,6-dimethylmorpholino)thiazol-4-yl)-6-((S)-3,4-dimethylpiperazin-1-yl)-2,4-difluorophenyl)-6-oxo-1,6-dihydropyridine-3-carboxamide
4-(2,6-Difluoro-3-(6-oxo-4-(trifluoromethyl)-1,6-dihydropyridine-3-carboxamido)-4-((3S,5R)-3,4,5-trimethylpiperazin-1-yl)phenyl)-5,6-dihydropyridine-1(2H)-carboxylate
3-(2,6-Difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate
3,3-Difluorocyclobutyl 4-(2,6-difluoro-3-(6-oxo-4-(trinfluoromethyl)-1,6-dihydropyridine-3-carboxamido)-4-((3S,5R)-3,4,5-trimethylpiperazin-1-yl)phenyl)-5,6-dihydropyridine-1(2H)-carboxylate
3,3-Difluorocyclobutyl 3-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate
N-(2,4-difluoro-3-(1-(5-methoxypyrimidin-2-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(5-methoxypyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(5-methoxypyrimidin-2-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(6-methoxypyrimidin-4-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide
1-Methylcyclobutyl 3-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate
N-(2,4-difluoro-3-(1-(6-methoxypyrimidin-4-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(6-methoxypyrimidin-4-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide
1-Methylcyclobutyl 4-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-5,6-dihydropyridine-1(2H)-carboxylate
N-(2,4-difluoro-3-(1-(5-fluoropyrimidin-2-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(5-fluoropyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(5-fluoropyrimidin-2-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide
N-(3-(1-(5-cyanothiazol-2-yl)-1,2,3,6-tetrahydropyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(1-(5-cyanothiazol-2-yl)-1,2,5,6-tetrahydropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(1-(5-cyanothiazol-2-yl)-2,5-dihydro-TH-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide
N-(3-(1-(2-cyanopyrimidin-4-yl)-1,2,3,6-tetrahydropyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
(S)-2-(difluoromethyl)-N-(6-(3,4-dimethylpiperazin-1-yl)-2,4-difluoro-3-(5-methyl-4-(pyrrolidine-1-carbonyl)thiazol-2-yl)phenyl)-4-fluorobenzamide
(S)—N-(6-(3,4-dimethylpiperazin-1-yl)-2,4-difluoro-3-(5-methyl-4-(pyrrolidine-1-carbonyl)thiazol-2-yl)phenyl)-4-fluoro-2-(trifluoromethyl)benzamide
(S)—N-(3-(2-(8-oxa-3-azabicyclo[3.2.1]octan-3-yl)pyrimidin-5-yl)-6-(3,4-dimethylpiperazin-1-yl)-2,4-difluorophenyl)-4-(difluoromethyl)-6-oxo-1,6-dihydropyridine-3-carboxamide
N-(3-(2-(8-oxa-3-azabicyclo[3.2.1]octan-3-yl)pyrimidin-5-yl)-2,4-difluoro-6-((3S,5R)-3,4,5-trimethylpiperazin-1-yl)phenyl)-4-(difluoromethyl)-6-oxo-1,6-dihydropyridine-3-carboxamide
2-(difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-4-fluorobenzamide
2-(difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-4-fluorobenzamide
(S)-4-(difluoromethyl)-N-(6-(3,4-dimethylpiperazin-1-yl)-2,4-difluoro-3-(1-(2-methylthiazole-4-carbonyl)-1,2,3,6-tetrahydropyridin-4-yl)phenyl)-6-oxo-1,6-dihydropyridine-3-carboxamide
4-(Difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide
4-(Difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide
N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2R,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2S,6S)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide formic acid salt
N-(3-(2-((2R,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide formic acid salt
N-(2,4-difluoro-3-(6-((R)-2-methylmorpholino)pyridin-3-yl)-6-((3S,5R)-3,4,5-trimethylpiperazin-1-yl)phenyl)-6-oxo-4-(trifluoromethyl)-1,6-dihydropyridine-3-carboxamide
N-(6-((S)-3,4-dimethylpiperazin-1-yl)-2,4-difluoro-3-(6-((R)-2-methylmorpholino)pyridin-3-yl)phenyl)-6-oxo-4-(trifluoromethyl)-1,6-dihydropyridine-3-carboxamide
N-(3-(6-((2S,6R)-2,6-dimethylmorpholino)pyridin-3-yl)-6-((S)-3,4-dimethylpiperazin-1-yl)-2,4-difluorophenyl)-6-oxo-4-(trifluoromethyl)-1,6-dihydropyridine-3-carboxamide
N-(6-((S)-3,4-dimethylpiperazin-1-yl)-2,4-difluoro-3-(6-((S)-2-methylmorpholino)pyridin-3-yl)phenyl)-6-oxo-4-(trifluoromethyl)-1,6-dihydropyridine-3-carboxamide
N-(6-((S)-3,4-dimethylpiperazin-1-yl)-2,4-difluoro-3-(6-((R)-2-methylmorpholino)pyridin-3-yl)phenyl)-4-fluoro-2-(trifluoromethyl)benzamide
(S)—N-(6-(3,4-dimethylpiperazin-1-yl)-2,4-difluoro-3-(2-(piperazin-1-yl)pyrimidin-5-yl)phenyl)-4-fluoro-2-(trifluoromethyl)benzamide
(S)—N-(6-(3,4-dimethylpiperazin-1-yl)-2,4-difluoro-3-(6-morpholinopyridin-3-yl)phenyl)-4-fluoro-2-(trifluoromethyl)benzamide
4-(Difluoromethyl)-N-(3-(6-((2S,6R)-2,6-dimethylmorpholino)pyridin-3-yl)-6-((S)-3,4-dimethylpiperazin-1-yl)-2,4-difluorophenyl)-1-methyl-6-oxo-1,6-dihydropyridine-3-carboxamide
N-(3-(6-((2S,6R)-2,6-dimethylmorpholino)pyridin-3-yl)-6-((S)-3,4-dimethylpiperazin-1-yl)-2,4-difluorophenyl)-4-fluoro-2-(trifluoromethyl)benzamide
2-(Difluoromethyl)-N-(6-((S)-3,4-dimethylpiperazin-1-yl)-2,4-difluoro-3-(6-((R)-2-methylmorpholino)pyridin-3-yl)phenyl)-4-fluorobenzamide
2-(Difluoromethyl)-N-(3-(6-((2S,6R)-2,6-dimethylmorpholino)pyridin-3-yl)-6-((S)-3,4-dimethylpiperazin-1-yl)-2,4-difluorophenyl)-4-fluorobenzamide
2-(Difluoromethyl)-N-(6-((S)-3,4-dimethylpiperazin-1-yl)-2,4-difluoro-3-(6-((S)-2-methylmorpholino)pyridin-3-yl)phenyl)-4-fluorobenzamide
(S)-2-(difluoromethyl)-N-(6-(3,4-dimethylpiperazin-1-yl)-2,4-difluoro-3-(6-morpholinopyridin-3-yl)phenyl)-4-fluorobenzamide
4-(Difluoromethyl)-N-(6-((S)-3,4-dimethylpiperazin-1-yl)-2,4-difluoro-3-(6-((R)-2-methylmorpholino)pyridin-3-yl)phenyl)-6-oxo-1,6-dihydropyridine-3-carboxamide
4-(Difluoromethyl)-N-(3-(6-((2S,6R)-2,6-dimethylmorpholino)pyridin-3-yl)-6-((S)-3,4-dimethylpiperazin-1-yl)-2,4-difluorophenyl)-6-oxo-1,6-dihydropyridine-3-carboxamide
4-(Difluoromethyl)-N-(6-((S)-3,4-dimethylpiperazin-1-yl)-2,4-difluoro-3-(6-((S)-2-methylmorpholino)pyridin-3-yl)phenyl)-6-oxo-1,6-dihydropyridine-3-carboxamide
(S)—N-(3-(benzo[d][1,3]dioxol-5-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(2,3-dihydrobenzo[b][1,4]dioxin-6-yl)-6-(3,4-dimethylpiperazin-1-yl)-2,4-difluorophenyl)-6-oxo-4-(trifluoromethyl)-1,6-dihydropyridine-3-carboxamide
(S)—N-(6-(3,4-dimethylpiperazin-1-yl)-2,4-difluoro-3-(6-((tetrahydro-2H-pyran-4-yl)oxy)pyridin-3-yl)phenyl)-6-oxo-4-(trifluoromethyl)-1,6-dihydropyridine-3-carboxamide
(S)-4-(difluoromethyl)-N-(3-(1-(2-((dimethylamino)methyl)thiazole-4-carbonyl)-1,2,3,6-tetrahydropyridin-4-yl)-6-(3,4-dimethylpiperazin-1-yl)-2,4-difluorophenyl)-6-oxo-1,6-dihydropyridine-3-carboxamide
N-(2,4-difluoro-3-(1-(2-methylthiazole-4-carbonyl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(3-(1-(2-((dimethylamino)methyl)thiazole-4-carbonyl)-1,2,5,6-tetrahydropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(6-((R)-2-methylmorpholino)pyridin-3-yl)-6-((3S,5R)-3,4,5-trimethylpiperazin-1-yl)phenyl)-4-(difluoromethyl)-6-oxo-1,6-dihydropyridine-3-carboxamide and
4-(Difluoromethyl)-N-(3-(6-((2S,6R)-2,6-dimethylmorpholino)pyridin-3-yl)-2,4-difluoro-6-((3S,5R)-3,4,5-trimethylpiperazin-1-yl)phenyl)-6-oxo-1,6-dihydropyridine-3-carboxamide
   and pharmaceutically acceptable salts and/or solvates thereof.

In some embodiments, the compound of Formula I is selected from:
N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide
N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide
N-(2,4-difluoro-3-(6-((R)-2-methylmorpholino)pyridin-3-yl)-6-((3S,5R)-3,4,5-trimethylpiperazin-1-yl)phenyl)-4-(difluoromethyl)-6-oxo-1,6-dihydropyridine-3-carboxamide
N-(3-(2-((2S,6R)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
1-Methylcyclobutyl 4-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate
N-(2,4-difluoro-3-(6-((R)-2-methylmorpholino)pyridin-3-yl)-6-((3S,5R)-3,4,5-trimethylpiperazin-1-yl)phenyl)-6-oxo-4-(trifluoromethyl)-1,6-dihydropyridine-3-carboxamide and
N-(2,4-difluoro-3-(6-((S)-2-methylmorpholino)pyridin-3-yl)-6-((3S,5R)-3,4,5-trimethylpiperazin-1-yl)phenyl)-6-oxo-4-(trifluoromethyl)-1,6-dihydropyridine-3-carboxamide
   and pharmaceutically acceptable salts and/or solvates thereof.

In some embodiments, the compound of Formula I is selected from:
N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide
N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide and
N-(2,4-difluoro-3-(6-((R)-2-methylmorpholino)pyridin-3-yl)-6-((3S,5R)-3,4,5-trimethylpiperazin-1-yl)phenyl)-4-(difluoromethyl)-6-oxo-1,6-dihydropyridine-3-carboxamide
   and pharmaceutically acceptable salts and/or solvates thereof.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, Pa., 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

A compound of the application is either used alone or in combination with other known agents useful for treating diseases, disorders or conditions that are mediated or treatable by inhibition of binding between WDR5 protein and its binding partners, and those that are treatable with a WDR5 inhibitor, such as the compounds disclosed herein. When used in combination with other agents useful in treating diseases, disorders or conditions mediated or treatable by inhibition of binding between WDR5 protein and its binding partners, it is an embodiment that a compound of the application is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, a compound of the present application is administered with another therapeutic agent simultaneously or sequentially in separate unit dosage forms or together in a single unit dosage form. Accordingly, the present application provides a single unit dosage form comprising one or more compounds of the application, an additional therapeutic agent, and a pharmaceutically acceptable carrier.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound of the application is administered initially in a suitable dosage that is adjusted as required, depending on the clinical response. Dosages will generally be selected to maintain a serum level of the compound of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the one or more compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. In embodiments of the application the one or more compounds of the application are administered in a single daily, weekly or monthly dose or the total daily dose is divided into two, three or four daily doses.

In the above, the term “a compound” also includes embodiments wherein one or more compounds are referenced.

### III. Methods and Uses of the Application Therapeutic Methods and Uses

The compounds of the application have been shown to be inhibitors of the binding of WDR5 to MLL1.

Accordingly, the present application includes a method for inhibition of binding of WDR5 to its binding partners in a cell, either in a biological sample or in a patient, comprising administering an effective amount of one or more compounds of the application to the cell. The application also includes a use of one or more compounds of the application for inhibition of binding of WDR5 to its binding partners in a cell as well as a use of one or more compounds of the application for the preparation of a medicament for inhibition of binding of WDR5 to its binding partners in a cell. The application further includes one or more compounds of the application for use to inhibit binding of WDR5 to its binding partners in a cell.

It is an embodiment of the present application, in all aspects, that the binding partner for WDR5 is MLL1, or a portion thereof. In some embodiments, the binding partner forWDR5 is the WDR5 interacting (WIN) motif, consisting of amino acid residues 3762-3773 next to the SET domain in the MLL1 protein, [J. Biol. Chem., 2008, 283(47):32158-32161; J Biol. Chem., 2008,283(50):35258-35264].

As the compounds of the application have been shown to be capable of inhibiting the binding of WDR5 to its binding partners, the compounds of the application are useful for treating diseases, disorders or conditions mediated or treatable by inhibition of binding between WDR5 protein and its binding partners. Therefore the compounds of the present application are useful as medicaments. Accordingly, the present application includes a compound of the application for use as a medicament.

The present application also includes a method of treating a disease, disorder or condition that is mediated or treatable by inhibition of binding between WDR5 protein and its binding partners comprising administering a therapeutically effective amount of one or more compounds of the application to a subject in need thereof. The present application also includes a use of one or more compounds of the application for treating a disease, disorder or condition mediated or treatable by inhibition of binding between WDR5 protein and its binding partners as well as a use of one or more compounds of the application for the preparation of a medicament for treating of a disease, disorder or condition mediated or treatable by inhibition of binding between WDR5 protein and its binding partners. The application further includes one or more compounds of the application for use in treating a disease, disorder or condition mediated or treatable by inhibition of binding between WDR5 protein and its binding partners.

In an embodiment, the disease, disorder or condition mediated or treatable by inhibition of binding between WDR5 protein and its binding partners is a neoplastic disorder. Accordingly, the present application also includes a method of treating a neoplastic disorder comprising administering a therapeutically effective amount of one or more compounds of the application to a subject in need thereof. The present application also includes a use of one or more compounds of the application for treatment of a neoplastic disorder as well as a use of one or more compounds of the application for the preparation of a medicament for treatment of a neoplastic disorder. The application further includes one or more compounds of the application for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

In another embodiment of the present application, the disease, disorder or condition mediated or treatable by inhibition of binding between WDR5 protein and its binding partners is cancer. Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer. In an embodiment, the compound is administered for the prevention of cancer in a subject such as a mammal having a predisposition for cancer.

In an embodiment, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-CeIl Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-CeIl; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-CeIl Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In an embodiment, the cancer is selected from solid cancer and leukemias. In another embodiment, the cancer is selected from leukaemia, lymphoma, non-Hodgkin's lymphoma, Burkitt lymphoma, MLL-fusion lymphoma, primary effusion leukemia and multiple myeloma. In a further embodiment of the present application, the cancer is selected from leukemia, melanoma, lung cancer, bladder cancer, colon cancer, brain cancer, ovarian cancer, breast cancer, prostate cancer, neuroblastoma and kidney cancer. In a further embodiment of the present application, the cancer is selected from leukemia, bladder cancer, brain cancer, prostate cancer and neuroblastoma. In a further embodiment, the cancer is selected from bladder cancer, gliomas, glioblastomas, acute myeloid leukemia (AML) and MYCN-amplified neuroblastoma.

In an embodiment, the disease, disorder or condition mediated or treatable by inhibition of binding between WDR5 protein and its binding partners is a disease, disorder or condition associated with an uncontrolled and/or abnormal cellular activity affected directly or indirectly by a binding of WDR5 to its binding partners. In another embodiment, the uncontrolled and/or abnormal cellular activity that is affected directly or indirectly by binding of WDR5 to its binding partners is proliferative activity in a cell. Accordingly, the application also includes a method of inhibiting proliferative activity in a cell, comprising administering an effective amount of one or more compounds of the application to the cell. The present application also includes a use of one or more compounds of the application for inhibition of proliferative activity in a cell as well as a use of one or more compounds of the application for the preparation of a medicament for inhibition of proliferative activity in a cell. The application further includes one or more compounds of the application for use in inhibiting proliferative activity in a cell.

The present application also includes a method of inhibiting uncontrolled and/or abnormal cellular activities mediated directly or indirectly by binding of WDR5 to its binding partners in a cell, either in a biological sample or in a subject, comprising administering an effective amount of one or more compounds of the application to the cell. The application also includes a use of one or more compounds of the application for inhibition of uncontrolled and/or abnormal cellular activities mediated directly or indirectly by binding of WDR5 to its binding partners in a cell as well as a use of one or more compounds of the application for the preparation of a medicament for inhibition of uncontrolled and/or abnormal cellular activities mediated directly or indirectly binding of WDR5 to its binding partners in a cell. The application further includes one or more compounds of the application for use in inhibiting uncontrolled and/or abnormal cellular activities mediated directly or indirectly by binding of WDR5 to its binding partners in a cell.

In further embodiments, the present application also includes a method of treating a disease, disorder or condition that is mediated or treatable by inhibition of binding between WDR5 protein and its binding partners comprising administering a therapeutically effective amount of one or more compounds of the application in combination with another known agent useful for treatment of a disease, disorder or condition mediated or treatable by inhibition of binding between WDR5 protein and its binding partners to a subject in need thereof. The present application also includes a use of one or more compounds of the application in combination with a known agent useful for treatment of a disease, disorder or condition mediated or treatable by inhibition of binding between WDR5 protein and its binding partners, for treatment of a disease, disorder or condition mediated or treatable by inhibition of binding between WDR5 protein and its binding partners.

In a further embodiment, the disease, disorder or condition mediated or treatable by inhibition of binding between WDR5 protein and its binding partners is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

### Methods of Preparing the Compounds of the Application

Compounds of the application may be prepared using methods known in the art, for example as described in the examples herein.

Scheme 1 illustrates one embodiment of a route to compounds of the application in which Suzuki or related coupling is performed on compounds A to afford compounds of Formula I.

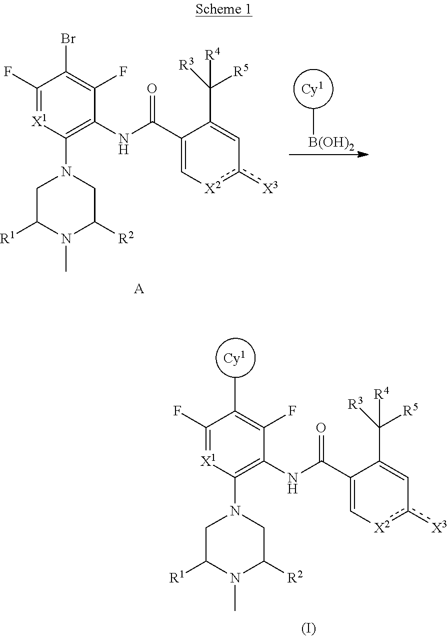

Compounds of Formula A are available, for example from commercially available 4-bromo-1,3,5-trifluorobenzene (X¹=CH) or from 3-bromo-2,4,6-trifluoropyridine (X¹=N), i.e. compounds of Formula B, as shown in Scheme 2. Therefore compounds of Formula B are nitrated to provide compounds of Formula C. Compounds of Formula C are reacted under basic conditions with compounds of Formula D to provide compounds of Formula E. Compounds of Formula E are methylated to provide compounds of Formule F, which are then reduced to provide compounds of Formula G. Compounds of Formula G are reacted with compounds of Formula H under standard amide bond forming conditions to provide compounds of Formula A.

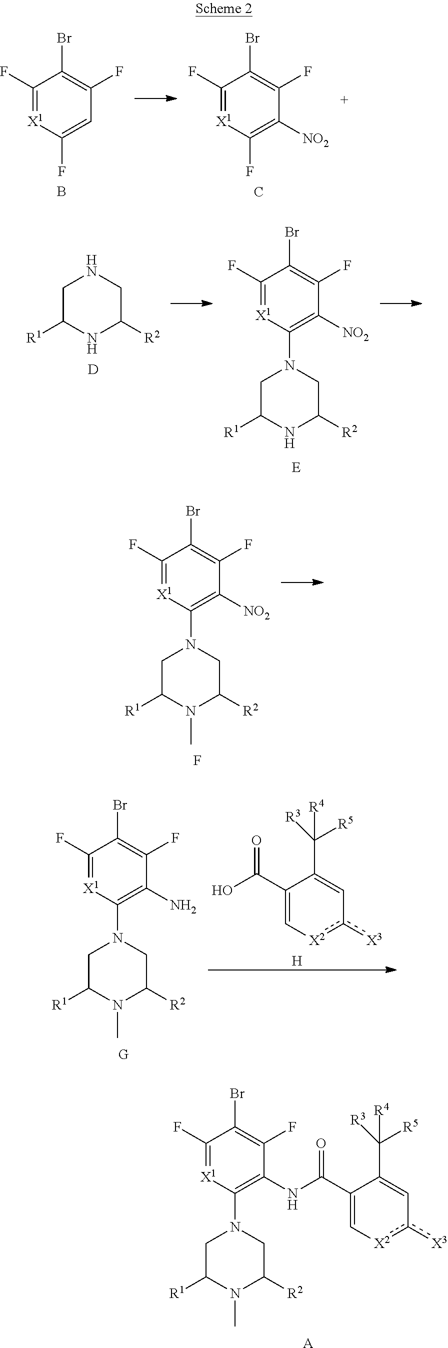

A person skilled in the art would understand that the order of addition of the Cy¹, piperidine and carboxylate groups onto the central aromatic core (e.g. compounds of Formula B) can be varied depending, for example, on the reactivity of substituents on each of Cy¹, the piperidine and carboxylate groups. Therefore, the Cy¹ group may be incorporated first, followed by the piperazine group followed by the carboxylate group. Alternatively, the piperizine group may be incorporated first followed by Cy¹ and the carboxylate.

Throughout the synthetic methods and processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### EXAMPLES

The following non-limiting examples are illustrative of the present application:

### A. General Methods

Exemplary compounds were synthesized using the methods described herein, or other methods, which are known in the art. Unless otherwise noted, reagents and solvents were obtained from commercial suppliers (e.g. Aldrich, Enamine, Combiblock, Bepharm, J&W PharmLab).

The compounds and/or intermediates were characterized by high performance liquid chromatography (HPLC) using a Waters ACQUITY UPLC system with a SQ (single quadrupole) MS and a photodiode array (PDA) detector (Milford, Mass.). The analytical columns were reversed phase Acqity UPLC BEH C18 (2.1×50 mm, 1.7 μm). A gradient elution was used (flow 0.4 mL/min), typically starting with mobile phase 0.1% formic acid in water (solvent A) and 0.1% formic acid in acetonitrile (solvent B). A gradient starting at 95% solvent A going to 5% in 1.8 min., holding for 0.5 min., going back to 95% in 0.5 min. and equilibrating the column for 0.5 min. Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel IB2-F flexible sheets. TLC results were readily detected visually under ultraviolet light, or by employing well-known iodine vapor and other various staining techniques.

The compounds and/or intermediates were characterized by LCMS. General conditions are as follows. Low and High resolution Mass spectra were acquired on LC/MS systems using electrospray ionization methods from a range of instruments of the following configurations: Low resolution—Waters ACQUITY UPLC system with a SQ (single quadrupole) MS; Waters ACQUITY UPLC H-Class system with a 3100 (single quadrupole) MS. High resolution—Waters ACQUITY UPLC II system equipped with a Synapt Xevo QTof and Waters ACQUITY UPLC II system equipped with a Synapt G2S QTof mass spectrometer with an atmospheric pressure ionization source. [M+H] refers to the protonated molecular ion of the chemical species.

Nuclear magnetic resonance (NMR) analysis was performed on a Bruker 500 MHz NMR spectrometer using ICON-NMR, under TopSpin program control. Spectra were measured at 298K, unless indicated otherwise and were referenced relative to the solvent chemical shift. The compounds of the application were prepared by conventional methods for chemical synthesis according to the procedures outlined in the schemes below. The starting materials for the processes described in the present application are known or may readily be prepared by conventional methods from commercially available chemicals.

### B. Synthesis of Compounds

### Example 1: Synthesis of N-(3-(6-(cycloproylmethoxy)pyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

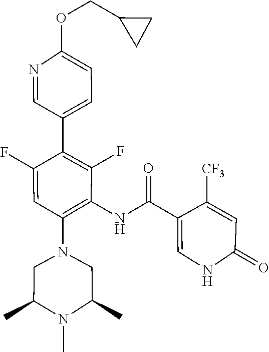 [SMILES: C[C@@H]1CN(c2cc(F)c(-c3ccc(OCC4CC4)nc3)c(F)c2NC(=O)c2c[nH]c(=O)cc2C(F)(F)F)C[C@H](C)N1C]

### Step 1: 2-Bromo-1,3,5-trifluoro-4-nitrobenzene

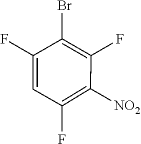 [SMILES: O=[N+]([O-])c1c(F)cc(F)c(Br)c1F]

A stirred solution of 2-bromo-1,3,5-trifluorobenzene (25 g, 119.1 mmol, 1 eq) in H₂SO₄ (120 mL) was cooled to 0° C. and HNO₃ (106 mL) was added dropwise. The reaction was stirred for 2 h at 0° C. TLC analysis indicated formation of non-polar spot. The reaction mixture was quenched with ice water (500 mL) and extracted with EtOAc (2×500 mL). The combined organic layer was washed with sat. NaHCO₃solution followed by brine solution, dried over Na₂SO₄ and concentrated under reduced pressure to give 2-bromo-1,3,5-trifluoro-4-nitrobenzene (28 g, 92.4% yield) as a yellow liquid. LCMS: [M−H]− 254.01.

### Step 2: (3R,5S)-1-(4-bromo-3,5-difluoro-2-nitrophenyl)-3,5-dimethylpiperazine

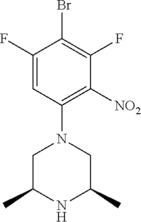 [SMILES: C[C@@H]1CN(c2cc(F)c(Br)c(F)c2[N+](=O)[O-])C[C@H](C)N1]

To a stirred solution of 2-bromo-1,3,5-trifluoro-4-nitrobenzene (28 g, 109.8 mmol, 1 eq) in ethanol (560 mL), was added DIPEA (60 mL, 329.4 mmol, 3 eq) followed by (2S,6R)-2,6-dimethylpiperazine (15 g, 131.7 mmol, 1.2 eq) and resulted reaction mixture was heated at 85° C. for 16h. TLC analysis indicated formation of polar spot. The reaction mixture was concentrated under reduced pressure. The crude compound was purified by column chromatography (silica gel 100-200) using 5% methanol in DCM as an eluent to give (3R,5S)-1-(4-bromo-3,5-difluoro-2-nitrophenyl)-3,5-dimethylpiperazine (28.5 g, 74% yield) as yellow solid. LCMS: [M+H]⁺ 350.15.

### Step 3: (2R,6S)-4-(4-bromo-3,5-difluoro-2-nitrophenyl)-1,2,6-trimethylpiperazine

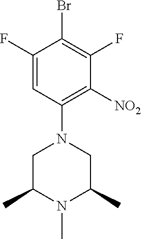 [SMILES: C[C@@H]1CN(c2cc(F)c(Br)c(F)c2[N+](=O)[O-])C[C@H](C)N1C]

A stirred solution of (3R,5S)-1-(4-bromo-3,5-difluoro-2-nitrophenyl)-3,5-dimethylpiperazine (53 g, 151.8 mmol, 1 eq) in DCM (530 mL) was cooled to 0° C. and 37% HCHO (63.5 mL, 607.4 mmol, 4eq) was added. The resulting reaction mixture was stirred at RT for 2h. The mixture was cooled to 0° C. and NaCNBH₃ (19 g, 303.7 mol, 2eq) was added portion wise. The reaction mixture was stirred at RT for 16 h. TLC analysis indicated formation of non-polar spot. The reaction mixture was quenched with sat. NaHCO₃ solution and extracted with DCM (2×500 mL). The combined organic layers were dried over Na₂SO₄ and concentrated under reduced pressure. The crude compound was purified by column chromatography (silica gel 100-200) using 0-10% methanol in DCM as an eluent to give (2R,6S)-4-(4-bromo-3,5-difluoro-2-nitrophenyl)-1,2,6-trimethylpiperazine (25 g, 45.45% yield) as yellow solid. LCMS: [M+H]⁺ 364.43.

### Step 4: 3-Bromo-2,4-difluoro-6-((3S,5R)-3,4,5-trimethylpiperazin-1-yl)aniline

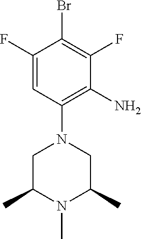 [SMILES: C[C@@H]1CN(c2cc(F)c(Br)c(F)c2N)C[C@H](C)N1C]

To a stirred solution of (2R,6S)-4-(4-bromo-3,5-difluoro-2-nitrophenyl)-1,2,6-trimethylpiperazine (5.5 g, 15.1 mmol, 1 eq) in ethanol: H₂O (82 mL: 11 mL) was added NH₄Cl (3.24 g, 60.6 mmol, 4 eq) followed by Fe powder (3.38 g, 60.6 mmol, 4 eq). The resulting mixture was stirred at RT for 16 h. The mixture was cooled to RT, filtered through celite, and washed with EtOAc (200 mL). The filtrate was dried over Na₂SO₄ and concentrated under reduced pressure to give the crude product which was purified by column chromatography (silica gel 100-200 mesh) using 0-10% methanol in DCM as an eluent to afford 3-bromo-2,4-difluoro-6-((3S,5R)-3,4,5-trimethylpiperazin-1-yl)aniline (2.6 g, 52% yield) as yellow solid. LCMS: [M+H]+ 334.41.

### Step 5: Synthesis of N-(3-bromo-2,4-difluoro-6-((3S,5R)-3,4,5-trimethylpiperazin-1-yl)phenyl)-6-oxo-4-(trifluoromethyl)-1,6-dihydropyridine-3-carboxamide

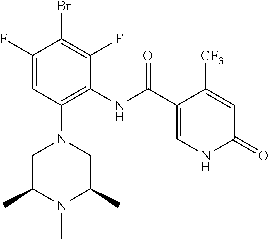 [SMILES: C[C@@H]1CN(c2cc(F)c(Br)c(F)c2NC(=O)c2c[nH]c(=O)cc2C(F)(F)F)C[C@H](C)N1C]

To a 50 mL round bottomed flask (RBF) charged with 6-chloro-4-(trifluoromethyl)nicotinic acid (595 mg, 2.64 mmol, 2.1 equiv) was added thionyl chloride (1.83 mL, 25.1 mmol). The resulting suspension was heated at 80° C. for 1 h. The solvent was evaporated to give a light yellow oil which was redissolved in DCM (10 mL). 3-Bromo-2,4-difluoro-6-((3S,5R)-3,4,5-trimethylpiperazin-1-yl)aniline (420 mg, 1.257 mmol) was added in one portion, followed by Et₃N (0.70 mL, 5.03 mmol). The resulting dark red solution was stirred at ambient temperature for 1.5 h. After basifying with sat. NaHCO₃ (30 mL), it was extracted with DCM (30 mL×2). The combined extracts were concentrated and dried under vacuum overnight to give a light pinkish white foam. A mixture of the above solid and NaOAc (309 mg, 3.77 mmol) in HOAc/H₂O (10 mL/3 mL) was then heated in microwave at 160° C. for 6 h. Solvents were removed using a rotovap at 60° C., and the residue was redissolved in DCM (30 mL) and MeOH (15 mL) and treated with sat. NaHCO₃ (30 mL). After stirring at RT for 10 min, the mixture was extracted with DCM (30 mL×2). The combined extracts were concentrated to give a brown solid which was purified by flash chromatography (gradient: EtOAc/Hex 0-100% then MeOH/DCM 0-10%) to give the title compound as a pale beige solid (512 mg, 75%). LCMS [M+H]+ 523.3.

### Step 6: Synthesis of N-(3-(6-(cyclopropylmethoxy)pyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

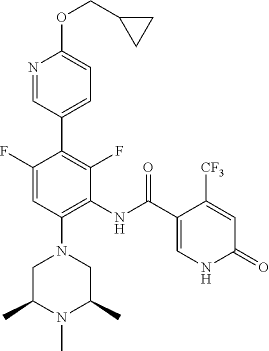 [SMILES: C[C@@H]1CN(c2cc(F)c(-c3ccc(OCC4CC4)nc3)c(F)c2NC(=O)c2c[nH]c(=O)cc2C(F)(F)F)C[C@H](C)N1C]

To a 5 mL microwave vial charged with N-(3-bromo-2-fluoro-6-((3S,5R)-3,4,5-trimethylpiperazin-1-yl)phenyl)-6-oxo-4-(trifluoromethyl)-1,6-dihydropyridine-3-carboxamide (52.2 mg, 0.1 mmol), 2-(cyclopropylmethoxy)-5-(4,4,5,5-tetramethyl-1,3,2-dioxaborolan-2-yl)pyridine (55 mg, 0.2 mmol), and Pd(dppf)Cl₂ (15 mg, 0.02 mmol, 20 mol %) was added dioxane (3 mL), followed by 1 M aq. K₃PO₄ (0.5 mL, 0.5 mmol). The resulting mixture was irradiated in a microwave apparatus at 110° C. for 2 h, diluted with H₂O (10 mL) and extracted with EtOAc (20 mL×2). The combined extracts were concentrated and purified by Biotage (SNAP KP-Sil 25 g column, gradient: EtOAc/hex 0-100% then MeOH/DCM 0-15%) to give the title compound the title compound as a brown solid (48.7 mg, 78%). LCMS [M+H]+ 592.3.

### Example 2: Synthesis of N-(2,4-difluoro-3-(2-morpholinopyrimidin-5-yl)-6-((3S,5R)-3,4,5-trimethylpiperazin-1-yl)phenyl)-6-oxo-4-(trifluoromethyl)-1,6-dihydropyridine-3-carboxamide

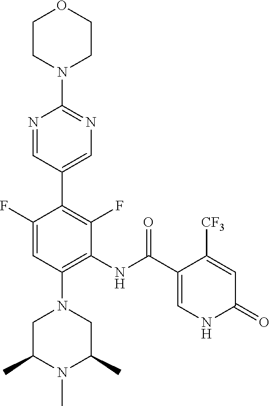 [SMILES: C[C@@H]1CN(c2cc(F)c(-c3cnc(N4CCOCC4)nc3)c(F)c2NC(=O)c2c[nH]c(=O)cc2C(F)(F)F)C[C@H](C)N1C]

The title compound (white solid, 34.8 mg, 57%) was prepared according to a procedure similar to Example 1, Step 6 using N-(3-bromo-2,4-difluoro-6-((3S,5R)-3,4,5-trimethylpiperazin-1-yl)phenyl)-6-oxo-4-(trifluoromethyl)-1,6-dihydropyridine-3-carboxamide (52.3 mg, 0.1 mmol) and 2-(4-morpholino)pyrimidine-5-boronic acid pinacol ester (58 mg, 0.2 mmol). LCMS [M+H]+ 608.3.

### Example 3: N-(3-(2-(cyclopropylmethoxy)pyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

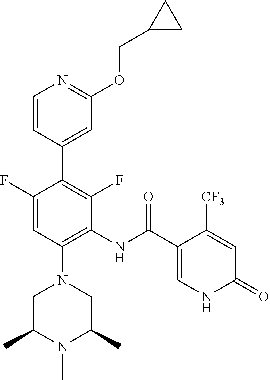 [SMILES: C[C@@H]1CN(c2cc(F)c(-c3ccnc(OCC4CC4)c3)c(F)c2NC(=O)c2c[nH]c(=O)cc2C(F)(F)F)C[C@H](C)N1C]

The title compound (white solid, 26.2 mg, 55%) was prepared according to a procedure similar to Example 1 Step 6 using N-(3-bromo-2-fluoro-6-((3S,5R)-3,4,5-trimethylpiperazin-1-yl)phenyl)-6-oxo-4-(trifluoromethyl)-1,6-dihydropyridine-3-carboxamide (41.9 mg, 0.08 mmol) and 2-(cyclopropylmethoxy)-4-(4,4,5,5-tetramethyl-1,3,2-dioxaborolan-2-yl)pyridine (44 mg, 0.16 mmol). LCMS [M+H]+ 592.4.

### Example 4: N-(2,4-difluoro-3-(6-morpholinopyridin-3-yl)-6-((3S,5R)-3,4,5-trimethylpiperazin-1-yl)phenyl)-6-oxo-4-(trifluoromethyl)-1,6-dihydropyridine-3-carboxamide

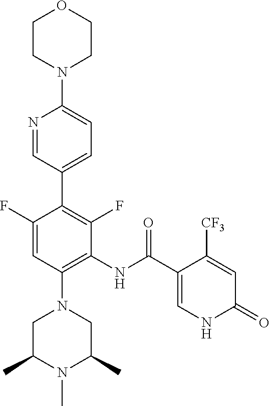

The title compound (white solid, 8.1 mg, 16%) was prepared according to a procedure similar to Example 1, Step 6 using N-(3-bromo-2-fluoro-6-((3S,5R)-3,4,5-trimethylpiperazin-1-yl)phenyl)-6-oxo-4-(trifluoromethyl)-1,6-dihydropyridine-3-carboxamide (41.9 mg, 0.08 mmol) and 4-[5-(4,4,5,5-tetramethyl-[1,3,2]dioxaborolan-2-yl)-pyridin-2-yl]-morpholine (46 mg, 0.16 mmol). LCMS [M+H]+ 607.4.

### Example 5: N-(2,6-difluoro-4′-morpholino-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide

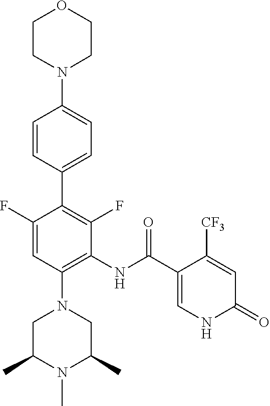

The title compound (white solid, 32.7 mg, 67%) was prepared according to a procedure similar to Example 1, Step 6 using N-(3-bromo-2-fluoro-6-((3S,5R)-3,4,5-trimethylpiperazin-1-yl)phenyl)-6-oxo-4-(trifluoromethyl)-1,6-dihydropyridine-3-carboxamide (41.9 mg, 0.08 mmol) and 4-(morpholino)phenylboronic acid (33 mg, 0.16 mmol). LCMS [M+H]+ 606.5.

### Example 6: N-(2,4-difluoro-3-(6-(2-methoxyethoxy)pyridin-3-yl)-6-((3S,5R)-3,4,5-trimethylpiperazin-1-yl)phenyl)-6-oxo-4-(trifluoromethyl)-1,6-dihydropyridine-3-carboxamide

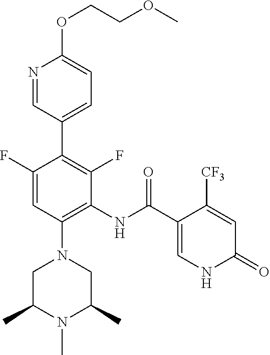 [SMILES: COCCOc1ccc(-c2c(F)cc(N3C[C@@H](C)N(C)[C@@H](C)C3)c(NC(=O)c3c[nH]c(=O)cc3C(F)(F)F)c2F)cn1]

The title compound (white solid, 27.8 mg, 57% yield) was prepared according to a procedure similar to Example 1, Step 6 using N-(3-bromo-2,4-difluoro-6-((3S,5R)-3,4,5-trimethylpiperazin-1-yl)phenyl)-6-oxo-4-(trifluoromethyl)-1,6-dihydropyridine-3-carboxamide (41.9 mg, 0.08 mmol) and 2-(2-methoxyethoxy)-5-(4,4,5,5-tetramethyl-1,3,2-dioxaborolan-2-yl)pyridine (45 mg, 0.16 mmol). LCMS [M+H]+596.4.

### Example 7: N-(4′-(cyclopropylmethoxy)-2,6-difluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide

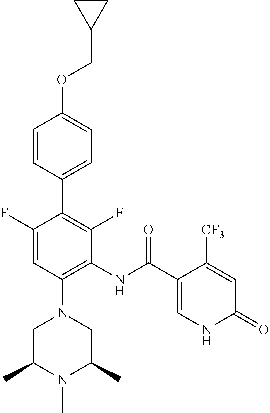 [SMILES: C[C@@H]1CN(c2cc(F)c(-c3ccc(OCC4CC4)cc3)c(F)c2NC(=O)c2c[nH]c(=O)cc2C(F)(F)F)C[C@H](C)N1C]

The title compound (white solid, 27.7 mg, 58% yield) was prepared according to a procedure similar to Example 1, Step 6 using N-(3-bromo-2-fluoro-6-((3S,5R)-3,4,5-trimethylpiperazin-1-yl)phenyl)-6-oxo-4-(trifluoromethyl)-1,6-dihydropyridine-3-carboxamide (41.9 mg, 0.08 mmol) and 4-(cyclopropylmethoxy)phenylboronic acid (31 mg, 0.16 mmol). LCMS [M+H]+ 591.4.

### Example 8: N-(3-(2-((2S,6R)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

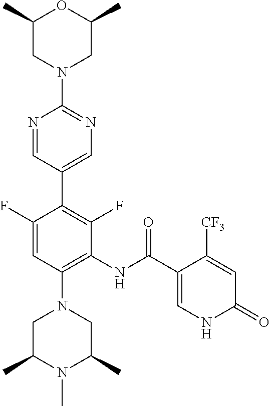 [SMILES: C[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)C[C@H](C)O1]

The title compound (white solid, 27.6 mg, 53%) was prepared according to a procedure similar to Example 1, Step 6 using N-(3-bromo-2-fluoro-6-((3S,5R)-3,4,5-trimethylpiperazin-1-yl)phenyl)-6-oxo-4-(trifluoromethyl)-1,6-dihydropyridine-3-carboxamide (41.9 mg, 0.08 mmol) and (2-((2S,6R)-2,6-dimethylmorpholino)pyrimidin-5-yl)boronic acid (38 mg, 0.16 mmol). LCMS [M+H]+ 636.4.

### Example 9:6-Oxo-N-(2,3′,6-trifluoro-4′-morpholino-4-((3S,5R)-3,4,5-trimethylpiperazin-1-yl)-[1,1′-biphenyl]-3-yl)-4-(trifluoromethyl)-1,6-dihydropyridine-3-carboxamide

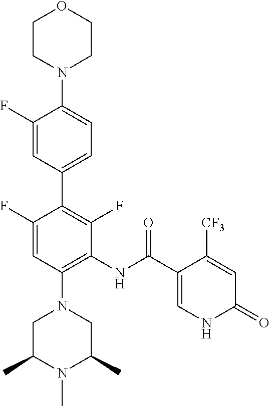

The title compound (white solid, 30.3 mg, 60%) was prepared according to a procedure similar to Example 1, Step 6 using N-(3-bromo-2-fluoro-6-((3S,5R)-3,4,5-trimethylpiperazin-1-yl)phenyl)-6-oxo-4-(trifluoromethyl)-1,6-dihydropyridine-3-carboxamide (41.9 mg, 0.08 mmol) and 3-fluoro-4-morpholinophenylboronic acid (36 mg, 0.16 mmol). LCMS [M+H]+ 624.3.

### Example 10: N-(2,4-difluoro-3-(2-morpholinopyrimidin-5-yl)-6-((3S,5R)-3,4,5-trimethylpiperazin-1-yl)phenyl)-4-fluoro-2-(trifluoromethyl)benzamideformic acid

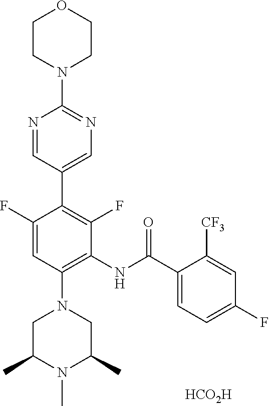

To a 20 mL microwave vial charged with 3-bromo-2,4-difluoro-6-((3S,5R)-3,4,5-trimethylpiperazin-1-yl)aniline (334 mg, 1 mmol), 2-(4-morpholino)pyrimidine-5-boronic acid pinacol ester (437 mg, 1.5 mmol) and Pd(dppf)Cl₂(59 mg, 0.08 mmol, 8 mol %) was added dioxane (8 mL), followed by 1 M aq K₃P04 (2 mL, 2 mmol). The resulting mixture was purged with N₂ and irritated in microwave at 110° C. for 3 h. After separation of the organic layer, the aqueous layer was extracted with EtOAc (5 mL×2). The combined organic layers were concentrated to give a dark greenish brown solid which was triturated with MeOH (10 mL), filtered and rinsed well with MeOH (5 mL) and dried under vacuum to give 2,4-difluoro-3-(2-morpholinopyrimidin-5-yl)-6-((3S,5R)-3,4,5-trimethylpiperazin-1-yl)aniline as a brown solid (374 mg, 86% based on 96.23% purity). LCMS [M+H]+ 419.5. To a solution of 4-fluoro-2-(trifluoromethyl)benzoyl chloride (0.045 mL, 0.3 mmol) in DCM (3 mL) at RT was added Et₃N (0.084 mL, 0.6 mmol). After addition, the resulting mixture was stirred at RT for 5 min, then a solution of 2,4-difluoro-3-(2-morpholinopyrimidin-5-yl)-6-((3S,5R)-3,4,5-trimethylpiperazin-1-yl)aniline (42 mg, 0.1 mmol) in DCM (2 mL) was added. The resulting mixture was stirred at RT for 2 h. After quenching with sat. NaHCO₃ (15 mL) and stirring for 10 min at RT, the mixture was extracted with DCM (20 mL×2). The combined extracts were combined, concentrated and purified by flash chromatography (gradient: EtOAc/hex 0-100% then MeOH/DCM 0-10%) and prep-HPLC to give the title compound as a beige solid (formic acid salt, 22.1 mg, 34%).

### Example 11: N-(2,4-difluoro-3-(2-morpholinopyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide

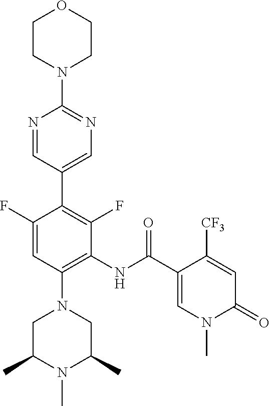

### Step 1: 2,4-difluoro-3-(2-morpholinopyrimidin-5-yl)-6-((3S,5R)-3,4,5-trimethylpiperazin-1-yl)aniline

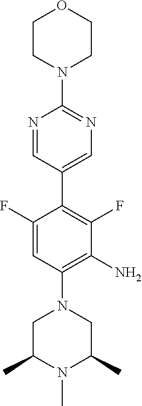

To a 20 mL microwave vial charged with 3-bromo-2,4-difluoro-6-((3S,5R)-3,4,5-trimethylpiperazin-1-yl)aniline (334 mg, 1 mmol, preparation described in Example 1), 2-(4-morpholino)pyrimidine-5-boronic acid pinacol ester (437 mg, 1.5 mmol) and Pd(dppf)Cl₂ (59 mg, 0.08 mmol, 8 mol %) was added dioxane (8 mL), followed by 1 M aq K₃P04 (2 mL, 2 mmol). The resulting mixture was purged with N₂ and irradiated in a microwave apparatus at 110° C. for 3 h. After separation of the organic layer, the aqueous layer was extracted with EtOAc (5 mL×2). The combined organic layers were concentrated to give a dark greenish brown solid which was triturated with MeOH (10 mL), filtered and washed with MeOH (5 mL). Drying under vacuum afforded the product as a brown solid (86% yield based on 96.2% purity). LCMS [M+H]⁺ 419.45.

### Step 2: N-(2,4-difluoro-3-(2-morpholinopyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide

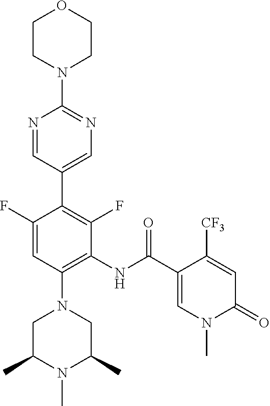

To a 25 mL RBF charged with 1-methyl-6-oxo-4-(trifluoromethyl)-1,6-dihydropyridine-3-carboxylic acid (88 mg, 0.4 mmol) was added thionyl chloride (0.58 mL, 8 mmol). The resulting suspension was heated at 80° C. for 1 h and evaporated to give a pale yellow oil which solidified to a white solid. It was treated with 2,4-difluoro-3-(2-morpholinopyrimidin-5-yl)-6-((3S,5R)-3,4,5-trimethylpiperazin-1-yl)aniline (84 mg, 0.2 mmol), DCM (5 mL), follow ed by Et₃N (0.11 mL, 0.8 mmol). The resulting red/brown solution was stirred at RT for 2 h. After quenching with sat. NaHCO₃(5 mL) and stirring for 10 min at RT, the mixture was extracted with DCM (10 mL×2). The combined extracts were combined, concentrated and purified by flash chromatography (gradient: EtOAc/hex 0-100% then MeOH/DCM 0-10%) to give the title compound as a beige crystalline solid (60 mg, 48%). LCMS [M+H]+ 622.4.

### Example 12: N-(2,4-difluoro-3-(2-((S)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide formic Acid

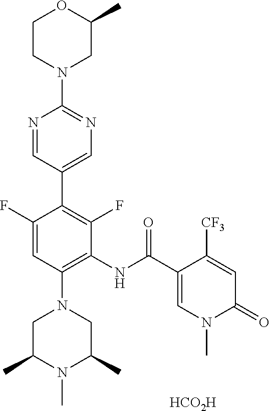 [SMILES: C[C@@H]1CN(c2cc(F)c(-c3cnc(N4CCO[C@@H](C)C4)nc3)c(F)c2NC(=O)c2cn(C)c(=O)cc2C(F)(F)F)C[C@H](C)N1C ; "HCO2H"]

### Step 1: (S)-(2-(2-methylmorpholino)pyrimidin-5-yl)boronic Acid

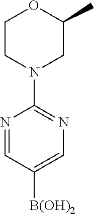 [SMILES: C[C@H]1CN(c2ncc(B(O)O)cn2)CCO1]

To a mixture of 2-chloropyrimidine-5-boronic acid (626 mg, 3.95 mmol) and (S)-2-methylmorpholine (440 mg, 4.35 mmol) in EtOH (12 mL) was added triethylamine (0.83 mL, 5.93 mmol). The resulting mixture (a cloudy suspension, never went to clear) was stirred at 80° C. for 1.5 h. Solvents were removed to give a yellow solid which was dried under high vacuum to give crude (S)-(2-(2-methylmorpholino)pyrimidin-5-yl)boronic acid as a yellow solid (1.092 g, 3.95 mmol, 80% purity assuming full conversion). LCMS [M+H]+ 224.3.

### Step 2: N-(3-bromo-2,4-difluoro-6-((3S,5R)-3,4,5-trimethylpiperazin-1-yl)phenyl)-1-methyl-6-oxo-4-(trifluoromethyl)-1,6-dihydropyridine-3-carboxamide

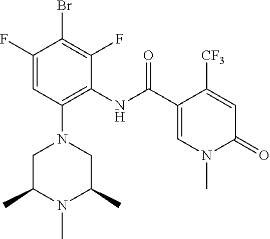 [SMILES: C[C@@H]1CN(c2cc(F)c(Br)c(F)c2NC(=O)c2cn(C)c(=O)cc2C(F)(F)F)C[C@H](C)N1C]

To a 25 mL RBF charged with 1-methyl-6-oxo-4-(trifluoromethyl)-1,6-dihydropyridine-3-carboxylic acid (166 mg, 0.75 mmol, preparation described in Example 1) was added thionyl chloride (1.09 mL, 30 mmol). The resulting suspension was heated at 80° C. for 1 h and evaporated to give a pale yellow oil which solidified to a white solid. Treatment with 3-bromo-2,4-difluoro-6-((3S,5R)-3,4,5-trimethylpiperazin-1-yl)aniline (167 mg, 0.5 mmol), DCM (10 mL), followed by Et₃N (0.28 mL, 2 mmol). The resulting red/brown solution was stirred at RT for 2 h. After quenching with sat. NaHCO₃ (15 mL) and stirring for 10 min at RT, the mixture was extracted with DCM (20 mL×2). The combined extracts were concentrated, loaded onto silica gel with DCM/MeOH and purified by Biotage (SNAP KP-Sil 50 g column, gradient: EtOAc/hex 0-100% then MeOH/DCM 0-10%) to give the product as a beige crystalline solid (80 mg, yield 28.3% based on 94.91% purity). LCMS [M+H]+ 537.3.

### Step 3: N-(2,4-difluoro-3-(2-((S)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide Formic Acid

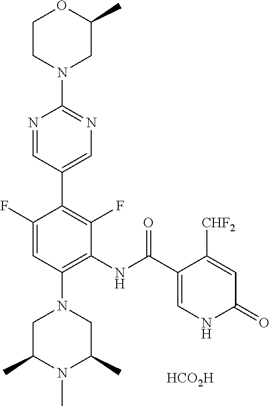 [SMILES: C[C@@H]1CN(c2cc(F)c(-c3cnc(N4CCO[C@@H](C)C4)nc3)c(F)c2NC(=O)c2c[nH]c(=O)cc2C(F)F)C[C@H](C)N1C ; "HCO2H"]

The title compound (formic acid salt, off white solid, 31.1 mg, 68%) was prepared by a procedure similar to Example 1, Step 6 using N-(3-bromo-2,4-difluoro-6-((3S,5R)-3,4,5-trimethylpiperazin-1-yl)phenyl)-1-methyl-6-oxo-4-(trifluoromethyl)-1,6-dihydropyridine-3-carboxamide (40 mg, 94.9% purity, 0.07 mmol) and (S)-(2-(2-methylmorpholino)pyrimidin-5-yl)boronic acid (0.3 mmol, crude).

### Example 13: Isopropyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate

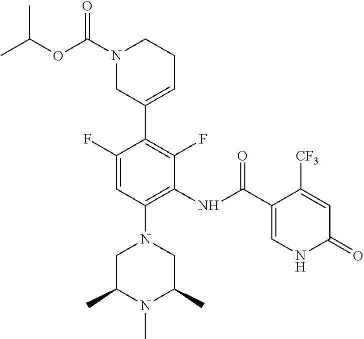 [SMILES: CC(C)OC(=O)N1CCC=C(c2c(F)cc(N3C[C@@H](C)N(C)[C@@H](C)C3)c(NC(=O)c3c[nH]c(=O)cc3C(F)(F)F)c2F)C1]

### Step 1: 5-Bromo-2-chloro-4-iodopyridine

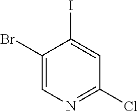

A stirring solution of DIPA (34 mL, 227.7 mmol, 1.1 eq) in dry THF (250 mL) was cooled to −78° C. and n-BuLi (85 mL, 207.9 mmol, 1.0 eq, 2.5 M in THF) was added dropwise. The resulting reaction mixture was stirred for 30 min. 5-Bromo-2-chloropyridine (40 g, 207.9 mmol, 1 eq.) in dry THF (450 mL) was added dropwise and the reaction mixture was stirred at −78° C. for 1 h. A solution of iodine (55 g, 207.9 mmol, 1eq) in THF (250 mL) was added dropwise and the mixture was stirred for 16 h at RT. TLC analysis indicated formation of non-polar spot. The mixture was quenched with sodium thiosulfate solution (500 mL) and extracted with EtOAc (2×500 mL). The combined organic layers were dried over Na₂SO₄ and concentrated under reduced pressure gave crude product which was recrystallized from ethanol (120 mL) to afford 5-bromo-2-chloro-4-iodopyridine (50 g, 75.4% yield) as an off white solid. LCMS: [M+H]₊ 320.15.

### Step 2: 5-Bromo-2-chloro-4-(trifluoromethyl)pyridine

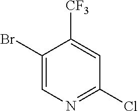 [SMILES: FC(F)(F)c1cc(Cl)ncc1Br]

To a stirred solution of 5-bromo-2-chloro-4-iodopyridine (40 g, 126.2 mmol, 1 eq) in DMF (400 mL) was added methyl 2,2-difluoro-2-(fluorosulfonyl)acetate (32.1 mL, 252.5 mmol, 2 eq) followed by CuI (48.2 g, 252.5 mmol, 2 eq). The resulting mixture was heated at 100° C. for 6 h. TLC analysis indicated formation of a non-polar spot. The reaction mixture was diluted with water (200 mL), filtered through a celite pad and washed with n-pentane (2×500 mL) and followed by cold water (3×1000 mL). Organic layers were separated, dried over Na₂SO₄ and concentrated under reduced pressure at 30° C. resulted 5-bromo-2-chloro-4-(trifluoromethyl)pyridine (21 g, 64% yield) as a liquid compound. TLC: 5% EtOAc in pet ether; R_{f}: 0.7

### Step 3: 6-Chloro-4-(trifluoromethyl)nicotinic Acid

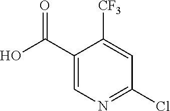 [SMILES: O=C(O)c1cnc(Cl)cc1C(F)(F)F]

A stirred solution of 20% n-butyl magnesium chloride (63 mL, 127.2 mmol, 1.1 eq) in THF (50 mL) was cooled to 0° C. and n-butyl lithium (48 mL, 115.8 mmol, 1 eq, 2.5M in hexane) was added. The resulting reaction mixture was stirred for 10 min, then diluted with THF (100 mL), cooled to −78° C. and a solution of 5-bromo-2-chloro-4-(trifluoromethyl)pyridine (30 g, 115.8 mmol, 1 eq) in THF (50 mL) was added. The reaction mixture was stirred for 1h at −78° C. The mixture was quenched with crushed dry ice and allowed to warm to RT and stirred for 16 h. TLC analysis indicated the formation of a polar spot. The reaction mixture was concentrated, acidified with 2N HCl (80 mL) and extracted with EtOAc (2×500 mL). The combined organic layers were dried over Na₂SO₄ and concentrated under reduced pressure to give the crude compound which was recrystallized from n-pentane (30 mL) and dried on high vacuum to give 6-chloro-4-(trifluoromethyl)nicotinic acid (14 g, 53.8% yield) as off white solid. LCMS: [M+H]+ 226.29.

### Step 4: Methyl 6-chloro-4-(trifluoromethyl)nicotinate

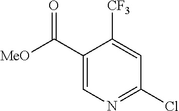 [SMILES: COC(=O)c1cnc(Cl)cc1C(F)(F)F]

A stirred solution of 6-chloro-4-(trifluoromethyl)nicotinic acid (41 g, 182.2 mmol, 1 eq.) in acetone (500 mL) was cooled to 0° C. and added potassium carbonate (38 g, 273.5 mmol, 1.5 eq.) was added, followed by dimethyl sulphate (26 mL, 273.5 mmol, 1.5 eq.) The resulting mixture was stirred at RT for 16 h. TLC analysis indicated formation of non-polar spot. The reaction mixture was diluted with water (500 mL) and extracted with EtOAc (2×200 mL). The combined organic layer was dried over Na₂SO₄ and concentrated under reduced pressure gave crude product. The crude compound was purified by column chromatography (silica gel 100-200 mesh) using 0-10% EtOAc in pet ether eluent to provide the methyl 6-chloro-4-(trifluoromethyl)nicotinate (35 g, 80.4% yield) as liquid. LCMS: [M+H]+ 240.33.

### Step 5: Methyl 4-(trifluoromethyl)-6-(2-(trimethylsilyl)ethoxy)nicotinate

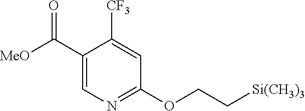

To a stirred solution of methyl 6-chloro-4-(trifluoromethyl)nicotinate (15 g, 62.7 mmol, 1eq) in toluene (150 mL) was added TMS-ethanol (5.56 mL, 62.76 mmol, 1 eq.), cesium carbonate (60.8 g, 184.1 mmol, 3 eq) and by BINAP (4.12 g, 6.23 mmol, 0.1 eq.) The resulting reaction mixture was degassed with nitrogen for 15 min. Pd(OAc)₂ (1.1 g, 4.9 mmol, 0.08 eq.) was added and the mixture was heated at 120° C. for 2 h. TLC analysis indicated formation of non-polar spot. The reaction mixture was diluted with EtOAc (500 mL), filtered through celite and washed with EtOAc. The filtrate was concentrated under reduced pressure to give the crude compound, which was purified by column chromatography (silica gel 100-200mesh) using 0-5% EtOAc in pet ether as eluent to afford methyl 4-(trifluoromethyl)-6-(2-(trimethylsilyl)ethoxy)nicotinate (15 g, 75% yield) as pale yellow liquid. TLC: 20% EtOAc in pet ether; R_{f}: 0.6.

### Step 6: 4-(Trifluoromethyl)-6-(2-(trimethylsilyl)ethoxy)nicotinic acid

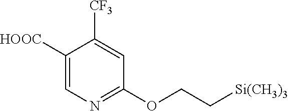

To a stirred solution of methyl 4-(trifluoromethyl)-6-(2-(trimethylsilyl)ethoxy)nicotinate (33.2 g, 103.4 mmol, 1 eq) in THF:MeOH:H₂O (170 mL:55 mL:70 mL) was added lithium hydroxide mono hydrate (17.3 g, 413.6 mmol, 4 eq). The resulting mixture was stirred at RT for 16 h. TLC analysis indicated the formation of polar spot. The reaction was concentrated under reduced pressure to give the crude product, which was acidified with 2N HCl (20 mL). The resulting solid precipitate was collected by filtration and washed with diethyl ether (50 mL) to give 4-(trifluoromethyl)-6-(2-(trimethylsilyl)ethoxy)nicotinic acid (29 g, 91.4% yield) as off white solid. TLC: 20% EtOAc in pet ether; R_{f}: 0.1.

### Step 7: N-(3-bromo-2,4-di fluoro-6-((3S,5R)-3,4,5-trimethylpiperazin-1-yl)phenyl)-4-(trifluoromethyl)-6-(2-(trimethylsilyl)ethoxy)nicotinamide

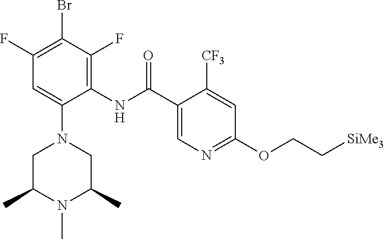 [SMILES: C[C@@H]1CN(c2cc(F)c(Br)c(F)c2NC(=O)c2cnc(OCC[Si](C)(C)C)cc2C(F)(F)F)C[C@H](C)N1C]

To a stirred solution of N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (10 g, 32.5 mmol, 1 eq) in THF (150 mL), was cooled to 0° C. and added DIPEA (45 mL, 163 mmol, 5eq), 3-bromo-2,4-difluoro-6-((3S,5R)-3,4,5-trimethylpiperazin-1-yl)aniline (10.8 g, 32.5 mmol, 1 eq) followed by T₃P (96 mL, 228 mmol, 7 eq.) and the resulting reaction mixture was stirred at RT for 72 h. TLC analysis indicated formation of a non-polar spot. The reaction mixture was quenched with ice water and extracted with EtOAc. The combined organic layers were dried over Na₂SO₄ and concentrated under reduced pressure. The crude product was purified by column chromatography (neutral alumina) using 0-5% methanol in DCM as an eluent resulted the title compound (11 g, 54.5% yield) as off white solid. LCMS: [M+H]⁺ 623.13.

### Step 8: N-(2,4-di fluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide

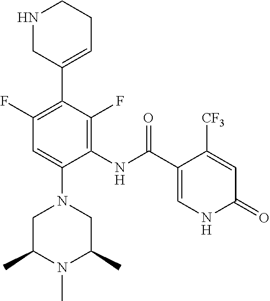 [SMILES: C[C@@H]1CN(c2cc(F)c(C3=CCCNC3)c(F)c2NC(=O)c2c[nH]c(=O)cc2C(F)(F)F)C[C@H](C)N1C]

N-(3-Bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (500 mg, 0.802 mmol), N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (500 mg, 0.802 mmol) and [1,1′-Bis(diphenylphosphino)ferrocene]dichloropalladium(II) (76 mg, 0.104 mmol) were mixed in 1,4-dioxane (12 mL). Potassium phosphate tribasic reagent grade, >=98% (4.01 ml, 4.01 mmol) was added and the vial was flushed with nitrogen. The mixture was heated in a microwave reactor to 100° C. for 3.25 h. The mixture was then partitioned with brine (10 mL) and 10 ml EtOAc, the org phase was separated, aq. phase was extracted with EtOAc (8 m L×2). The combined extracts were dried over Na₂SO₄, concentrated and purified by sgc, eluting with hexanes containing 0-50% EtOAc to afford the Boc protected intermediate as an off white foam (303 mg). TFA (0.75 mL) was added to a solution of this material in DCM (2.5 mL) at RT and the mixture was stirred at RT for 10 min. The mixture was concentrated to dryness, the residue was dissolved in MeOH and passed through a cation exchange resin cartridge (Porapak Rxn CX 20 cc). A solution of 3% NH₃ in MeOH was used to elute the desired product as the free base as an off white solid. (220 mg). LCMS [M+H]⁺=526.6

### Step 9: Isopropyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate

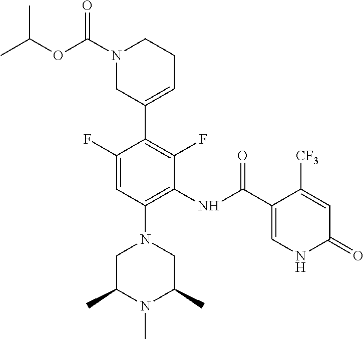 [SMILES: CC(C)OC(=O)N1CCC=C(c2c(F)cc(N3C[C@@H](C)N(C)[C@@H](C)C3)c(NC(=O)c3c[nH]c(=O)cc3C(F)(F)F)c2F)C1]

N-(2,4-Difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide (28 mg, 0.053 mmol) and N,N-diisopropylethylamine (0.019 mL, 0.107 mmol) in DCM (4 mL) at RT, was added isopropyl chloroformate (0.050 ml, 0.050 mmol) dropwise over a period of 10 min. After approximately 5 min, the mixture was partitioned between DCM (2 mL) and water (4 mL), the org phase was separated, aq. phase was extracted with DCM (3 mL), the combined org phase was washed with brine, dried over Na₂SO₄, concentrated onto celite and purified by silica gel chromatography, eluting with DCM containing 0-5% MeOH and 0-0.5% NH₄OH. The title compound was isolated as an off-white powder (25 mg, 73% yield).

### Example 14:1-Methylcyclobutyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate

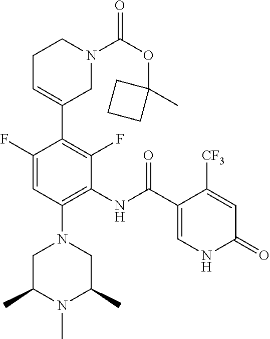 [SMILES: C[C@@H]1CN(c2cc(F)c(C3=CCCN(C(=O)OC4(C)CCC4)C3)c(F)c2NC(=O)c2c[nH]c(=O)cc2C(F)(F)F)C[C@H](C)N1C]

A solution of the TFA salt of N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide (28 mg, 0.044 mmol, prepared according to the procedure described in Example 1, Step 6), 1-methylcyclobutyl (4-nitrophenyl) carbonate (28.00 mg, 0.111 mmol) was charged with pyridine (0.5 ml) followed by N,N-diisopropylethylamine (16.98 mg, 0.131 mmol). The mixture was stirred at 90° C. in a heating block for approximately 30 min. The mixture was concentrated with celite and purified by reverse phase ACN/water and lyophilized to obtain the desired product (0.021 mmol, 47.6% yield), as a pale yellow solid Example 15: N-(2,4-difluoro-3-(1-(pyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide

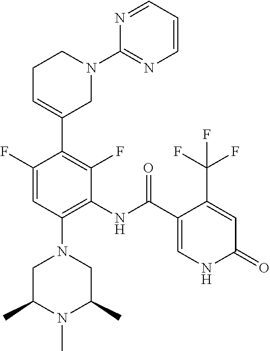 [SMILES: C[C@@H]1CN(c2cc(F)c(C3=CCCN(c4ncccn4)C3)c(F)c2NC(=O)c2c[nH]c(=O)cc2C(F)(F)F)C[C@H](C)N1C]

A solution of the TFA salt of N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide (32 mg, 0.050 mmol, prepared as described in Example 13) and 2-bromopyrimidine 95% (11.93 mg, 0.075 mmol) in isopropanol (2 ml) and N,N-diisopropylethylamine (19.40 mg, 0.150 mmol) was heated in a microwave at 100° C. for 45 min. The mixture was concentrated and purified by reverse phase ACN/water to afford the title compound (0.038 mmol, 75% yield), as a pale yellow powder.

### Example 16: N-(2,4-difluoro-3-(2-((S)-2-methylmorpholino)pyrimidin-5-yl)-6-((3S,5R)-3,4,5-trimethylpiperazin-1-yl)phenyl)-4-fluoro-2-(trifluoromethyl)benzamide formic acid

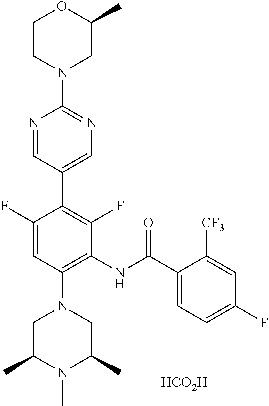 [SMILES: C[C@@H]1CN(c2cc(F)c(-c3cnc(N4CCO[C@@H](C)C4)nc3)c(F)c2NC(=O)c2ccc(F)cc2C(F)(F)F)C[C@H](C)N1C ; "HCO2H"]

### Step 1: N-(3-bromo-2,4-difluoro-6-((3S,5R)-3,4,5-trimethylpiperazin-1-yl)phenyl)-4-fluoro-2-(trifluoromethyl)benzamide

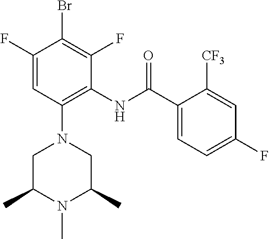 [SMILES: C[C@@H]1CN(c2cc(F)c(Br)c(F)c2NC(=O)c2ccc(F)cc2C(F)(F)F)C[C@H](C)N1C]

To a solution of 4-fluoro-2-(trifluoromethyl)benzoyl chloride (0.23 mL, 1.5 mmol) in DCM (10 mL) at RT was added Et₃N (0.42 mL, 3 mmol). After addition, the resulting mixture was stirred at RT for 5 min, before a solution of 3-bromo-2,4-difluoro-6-((3S,5R)-3,4,5-trimethylpiperazin-1-yl)aniline (167 mg, 0.5 mmol) in DCM (10 mL) was added. The resulting mixture was stirred at RT for 2.5 h. After quenching with sat. NaHCO₃ (15 mL) and stirring for 10 min at RT, the mixture was extracted with DCM (20 mL×2). The extracts were combined, concentrated and purified by flash chromatography (gradient: EtOAc/hex 0-100% then MeOH/DCM 0-10%) to give N-(3-bromo-2,4-difluoro-6-((3S,5R)-3,4,5-trimethylpiperazin-1-yl)phenyl)-4-fluoro-2-(trifluoromethyl)benzamide as a light brown solid (240 mg, 84% based on 92.2% purity). LCMS [M+H]⁺ 524.3.

### Step 2: N-(2,4-difluoro-3-(2-((S)-2-methylmorpholino)pyrimidin-5-yl)-6-((3S,5R)-3,4,5-trimethylpiperazin-1-yl)phenyl)-4-fluoro-2-(trifluoromethyl)benzamideformic Acid

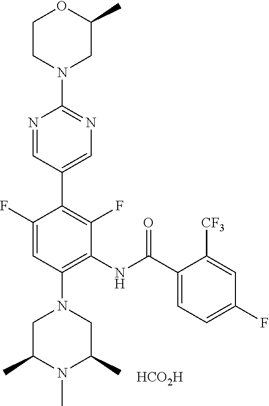 [SMILES: C[C@@H]1CN(c2cc(F)c(-c3cnc(N4CCO[C@@H](C)C4)nc3)c(F)c2NC(=O)c2ccc(F)cc2C(F)(F)F)C[C@H](C)N1C ; "HCO2H"]

The title compound (formic acid salt, pale beige solid, 28.0 mg, 42%) was prepared according to a method similar that described in Example 1, Step 6 using crude (S)-(2-(2-methylmorpholino)pyrimidin-5-yl)boronic acid (0.3 mmol×2) and N-(3-bromo-2,4-difluoro-6-((3S,5R)-3,4,5-trimethylpiperazin-1-yl)phenyl)-4-fluoro-2-(trifluoromethyl)benzamide (57 mg, 92.2% purity, 0.1 mmol). LCMS [M+H]⁺ 623.4.

### Example 17: N-(2,4-difluoro-3-(2-((R)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide formic acid

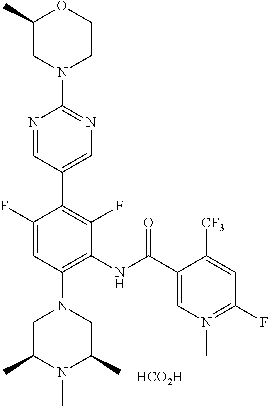

### Step 1: N-(2,4-difluoro-3-(2-((R)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide Formic Acid

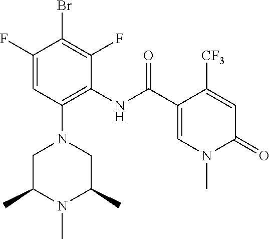 [SMILES: C[C@@H]1CN(c2cc(F)c(Br)c(F)c2NC(=O)c2cn(C)c(=O)cc2C(F)(F)F)C[C@H](C)N1C]

To a 25 mL RBF charged with 1-methyl-6-oxo-4-(trifluoromethyl)-1,6-dihydropyridine-3-carboxylic acid (166 mg, 0.75 mmol) was added thionyl chloride (1.09 mL, 30 mmol). The resulting suspension was heated at 80° C. for 1 h and evaporated to give a pale yellow oil which solidified to a white solid. This material was treated with 3-bromo-2,4-difluoro-6-((3S,5R)-3,4,5-trimethylpiperazin-1-yl)aniline (167 mg, 0.5 mmol), DCM (10 mL), followed by Et₃N (0.28 mL, 2 mmol). The resulting red/brown solution was stirred at RT for 2 h. After quenching with sat. NaHCO₃ (15 mL) and stirring for 10 min at RT, the mixture was extracted with DCM (20 mL×2). The extracts were combined, concentrated and purified by flash chromatography (gradient: EtOAc/hex 0-100% then MeOH/DCM 0-10%) to give a beige crystalline solid (80 mg, 28% based on 94.9% purity). LCMS [M+H]⁺ 537.3.

### Step 2: N-(2,4-difluoro-3-(2-((R)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide Formic Acid

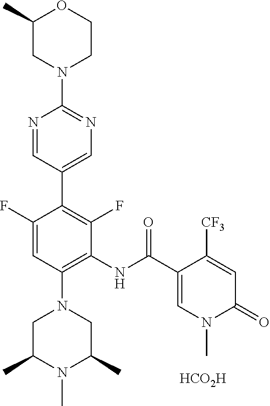 [SMILES: C[C@@H]1CN(c2ncc(-c3c(F)cc(N4C[C@@H](C)N(C)[C@@H](C)C4)c(NC(=O)c4cn(C)c(=O)cc4C(F)(F)F)c3F)cn2)CCO1 ; "HCO2H"]

The title compound (formic acid salt, pale beige solid, 26.0 mg, 54%) was prepared according to a procedure similar to Example 1, Step 6 using crude (R)-(2-(2-methylmorpholino)pyrimidin-5-yl)boronic acid (0.3 mmol×2) and N-(3-bromo-2,4-difluoro-6-((3S,5R)-3,4,5-trimethylpiperazin-1-yl)phenyl)-1-methyl-6-oxo-4-(trifluoromethyl)-1,6-dihydropyridine-3-carboxamide (40 mg, 94.9% purity, 0.07 mmol).

### Example 18: N-(2,4-difluoro-3-(2-((R)-2-methylmorpholino)pyrimidin-5-yl)-6-((3S,5R)-3,4,5-trimethylpiperazin-1-yl)phenyl)-4-fluoro-2-(trifluoromethyl)benzamide formic acid

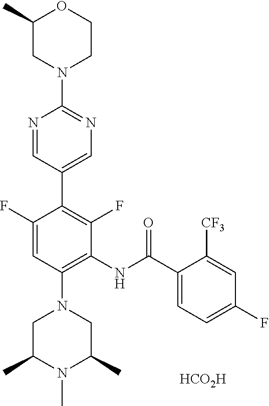 [SMILES: C[C@@H]1CN(c2ncc(-c3c(F)cc(N4C[C@@H](C)N(C)[C@@H](C)C4)c(NC(=O)c4ccc(F)cc4C(F)(F)F)c3F)cn2)CCO1 ; "HCO2H"]

The title compound (formic acid salt, light beige solid, 17.6 mg, 26%) was prepared according to the procedure described in Example 1, Step 6 using crude (R)-(2-(2-methylmorpholino)pyrimidin-5-yl)boronic acid (0.3 mmol×2) and N-(3-bromo-2,4-difluoro-6-((3S,5R)-3,4,5-trimethylpiperazin-1-yl)phenyl)-4-fluoro-2-(trifluoromethyl)benzamide (57 mg, 92.2% purity, 0.1 mmol, prepared according to Example 16).

### Example 19: N-(2,4-difluoro-3-(1-pivaloyl-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide

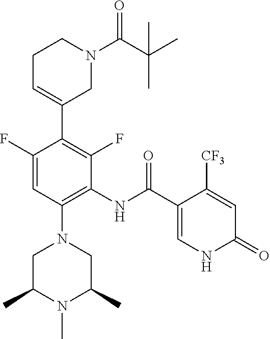 [SMILES: C[C@@H]1CN(c2cc(F)c(C3=CCCN(C(=O)C(C)(C)C)C3)c(F)c2NC(=O)c2c[nH]c(=O)cc2C(F)(F)F)C[C@H](C)N1C]

A solution of the TFA salt of N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide (25 mg, 0.039 mmol), pivaloyl chloride (4.71 mg, 0.039 mmol) in pyridine (0.5 mL) and N,N-diisopropylethylamine (5.05 mg, 0.039 mmol) was stirred at 23° C. for 15 min. The material was absorbed onto celite, concentrated and purified by reverse phase chromatography, eluting with ACN/water to afford after lyophilization the desired product (12.5 mg, 49.8% yield) as a white solid.

### Example 20: 3,3-Difluorocyclobutyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate

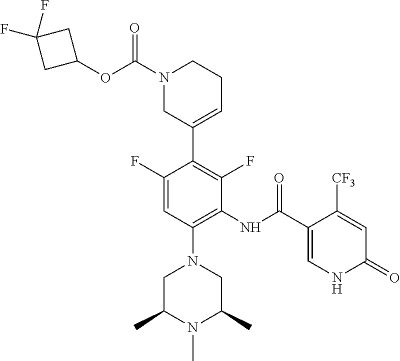 [SMILES: C[C@@H]1CN(c2cc(F)c(C3=CCCN(C(=O)OC4CC(F)(F)C4)C3)c(F)c2NC(=O)c2c[nH]c(=O)cc2C(F)(F)F)C[C@H](C)N1C]

The procedure was similar to Example 14 using N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide (25 mg, 0.048 mmol) and 3,3-difluorocyclobutyl (4-nitrophenyl) carbonate (14.30 mg, 0.052 mmol). The title compound was isolated as an off-white powder (27.5 mg, 83%).

### Example 21: N-(2,4-difluoro-3-(1-(pyrimidin-2-yl)-1,2,5,6-tetrahydropyridin-3-yl)-6-((3S,5R)-3,4,5-trimethylpiperazin-1-yl)phenyl)-4-(difluoromethyl)-6-oxo-1,6-dihydropyridine-3-carboxamide

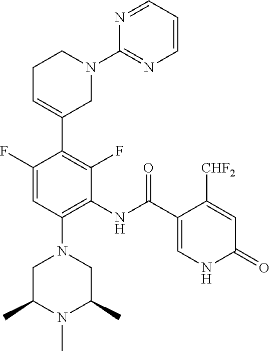 [SMILES: C[C@@H]1CN(c2cc(F)c(C3=CCCN(c4ncccn4)C3)c(F)c2NC(=O)c2c[nH]c(=O)cc2C(F)F)C[C@H](C)N1C]

### Step 1: 5-Bromo-2-(2-(trimethylsilyl)ethoxy)pyridine

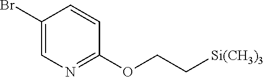

To a stirred solution of TMS ethanol (16.23 ml, 194.8 mmol, 1.5 eq) in dry THF (500 ml) was added NaH (4.68 g, 195.0 mmol, 1.5 eq) at 0° C. under argon. The mixture was stirred for 30 min and 5-bromo-2-chloropyridine (25 g, 130.2 mmol, 1 eq) in dry THF (125 ml) was added. The mixture was then slowly warmed and heated at reflux for 24 h. TLC analysis indicated formation of less polar spot along with 10% of SM. The reaction mixture cooled to RT, poured into ice water, extracted with EtOAc (2×500 ml) and washed with water (2×250 ml) followed by brine (2×250 mL). The organic layers were combined and dried over Na₂SO₄ and concentrated under reduced pressure to give the crude product which was purified by silica gel chromatography (260-400 mesh) using 100% pet ether as an eluent to give 5-bromo-2-(2-(trimethylsilyl)ethoxy)pyridine (22 g, 64% yield) as a pale yellow liquid. TLC: 10% EtOAc in Pet Ether; R_{f}: 0.8

### Step 2: 5-Bromo-2-(2-(trimethylsilyl)ethoxy)isonicotinaldehyde

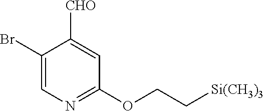

To a solution of DiPA (5.76 ml, 57.0 mmol, 1.5 eq) in dry THF (30 ml) was added n-BuLi (2.5M in n-hexane, 15.2 ml, 38.09 mmol, 1.3 eq.) at −78° C. The mixture was allowed to −30° C. over 30 min. Freshly prepared LDA was added a solution of 5-bromo-2-(2-(trimethylsilyl)ethoxy)pyridine (8 g, 29.3 mmol, 1eq) in dry THF (200 mL) at −78° C. under an Argon atm and maintained for 1 h. The mixture was then quenched by the dropwise addition of DMF (2.38 g, 32.23 mmol, 1.1 eq) over 10 min. TLC analysis indicated formation of polar spots. The reaction mixture was quenched with sat.NH₄Cl (50 mL) and extracted with EtOAc (4×200 mL) washed with water and brine. The combined organic layers were dried over Na₂SO₄ and concentrated under reduced pressure to give crude 5-bromo-2-(2-(trimethylsilyl)ethoxy)isonicotinaldehyde (7.8 g, 88.6% yield) as a pale yellow liquid. The crude product was used without further purification. TLC: 5% EtOAc in pet ether; R_{f}: 0.6

### Step 3: Methyl 4-formyl-6-(2-(trimethylsilyl)ethoxy)nicotinate

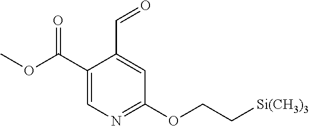

To a stirred solution of 5-bromo-2-(2-(trimethylsilyl)ethoxy)isonicotinaldehyde (7.8 g, 25.91 mmol, 1 eq.) in methanol (80 mL) was added TEA (36.35 ml, 259.1 mmol, 10 eq) at RT in a steel bomb degassed with argon for 10 min, then Pd₂(dppf)Cl₂DCM (2.11 g, 2.59 mmol, 0.1 eq) was added and the mixture was heated to 70° C. under 250 Psi (CO gas) for 16 h. TLC analysis indicated formation of polar spots. The reaction mixture was filtered through celite bed washed with methanol; the filtrate was evaporated under reduced pressure. The crude compound was purified by flash chromatography using 5% EtOAc in pet ether as an eluent to afford methyl 4-formyl-6-(2-(trimethylsilyl)ethoxy)nicotinate (3.1 g, 39.7%) as a pale yellow liquid. TLC: 5% EtOAc in pet ether; R_{f}: 0.5

### Step 4: Methyl 4-(difluoromethyl)-6-(2-(trimethylsilyl)ethoxy)nicotinate

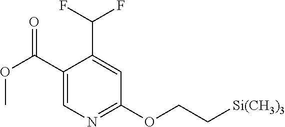

To a stirred solution of methyl 4-formyl-6-(2-(trimethylsilyl)ethoxy)nicotinate (6.1 g, 21.7 mmol, 1 eq) in DCM (60 mL) was added DAST (5.24 g, 32.56 mmol, 1.5 eq) at −78° C. under argon then slowly warmed to RT stirred for 16h. TLC analysis indicated formation of less polar spots. The reaction mixture was cooled to 0° C., quenched with satd.NaHCO₃ solution, extracted with DCM (2×200 mL), washed with water (2×100 ml) and brine (2×100 ml). The combined organic layers were dried over Na₂SO₄ and concentrated under reduced pressure to give the crude methyl 4-(difluoromethyl)-6-(2-(trimethylsilyl)ethoxy)nicotinate (6 g, 92.9% yield) as a pale yellow color liquid. The crude product was used without further purification. TLC: 5% EtOAc in pet ether; R_{f}: 0.6.

### Step 5: 4-(Difluoromethyl)-6-(2-(trimethylsilyl)ethoxy)nicotinic acid

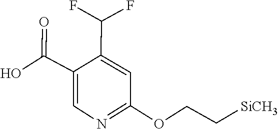

To a stirred solution of methyl 4-(difluoromethyl)-6-(2-(trimethylsilyl)ethoxy)nicotinate (6 g, 19.8 mmol, 1 eq.) in MeOH:THF:H₂O (30:30:10 mL) was added LiOH (1.66 g, 39.6 mmol, 2 eq.) at RT. The mixture was stirred for 16 h. TLC analysis indicated formation of polar spot. The solvent was evaporated under reduced pressure, the reaction mixture was cooled to 0° C. and acidified with 2N HCl. The mixture was extracted with EtOAc (2×100 ml) washed with water (2×50 mL) and brine (2×50 mL). The combined organic layers were dried over Na₂SO₄ and concentrated under reduced pressure to give crude product. The crude product was washed with pentane to obtain pure 4-(difluoromethyl)-6-(2-(trimethylsilyl)ethoxy)nicotinic acid (4.5 g, 78.7% yield) as an off white solid. TLC: 5% MeOH in DCM; R_{f}: 0.1.

### Step 6: N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide

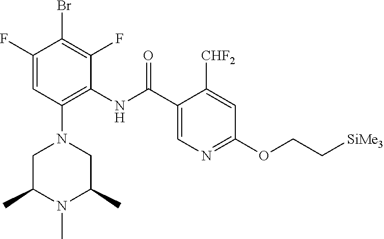 [SMILES: C[C@@H]1CN(c2cc(F)c(Br)c(F)c2NC(=O)c2cnc(OCC[Si](C)(C)C)cc2C(F)F)C[C@H](C)N1C]

To a stirred solution of 4-(difluoromethyl)-6-(2-(trimethylsilyl)ethoxy)nicotinic acid (270 mg, 0.934 mmol), propylphosphonic anhydride solution (743 mg, 1.167 mmol) in pyridine (1 mL) was added N,N-diisopropylethylamine (402 mg, 3.11 mmol). After stirring the mixture at 55° C. for 40 min, a dilute solution of 3-bromo-2,4-difluoro-6-((3S,5R)-3,4,5-trimethylpiperazin-1-yl)aniline (260 mg, 0.778 mmol) in pyridine (1 mL) was added. The mixture was then stirred at 80° C. for 30 min. Additional portions (3) of propylphosphonic anhydride (743 mg, 1.167 mmol) were added with continued LCMS monitoring until complete conversion of product was seen. The mixture was allowed to cool to 23° C., worked up with EtOAc (20 mL) and ice-cold Na₂CO₃ (saturated aqueous) and water. The organic layer was washed with water and concentrated to dryness. Purification by reverse phase (ACN/water) chromatography afforded the desired product (0.548 mmol, 70.5% yield) as a brown solid.

### Step 7: N-(2,4-Difluoro-3-(1,2,5,6-tetrahydropyridin-3-yl)-6-((3S,5R)-3,4,5-trimethylpiperazin-1-yl)phenyl)-4-(difluoromethyl)-6-oxo-1,6-dihydropyridine-3-carboxamide-TFA salt

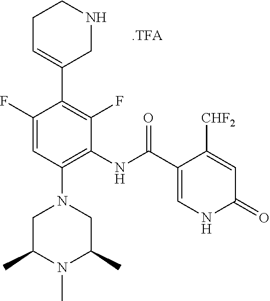 [SMILES: C[C@@H]1CN(c2cc(F)c(C3=CCCNC3)c(F)c2NC(=O)c2c[nH]c(=O)cc2C(F)F)C[C@H](C)N1C]

To a mixture of 1-boc-5,6-dihydro-2H-pyridine-3-boronic acid, pinacol ester (276 mg, 0.892 mmol), N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (360 mg, 0.595 mmol), bis(di-tert-butyl(4-dimethylaminophenyl)phosphine)dichloropalladium(II) (42.1 mg, 0.059 mmol) and 1,4-dioxane (15 mL) was added a solution of potassium phosphate tribasic reagent grade, >=98% (252 mg, 1.189 mmol) in water (2 mL). The mixture was degassed for 5 min and heated in a microwave at 100° C. for 45 min. After cooling to RT, the mixture was diluted with EtOAc (10 mL), the organic layer was concentrated and the crude product was purified by reverse phase chromatography (ACN/water 13 g column, 30 min elution) to provide the intermediate tert-butyl 5-(3-(4-(difluoromethyl)-6-(2-(trimethylsilyl)ethoxy)nicotinamido)-2,6-difluoro-4-((3S,5R)-3,4,5-trimethylpiperazin-1-yl)phenyl)-3,6-dihydropyridine-1(2H)-carboxylate (0.247 mmol, 41.5% yield), as a brown solid. This material was treated with 2 mL of 1/1 TFA/DCM at RT for 2 h to afford, after silica gel chromatography, the desired product (0.168 mmol, 28.3% yield) as a pale brown solid.

### Step 8: N-(2,4-difluoro-3-(1-(pyrimidin-2-yl)-1,2,5,6-tetrahydropyridin-3-yl)-6-((3S,5R)-3,4,5-trimethylpiperazin-1-yl)phenyl)-4-(difluoromethyl)-6-oxo-1,6-dihydropyridine-3-carboxamide

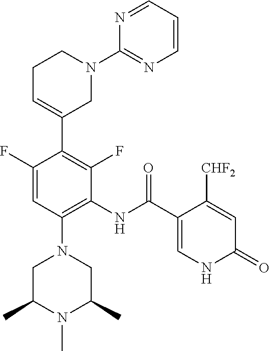 [SMILES: C[C@@H]1CN(c2cc(F)c(C3=CCCN(c4ncccn4)C3)c(F)c2NC(=O)c2c[nH]c(=O)cc2C(F)F)C[C@H](C)N1C]

A mixture of the TFA salt of N-(2,4-difluoro-3-(1,2,5,6-tetrahydropyridin-3-yl)-6-((3S,5R)-3,4,5-trimethylpiperazin-1-yl)phenyl)-4-(difluoromethyl)-6-oxo-1,6-dihydropyridine-3-carboxamide (28 mg, 0.045 mmol, prepared in Step 1 above), 2-bromopyrimidine 95% (10.74 mg, 0.068 mmol), 2-propanol (2 mL) and N,N-diisopropylethylamine (17.47 mg, 0.135 mmol) was heated at 100° C. using a heating block for 45 min. The mixture was absorbed onto celite, concentrated and purified by reverse phase ACN/water to get the desired product (0.023 mmol, 50.4% yield) as a white solid. LCMS [M+H]⁺ 586.5.

### Example 22: 1-Methylcyclobutyl 5-(3-(4-(difluoromethyl)-6-oxo-1,6-dihydropyridine-3-carboxamido)-2,6-difluoro-4-((3S,5R)-3,4,5-trimethylpiperazin-1-yl)phenyl)-3,6-dihydropyridine-1(2H)-carboxylate

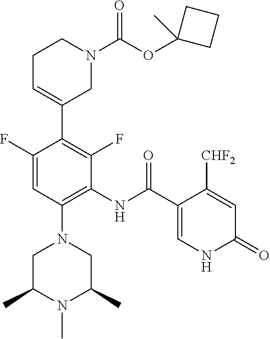 [SMILES: C[C@@H]1CN(c2cc(F)c(C3=CCCN(C(=O)OC4(C)CCC4)C3)c(F)c2NC(=O)c2c[nH]c(=O)cc2C(F)F)C[C@H](C)N1C]

A solution of the TFA salt of N-(2,4-difluoro-3-(1,2,5,6-tetrahydropyridin-3-yl)-6-((3S,5R)-3,4,5-trimethylpiperazin-1-yl)phenyl)-4-(difluoromethyl)-6-oxo-1,6-dihydropyridine-3-carboxamide (28 mg, 0.045 mmol, preparation in Example 21), 1-methylcyclobutyl (4-nitrophenyl) carbonate (11.3 mg, 0.045 mmol), pyridine (0.5 ml) and N,N-diisopropylethylamine (17.47 mg, 0.135 mmol) was employed in a procedure similar to Example 14 to afford the desired product (0.023 mmol, 51.0% yield), as a white solid. LCMS [M+H]⁺ 620.6.

### Example 23: Isopropyl 5-(3-(4-(difluoromethyl)-6-oxo-1,6-dihydropyridine-3-carboxamido)-2,6-difluoro-4-((3S,5R)-3,4,5-trimethylpiperazin-1-yl)phenyl)-3,6-dihydropyridine-1(2H)-carboxylate

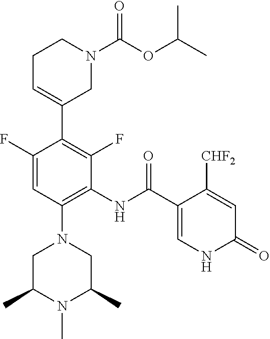 [SMILES: CC(C)OC(=O)N1CCC=C(c2c(F)cc(N3C[C@@H](C)N(C)[C@@H](C)C3)c(NC(=O)c3c[nH]c(=O)cc3C(F)F)c2F)C1]

The procedure was similar to Example 19 using N-(2,4-difluoro-3-(1,2,5,6-tetrahydropyridin-3-yl)-6-((3S,5R)-3,4,5-trimethylpiperazin-1-yl)phenyl)-4-(difluoromethyl)-6-oxo-1,6-dihydropyridine-3-carboxamide TFA salt (28 mg, 0.045 mmol), isopropyl chloroformate (5.52 mg, 0.045 mmol) and pyridine (0.5 mL) in N,N-diisopropylethylamine (17.47 mg, 0.135 mmol) to give the title compound (10.72 μmol, 23.80% yield), as a white solid. LCMS [M+H]⁺ 594.6.

### Example 24: 6-Oxo-N-(2,3′,6-trifluoro-4′-(methylcarbamoyl)-4-((3S,5R)-3,4,5-trimethylpiperazin-1-yl)-[1,1′-biphenyl]-3-yl)-4-(trifluoromethyl)-1,6-dihydropyridine-3-carboxamide

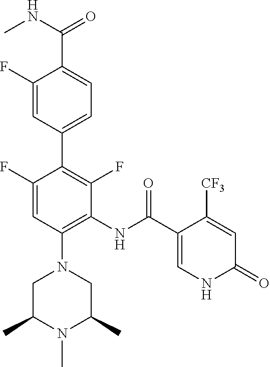 [SMILES: CNC(=O)c1ccc(-c2c(F)cc(N3C[C@@H](C)N(C)[C@@H](C)C3)c(NC(=O)c3c[nH]c(=O)cc3C(F)(F)F)c2F)cc1F]

N-(3-Bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (50 mg, 0.080 mmol), 3-Fluoro-4-(methylcarbamoyl)phenylboronic acid (31.6 mg, 0.160 mmol) and potassium phosphate tribasic reagent grade, >=98% (51.1 mg, 0.241 mmol) were placed in a small microwave vial. 1,4-Dioxane (4 ml) and water (1.000 ml) were added and the mixture was stirred at ambient temperature, followed by addition of bis(di-tert-butyl(4-dimethylaminophenyl)phosphine)dichloropalladium(II) (5.68 mg, 8.02 μmol). The reaction mixture was purged with N₂ the vial was sealed and the mixture was heated in the microwave at 100° C. for 1 h. The mixture was loaded on celite, concentrated and purified by reverse phase chromatography (C18 13.3 g cartridge eluent:10%, 10-100%, then 100% AcCN/water) to afford the intermediate (43.4 mg, 0.062 mmol) as an off-white solid. This material was dissolved in DCM (1.5 mL) then TFA (1.5 ml) was added. The mixture was stirred at RT for 30 min. The solvents were evaporated off in the rotavap. The residue was taken in some acetonitrile, some water was added. It was freezed then lyophilized to afford the product (53 mg, 97% yield) as a white fluffy powder.

### Example 25: N-(4′-carbamoyl-2,3′,6-trifluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide

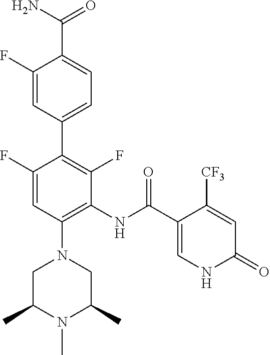 [SMILES: C[C@@H]1CN(c2cc(F)c(-c3ccc(C(N)=O)c(F)c3)c(F)c2NC(=O)c2c[nH]c(=O)cc2C(F)(F)F)C[C@H](C)N1C]

N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (50 mg, 0.080 mmol), 4-carbamoyl-3-fluorophenylboronic acid, 96% (29.3 mg, 0.160 mmol) and potassium phosphate tribasic reagent grade, >=98% (51.1 mg, 0.241 mmol) were charged in a small microwave vial. 1,4-Dioxane (4 mL) and water (1.0 mL) were added then the mixture was stirred at rt. Bis(di-tert-butyl(4-dimethylaminophenyl)phosphine)dichloropalladium(II) (5.68 mg, 8.02 μmol) was added, the mixture was purged with N₂ then the vial was sealed and heated in a microwave at 100° C. for 1h. Purification using reverse phase chromatography (C18 13.3 g cartridge eluent:10%, 10-100%, then 100% AcCN/water) afforded the silyloxy intermediate (42 mg) as a beige foamy solid. This material was dissolved in DCM (1.5 ml) then TFA (1.5 ml) was added. The mixture was stirred at RT for 30 min. Isolation using methods described in earlier examples afforded the title compound (48.2 mg, 92% yield) as a light purple fluffy powder.

### Example 26: N-(4′-carbamoyl-2,2′,3′,6-tetrafluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide

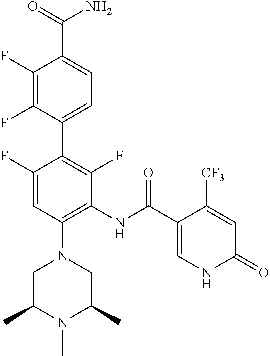 [SMILES: C[C@@H]1CN(c2cc(F)c(-c3ccc(C(N)=O)c(F)c3F)c(F)c2NC(=O)c2c[nH]c(=O)cc2C(F)(F)F)C[C@H](C)N1C]

N-(3-Bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (50 mg, 0.080 mmol), 2,3-difluoro-4-(4,4,5,5-tetramethyl-1,3,2-dioxaborolan-2-yl)-N-(2,4,4-trimethylpentan-2-yl)benzamide (69.7 mg, 0.176 mmol) and potassium phosphate tribasic reagent grade, >=98% (51.1 mg, 0.241 mmol) were placed in a small microwave vial. 1,4-Dioxane (4 mL) and water (1.0 mL) were added and the mixture was stirred at ambient temperature. Bis(di-tert-butyl(4-dimethylaminophenyl)phosphine)dichloropalladium(II) (5.68 mg, 8.02 μmol) was added, the reaction vessel was purged with N₂ and the vial was sealed. It was then heated in the microwave at 100° C. for 1h. The silyloxy intermediate (42 mg, 0.052 mmol), which was isolated using methods similar to Example 25 was dissolved in DCM (1.5 ml) then TFA (1.5 ml) was added. The mixture was heated at 62° C. for about 4 h. The solvents were removed and the residue was dissolved in acetonitrile-water mixture and lyophilized to afford the title compound (TFA salt) as an off-white solid (39.9 mg, 0.050 mmol, 97% yield); LCMS [M+H]⁺ 600.

### Example 27:6-Oxo-N-(2,2′,3′,6-tetrafluoro-4′-((2,4,4-trimethylpentan-2-yl)carbamoyl)-4-((3S,5R)-3,4,5-trimethylpiperazin-1-yl)-[,1′-biphenyl]-3-yl)-4-(trifluoromethyl)-1,6-dihydropyridine-3-carboxamide

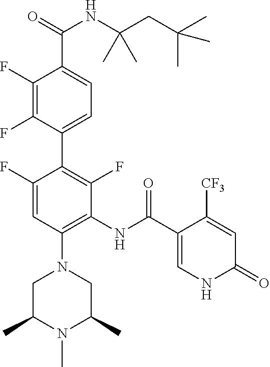 [SMILES: C[C@@H]1CN(c2cc(F)c(-c3ccc(C(=O)NC(C)(C)CC(C)(C)C)c(F)c3F)c(F)c2NC(=O)c2c[nH]c(=O)cc2C(F)(F)F)C[C@H](C)N1C]

N-(2,2′,3′,6-Tetrafluoro-4′-((2,4,4-trimethylpentan-2-yl)carbamoyl)-4-((3S,5R)-3,4,5-trimethylpiperazin-1-yl)-[1,1′-biphenyl]-3-yl)-4-(trifluoromethyl)-6-(2-(trimethylsilyl)ethoxy)nicotinamide (13.9 mg, 0.017 mmol, obtained as an intermediate in Example 26) was dissolved in DCM (1 ml) then TFA (0.5 ml) was added. The mixture was stirred at RT for 10 min. The solvents were evaporated and the residue was dissolved in an acetonitrile-water mixture and lyophilized to afford the title compound (TFA salt) as a white fluffy powder (11.3 mg, 76% yield). LCMS [M+H]⁺ 712.

### Example 28: N-(3′-carbamoyl-2,4′,6-trifluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide

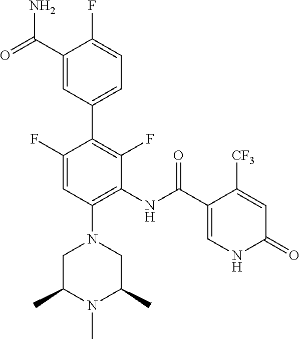 [SMILES: C[C@@H]1CN(c2cc(F)c(-c3ccc(F)c(C(N)=O)c3)c(F)c2NC(=O)c2c[nH]c(=O)cc2C(F)(F)F)C[C@H](C)N1C]

The procedure followed was similar to Example 25 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (53 mg, 0.085 mmol) and 3-carbamoyl-4-fluorophenylboronic acid, 97% (31.1 mg, 0.170 mmol) to give the silyloxy intermediate (32.7 mg). Deprotection with TFA in a manner similar to that described in Example 25 afforded the title compound as off-white fluffy powder (35 mg, 0.046 mmol, 96% yield). LCMS [M+H]⁺ 582.

### Example 29: 6-Oxo-N-(2,4′,6-trifluoro-3′-(methylcarbamoyl)-4-((3S,5R)-3,4,5-trimethylpiperazin-1-yl)-[1,1′-biphenyl]-3-yl)-4-(trifluoromethyl)-1,6-dihydropyridine-3-carboxamide

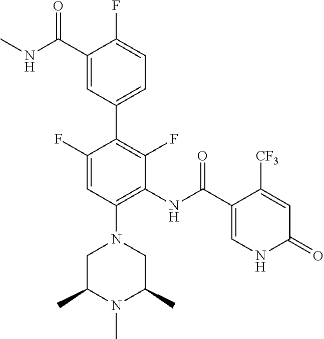 [SMILES: CNC(=O)c1cc(-c2c(F)cc(N3C[C@@H](C)N(C)[C@@H](C)C3)c(NC(=O)c3c[nH]c(=O)cc3C(F)(F)F)c2F)ccc1F]

A procedure similar to that of Example 25 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (53 mg, 0.085 mmol) and N-methyl-5-borono-2-fluorobenzamide (33.5 mg, 0.170 mmol) to give the silyloxy intermediate (47 mg). Deprotection with TFA and purification as described in Example 25 afforded the title compound as an off-white fluffy powder (50.3 mg, 0.054 mmol, 79% yield). LCMS [M+H]⁺ 596.

### Example 30: N-(5′-carbamoyl-2,2′,4′,6-tetrafluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide

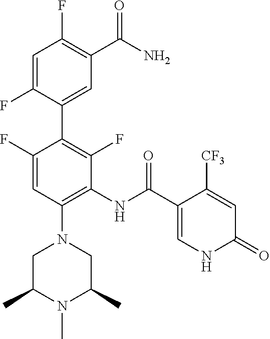 [SMILES: C[C@@H]1CN(c2cc(F)c(-c3cc(C(N)=O)c(F)cc3F)c(F)c2NC(=O)c2c[nH]c(=O)cc2C(F)(F)F)C[C@H](C)N1C]

A procedure similar to Example 25 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (53 mg, 0.085 mmol), 2,4-difluoro-5-(4,4,5,5-tetramethyl-1,3,2-dioxaborolan-2-yl)-N-(2,4,4-trimethylpentan-2-yl)benzamide (67.2 mg, 0.170 mmol) to afford the intermediate N-(2,2′,4′,6-tetrafluoro-5′-((2,4,4-trimethylpentan-2-yl)carbamoyl)-4-((3S,5R)-3,4,5-trimethylpiperazin-1-yl)-[1,1′-biphenyl]-3-yl)-4-(trifluoromethyl)-6-(2-(trimethylsilyl)ethoxy)nicotinamide (7 mg, 8.62 μmol). This material was dissolved in DCM (1 mL) then TFA (1 mL) was added. The mixture was stirred for 3 h at 62° C. The solvents were evaporated under reduced pressure. The residue was dissolved in an acetonitrile-water mixture and lyophilized to afford the title compound (TFA salt) as an off-white fluffy solid (5.7 mg, 7.59 μmol, 88% yield for the last step). LCMS [M+H]⁺ 600.

### Example 31: N-(2,4-difluoro-3-(2-morpholinopyrimidin-5-yl)-6-((3S,5R)-3,4,5-trimethylpiperazin-1-yl)phenyl)-2-(difluoromethyl)-4-fluorobenzamideformic Acid

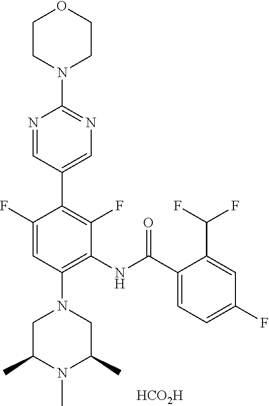 [SMILES: C[C@@H]1CN(c2cc(F)c(-c3cnc(N4CCOCC4)nc3)c(F)c2NC(=O)c2ccc(F)cc2C(F)F)C[C@H](C)N1C ; "HCO2H"]

To a mixture of 2-(difluoromethyl)-4-fluorobenzoic acid (34 mg, 0.18 mmol) and propylphosphonic anhydride solution (50% wt % in EtOAc, 0.12 mL, 0.2 mmol) in pyridine (0.2 mL) was added iPr₂NEt (0.070 mL, 0.4 mmol). The resulting mixture was stirred for 15 min at 55° C. before 2,4-difluoro-3-(2-morpholinopyrimidin-5-yl)-6-((3S,5R)-3,4,5-trimethylpiperazin-1-yl)aniline (42 mg, 0.1 mmol) was added in one portion. It was heated at 80° C. for 30 min. After quenching with sat. NaHCO₃ (3 mL), the mixture was extracted with EtOAc (3 mL×2). The combined extracts were concentrated and the residue was redissolved in DMSO (2 mL) with 3 drops of formic acid. It was filtered and purified using a Waters PREP-HPLC, column XSelect Prep C18 5 μM, 19×100 mm (column 1). The resulting residue collected from concentration of fractions showing pure product were dissolved in MeOH (10 mL) and treated with 2 drops of formic acid, concentrated and dried to give a white solid. LCMS [M+H]⁺ 519.3.

### Example 32: 2-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-4-fluorobenzamide Formic Acid

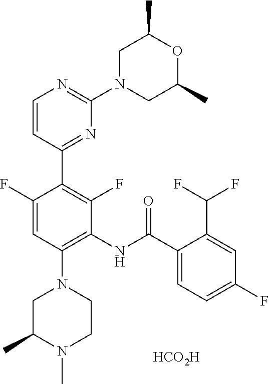 [SMILES: C[C@@H]1CN(c2nccc(-c3c(F)cc(N4CCN(C)[C@@H](C)C4)c(NC(=O)c4ccc(F)cc4C(F)F)c3F)n2)C[C@H](C)O1 ; "HCO2H"]

To a 20 mL microwave vial charged with (S)-3-bromo-6-(3,4-dimethylpiperazin-1-yl)-2,4-difluoroaniline (160 mg, 0.5 mmol), bis(pinacolato)diboron (254 mg, 1 mmol), Pd(dppf)Cl₂(18 mg, 0.025 mmol) and KOAc (147 mg, 1.5 mmol) was added dioxane (3 mL) and the resulting mixture was heated at 110° C. in microwave for 16 h. To the above mixture was added a solution of (2R,6S)-4-(4-bromopyrimidin-2-yl)-2,6-dimethylmorpholine (204 mg, 0.75 mmol), bis(di-tert-butyl(4-dimethylaminophenyl)phosphine)dichloropalladium(II) (18 mg, 0.05 mmol) and 1 M K₃PO₄ (1 mL, 1 mmol). The resulting mixture was heated in microwave at 110° C. for 2 h. After diluting with brine (10 mL), the mixture was extracted with EtOAc (30 mL×2). The combined extracts were concentrated and purified by Biotage SNAP KP-Sil 25 g (gradient: EtOAc/hex 0-100% then MeOH/DCM 0-20%) to give 3-(2-((2S,6R)-2,6-dimethylmorpholino)pyrimidin-4-yl)-6-((S)-3,4-dimethylpiperazin-1-yl)-2,4-difluoroaniline as a dark brown foam (110 mg, 46% based on 90.32% purity). LCMS [M+H]⁺ 433.3. The title compound (formic acid salt, beige solid, 32.7 mg, 40%) was prepared from 3-(2-((2S,6R)-2,6-dimethylmorpholino)pyrimidin-4-yl)-6-((S)-3,4-dimethylpiperazin-1-yl)-2,4-difluoroaniline (55 mg, 90.32% purity, 0.13 mmol), 2-(difluoro methyl)-4-fluorobenzoic acid (44 mg, 0.23 mmol) and T3P (50% wt % in EtOAc, 0.15 mL, 0.25 mmol) in a procedure similar to that of Example 31. LCMS [M+H]⁺ 605.4.

### Example 33:4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide

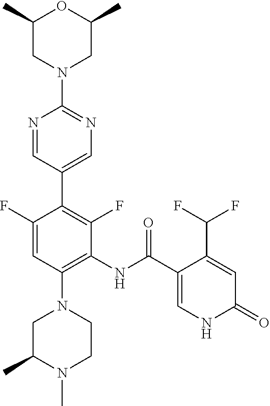 [SMILES: C[C@@H]1CN(c2ncc(-c3c(F)cc(N4CCN(C)[C@@H](C)C4)c(NC(=O)c4c[nH]c(=O)cc4C(F)F)c3F)cn2)C[C@H](C)O1]

### Step 1: 2-Bromo-1,3,5-trifluoro-4-nitrobenzene

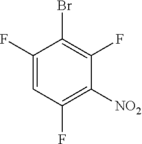 [SMILES: O=[N+]([O-])c1c(F)cc(F)c(Br)c1F]

A stirred solution of 2-bromo-1,3,5-trifluorobenzene (21 g, 100.03 mmol, 1 eq) in H₂SO₄ (105 mL) was cooled to 0° C. and HNO₃ (84 mL) was added dropwise. The resulting mixture was stirred for 2 h. TLC analysis indicated formation of non-polar spot. The mixture was quenched with ice water (500 mL) and extracted with ethyl acetate (2×500 mL). The combined organic layer was washed with sat. NaHCO₃ solution followed by brine solution, dried over Na₂SO₄ and concentrated under reduced pressure to afford 2-bromo-1,3,5-trifluoro-4-nitrobenzene (21 g, 82.3% yield) as yellow liquid. TLC: 10% EtOAc in pet ether: R_{f}: 0.4.

### Step 2: (S)-1-(4-Bromo-3,5-difluoro-2-nitrophenyl)-3-methylpiperazine

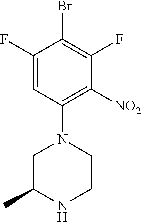 [SMILES: C[C@H]1CN(c2cc(F)c(Br)c(F)c2[N+](=O)[O-])CCN1]

To a stirred solution of 1,3,5-trifluoro-4-nitrobenzene (21 g, 82.38 mmol, 1 eq.) in ethanol (420 mL), was added DIPEA (42.86 mL, 296.09 mmol, 3 eq.) followed by (S)-2-methylpiperazine (9.86 g, 98.43 mmol, 1.2 eq) The resulting reaction mixture was heated at 85° C. for 16 h. TLC analysis indicated formation of a polar spot. The reaction mixture was concentrated under reduced pressure and the crude compound was purified by column chromatography (silica gel 100-200) using 5% methanol in DCM as an eluent resulted (S)-1-(4-bromo-3,5-difluoro-2-nitrophenyl)-3-methylpiperazine (21 g, 76.3% yield) as yellow solid. LCMS: [M+H]⁺ 335.97.

### Step 3: (S)-4-(4-bromo-3,5-difluoro-2-nitrophenyl)-1,2-dimethylpiperazine

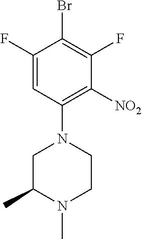 [SMILES: C[C@H]1CN(c2cc(F)c(Br)c(F)c2[N+](=O)[O-])CCN1C]

A stirred solution of (S)-1-(4-bromo-3,5-difluoro-2-nitrophenyl)-3-methylpiperazine (30 g, 89.24 mmol, 1eq) in DCM (510 mL) was cooled to 0° C. and 37% HCHO (36.48 mL, 356.9 mmol, 4 eq.) was added. The resulting mixture was stirred at RT for 2 h and cooled to 0° C. NaCNBH₃ (11.2 g, 178.4 mol, 2 eq.) was added portion wise and the mixture was stirred at RT for 16 h. TLC analysis indicated formation of non-polar spot. The reaction mixture was quenched with sat. NaHCO₃ solution and extracted with DCM (2×500 mL). The combined organic layers were dried over Na₂SO₄ and concentrated under reduced pressure. The crude compound was purified by column chromatography (silica gel 100-200) using 0-10% methanol in DCM as an eluent to afford (S)-4-(4-bromo-3,5-difluoro-2-nitrophenyl)-1,2-dimethylpiperazine (20 g, 63.5% yield) as yellow solid. TLC: 5% Methanol in DCM; R_{f}: 0.3.

### Step 4: (S)-3-bromo-6-(3,4-dimethylpiperazin-1-yl)-2,4-difluoroaniline

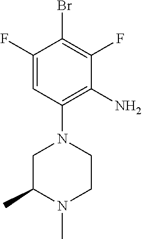 [SMILES: C[C@H]1CN(c2cc(F)c(Br)c(F)c2N)CCN1C]

To a stirred solution of (S)-4-(4-bromo-3,5-difluoro-2-nitrophenyl)-1,2-dimethylpiperazine (20 g, 57.1 mmol, 1 eq.) in ethanol: H₂O (340 mL: 60 mL) was added NH₄Cl (12.22 g, 228.4 mmol, 4 eq) followed by Fe powder (12.7 g, 228.4 mmol, 4eq). The resulting reaction mixture was stirred at RT for 16 h. TLC analysis indicated formation of non-polar spot. The reaction mixture was cooled to RT, filtered through celite and washed with EtOAc (200 mL). The filtrate was dried over Na₂SO₄ and concentrated under reduced pressure to give crude product which was purified by column chromatography (silica gel 100-200 mesh) using 0-10% methanol in DCM as an eluent to afford (S)-3-bromo-6-(3,4-dimethylpiperazin-1-yl)-2,4-difluoroaniline (12 g, 65.9% yield) as yellow solid. LCMS: [M+H]⁺ 320.44.

### Step 5: Synthesis of (S)—N-(3-bromo-6-(3,4-dimethylpiperazin-1-yl)-2,4-difluorophenyl)-4-(difluoromethyl)-6-(2-(trimethylsilyl)ethoxy)nicotinamide

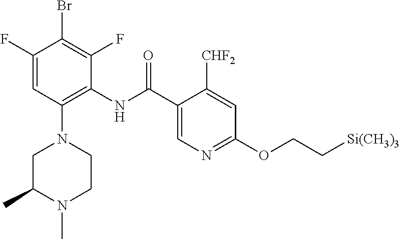

Propylphosphonic anhydride solution (1.67 ml, 2.81 mmol) was added dropwise to a solution of 4-(difluoromethyl)-6-(2-(trimethylsilyl)ethoxy)nicotinic acid (352 mg, 1.218 mmol) in pyridine (2 mL) under N₂ at RT. After stirring for 30 min at 50° C., (S)-3-bromo-6-(3,4-dimethylpiperazin-1-yl)-2,4-difluoroaniline (300 mg, 0.937 mmol) was added and the reaction mixture was stirred at 70° C. for 75 min. The reaction mixture was allowed to cool to RT, concentrated and partitioned between EtOAc and water. The organic phase was separated, aq. phase was extracted with EtOAc (×2), the combined organic phase was washed with 1N NaOH solution, brine, dried over Na₂SO₄ and concentrated to yield the title compound as a light brown solid (520 mg, 94%). LCMS [M+H]⁺ 593.4.

### Step 6:4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide

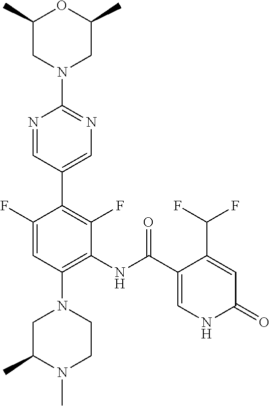 [SMILES: C[C@@H]1CN(c2ncc(-c3c(F)cc(N4CCN(C)[C@@H](C)C4)c(NC(=O)c4c[nH]c(=O)cc4C(F)F)c3F)cn2)C[C@H](C)O1]

The title compound (pale beige solid, 39.0 mg, 61% yield) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (2-((2S,6R)-2,6-dimethylmorpholino)pyrimidin-5-yl)boronic acid (47.4 mg, 0.2 mmol) followed by TFA deprotection (1 mL TFA/10 mL DCM). LCMS [M+H]⁺ 604.4.

### Example 34: Isopropyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

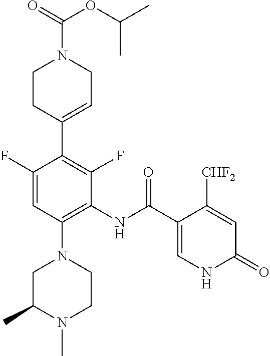 [SMILES: CC(C)OC(=O)N1CC=C(c2c(F)cc(N3CCN(C)[C@@H](C)C3)c(NC(=O)c3c[nH]c(=O)cc3C(F)F)c2F)CC1]

### Step 1: tert-Butyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

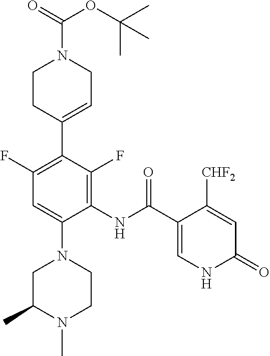 [SMILES: C[C@H]1CN(c2cc(F)c(C3=CCN(C(=O)OC(C)(C)C)CC3)c(F)c2NC(=O)c2c[nH]c(=O)cc2C(F)F)CCN1C]

(S)—N-(3-Bromo-6-(3,4-dimethylpiperazin-1-yl)-2,4-difluorophenyl)-4-(difluoromethyl)-6-(2-(trimethylsilyl)ethoxy)nicotinamide (147 mg, 0.249 mmol, preparation described in Example 33), tert-butyl4-(4,4,5,5-tetramethyl-1,3,2-dioxaborolan-2-yl)-1,2,3,6-tetrahydropyridine-1-carboxylate (100 mg, 0.323 mmol) and [1,1′-bis(diphenylphosphino)ferrocene]dichloropalladium(II) (23.64 mg, 0.032 mmol) were mixed in 1,4-dioxane (3 mL). Potassium phosphate tribasic reagent grade, >=98% (1.24 ml, 1.24 mmol) was added and the vial was flushed with nitrogen. The mixture was heated in a microwave reactor to 110° C. for 1.75 h. The mixture was worked up by partitioning with brine (10 mL) and 10 ml EtOAc, the org phase was separated, the aq. phase was extracted with EtOAc (8 m L×2). The combined extracts were dried over Na₂SO₄, concentrated and purified on sg column (4 g), eluting with hexanes containing 0-50% EA. The desired fractions were combined and concentrated to get the title compound as an off white foam (137 mg, 79% yield). LCMS [M+H]⁺ 512.32.

### Step 2: (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide

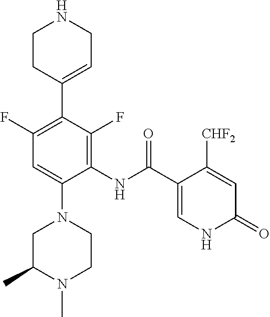 [SMILES: C[C@H]1CN(c2cc(F)c(C3=CCNCC3)c(F)c2NC(=O)c2c[nH]c(=O)cc2C(F)F)CCN1C]

TFA (0.5 mL) was added to a solution of tert-butyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate in DCM (1.5 mL) at RT and the mixture was stirred at RT. LCMS after 1 h showed completion of the reaction. The mixture was concentrated to dryness, the residue was dissolved in MeOH and passed through a cation exchange resin cartridge (Porapak Rxn CX 20 cc). A solution of 3% NH₃ in MeOH was used to elute the desired product as the free base. The desired fractions were combined and concentrated to get the title compound as an off white solid. (92 mg, 94% yield). LCMS [M−H]⁻ 492.4.

### Step 3: Isopropyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

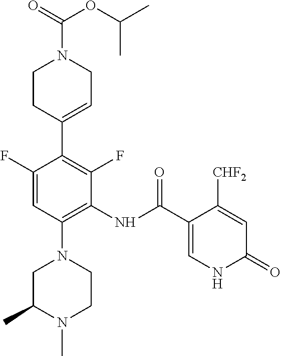 [SMILES: CC(C)OC(=O)N1CC=C(c2c(F)cc(N3CCN(C)[C@@H](C)C3)c(NC(=O)c3c[nH]c(=O)cc3C(F)F)c2F)CC1]

To a solution of the (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (25 mg, 0.051 mmol) and N,N-diisopropylethylamine (0.018 ml, 0.101 mmol) in dichloromethane (4 ml) at RT, was added isopropyl chloroformate (0.048 ml, 0.048 mmol) dropwise over a period of 10 min. Standard workup and purification afforded the title compound as a beige solid (22 mg, 71% yield). LCMS [M+1]⁺=580.34.

### Example 35: Isopropyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

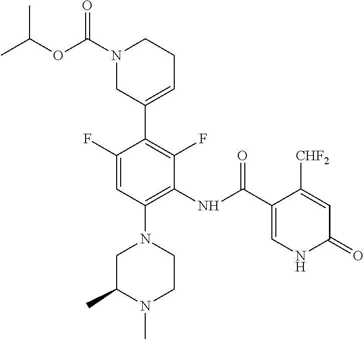 [SMILES: CC(C)OC(=O)N1CCC=C(c2c(F)cc(N3CCN(C)[C@@H](C)C3)c(NC(=O)c3c[nH]c(=O)cc3C(F)F)c2F)C1]

### Step 1: tert-Butyl (S)-5-(3-(4-(difluoromethyl)-6-(2-(trimethylsilyl)ethoxy)nicotinamido)-4-(3,4-dimethylpiperazin-1-yl)-2,6-difluorophenyl)-3,6-dihydropyridine-1(2H)-carboxylate

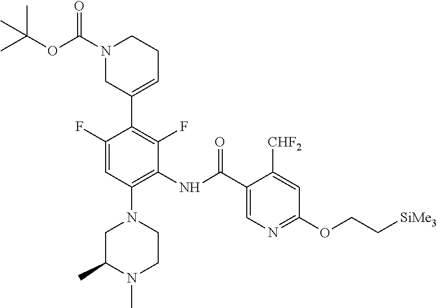 [SMILES: C[C@H]1CN(c2cc(F)c(C3=CCCN(C(=O)OC(C)(C)C)C3)c(F)c2NC(=O)c2cnc(OCC[Si](C)(C)C)cc2C(F)F)CCN1C]

(S)—N-(3-Bromo-6-(3,4-dimethylpiperazin-1-yl)-2,4-difluorophenyl)-4-(difluoromethyl)-6-(2-(trimethylsilyl)ethoxy)nicotinamide (142 mg, 0.240 mmol, preparation described in Example 34), 1-Boc-5,6-dihydro-2H-pyridine-3-boronic acid, pinacol ester (96 mg, 0.312 mmol) and [1,1′-Bis(diphenylphosphino)ferrocene]dichloropalladium(II) (22.84 mg, 0.031 mmol) were mixed in 1,4-dioxane (3 mL). Potassium phosphate tribasic reagent grade, >=98% (1.200 ml, 1.200 mmol) was added and the vial was flushed with nitrogen. The r×n mixture was heated in a microwave reactor to 110° C. for 1.75 h. The mixture was mixed with brine (5 mL) and 5 ml EtOAc, the org phase was separated, aq. phase was extracted with EA (5 m L×2). The combined extract was dried over Na₂SO₄, concentrated and purified on Isco column (4 g), eluting with hexanes containing 0-50% EtOAc. The desired fractions were combined and concentrated to get the title compound as an pale yellow solid (154 mg). LCMS [M+H]⁺ 694.6.

### Step 2: (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide

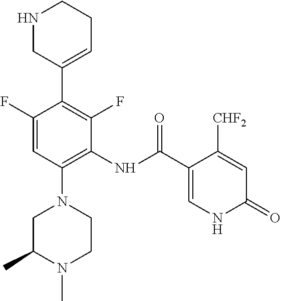 [SMILES: C[C@H]1CN(c2cc(F)c(C3=CCCNC3)c(F)c2NC(=O)c2c[nH]c(=O)cc2C(F)F)CCN1C]

TFA (0.5 mL) was added to a solution of the starting material in DCM (1.5 ml) at RT and the mixture was stirred at RT for 1 h, concentrated to dryness, dissolved in MeOH and passed through a cation exchange resin cartridge (Porapak Rxn CX 20 cc). A solution of 3% NH₃ in MeOH was used to elute the desired product as the free base. The desired fractions were combined and concentrated to get the title compound as an off white solid. (98 mg, 89% yield). LCMS [M−H]⁻ 492.5.

### Step 3: Isopropyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

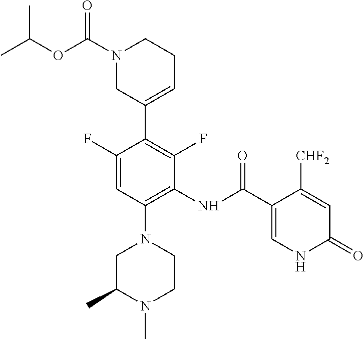 [SMILES: CC(C)OC(=O)N1CCC=C(c2c(F)cc(N3CCN(C)[C@@H](C)C3)c(NC(=O)c3c[nH]c(=O)cc3C(F)F)c2F)C1]

The title compound was prepared as a beige solid (22 mg, 71% yield) from (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide using a procedure that was similar to Example 34. LCMS [M+1]⁺ 580.34.

### Example 36: Isopropyl (S)-3-(3-(4-(difluoromethyl)-6-oxo-1,6-dihydropyridine-3-carboxamido)-4-(3,4-dimethylpiperazin-1-yl)-2,6-difluorophenyl)-2,5-dihydro-H-pyrrole-1-carboxylate

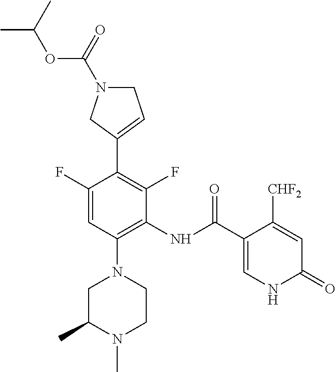 [SMILES: CC(C)OC(=O)N1CC=C(c2c(F)cc(N3CCN(C)[C@@H](C)C3)c(NC(=O)c3c[nH]c(=O)cc3C(F)F)c2F)C1]

### Step 1: (S)-4-(difluoromethyl)-N-(3-(2,5-dihydro-1H-pyrrol-3-yl)-6-(3,4-dimethylpiperazin-1-yl)-2,4-difluorophenyl)-6-oxo-1,6-dihydropyridine-3-carboxamide

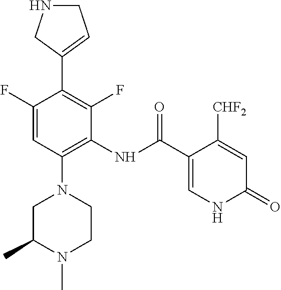 [SMILES: C[C@H]1CN(c2cc(F)c(C3=CCNC3)c(F)c2NC(=O)c2c[nH]c(=O)cc2C(F)F)CCN1C]

The sequence followed was similar to that described in Example 34 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (162 mg, 0.274 mmol) and 1-Boc-2,5-dihydro-1H-pyrrole-3-boronic acid, pinacol ester (105 mg, 0.356 mmol) to obtain the N-Boc intermediate as an off white foam (246 mg). Deprotection with TFA (0.5 mL) in DCM (1.5 mL) at RT afforded, after purification, the title compound as an off white solid (96 mg, 95% yield for last step). LCMS [M+H]⁺ 480.3.

### Step 2: Isopropyl (S)-3-(3-(4-(difluoromethyl)-6-oxo-1,6-dihydropyridine-3-carboxamido)-4-(3,4-dimethylpiperazin-1-yl)-2,6-difluorophenyl)-2,5-dihydro-H-pyrrole-1-carboxylate

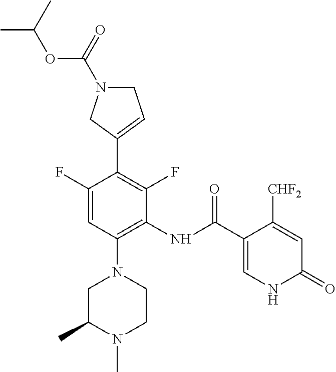 [SMILES: CC(C)OC(=O)N1CC=C(c2c(F)cc(N3CCN(C)[C@@H](C)C3)c(NC(=O)c3c[nH]c(=O)cc3C(F)F)c2F)C1]

The title compound from intermediate (S)-4-(difluoromethyl)-N-(3-(2,5-dihydro-1H-pyrrol-3-yl)-6-(3,4-dimethylpiperazin-1-yl)-2,4-difluorophenyl)-6-oxo-1,6-dihydropyridine-3-carboxamide. The title compound was isolated as a beige solid (18 mg, 58% yield LCMS [M+1]⁺ 566.34.

### Example 37: Isopropyl (S)-3-(4-(3,4-dimethylpiperazin-1-yl)-2,6-difluoro-3-(6-oxo-4-(trifluoromethyl)-1,6-dihydropyridine-3-carboxamido)phenyl)-2,5-dihydro-1H-pyrrole-1-carboxylate

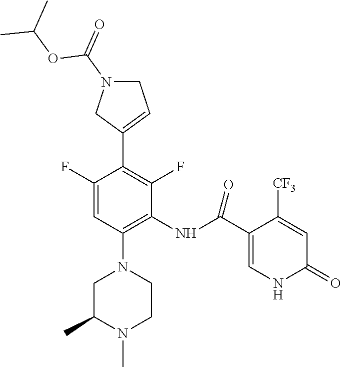 [SMILES: CC(C)OC(=O)N1CC=C(c2c(F)cc(N3CCN(C)[C@@H](C)C3)c(NC(=O)c3c[nH]c(=O)cc3C(F)(F)F)c2F)C1]

### Step 1: tert-Butyl (S)-3-(3-amino-4-(3,4-dimethylpiperazin-1-yl)-2,6-difluorophenyl)-2,5-dihydro-1H-pyrrole-1-carboxylate

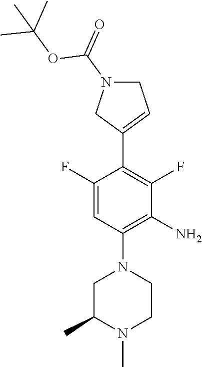 [SMILES: C[C@H]1CN(c2cc(F)c(C3=CCN(C(=O)OC(C)(C)C)C3)c(F)c2N)CCN1C]

1-Boc-2,5-dihydro-1H-pyrrole-3-boronic acid pinacol ester (300 mg, 1.015 mmol),1-Boc-2,5-dihydro-1H-pyrrole-3-boronic acid, pinacol ester (300 mg, 1.015 mmol) and [1,1′-Bis(diphenylphosphino)ferrocene]dichloropalladium(II) (74.3 mg, 0.102 mmol) were mixed in 1,4-dioxane (10 mL). Potassium phosphate tribasic reagent grade, >=98% (3.90 ml, 3.90 mmol) was added and the vial was flushed with nitrogen. The mixture was heated in a microwave reactor to 110° C. for 1.5 h. Complete disappearance of the starting material and formation of the desired product was observed. The mixture was partitioned between brine (20 mL) and 15 ml EtOAc, the org phase was separated, aq. phase was extracted with EA (15 m L×2). The combined extract was dried over Na₂SO₄, concentrated and purified on sg column (12 g), eluting with hexanes containing 0-50% EtOAc. The desired fractions were combined and concentrated to get the title compound as an off white foam (264 mg). LCMS [M+H]⁺ 409.5.

### Step 2: tert-Butyl (S)-3-(4-(3,4-dimethylpiperazin-1-yl)-2,6-difluoro-3-(4-(trifluoromethyl)-6-(2-(trimethylsilyl)ethoxy)nicotinamido)phenyl)-2,5-dihydro-1H-pyrrole-1-carboxylate

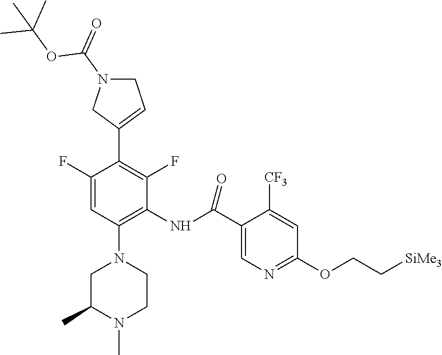 [SMILES: C[C@H]1CN(c2cc(F)c(C3=CCN(C(=O)OC(C)(C)C)C3)c(F)c2NC(=O)c2cnc(OCC[Si](C)(C)C)cc2C(F)(F)F)CCN1C]

Propylphosphonic anhydride solution (1.137 ml, 1.909 mmol) was added to a solution of 4-(trifluoromethyl)-6-(2-(trimethylsilyl)ethoxy)nicotinic acid (254 mg, 0.827 mmol) in pyridine (1.5 mL) under N₂ at RT. After stirring for 30 min at 50° C., tert-butyl (S)-3-(3-amino-4-(3,4-dimethylpiperazin-1-yl)-2,6-difluorophenyl)-2,5-dihydro-1H-pyrrole-1-carboxylate (260 mg, 0.636 mmol) was added as a solution in pyridine (1 mL) and the mixture was stirred at 80° C. for 1.25 h. The mixture was allowed to cool to RT, concentrated, and the residue was taken up in DCM and water. The organic phase was separated, aq. phase was extracted with DCM (×3), the combined org phase was washed with 1 N NaOH soln (×3), water, brine, dried over Na₂SO₄ and concentrated to get the crude (sm/product ratio 31/69) as a brown solid. The product was purified on sg column (12 G) eluting with hexanes containing 0-50% EtOAc to yield the title compound as a light peach colored solid (240 mg). LCMS [M+H]⁺. 698.6.

### Step 3: (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide

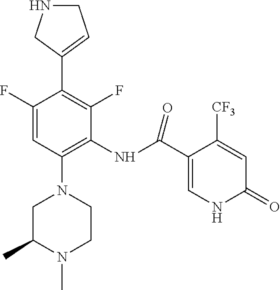 [SMILES: C[C@H]1CN(c2cc(F)c(C3=CCNC3)c(F)c2NC(=O)c2c[nH]c(=O)cc2C(F)(F)F)CCN1C]

TFA (0.5 mL) was added to a solution of the tert-butyl (S)-3-(4-(3,4-dimethylpiperazin-1-yl)-2,6-difluoro-3-(4-(trifluoromethyl)-6-(2-(trimethylsilyl)ethoxy)nicotinamido)phenyl)-2,5-dihydro-1H-pyrrole-1-carboxylate in DCM (1.5 ml) and the mixture was stirred at RT. LCMS after 1 h showed completion of the reaction. The mixture was concentrated to dryness, the residue was dissolved in MeOH and passed through a cation exchange resin cartridge (Porapak Rxn CX 20 cc). A solution of 3% NH₃ in MeOH was used to elute the desired product as the free base. The desired fractions were combined and concentrated to get the title compound as an off white solid. (86 mg). LCMS [M+H]⁺=498.4.

### Step 3: Isopropyl (S)-3-(4-(3,4-dimethylpiperazin-1-yl)-2,6-difluoro-3-(6-oxo-4-(trifluoromethyl)-1,6-dihydropyridine-3-carboxamido)phenyl)-2,5-dihydro-1H-pyrrole-1-carboxylate

To a solution of (R)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (30 mg, 0.060 mmol) and N,N-diisopropylethylamine (0.021 ml, 0.121 mmol) in DCM (4 ml) at RT, was added isopropyl chloroformate (0.057 ml, 0.057 mmol) dropwise over a period of 10 min. The mixture was concentrated onto celite and purified in a manner similar to Example 19 to afford the title compound was isolated as an off white powder (24.5 mg, 66%). LCMS [M+1]⁺ 584.5.

### Example 38: Isopropyl (S)-5-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate

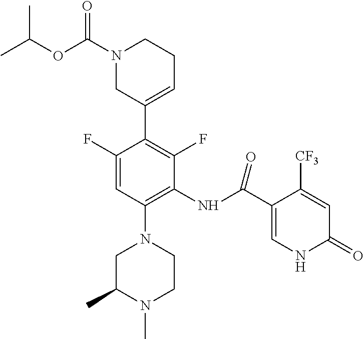 [SMILES: CC(C)OC(=O)N1CCC=C(c2c(F)cc(N3CCN(C)[C@@H](C)C3)c(NC(=O)c3c[nH]c(=O)cc3C(F)(F)F)c2F)C1]

### Step 1: tert-Butyl (S)-5-(3-amino-4-(3,4-dimethylpiperazin-1-yl)-2,6-difluorophenyl)-3,6-dihydropyridine-1(2H)-carboxylate

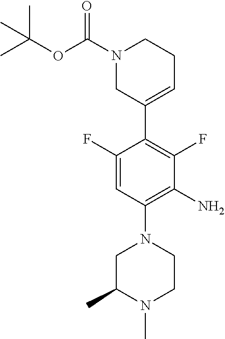 [SMILES: C[C@H]1CN(c2cc(F)c(C3=CCCN(C(=O)OC(C)(C)C)C3)c(F)c2N)CCN1C]

The procedure was similar to Example 37, Step 1 using 1-Boc-5,6-dihydro-2H-pyridine-3-boronic acid pinacol ester (314 mg, 1.015 mmol) and 1-Boc-5,6-dihydro-2H-pyridine-3-boronic acid, pinacol ester (314 mg, 1.015 mmol) to afford after workup and purification the title compound as an off-white foam (246 mg, 75% yield). LCMS [M+H]⁺ 423.5.

### Step 2: tert-Butyl (S)-5-(4-(3,4-dimethylpiperazin-1-yl)-2,6-difluoro-3-(4-(trifluoromethyl)-6-(2-(trimethylsilyl)ethoxy)nicotinamido)phenyl)-3,6-dihydropyridine-1(2H)-carboxylate

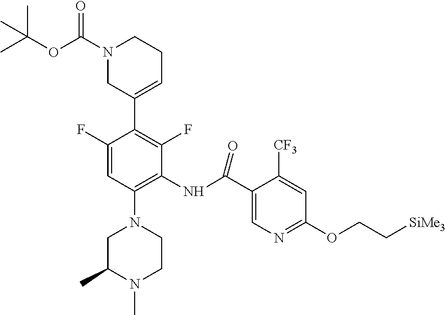 [SMILES: C[C@H]1CN(c2cc(F)c(C3=CCCN(C(=O)OC(C)(C)C)C3)c(F)c2NC(=O)c2cnc(OCC[Si](C)(C)C)cc2C(F)(F)F)CCN1C]

Propylphosphonic anhydride solution (1.036 ml, 1.740 mmol) was added dropwise to a solution of 4-(trifluoromethyl)-6-(2-(trimethylsilyl)ethoxy)nicotinic acid (214 mg, 0.696 mmol) and Pyridine (0.187 ml, 2.319 mmol) in dry THF (10 ml) under N₂ at RT. A clear light peach coloured solution was obtained. After stirring for 1.5 h at RT, tert-butyl (S)-5-(3-amino-4-(3,4-dimethylpiperazin-1-yl)-2,6-difluorophenyl)-3,6-dihydropyridine-1(2H)-carboxylate (245 mg, 0.580 mmol) was added as a solution in 5 ml THF and the mixture was stirred at 80° C., Another eq. of propylphosphonic anhydride solution was added and heating was continued at 80° C. for 30 h. The mixture was allowed to cool to RT, and workup afforded the title compound as an off white foam (199 mg). LCMS [M+H]⁺=712.6.

### Step 3: (S)—N-(6-(3,4-Dimethylpiperazin-1-yl)-2,4-difluoro-3-(1,2,5,6-tetrahydropyridin-3-yl)phenyl)-6-oxo-4-(trifluoromethyl)-1,6-dihydropyridine-3-carboxamide

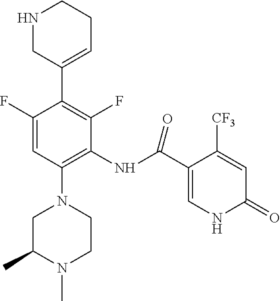 [SMILES: C[C@H]1CN(c2cc(F)c(C3=CCCNC3)c(F)c2NC(=O)c2c[nH]c(=O)cc2C(F)(F)F)CCN1C]

TFA (0.5 ml) was added to a solution of the SM in DCM (1.5 ml) at RT and the rxn mix was stirred at RT. LCMS after 10 min showed completion of the rxn. The mixture was concentrated to dryness, the residue was dissolved in MeOH and passed through a cation exchange resin cartridge (Porapak Rxn CX 20 cc). A solution of 3% NH₃ in MeOH was used to elute the desired product as the free base. The desired fractions were combined and concentrated to get the title compound as an off white solid. (139 mg). LCMS [M+H]⁺ 512.5.

### Step 4: Isopropyl (S)-5-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate

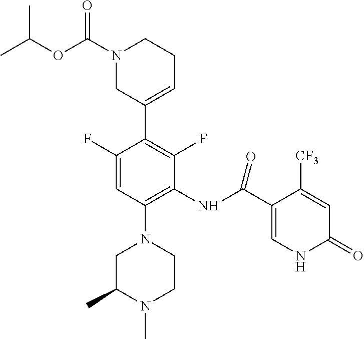 [SMILES: CC(C)OC(=O)N1CCC=C(c2c(F)cc(N3CCN(C)[C@@H](C)C3)c(NC(=O)c3c[nH]c(=O)cc3C(F)(F)F)c2F)C1]

To a solution of (R)—N-(6-(3,4-dimethylpiperazin-1-yl)-2,4-difluoro-3-(1,2,5,6-tetrahydropyridin-3-yl)phenyl)-6-oxo-4-(trifluoromethyl)-1,6-dihydropyridine-3-carboxamide (30 mg, 0.059 mmol) and N,N-diisopropylethylamine (0.020 ml, 0.117 mmol) in DCM (4 ml) at RT, was added isopropyl chloroformate (0.056 ml, 0.056 mmol) dropwise over a period of 10 min. The starting material was not completely soluble at the beginning of the reaction. The reaction was complete immediately upon completion of the addition. The mixture was concentrated onto celite and purified on prep column, eluting with water (containing 0.1% HCOOH)/acetonitrile. The title compound was isolated as a beige solid (27 mg, 73% yield). LCMS [M+1]f=598.6.

### Example 39: Isopropyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate

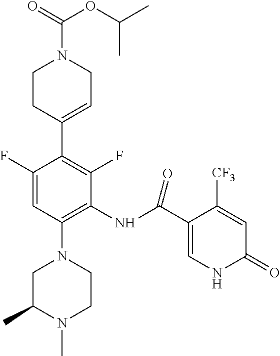 [SMILES: CC(C)OC(=O)N1CC=C(c2c(F)cc(N3CCN(C)[C@@H](C)C3)c(NC(=O)c3c[nH]c(=O)cc3C(F)(F)F)c2F)CC1]

The procedure followed was similar to Example 34 using (S)—N-(6-(3,4-dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide and isopropyl chloroformate. The title compound was isolated as a beige solid (27 mg, 73% yield). LCMS [M+1]⁺ 598.6.

### Example 40: N-(2,4-difluoro-3-(2-morpholinopyrimidin-5-yl)-6-((3S,5R)-3,4,5-trimethylpiperazin-1-yl)phenyl)-4-(difluoromethyl)-1-methyl-6-oxo-1,6-dihydropyridine-3-carboxamide

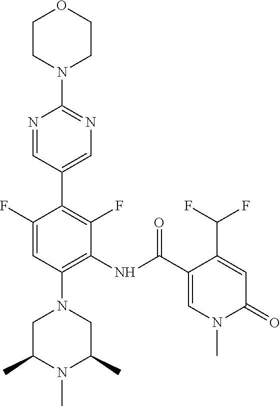 [SMILES: C[C@@H]1CN(c2cc(F)c(-c3cnc(N4CCOCC4)nc3)c(F)c2NC(=O)c2cn(C)c(=O)cc2C(F)F)C[C@H](C)N1C]

The title compound (white solid, 17.4 mg, 29%) was prepared by a procedure similar to Example 13, Step 7 using 2,4-difluoro-3-(2-morpholinopyrimidin-5-yl)-6-((3S,5R)-3,4,5-trimethylpiperazin-1-yl)aniline (42 mg, 0.1 mmol), 4-(difluoromethyl)-1-methyl-6-oxo-1,6-dihydropyridine-3-carboxylic acid (37 mg, 0.18 mmol) and T₃P (50% wt in EtOAc, 0.12 mL, 0.2 mmol). LCMS [M+H]⁺ 604.3.

### Example 41: N-(2,4-difluoro-3-(2-morpholinopyrimidin-5-yl)-6-((3S,5R)-3,4,5-trimethylpiperazin-1-yl)phenyl)-4-(difluoromethyl)-6-oxo-1,6-dihydropyridine-3-carboxamide

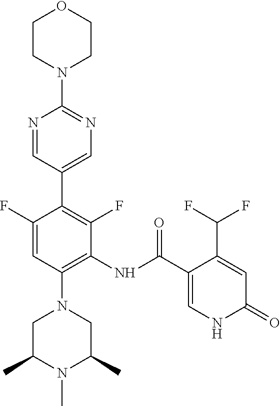 [SMILES: C[C@@H]1CN(c2cc(F)c(-c3cnc(N4CCOCC4)nc3)c(F)c2NC(=O)c2c[nH]c(=O)cc2C(F)F)C[C@H](C)N1C]

The title compound (white solid, 33.2 mg, 56%) was prepared by a procedure similar to Example 13, Step 7 using 2,4-difluoro-3-(2-morpholinopyrimidin-5-yl)-6-((3S,5R)-3,4,5-trimethylpiperazin-1-yl)aniline (42 mg, 0.1 mmol), 4-(difluoromethyl)-6-(2-(trimethylsilyl)ethoxy)nicotinic acid (52 mg, 0.18 mmol, preparation described in Example 33) and T₃P (50% wt % in EtOAc, 0.12 mL, 0.2 mmol) followed by TFA deprotection (1 mL TFA/10 mL DCM). LCMS [M+H]⁺ 590.4.

### Example 42: 3,3-Difluorocyclobutyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

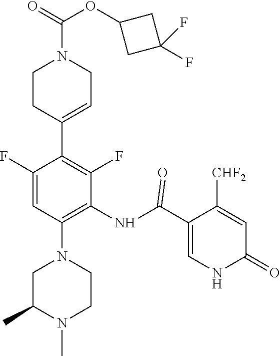 [SMILES: C[C@H]1CN(c2cc(F)c(C3=CCN(C(=O)OC4CC(F)(F)C4)CC3)c(F)c2NC(=O)c2c[nH]c(=O)cc2C(F)F)CCN1C]

### Step 1: tert-Butyl (S)-4-(3-(4-(difluoromethyl)-6-(2-(trimethylsilyl)ethoxy)nicotinamido)-4-(3,4-dimethylpiperazin-1-yl)-2,6-difluorophenyl)-3,6-dihydropyridine-1(2H)-carboxylate

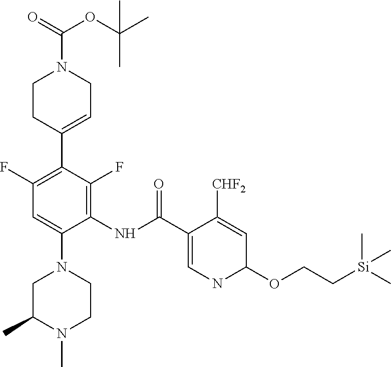

(S)—N-(3-Bromo-6-(3,4-dimethylpiperazin-1-yl)-2,4-difluorophenyl)-4-(difluoromethyl)-6-(2-(trimethylsilyl)ethoxy)nicotinamide (147 mg, 0.249 mmol preparation described in Example 33), tert-butyl4-(4,4,5,5-tetramethyl-1,3,2-dioxaborolan-2-yl)-1,2,3,6-tetrahydropyridine-1-carboxylate (100 mg, 0.323 mmol) and [1,1′-Bis(diphenylphosphino)ferrocene]dichloropalladium(II) (23.64 mg, 0.032 mmol) were mixed in 1,4-dioxane (3 mL). Potassium phosphate tribasic reagent grade, >=98% (1.24 ml, 1.24 mmol) was added and the vial was flushed with nitrogen. The rxn mixture was heated in a microwave reactor at 110° C. for 1.75 h. Standard workup and purification by flash chromatography on silica gel (0-50% EtOAc in hexanes) afforded the title compound as an off white foam (137 mg, 79%). LCMS [M+H]⁺ 694.6.

### Step 2: (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide

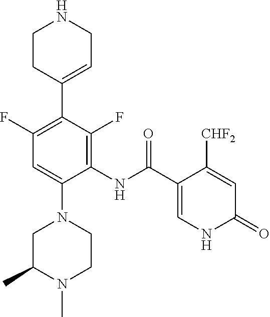 [SMILES: C[C@H]1CN(c2cc(F)c(C3=CCNCC3)c(F)c2NC(=O)c2c[nH]c(=O)cc2C(F)F)CCN1C]

Trifluoroacetic acid (0.5 mL) was added to a solution of tert-butyl (S)-4-(3-(4-(difluoromethyl)-6-(2-(trimethylsilyl)ethoxy)nicotinamido)-4-(3,4-dimethylpiperazin-1-yl)-2,6-difluorophenyl)-3,6-dihydropyridine-1(2H)-carboxylate (137 mg, 0.197 mmol) in dichloromethane (1.5 ml) at RT and the reaction mixture was stirred at RT for 1 h. It was concentrated to dryness, the residue was dissolved in MeOH and passed through a cation exchange resin cartridge (Porapak Rxn CX 6 cc). A solution of 3% NH₃ in MeOH was used to elute the desired product as the free base. The desired fractions were combined and concentrated to yield the title compound was an off white solid. (92 mg, 94%). LCMS [M+H]⁺=494.4.

### Step 3: 3,3-Difluorocyclobutyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

To a solution of (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (33 mg, 0.067 mmol) and 3,3-difluorocyclobutyl (4-nitrophenyl) carbonate (20.09 mg, 0.074 mmol) in DCM (4 mL) was added anhydrous pyridine (0.022 ml, 0.267 mmol) and the reaction mixture was heated at 90° C. for 1 h. The mixture was then purified on preparatory column eluting with water (containing 0.1% HCOOH)/acetonitrile (containing 0.1% HCOOH) gradient (85/55). The title compound was isolated as a beige solid (33.5 mg, 76%). LCMS [M+1]⁺ 528.6.

### Example 43: 3,3-Difluorocyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

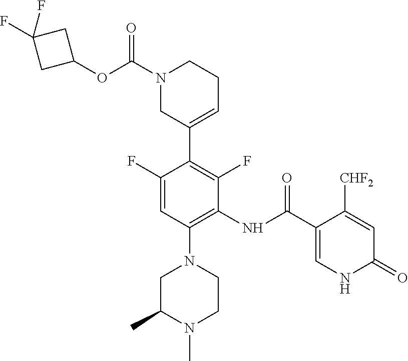 [SMILES: C[C@H]1CN(c2cc(F)c(C3=CCCN(C(=O)OC4CC(F)(F)C4)C3)c(F)c2NC(=O)c2c[nH]c(=O)cc2C(F)F)CCN1C]

The procedure was similar to the last step of Example 42 using (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide. The title compound was isolated as a beige powder (24 mg, 72% yield). LCMS [M+1]⁺ 628.6.

### Example 44: 3,3-Difluorocyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

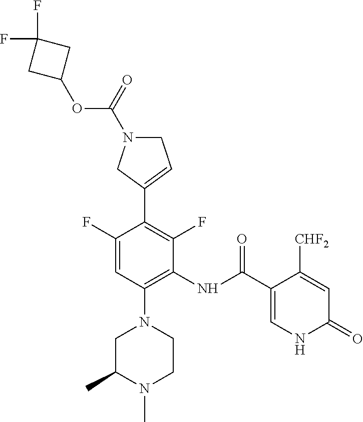 [SMILES: C[C@H]1CN(c2cc(F)c(C3=CCN(C(=O)OC4CC(F)(F)C4)C3)c(F)c2NC(=O)c2c[nH]c(=O)cc2C(F)F)CCN1C]

The procedure used was similar to Example 42 using (S)-4-(difluoromethyl)-N-(3-(2,5-dihydro-1H-pyrrol-3-yl)-6-(3,4-dimethylpiperazin-1-yl)-2,4-difluorophenyl)-6-oxo-1,6-dihydropyridine-3-carboxamide. The title compound was isolated as an off-white powder (20.5 mg, 61% yield). LCMS [M+1]⁺ 614.6.

### Example 45: 3,3-Difluorocyclobutyl (S)-3-(4-(3,4-dimethylpiperazin-1-yl)-2,6-difluoro-3-(6-oxo-4-(trifluoromethyl)-1,6-dihydropyridine-3-carboxamido)phenyl)-2,5-dihydro-1H-pyrrole-1-carboxylate

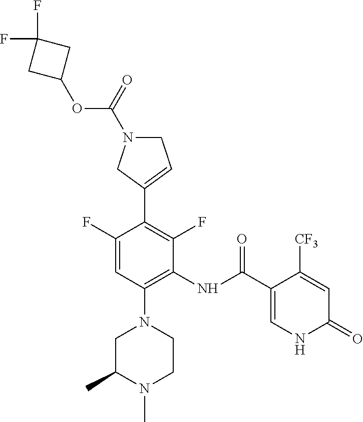 [SMILES: C[C@H]1CN(c2cc(F)c(C3=CCN(C(=O)OC4CC(F)(F)C4)C3)c(F)c2NC(=O)c2c[nH]c(=O)cc2C(F)(F)F)CCN1C]

The procedure was similar to the last step of Example 42 using (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (preparation described in Example 37). The title compound was isolated as a beige powder (24 mg, 67%). LCMS [M+1]⁺ 632.6.

### Example 46: 3,3-Difluorocyclobutyl (S)-5-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate

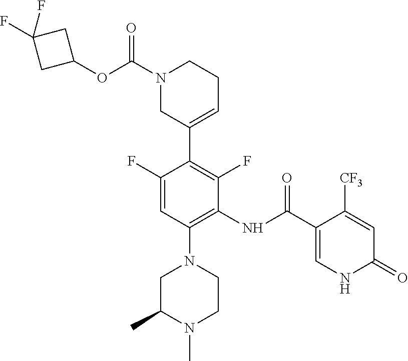 [SMILES: C[C@H]1CN(c2cc(F)c(C3=CCCN(C(=O)OC4CC(F)(F)C4)C3)c(F)c2NC(=O)c2c[nH]c(=O)cc2C(F)(F)F)CCN1C]

The procedure was similar to Example 42 using (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (preparation described in Example 38). The title compound was isolated as an off white powder (25 mg, 63% yield). LCMS [M+1]⁺ 646.4.

### Example 47: 3,3-Difluorocyclobutyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate

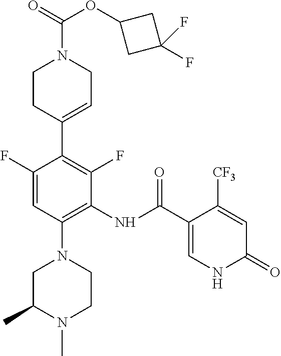 [SMILES: C[C@H]1CN(c2cc(F)c(C3=CCN(C(=O)OC4CC(F)(F)C4)CC3)c(F)c2NC(=O)c2c[nH]c(=O)cc2C(F)(F)F)CCN1C]

### Step 1: (S)—N-(3-bromo-6-(3,4-dimethylpiperazin-1-yl)-2,4-difluorophenyl)-4-(trifluoromethyl)-6-(2-(trimethylsilyl)ethoxy)nicotinamide

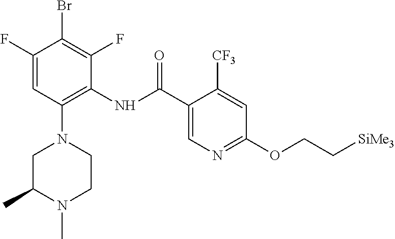 [SMILES: C[C@H]1CN(c2cc(F)c(Br)c(F)c2NC(=O)c2cnc(OCC[Si](C)(C)C)cc2C(F)(F)F)CCN1C]

A stirred solution of 4-(trifluoromethyl)-6-(2-(trimethylsilyl)ethoxy)nicotinic acid (28.2 g, 107.7 mmol, 2 eq, preparation described in Example 13) in THF (100 mL), was cooled to 0° C. and DIPEA (21.2 mL, 107.7 mmol, 2 eq) was added, followed by (S)-3-bromo-6-(3,4-dimethylpiperazin-1-yl)-2,4-difluoroaniline (9.35 g, 29.31 mmol, 0.9 eq, preparation described in Example 33) and T₃P (51.79 g, 162.8 mmol, 5 eq). The resulting reaction mixture was stirred at RT for 72 h. TLC analysis indicated formation of non-polar spot. The reaction mixture was quenched with ice water and extracted with EtOAc. The combined organic layers were dried over Na₂SO₄ and concentrated under reduced pressure. The crude product was purified by column chromatography (neutral alumina) using 30% EtOAc in pet ether to give (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (17 g, 67% yield) as off white solid. LCMS: [M+H]⁺ 609.31.

### Step 2: tert-Butyl (S)-4-(4-(3,4-dimethylpiperazin-1-yl)-2,6-difluoro-3-(4-(trifluoromethyl)-6-(2-(trimethylsilyl)ethoxy)nicotinamido)phenyl)-3,6-dihydropyridine-1(2H)-carboxylate

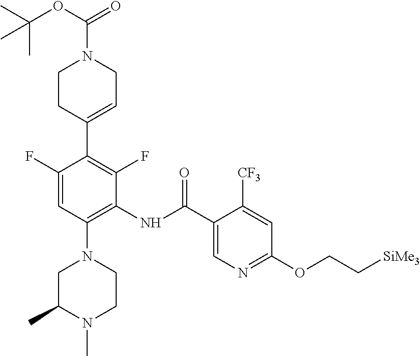 [SMILES: C[C@H]1CN(c2cc(F)c(C3=CCN(C(=O)OC(C)(C)C)CC3)c(F)c2NC(=O)c2cnc(OCC[Si](C)(C)C)cc2C(F)(F)F)CCN1C]

(S)—N-(3-bromo-6-(3,4-dimethylpiperazin-1-yl)-2,4-difluorophenyl)-4-(trifluoromethyl)-6-(2-(trimethylsilyl)ethoxy)nicotinamide (151 mg, 0.248 mmol) and tert-butyl-4-(4,4,5,5-tetramethyl-1,3,2-dioxaborolan-2-yl)-1,2,3,6-tetrahydropyridine-1-carboxylate (100 mg, 0.322 mmol) and [1,1′-bis(diphenylphosphino)ferrocene]dichloropalladium(II) (23.57 mg, 0.032 mmol) were mixed in dioxane (3 mL). Potassium phosphate tribasic reagent grade, >=98% (1.239 ml, 1.239 mmol) was added and the vial was flushed with nitrogen. The mixture was heated in a microwave reactor at 100° C. for 1.75 h. Workup and purification in a manner similar to earlier examples gave the title compound (140 mg, 79% yield); LCMS [M+H]⁺ 712.5.

### Step 3: (S)—N-(6-(3,4-Dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide

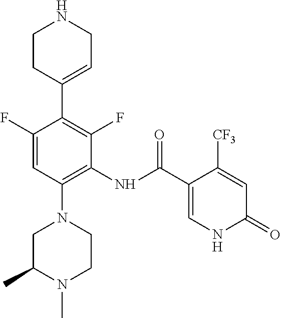 [SMILES: C[C@H]1CN(c2cc(F)c(C3=CCNCC3)c(F)c2NC(=O)c2c[nH]c(=O)cc2C(F)(F)F)CCN1C]

(S)—N-(3-Bromo-6-(3,4-dimethylpiperazin-1-yl)-2,4-difluorophenyl)-4-(trifluoromethyl)-6-(2-(trimethylsilyl)ethoxy)nicotinamide (151 mg, 0.248 mmol), tert-butyl-4-(4,4,5,5-tetramethyl-1,3,2-dioxaborolan-2-yl)-1,2,3,6-tetrahydropyridine-1-carboxylate (100 mg, 0.322 mmol) and [1,1′-bis(diphenylphosphino)ferrocene]dichloropalladium(II) (23.57 mg, 0.032 mmol) were mixed in dioxane (3 mL). Potassium phosphate tribasic reagent grade, >=98% (1.239 ml, 1.239 mmol) was added and the vial was flushed with nitrogen. The mixture was heated in a microwave reactor at 100° C. for 1.75 h. The mixture was mixed with brine (7 mL) and EtOAc (5 mL), the organic phase was separated, the aqueous phase was extracted with EtOAc (5 m L×2) and the combined extracts were dried over Na₂SO₄, concentrated and purified on sg column (4 g), eluting with hexanes containing 0-50% EtOAc. The desired fractions were combined and concentrated to afford the title compound as an off white foam (140 mg). Workup and purification in a manner similar to earlier examples afforded the title compound as an off-white solid (96 mg, 95% yield). LCMS [M+H]⁺ 510.1.

### Step 4: 3,3-Difluorocyclobutyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate

The procedure was similar to Example 42 using (S)—N-(6-(3,4-dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide. The title compound was isolated as an off white powder (29.5 mg, 69% yield). LCMS [M+1]⁺ 646.5.

### Example 48: (S)-4-(difluoromethyl)-N-(6-(3,4-dimethylpiperazin-1-yl)-2,4-difluoro-3-(1-(5-methoxypyrimidin-2-yl)-1,2,3,6-tetrahydropyridin-4-yl)phenyl)-6-oxo-1,6-dihydropyridine-3-carboxamide

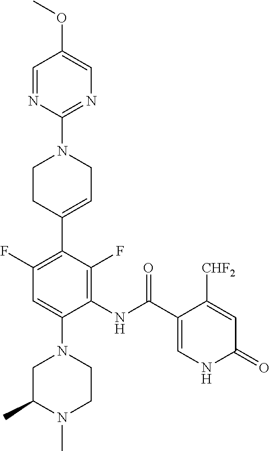 [SMILES: COc1cnc(N2CC=C(c3c(F)cc(N4CCN(C)[C@@H](C)C4)c(NC(=O)c4c[nH]c(=O)cc4C(F)F)c3F)CC2)nc1]

To a solution of (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (31.5 mg, 0.064 mmol, preparation described in Example 34) and 2-bromo-5-methoxypyrimidine (16.89 mg, 0.089 mmol) in 2-propanol (2.5 mL) at RT was added N,N-diisopropylethylamine (0.022 ml, 0.128 mmol). After heating in a microwave reactor at 170° C. for 2 h, the reaction mixture was purified on preparatory column eluting with water (containing 0.1% HCOOH)/acetonitrile (containing 0.1% HCOOH) gradient (85/55). The title compound was isolated as an yellow powder (20 mg, 50%). LCMS [M+1]⁺ 602.5.

### Example 49: (S)-4-(difluoromethyl)-N-(6-(3,4-dimethylpiperazin-1-yl)-2,4-difluoro-3-(1-(5-methoxypyrimidin-2-yl)-1,2,5,6-tetrahydropyridin-3-yl)phenyl)-6-oxo-1,6-dihydropyridine-3-carboxamide

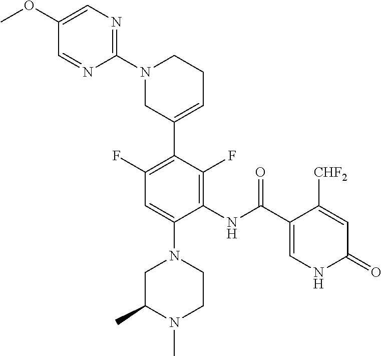 [SMILES: COc1cnc(N2CCC=C(c3c(F)cc(N4CCN(C)[C@@H](C)C4)c(NC(=O)c4c[nH]c(=O)cc4C(F)F)c3F)C2)nc1]

The procedure was similar to Example 48 using (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide and 2-bromo-5-methoxypyrimidine to afford the title compound as a beige powder (13 mg, 46% yield). LCMS [M+1]⁺ 602.5.

### Example 50: (S)-4-(difluoromethyl)-N-(6-(3,4-dimethylpiperazin-1-yl)-2,4-difluoro-3-(1-(5-methoxypyrimidin-2-yl)-2,5-dihydro-1H-pyrrol-3-yl)phenyl)-6-oxo-1,6-dihydropyridine-3-carboxamide

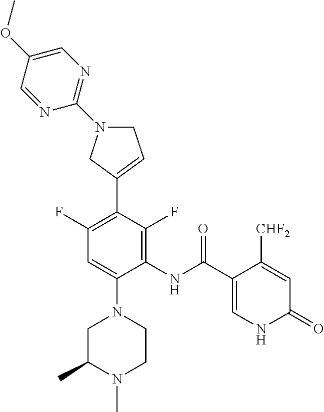 [SMILES: COc1cnc(N2CC=C(c3c(F)cc(N4CCN(C)[C@@H](C)C4)c(NC(=O)c4c[nH]c(=O)cc4C(F)F)c3F)C2)nc1]

The procedure was similar to Example 48 using (S)-4-(difluoromethyl)-N-(3-(2,5-dihydro-1H-pyrrol-3-yl)-6-(3,4-dimethylpiperazin-1-yl)-2,4-difluorophenyl)-6-oxo-1,6-dihydropyridine-3-carboxamide (preparation described in Example 36) and 2-bromo-5-methoxypyrimidine. The title compound was isolated as a beige powder (10.5 mg, 37%). LCMS [M+1]⁺ 588.6.

### Example 51: (S)—N-(6-(3,4-dimethylpiperazin-1-yl)-2,4-difluoro-3-(1-(5-methoxypyrimidin-2-yl)-2,5-dihydro-1H-pyrrol-3-yl)phenyl)-6-oxo-4-(trifluoromethyl)-1,6-dihydropyridine-3-carboxamide

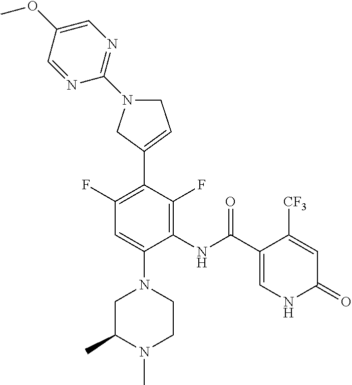 [SMILES: COc1cnc(N2CC=C(c3c(F)cc(N4CCN(C)[C@@H](C)C4)c(NC(=O)c4c[nH]c(=O)cc4C(F)(F)F)c3F)C2)nc1]

The procedure was similar to Example 48 using (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (preparation described in Example 37) and 2-bromo-5-methoxypyrimidine. The title compound was isolated as an off white powder (15.5 mg, 45%). LCMS [M+1]⁺ 606.5.

### Example 52: (S)—N-(6-(3,4-dimethylpiperazin-1-yl)-2,4-difluoro-3-(1-(5-methoxypyrimidin-2-yl)-1,2,5,6-tetrahydropyridin-3-yl)phenyl)-6-oxo-4-(trifluoromethyl)-1,6-dihydropyridine-3-carboxamide

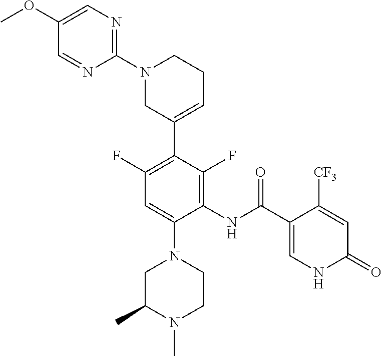 [SMILES: COc1cnc(N2CCC=C(c3c(F)cc(N4CCN(C)[C@@H](C)C4)c(NC(=O)c4c[nH]c(=O)cc4C(F)(F)F)c3F)C2)nc1]

The procedure was similar to Example 48 using (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (preparation described in Example 38) and 2-bromo-5-methoxypyrimidine. The title compound was isolated as a beige powder (15.5 mg, 49% yield). LCMS [M+1]⁺ 620.5.

### Example 53: (S)—N-(6-(3,4-dimethylpiperazin-1-yl)-2,4-difluoro-3-(1-(5-methoxypyrimidin-2-yl)-1,2,3,6-tetrahydropyridin-4-yl)phenyl)-6-oxo-4-(trifluoromethyl)-1,6-dihydropyridine-3-carboxamide

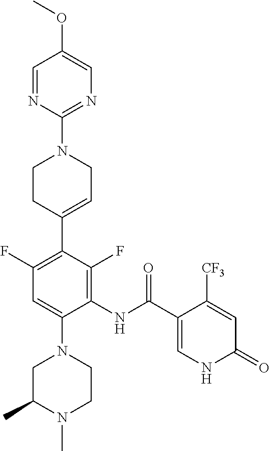 [SMILES: COc1cnc(N2CC=C(c3c(F)cc(N4CCN(C)[C@@H](C)C4)c(NC(=O)c4c[nH]c(=O)cc4C(F)(F)F)c3F)CC2)nc1]

The procedure was similar to Example 48 using (S)—N-(6-(3,4-dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide (preparation described in Example 47) and 2-bromo-5-methoxypyrimidine. The title compound was isolated as a yellow powder (17.5 mg, 65%). LCMS [M+1]⁺=620.5.

### Example 54: (S)-4-(difluoromethyl)-N-(6-(3,4-dimethylpiperazin-1-yl)-2,4-difluoro-3-(1-(pyrimidin-2-yl)-1,2,5,6-tetrahydropyridin-3-yl)phenyl)-6-oxo-1,6-dihydropyridine-3-carboxamide

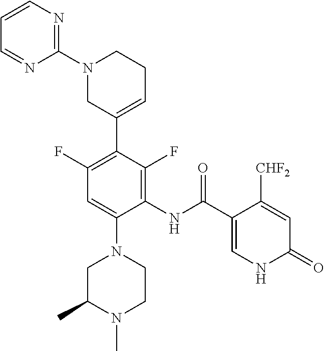 [SMILES: C[C@H]1CN(c2cc(F)c(C3=CCCN(c4ncccn4)C3)c(F)c2NC(=O)c2c[nH]c(=O)cc2C(F)F)CCN1C]

A solution of intermediate (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide (22 mg, 0.045 mmol, preparation described in Example 43), 2-bromopyrimidine 95% (7.80 mg, 0.049 mmol) and N,N-diisopropylethylamine (0.024 ml, 0.138 mmol) in ethanol (3 ml) was heated in a microwave reactor at 100° C. for 2 h. The reaction mixture was purified on preparatory column, eluting with water (containing 0.1% HCOOH)/acetonitrile (containing 0.1% HCOOH) gradient (85/55). The title compound was isolated as a beige powder (14 mg, 52%). LCMS [M+1]⁺ 572.5.

### Example 55: N-(3-(2-((2S,6R)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide

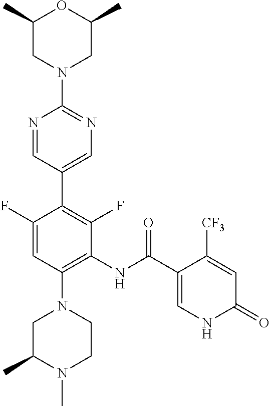 [SMILES: C[C@@H]1CN(c2ncc(-c3c(F)cc(N4CCN(C)[C@@H](C)C4)c(NC(=O)c4c[nH]c(=O)cc4C(F)(F)F)c3F)cn2)C[C@H](C)O1]

The title compound (pale beige solid, 42.6 mg, 67%) was prepared according to a procedure described in Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (2-((2S,6R)-2,6-dimethylmorpholino)pyrimidin-5-yl)boronic acid (47 mg, 0.2 mmol) followed by TFA deprotection (1 mL TFA/10 mL DCM). LCMS [M+H]⁺ 622.4.

### Example 56: N-(3-(2-((2S,6R)-2,6-dimethylmorpholino)pyrimidin-4-yl)-6-((S)-3,4-dimethylpiperazin-1-yl)-2,4-difluorophenyl)-1-methyl-6-oxo-4-(trifluoromethyl)-1,6-dihydropyridine-3-carboxamide

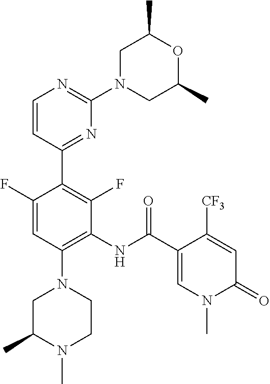 [SMILES: C[C@@H]1CN(c2nccc(-c3c(F)cc(N4CCN(C)[C@@H](C)C4)c(NC(=O)c4cn(C)c(=O)cc4C(F)(F)F)c3F)n2)C[C@H](C)O1]

Starting from (S)-3-bromo-6-(3,4-dimethylpiperazin-1-yl)-2,4-difluoroaniline, the title compound (beige solid, 16.8 mg, 26%) was prepared according to by a procedure similar to that described in Example 13 using 3-(2-((2S,6R)-2,6-dimethylmorpholino)pyrimidin-4-yl)-6-((S)-3,4-dimethylpiperazin-1-yl)-2,4-difluoroaniline (47 mg, 91.5% purity, 0.1 mmol), 1-methyl-6-oxo-4-(trifluoromethyl)-1,6-dihydropyridine-3-carboxylic acid (40 mg, 0.18 mmol) and T₃P (50% wt % in EtOAc, 0.12 mL, 0.2 mmol). LCMS [M+H]⁺ 636.5.

### Example 57:4-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-1-methyl-6-oxo-1,6-dihydropyridine-3-carboxamide

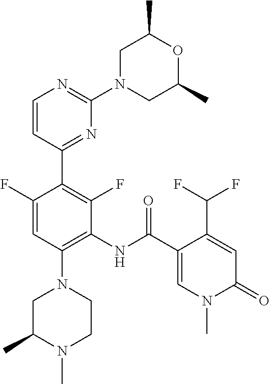 [SMILES: C[C@@H]1CN(c2nccc(-c3c(F)cc(N4CCN(C)[C@@H](C)C4)c(NC(=O)c4cn(C)c(=O)cc4C(F)F)c3F)n2)C[C@H](C)O1]

Starting from (S)-3-bromo-6-(3,4-dimethylpiperazin-1-yl)-2,4-difluoroaniline, the title compound (beige solid, 15.3 mg, 25%) was prepared using 3-(2-((2S,6R)-2,6-dimethylmorpholino)pyrimidin-4-yl)-6-((S)-3,4-dimethylpiperazin-1-yl)-2,4-difluoroaniline (47 mg, 91.54% purity, 0.1 mmol), 4-(difluoromethyl)-1-methyl-6-oxo-1,6-dihydropyridine-3-carboxylic acid (37 mg, 0.18 mmol) and T₃P (50% wt % in EtOAc, 0.12 mL, 0.2 mmol) by a procedure similar that of Example 13. LCMS [M+H]⁺ 618.4.

### Example 58: N-(3-(2-((2S,6R)-2,6-dimethylmorpholino)pyrimidin-4-yl)-6-((S)-3,4-dimethylpiperazin-1-yl)-2,4-difluorophenyl)-6-oxo-4-(trifluoromethyl)-1,6-dihydropyridine-3-carboxamide

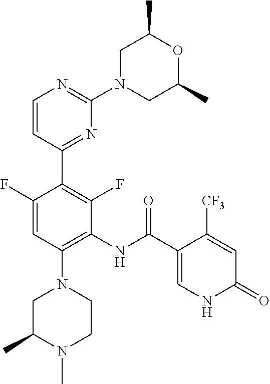 [SMILES: C[C@@H]1CN(c2nccc(-c3c(F)cc(N4CCN(C)[C@@H](C)C4)c(NC(=O)c4c[nH]c(=O)cc4C(F)(F)F)c3F)n2)C[C@H](C)O1]

Starting from (S)-3-bromo-6-(3,4-dimethylpiperazin-1-yl)-2,4-difluoroaniline, the title compound (off white solid, 11.9 mg, 19%) was prepared according to a procedure similar to Example 13 using 3-(2-((2S,6R)-2,6-dimethylmorpholino)pyrimidin-4-yl)-6-((S)-3,4-dimethylpiperazin-1-yl)-2,4-difluoroaniline (47 mg, 91.54% purity, 0.1 mmol), 4-(trifluoromethyl)-6-(2-(trimethylsilyl)ethoxy)nicotinic acid (55 mg, 0.18 mmol) and T₃P (50% wt % in EtOAc, 0.12 mL, 0.2 mmol) followed by TFA deprotection (1 mL TFA/10 mL DCM). LCMS [M+H]⁺ 622.5.

### Example 59:4-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-6-oxo-1,6-dihydropyridine-3-carboxamide

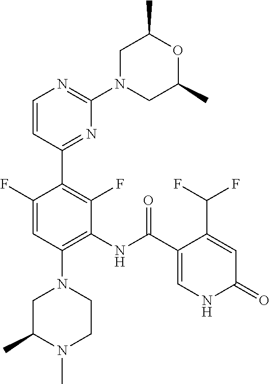 [SMILES: C[C@@H]1CN(c2nccc(-c3c(F)cc(N4CCN(C)[C@@H](C)C4)c(NC(=O)c4c[nH]c(=O)cc4C(F)F)c3F)n2)C[C@H](C)O1]

Starting from (S)-3-bromo-6-(3,4-dimethylpiperazin-1-yl)-2,4-difluoroaniline, the title compound (pale beige solid, 18.9 mg, 31%) was prepared by a procedure similar to Example 13 using 3-(2-((2S,6R)-2,6-dimethylmorpholino)pyrimidin-4-yl)-6-((S)-3,4-dimethylpiperazin-1-yl)-2,4-difluoroaniline (47 mg, 91.54% purity, 0.1 mmol), 4-(difluoromethyl)-6-(2-(trimethylsilyl)ethoxy)nicotinic acid (52 mg, 0.18 mmol, preparation described in Example 21) and T₃P (50% wt in EtOAc, 0.12 mL, 0.2 mmol) followed by TFA deprotection (1 mL TFA/10 mL DCM). LCMS [M+H]⁺ 604.4.

### Example 60: N-(6-((S)-3,4-dimethylpiperazin-1-yl)-2,4-difluoro-3-(2-((S)-2-methylmorpholino)pyrimidin-5-yl)phenyl)-6-oxo-4-(trifluoromethyl)-1,6-dihydropyridine-3-carboxamide

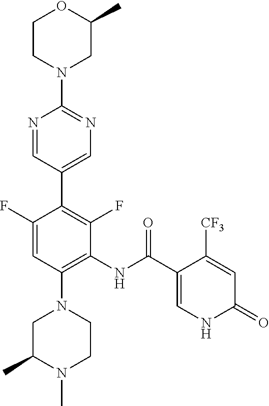 [SMILES: C[C@H]1CN(c2ncc(-c3c(F)cc(N4CCN(C)[C@@H](C)C4)c(NC(=O)c4c[nH]c(=O)cc4C(F)(F)F)c3F)cn2)CCO1]

The title compound (orange solid, 35.1 mg, 56%) was prepared by a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (S)-(2-(2-methylmorpholino)pyrimidin-5-yl)boronic acid (84 mg, 0.3 mmol) followed by TFA deprotection (1 mL TFA/10 mL DCM). LCMS [M+H]⁺ 608.4.

### Example 61: N-(3-(2-(4,4-difluoropiperidin-1-yl)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

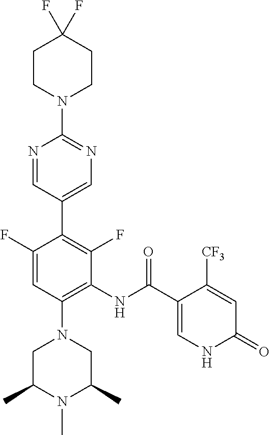 [SMILES: C[C@@H]1CN(c2cc(F)c(-c3cnc(N4CCC(F)(F)CC4)nc3)c(F)c2NC(=O)c2c[nH]c(=O)cc2C(F)(F)F)C[C@H](C)N1C]

### Step 1: N-(3-(2-(4,4-difluoropiperidin-1-yl)pyrimidin-5-yl)-2,4-difluoro-6-((3S,5R)-3,4,5-trimethylpiperazin-1-yl)phenyl)-4-(trifluoromethyl)-6-(2-(trimethylsilyl)ethoxy)nicotinamide

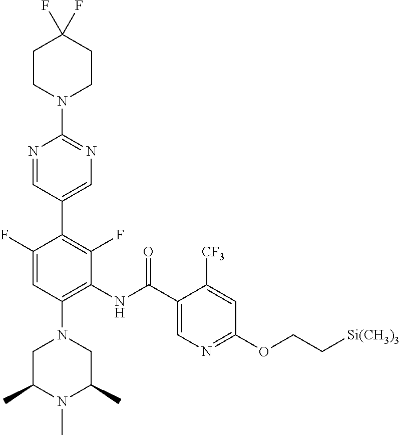

A procedure similar to Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (35.5 mg, 0.057 mmol, preparation described in Example 13), 2-(4,4-difluoropiperidin-1-yl)-5-(4,4,5,5-tetramethyl-1,3,2-dioxaborolan-2-yl)pyrimidine (27.8 mg, 0.085 mmol) afforded, after purification, the title compound that was used as is in for the next transformation. LCMS [M+H]⁺ 742.11.

### Step 2: N-(3-(2-(4,4-difluoropiperidin-1-yl)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

N-(3-(2-(4,4-difluoropiperidin-1-yl)pyrimidin-5-yl)-2,4-difluoro-6-((3S,5R)-3,4,5-trimethylpiperazin-1-yl)phenyl)-4-(trifluoromethyl)-6-(2-(trimethylsilyl)ethoxy)nicotinamide from step 1 was dissolved in 2 mL of DCM and TFA (130 mg, 1.139 mmol) was added. The purple solution was stirred for 1 hour and the solvent was evaporated. The residue was purified using prep HPLC (20%-90%, H₂O/acetonitrile) followed by a cation exchange column eluting with MeOH:NH₄OH and freeze dried for 2 days to afford the title compound (20.72 mg, over two steps 57%) as a white powder. LCMS [M+1]⁺=642.34.

### Example 62: N-(3-(6-((2S,6R)-2,6-dimethylmorpholino)pyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

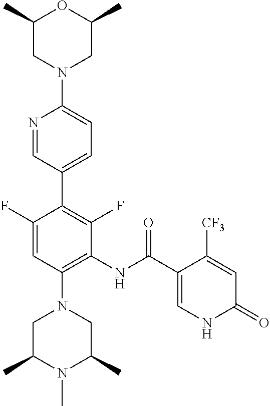 [SMILES: C[C@@H]1CN(c2ccc(-c3c(F)cc(N4C[C@@H](C)N(C)[C@@H](C)C4)c(NC(=O)c4c[nH]c(=O)cc4C(F)(F)F)c3F)cn2)C[C@H](C)O1]

The title compound (white solid, 17 mg, 47%) was prepared according to the sequence described above for the preparation of Example 61 using (2S,6R)-2,6-dimethyl-4-(5-(4,4,5,5-tetramethyl-1,3,2-dioxaborolan-2-yl)pyridin-2-yl)morpholine (27.1 mg, 0.085 mmol) in place of 2-(4,4-difluoropiperidin-1-yl)-5-(4,4,5,5-tetramethyl-1,3,2-dioxaborolan-2-yl)pyrimidine. LCMS [M+1]⁺ 635.34.

### Example 63: N-(3-(6-(dimethylamino)-5-fluoropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

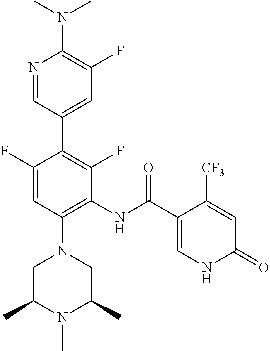 [SMILES: C[C@@H]1CN(c2cc(F)c(-c3cnc(N(C)C)c(F)c3)c(F)c2NC(=O)c2c[nH]c(=O)cc2C(F)(F)F)C[C@H](C)N1C]

The title compound (white solid, 14 mg, 44%) was prepared according to the sequence described above for the preparation of Example 61 using 2-(N,N-dimethylamino)-3-fluoropyridine-5-boronic acid pinacol ester hydrochloride (24.75 mg, 0.082 mmol) in place of 2-(4,4-difluoropiperidin-1-yl)-5-(4,4,5,5-tetramethyl-1,3,2-dioxaborolan-2-yl)pyrimidine. LCMS [M+1]⁺ 583.35.

### Example 64: N-(3-(5-cyano-6-morpholinopyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

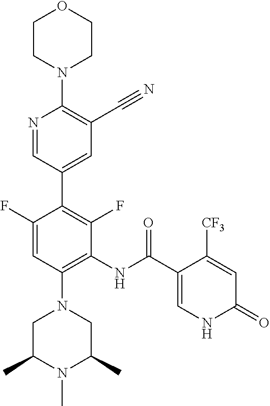 [SMILES: C[C@@H]1CN(c2cc(F)c(-c3cnc(N4CCOCC4)c(C#N)c3)c(F)c2NC(=O)c2c[nH]c(=O)cc2C(F)(F)F)C[C@H](C)N1C]

The title compound (white solid, 25 mg, 68%) was prepared according to the sequence described above for the preparation of Example 61 using 3-cyano-2-morpholinopyridine-5-boronic acid, pinacol ester (26.4 mg, 0.084 mmol), in place of 2-(4,4-difluoropiperidin-1-yl)-5-(4,4,5,5-tetramethyl-1,3,2-dioxaborolan-2-yl)pyrimidine.

### Example 65: N-(2,4-difluoro-3-(6-((tetrahydro-2H-pyran-4-yl)oxy)pyridin-3-yl)-6-((3S,5R)-3,4,5-trimethylpiperazin-1-yl)phenyl)-6-oxo-4-(trifluoromethyl)-1,6-dihydropyridine-3-carboxamide

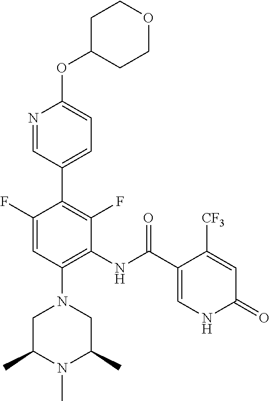 [SMILES: C[C@@H]1CN(c2cc(F)c(-c3ccc(OC4CCOCC4)nc3)c(F)c2NC(=O)c2c[nH]c(=O)cc2C(F)(F)F)C[C@H](C)N1C]

The title compound (white solid, 18.3 mg, 52%) was prepared according to the sequence described above for the preparation of Example 61 using 2-(tetrahydropyran-4-yloxy)-5-(4,4,5,5-tetramethyl-1,3,2-dioxaborolan-2-yl)pyridine (25.9 mg, 0.085 mmol) in place of 2-(4,4-difluoropiperidin-1-yl)-5-(4,4,5,5-tetramethyl-1,3,2-dioxaborolan-2-yl)pyrimidine. LCMS [M+1]⁺ 622.40.

### Example 66: N-(3-(2-((2S,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide Formic Acid

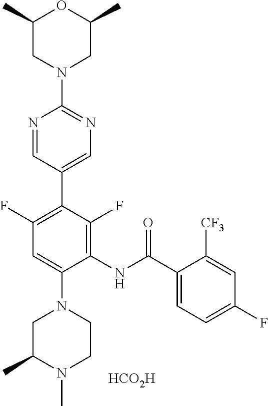 [SMILES: C[C@@H]1CN(c2ncc(-c3c(F)cc(N4CCN(C)[C@@H](C)C4)c(NC(=O)c4ccc(F)cc4C(F)(F)F)c3F)cn2)C[C@H](C)O1 ; "HCO2H"]

Employing a sequence similar to Example 16 starting with (S)-3-bromo-6-(3,4-dimethylpiperazin-1-yl)-2,4-difluoroaniline and 4-fluoro-2-(trifluoromethyl)benzoic acid, the title compound (formic acid salt, white solid, 35.5 mg, 53%) was prepared according to a procedure similar to that described in Example 1, Step 6using (S)—N-(3-bromo-6-(3,4-dimethylpiperazin-1-yl)-2,4-difluorophenyl)-4-fluoro-2-(trifluoromethyl)benzamide (67 mg, 75.67% purity, 0.1 mmol) and (2-((2S,6R)-2,6-dimethylmorpholino)pyrimidin-5-yl)boronic acid (47 mg, 0.2 mmol, 20 mol %). LCMS [M+H]⁺ 623.5.

### Example 67: (S)-4-(difluoromethyl)-N-(6-(3,4-dimethylpiperazin-1-yl)-2,4-difluoro-3-(1-(6-methoxypyrimidin-4-yl)-1,2,3,6-tetrahydropyridin-4-yl)phenyl)-6-oxo-1,6-dihydropyridine-3-carboxamide

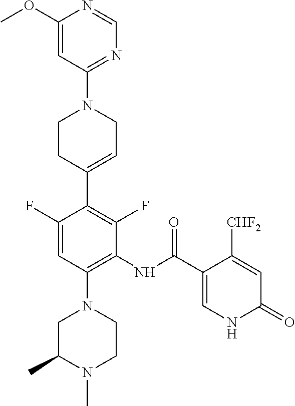 [SMILES: COc1cc(N2CC=C(c3c(F)cc(N4CCN(C)[C@@H](C)C4)c(NC(=O)c4c[nH]c(=O)cc4C(F)F)c3F)CC2)ncn1]

The procedure followed was similar to Example 48 using (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (40 mg, 0.073 mmol, preparation described in Example 42) and 4-iodo-6-methoxy-pyrimidine (24.24 mg, 0.103 mmol) to afford, after purification, the title compound as an orange powder (26 mg, 59% yield). LCMS [M+1]⁺ 602.5.

### Example 68: (S)—N-(6-(3,4-dimethylpiperazin-1-yl)-2,4-difluoro-3-(1-(6-methoxypyrimidin-4-yl)-1,2,5,6-tetrahydropyridin-3-yl)phenyl)-6-oxo-4-(trifluoromethyl)-1,6-dihydropyridine-3-carboxamide

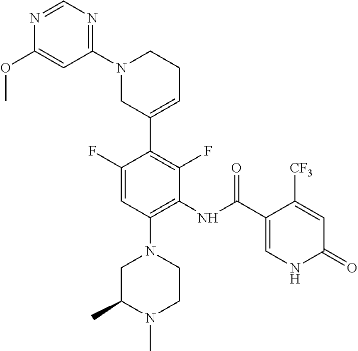 [SMILES: COc1cc(N2CCC=C(c3c(F)cc(N4CCN(C)[C@@H](C)C4)c(NC(=O)c4c[nH]c(=O)cc4C(F)(F)F)c3F)C2)ncn1]

The procedure was similar to Example 48 using (S)—N-(6-(3,4-dimethylpiperazin-1-yl)-2,4-difluoro-3-(1,2,5,6-tetrahydropyridin-3-yl)phenyl)-6-oxo-4-(trifluoromethyl)-1,6-dihydropyridine-3-carboxamide (26 mg, 0.051 mmol) and 4-iodo-6-methoxy pyrimidine (16.80 mg, 0.071 mmol) to give the title compound as an off white powder (15.5 mg, 47%). LCMS [M+1]⁺ 620.6.

### Example 69: N-(2,4-difluoro-3-(2-morpholinopyridin-4-yl)-6-((3S,5R)-3,4,5-trimethylpiperazin-1-yl)phenyl)-6-oxo-4-(trifluoromethyl)-1,6-dihydropyridine-3-carboxamide

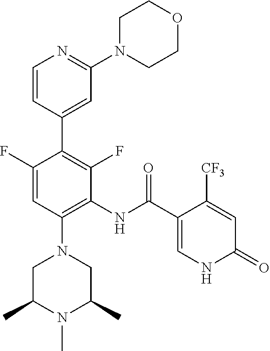 [SMILES: C[C@@H]1CN(c2cc(F)c(-c3ccnc(N4CCOCC4)c3)c(F)c2NC(=O)c2c[nH]c(=O)cc2C(F)(F)F)C[C@H](C)N1C]

### Step 1: N-(2,4-difluoro-3-(2-morpholinopyridin-4-yl)-6-((3S,5R)-3,4,5-trimethylpiperazin-1-yl)phenyl)-4-(trifluoromethyl)-6-(2-(trimethylsilyl)ethoxy)nicotinamide

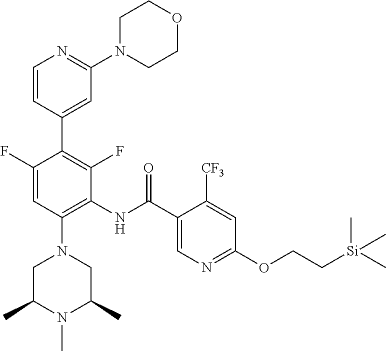 [SMILES: C[C@@H]1CN(c2cc(F)c(-c3ccnc(N4CCOCC4)c3)c(F)c2NC(=O)c2cnc(OCC[Si](C)(C)C)cc2C(F)(F)F)C[C@H](C)N1C]

In a 5 mL microwave vial 2-morpholinopyridine-4-boronic acid, pinacol ester (24.07 mg, 0.083 mmol), N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (34.48 mg, 0.055 mmol), [1,12-bis(diphenylphosphino)ferrocene]dichloropalladium(II), DCM complex (4.52 mg, 5.53 μmol) and potassium phosphate tribasic reagent grade (23.48 mg, 0.111 mmol) were dissolved in water (55.3 μl)/1,4-dioxane (498 μl) to give a white suspension. That was stirred for 5 min, degassed, purged with N₂, and microwaved for 180 min at 120° C. The solvent was evaporated and 15 mL of DCM were added. The suspension was sonicated and extracted from water (15 mL). The solvent was evaporated in vacuo yielding the crude product that was purified by flash column chromatography on silica gel (0-100%, 89% CH₂Cl₂, 10% MeOH, 1% NH₄Ac/CH₂Cl₂) to afford the title compound, that was used as is in for the following transformation. LCMS [M+H]⁺ 707.57.

### Step 2: N-(2,4-difluoro-3-(2-morpholinopyridin-4-yl)-6-((3S,5R)-3,4,5-trimethylpiperazin-1-yl)phenyl)-6-oxo-4-(trifluoromethyl)-1,6-dihydropyridine-3-carboxamide

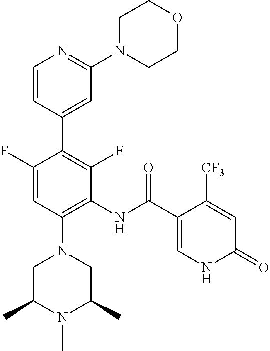 [SMILES: C[C@@H]1CN(c2cc(F)c(-c3ccnc(N4CCOCC4)c3)c(F)c2NC(=O)c2c[nH]c(=O)cc2C(F)(F)F)C[C@H](C)N1C]

N-(2,4-difluoro-3-(2-morpholinopyridin-4-yl)-6-((3S,5R)-3,4,5-trimethylpiperazin-1-yl)phenyl)-4-(trifluoromethyl)-6-(2-(trimethylsilyl)ethoxy)nicotinamide from step 1 was dissolved in 2 mL of dichloromethane and trifluoroacetic acid (126 mg, 1.106 mmol) was added. The purple solution was stirred for 1 hour and the solvent was evaporated. The residue was purified by cation exchange column eluting with MeOH:NH₄OH and lyophilized to afford the title compound (24.14 mg, over two steps 69% yield) as an off white powder. LCMS [M+1]⁺ 607.43.

### Example 70: N-(3-(2-(dimethylamino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

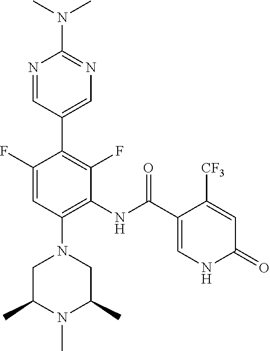 [SMILES: C[C@@H]1CN(c2cc(F)c(-c3cnc(N(C)C)nc3)c(F)c2NC(=O)c2c[nH]c(=O)cc2C(F)(F)F)C[C@H](C)N1C]

The title compound (off white solid, 12 mg, 36%) was prepared according to the sequence described above for the preparation of Example 69 using N,N-dimethyl-5-(4,4,5,5-tetramethyl-1,3,2-dioxaborolan-2-yl)pyrimidin-2-amine (20.86 mg, 0.084 mmol), in place of 2-morpholinopyridine-4-boronic acid, pinacol ester. LCMS [M+1]⁺ 566.64.

### Example 71: N-(3-(benzo[d][1,3]dioxol-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

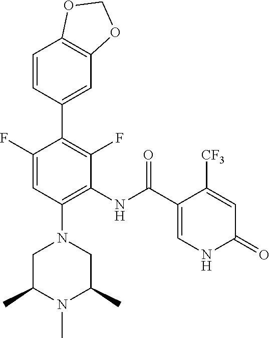 [SMILES: C[C@@H]1CN(c2cc(F)c(-c3ccc4c(c3)OCO4)c(F)c2NC(=O)c2c[nH]c(=O)cc2C(F)(F)F)C[C@H](C)N1C]

The title compound (pink solid, 22 mg, 56%) was prepared according to the sequence described above for the preparation of Example 69 using 3,4-methylenedioxyphenylboronic acid (14.33 mg, 0.086 mmol), in place of 2-morpholinopyridine-4-boronic acid, pinacol ester. LCMS [M+1]⁺ 565.44.

### Example 72: N-(3-(2,3-dihydrobenzo[b][1,4]dioxin-6-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

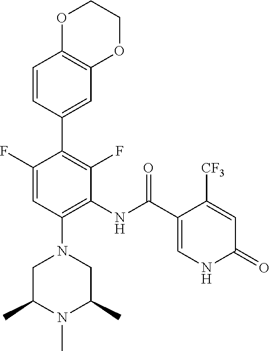 [SMILES: C[C@@H]1CN(c2cc(F)c(-c3ccc4c(c3)OCCO4)c(F)c2NC(=O)c2c[nH]c(=O)cc2C(F)(F)F)C[C@H](C)N1C]

The title compound (white solid, 8 mg, 25%) was prepared according to the sequence described above for the preparation of Example 69 using 2,3-dihydro-1,4-benzodioxin-6-ylboronic acid (14.98 mg, 0.083 mmol), in place of 2-morpholinopyridine-4-boronic acid, pinacol ester. LCMS [M+1]⁺=579.44.

### Example 73: N-(3-(2,3-dihydro-[1,4]dioxino[2,3-b]pyridin-7-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

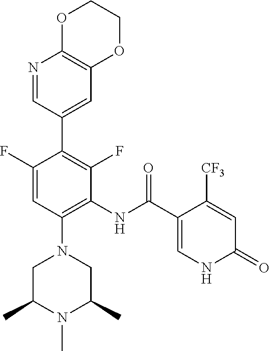 [SMILES: C[C@@H]1CN(c2cc(F)c(-c3cnc4c(c3)OCCO4)c(F)c2NC(=O)c2c[nH]c(=O)cc2C(F)(F)F)C[C@H](C)N1C]

The title compound (white solid, 8 mg, 25%) was prepared according to the sequence described above for the preparation of Example 69 using 7-(4,4,5,5-tetramethyl-1,3,2-dioxaborolan-2-yl)-2,3-dihydro-[1,4]dioxino[2,3-b]pyridine (22.30 mg, 0.085 mmol), in place of 2-morpholinopyridine-4-boronic acid, pinacol ester. LCMS [M+1]⁺=580.49.

### Example 74: (S)-4-(difluoromethyl)-N-(6-(3,4-dimethylpiperazin-1-yl)-2,4-difluoro-3-(1-(6-methoxypyrimidin-4-yl)-2,5-dihydro-1H-pyrrol-3-yl)phenyl)-6-oxo-1,6-dihydropyridine-3-carboxamide

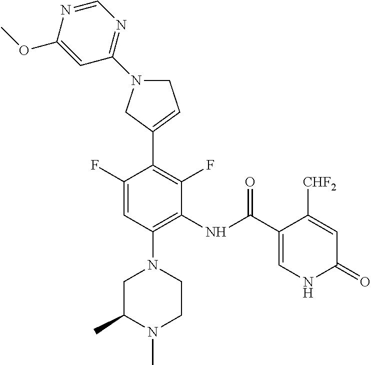 [SMILES: COc1cc(N2CC=C(c3c(F)cc(N4CCN(C)[C@@H](C)C4)c(NC(=O)c4c[nH]c(=O)cc4C(F)F)c3F)C2)ncn1]

A procedure similar to Example 48 using (S)-4-(difluoromethyl)-N-(3-(2,5-dihydro-1H-pyrrol-3-yl)-6-(3,4-dimethylpiperazin-1-yl)-2,4-difluorophenyl)-6-oxo-1,6-dihydropyridine-3-carboxamide (25 mg, 0.052 mmol, preparation described in Example 36) and 4-iodo-6-methoxy pyrimidine (17.23 mg, 0.073 mmol) afforded the title compound as a beige powder (13 mg, 40%). LCMS [M+1]⁺ 588.6.

### Example 75: (S)—N-(6-(3,4-dimethylpiperazin-1-yl)-2,4-difluoro-3-(1-(6-methoxypyrimidin-4-yl)-2,5-dihydro-1H-pyrrol-3-yl)phenyl)-6-oxo-4-(trifluoromethyl)-1,6-dihydropyridine-3-carboxamide

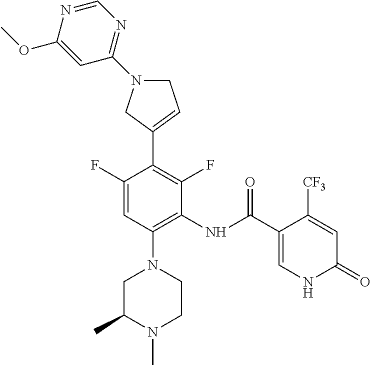 [SMILES: COc1cc(N2CC=C(c3c(F)cc(N4CCN(C)[C@@H](C)C4)c(NC(=O)c4c[nH]c(=O)cc4C(F)(F)F)c3F)C2)ncn1]

A procedure similar to Example 48 using (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (20 mg, 0.040 mmol, preparation described in Example 37) and 4-iodo-6-methoxy pyrimidine (13.28 mg, 0.056 mmol) afforded the title compound as a white powder (8 mg, 31%). LCMS [M+1]⁺ 606.7.

### Example 76: (S)—N-(6-(3,4-dimethylpiperazin-1-yl)-2,4-difluoro-3-(1-(6-methoxypyrimidin-4-yl)-1,2,3,6-tetrahydropyridin-4-yl)phenyl)-6-oxo-4-(trifluoromethyl)-1,6-dihydropyridine-3-carboxamide

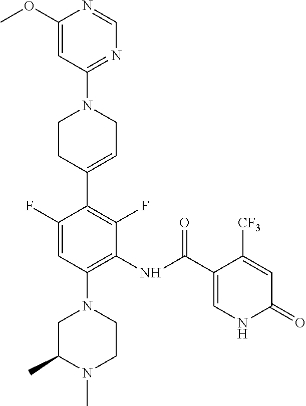 [SMILES: COc1cc(N2CC=C(c3c(F)cc(N4CCN(C)[C@@H](C)C4)c(NC(=O)c4c[nH]c(=O)cc4C(F)(F)F)c3F)CC2)ncn1]

A procedure similar to Example 48 using (S)—N-(6-(3,4-dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide (24 mg, 0.047 mmol, preparation described in Example 47) and 4-iodo-6-methoxy pyrimidine (15.50 mg, 0.066 mmol) afforded the title compound as an off white powder (7.5 mg, 25%). LCMS [M+1]⁺=620.6.

### Example 77: N-(3-(2-((2S,6R)-2,6-dimethylmorpholino)pyrimidin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

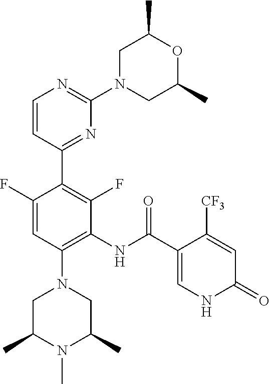 [SMILES: C[C@@H]1CN(c2nccc(-c3c(F)cc(N4C[C@@H](C)N(C)[C@@H](C)C4)c(NC(=O)c4c[nH]c(=O)cc4C(F)(F)F)c3F)n2)C[C@H](C)O1]

3-(2-((2S,6R)-2,6-Dimethylmorpholino)pyrimidin-4-yl)-2,4-difluoro-6-((3S,5R)-3,4,5-trimethylpiperazin-1-yl)aniline (457 mg, 89% based on 87.11% purity) was prepared according to the method used for the preparation of 3-(2-((2S,6R)-2,6-dimethylmorpholino)pyrimidin-4-yl)-6-((S)-3,4-dimethylpiperazin-1-yl)-2,4-difluoroaniline by replacing (S)-3-bromo-6-(3,4-dimethylpiperazin-1-yl)-2,4-difluoroaniline with 3-bromo-2,4-difluoro-6-((3S,5R)-3,4,5-trimethylpiperazin-1-yl)aniline (334 mg, 1 mmol). LCMS [M+H]⁺ 447.4. The title compound (white solid, 8.5 mg, 7%) was prepared according to a procedure similar to that described in Example 13 using 3-(2-((2S,6R)-2,6-dimethylmorpholino)pyrimidin-4-yl)-2,4-difluoro-6-((3S,5R)-3,4,5-trimethylpiperazin-1-yl)aniline (100 mg, 89% purity, 0.2 mmol), 4-(trifluoromethyl)-6-(2-(trimethylsilyl)ethoxy)nicotinic acid (111 mg, 0.36 mmol) and T3P (50% wt % in EtOAc, 0.24 mL, 0.4 mmol) followed by TFA deprotection (1 mL TFA/10 mL DCM). LCMS [M+H]⁺ 636.5.

### Example 78:4-(Difluoromethyl)-N-(3-(2-((2S,6R)-2,6-dimethylmorpholino)pyrimidin-4-yl)-2,4-difluoro-6-((3S,5R)-3,4,5-trimethylpiperazin-1-yl)phenyl)-6-oxo-1,6-dihydropyridine-3-carboxamide

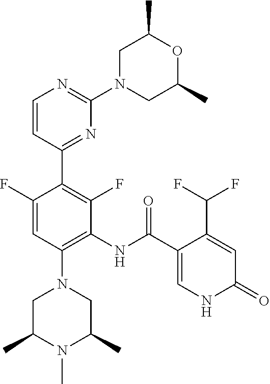 [SMILES: C[C@@H]1CN(c2nccc(-c3c(F)cc(N4C[C@@H](C)N(C)[C@@H](C)C4)c(NC(=O)c4c[nH]c(=O)cc4C(F)F)c3F)n2)C[C@H](C)O1]

The title compound (white solid, 7.5 mg, 6%) was prepared according to a procedure similar to Example 13 using 3-(2-((2S,6R)-2,6-dimethylmorpholino)pyrimidin-4-yl)-2,4-difluoro-6-((3S,5R)-3,4,5-trimethylpiperazin-1-yl)aniline (100 mg, 89% purity, 0.2 mmol), 4-(difluoromethyl)-6-(2-(trimethylsilyl)ethoxy)nicotinic acid (104 mg, 0.36 mmol) and T3P (50% wt % in EtOAc, 0.24 mL, 0.4 mmol) followed by TFA deprotection (1 mL TFA/10 mL DCM). LCMS [M+H]⁺ 618.4.

### Example 79: (S)-4-(difluoromethyl)-N-(6-(3,4-dimethylpiperazin-1-yl)-2,4-difluoro-3-(1-(6-methoxypyrimidin-4-yl)-1,2,5,6-tetrahydropyridin-3-yl)phenyl)-6-oxo-1,6-dihydropyridine-3-carboxamide

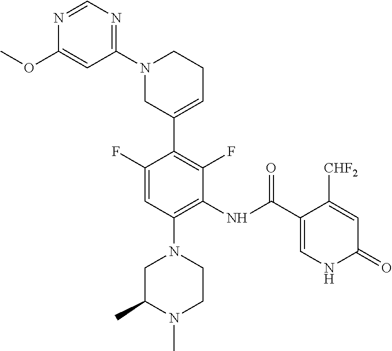 [SMILES: COc1cc(N2CCC=C(c3c(F)cc(N4CCN(C)[C@@H](C)C4)c(NC(=O)c4c[nH]c(=O)cc4C(F)F)c3F)C2)ncn1]

A procedure similar to Example 48 using (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide (25 mg, 0.051 mmol) and 4-iodo-6-methoxy pyrimidine (16.74 mg, 0.071 mmol) afforded the title compound as an off white powder (21.5 mg, 67%). LCMS [M+1]⁺ 602.6.

### Example 80: (S)—N-(6-(3,4-dimethylpiperazin-1-yl)-2,4-difluoro-3-(1-(5-fluoropyrimidin-2-yl)-2,5-dihydro-1H-pyrrol-3-yl)phenyl)-6-oxo-4-(trifluoromethyl)-1,6-dihydropyridine-3-carboxamide

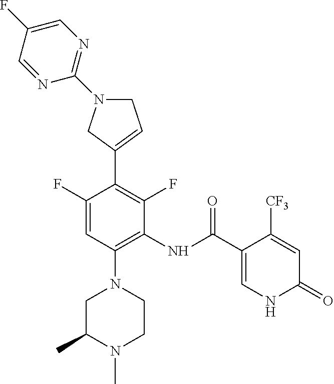 [SMILES: C[C@H]1CN(c2cc(F)c(C3=CCN(c4ncc(F)cn4)C3)c(F)c2NC(=O)c2c[nH]c(=O)cc2C(F)(F)F)CCN1C]

A procedure similar to Example 48 using (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (20 mg, 0.040 mmol) and 2-bromo-5-fluoropyrimidine (7.12 mg, 0.040 mmol) afforded the title compound as a white powder (9.5 mg, 38%). LCMS [M+1]⁺ 594.6.

### Example 81: (S)-4-(difluoromethyl)-N-(6-(3,4-dimethylpiperazin-1-yl)-2,4-difluoro-3-(1-(5-fluoropyrimidin-2-yl)-1,2,3,6-tetrahydropyridin-4-yl)phenyl)-6-oxo-1,6-dihydropyridine-3-carboxamide

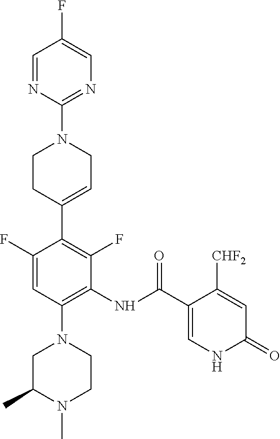 [SMILES: C[C@H]1CN(c2cc(F)c(C3=CCN(c4ncc(F)cn4)CC3)c(F)c2NC(=O)c2c[nH]c(=O)cc2C(F)F)CCN1C]

The procedure followed was similar to Example 48 using (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (25 mg, 0.051 mmol) and 2-bromo-5-fluoropyrimidine (8.97 mg, 0.051 mmol) to afford the title compound as a beige powder (20 mg, 64% yield). LCMS [M+1]⁺ 590.6.

### Example 82: (S)—N-(6-(3,4-dimethylpiperazin-1-yl)-2,4-difluoro-3-(1-(5-fluoropyrimidin-2-yl)-1,2,3,6-tetrahydropyridin-4-yl)phenyl)-6-oxo-4-(trifluoromethyl)-1,6-dihydropyridine-3-carboxamide

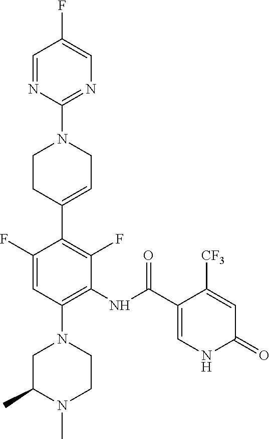 [SMILES: C[C@H]1CN(c2cc(F)c(C3=CCN(c4ncc(F)cn4)CC3)c(F)c2NC(=O)c2c[nH]c(=O)cc2C(F)(F)F)CCN1C]

The procedure followed was similar to Example 48 using (S)—N-(6-(3,4-dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide (25 mg, 0.049 mmol, preparation described in Example 47) and 2-bromo-5-fluoropyrimidine (8.65 mg, 0.049 mmol). The title compound was isolated as a pale yellow powder (21.5 mg, 69%). LCMS [M+1]⁺ 608.7.

### Example 83: (S)-4-(difluoromethyl)-N-(6-(3,4-dimethylpiperazin-1-yl)-2,4-difluoro-3-(1-(5-fluoropyrimidin-2-yl)-1,2,5,6-tetrahydropyridin-3-yl)phenyl)-6-oxo-1,6-dihydropyridine-3-carboxamide

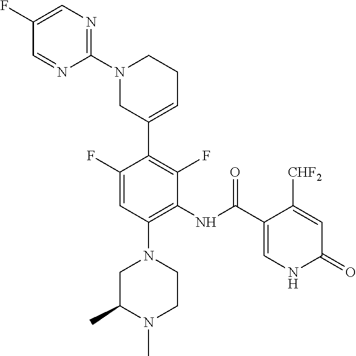 [SMILES: C[C@H]1CN(c2cc(F)c(C3=CCCN(c4ncc(F)cn4)C3)c(F)c2NC(=O)c2c[nH]c(=O)cc2C(F)F)CCN1C]

A procedure similar to Example 48 using (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide (25 mg, 0.051 mmol, preparation described in Example 35) and 2-bromo-5-fluoropyrimidine (8.97 mg, 0.051 mmol) afforded the title compound as an off white powder (22 mg, 70%). LCMS [M+1]⁺ 690.6.

### Example 84: (S)—N-(6-(3,4-dimethylpiperazin-1-yl)-2,4-difluoro-3-(1-(5-fluoropyrimidin-2-yl)-1,2,5,6-tetrahydropyridin-3-yl)phenyl)-6-oxo-4-(trifluoromethyl)-1,6-dihydropyridine-3-carboxamide

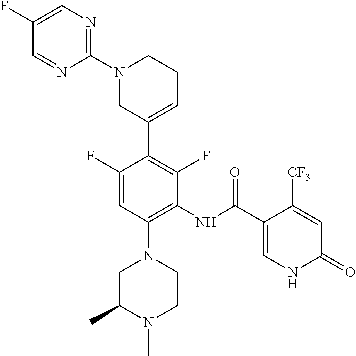 [SMILES: C[C@H]1CN(c2cc(F)c(C3=CCCN(c4ncc(F)cn4)C3)c(F)c2NC(=O)c2c[nH]c(=O)cc2C(F)(F)F)CCN1C]

A procedure similar to Example 48 using (S)—N-(6-(3,4-dimethylpiperazin-1-yl)-2,4-difluoro-3-(1,2,5,6-tetrahydropyridin-3-yl)phenyl)-6-oxo-4-(trifluoromethyl)-1,6-dihydropyridine-3-carboxamide (25 mg, 0.049 mmol, preparation described in Example 38) and 2-bromo-5-fluoropyrimidine (8.65 mg, 0.049 mmol). The title compound was isolated as an off white powder (21 mg, 67%). LCMS [M+1]⁺ 608.7.

### Example 85: N-(3-(2-((2R,6R)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide

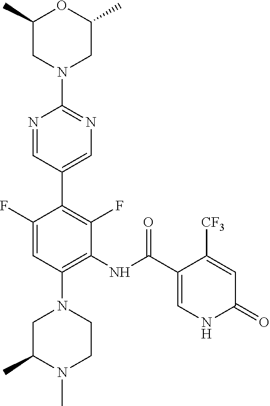 [SMILES: C[C@@H]1CN(c2ncc(-c3c(F)cc(N4CCN(C)[C@@H](C)C4)c(NC(=O)c4c[nH]c(=O)cc4C(F)(F)F)c3F)cn2)C[C@@H](C)O1]

To a mixture of 2-chloropyrimidine-5-boronic acid (317 mg, 2 mmol) and (2R,6R)-2,6-dimethyl-morpholine (242 mg, 2.1 mmol) in EtOH (5 mL) was added triethylamine (0.70 mL, 5 mmol). The resulting mixture was stirred at 75° C. for 1.5 h. Solvents were removed and the residue was dried under high vacuum to give crude (2-((2R,6R)-2,6-dimethylmorpholino)pyrimidin-5-yl)boronic acid as a light yellow solid (785 mg, 2 mmol, 60% purity assuming full conversion). LCMS [M+H]⁺ 238.3. The title compound (beige solid, 36.4 mg, 58%) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (2-((2R,6R)-2,6-dimethylmorpholino)pyrimidin-5-yl)boronic acid (0.2 mmol, crude) followed by TFA deprotection (1 mL TFA/6 mL DCM). LCMS [M+H]⁺ 622.6.

### Example 86: N-(3-(2-((2R,6R)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

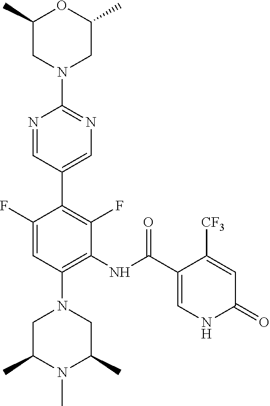 [SMILES: C[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)C[C@@H](C)O1]

The title compound (light brown solid, 37.0 mg, 58%) was prepared according to a procedure similar to Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (62 mg, 0.1 mmol) and (2-((2R,6R)-2,6-dimethylmorpholino)pyrimidin-5-yl)boronic acid (0.2 mmol, crude) followed by TFA deprotection (1 mL TFA/10 mL DCM). LCMS [M+H]⁺ 636.5.

### Example 87: N-(3-(2-((2S,6S)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide

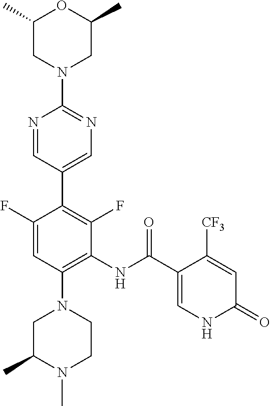 [SMILES: C[C@H]1CN(c2ncc(-c3c(F)cc(N4CCN(C)[C@@H](C)C4)c(NC(=O)c4c[nH]c(=O)cc4C(F)(F)F)c3F)cn2)C[C@H](C)O1]

To a mixture of 2-chloropyrimidine-5-boronic acid (317 mg, 2 mmol) and (2S,6S)-2,6-dimethyl-morpholine (242 mg, 2.1 mmol) in EtOH (5 mL) was added triethylamine (0.70 mL, 5 mmol). The resulting mixture was stirred at 75° C. for 1.5 h. Solvents were removed and the residue was dried under high vacuum to give crude (2-((2S,6S)-2,6-dimethylmorpholino)pyrimidin-5-yl)boronic acid as a light yellow solid (787 mg, 2 mmol, 60% purity assuming full conversion). LCMS [M+H]⁺ 238.2. The title compound (beige solid, 33.8 mg, 54%) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol, preparation described in Example 47), (2-((2S,6S)-2,6-dimethylmorpholino)pyrimidin-5-yl)boronic acid (0.2 mmol, crude) followed by TFA deprotection (1 mL TFA/6 mL DCM). LCMS [M+H]⁺ 622.6.

### Example 88: N-(3-(2-((2S,6S)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

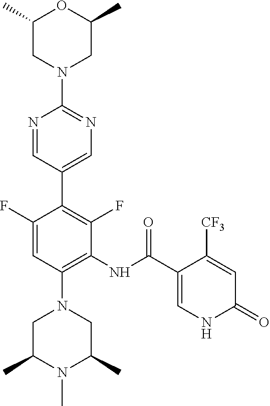 [SMILES: C[C@@H]1CN(c2cc(F)c(-c3cnc(N4C[C@H](C)O[C@@H](C)C4)nc3)c(F)c2NC(=O)c2c[nH]c(=O)cc2C(F)(F)F)C[C@H](C)N1C]

The title compound (light beige solid, 31.2 mg, 49%) was prepared according to a procedure similar to Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (62 mg, 0.1 mmol, preparation described in Example 13), (2-((2S,6S)-2,6-dimethylmorpholino)pyrimidin-5-yl)boronic acid (0.2 mmol, crude) followed by TFA deprotection (1 mL TFA/6 mL DCM). LCMS [M+H]⁺ 636.5.

### Example 89: N-(3-(2-((1R,5S)-8-oxa-3-azabicyclo[3.2.1]octan-3-yl)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide

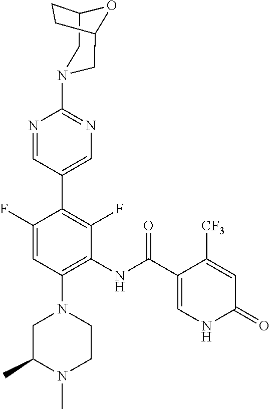 [SMILES: C[C@H]1CN(c2cc(F)c(-c3cnc(N4CC5CCC(C4)O5)nc3)c(F)c2NC(=O)c2c[nH]c(=O)cc2C(F)(F)F)CCN1C]

To a mixture of 2-chloropyrimidine-5-boronic acid (475 mg, 3 mmol) and 8-oxa-3-aza-bicyclo[3.2.1]octane (356 mg, 3.15 mmol) in EtOH (5 mL) was added triethylamine (1.05 mL, 7.5 mmol). The resulting mixture was stirred at 75° C. for 1.5 h. Solvents were removed and the residue was dried under high vacuum to give crude (2-((1R,5S)-8-oxa-3-azabicyclo[3.2.1]octan-3-yl)pyrimidin-5-yl)boronic acid as alight yellow solid (939 mg, 3 mmol, 75% purity assuming full conversion). LCMS [M+H]⁺ 236.2. The title compound (beige solid, 13.5 mg, 21%) was prepared according to a procedure similar to that described in Example 1, Step 6 using ((S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (2-((1R,5S)-8-oxa-3-azabicyclo[3.2.1]octan-3-yl)pyrimidin-5-yl)boronic acid (0.2 mmol, crude) followed by TFA deprotection (1 mL TFA/6 mL DCM). LCMS [M+H]⁺ 620.4.

### Example 90: N-(3-(2-((R,5S)-8-oxa-3-azabicyclo[3.2.1]octan-3-yl)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

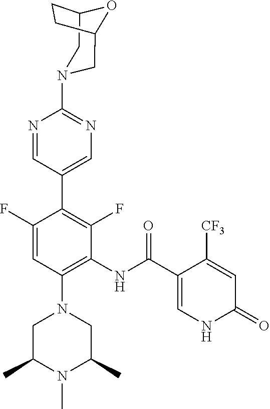 [SMILES: C[C@@H]1CN(c2cc(F)c(-c3cnc(N4CC5CCC(C4)O5)nc3)c(F)c2NC(=O)c2c[nH]c(=O)cc2C(F)(F)F)C[C@H](C)N1C]

The title compound (beige solid, 13.9 mg, 22% yield) was prepared according to a procedure similar to Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (62 mg, 0.1 mmol, preparation described in Example 13) and (2-((1R,5S)-8-oxa-3-azabicyclo[3.2.1]octan-3-yl)pyrimidin-5-yl)boronic acid (0.2 mmol, crude) followed by TFA deprotection (1 mL TFA/6 mL DCM). LCMS [M+H]⁺ 634.5.

### Example 91: (S)-4-(difluoromethyl)-N-(6-(3,4-dimethylpiperazin-1-yl)-2,4-difluoro-3-(1-(5-fluoropyrimidin-2-yl)-2,5-dihydro-1H-pyrrol-3-yl)phenyl)-6-oxo-1,6-dihydropyridine-3-carboxamide

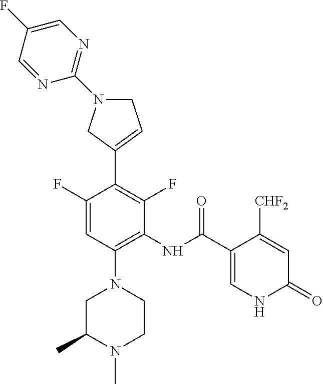 [SMILES: C[C@H]1CN(c2cc(F)c(C3=CCN(c4ncc(F)cn4)C3)c(F)c2NC(=O)c2c[nH]c(=O)cc2C(F)F)CCN1C]

To a solution of the intermediate (S)-4-(difluoromethyl)-N-(3-(2,5-dihydro-1H-pyrrol-3-yl)-6-(3,4-dimethylpiperazin-1-yl)-2,4-difluorophenyl)-6-oxo-1,6-dihydropyridine-3-carboxamide (25 mg, 0.052 mmol, preparation described in Example 33) and 2-Bromo-5-fluoropyrimidine (9.23 mg, 0.052 mmol) in isopropanol (2.5 mL) at RT was added N,N-diisopropylethylamine (0.018 ml, 0.104 mmol). After heating in a microwave reactor at 150° C. for 1.5 h, the reaction mixture was purified on preparatory column eluting with water (containing 0.1% HCOOH)/acetonitrile (containing 0.1% HCOOH) gradient (90/70). The title compound was isolated as an off white solid (18 mg, 57%). LCMS [M+1]⁺=576.6.

### Example 92: 1-Methylcyclobutyl(S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

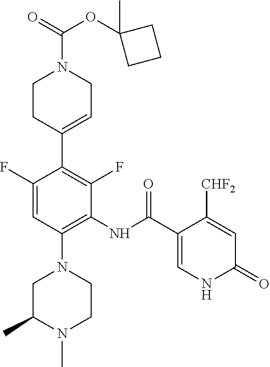 [SMILES: C[C@H]1CN(c2cc(F)c(C3=CCN(C(=O)OC4(C)CCC4)CC3)c(F)c2NC(=O)c2c[nH]c(=O)cc2C(F)F)CCN1C]

A solution of (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (25 mg, 0.051 mmol, preparation described in Example 34), 1-methylcyclobutyl (4-nitrophenyl) carbonate (19.09 mg, 0.061 mmol) in pyridine, anhydrous (16.03 mg, 0.203 mmol) was heated in a tightly capped vial at 60° C. for 45 minutes. The reaction mixture was concentrated and purified by sg chromatography, eluting with DCM containing 0-5% MeOH and 0-0.5% NH₄OH. The title compound was isolated as an off white solid (24.5 mg, 76%). LCMS [M+1]⁺ 606.7.

### Example 93:1-Methylcyclobutyl(S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate

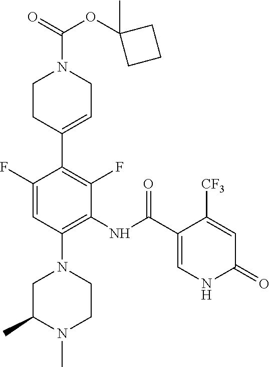 [SMILES: C[C@H]1CN(c2cc(F)c(C3=CCN(C(=O)OC4(C)CCC4)CC3)c(F)c2NC(=O)c2c[nH]c(=O)cc2C(F)(F)F)CCN1C]

The procedure was similar to Example 92 using (S)—N-(6-(3,4-dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide (preparation described in Example 47). The title compound was isolated as an off white powder (26.5 mg, 83%). LCMS [M+1]⁺ 624.7.

### Example 94: 1-Methylcyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate

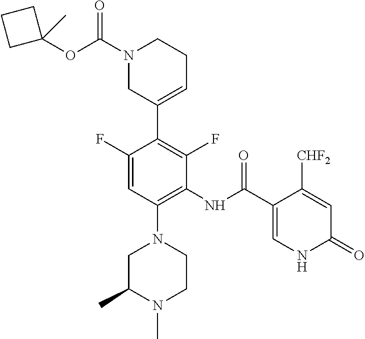 [SMILES: C[C@H]1CN(c2cc(F)c(C3=CCCN(C(=O)OC4(C)CCC4)C3)c(F)c2NC(=O)c2c[nH]c(=O)cc2C(F)F)CCN1C]

The procedure was similar to Example 92 using (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide (preparation described in Example 35). The title compound was isolated as a white powder (27 mg, 84% yield). LCMS [M+1]⁺606.7.

### Example 95: (S)—N-(3-(1-(5-cyanothiazol-2-yl)-2,5-dihydro-1H-pyrrol-3-yl)-6-(3,4-dimethylpiperazin-1-yl)-2,4-difluorophenyl)-4-(difluoromethyl)-6-oxo-1,6-dihydropyridine-3-carboxamide

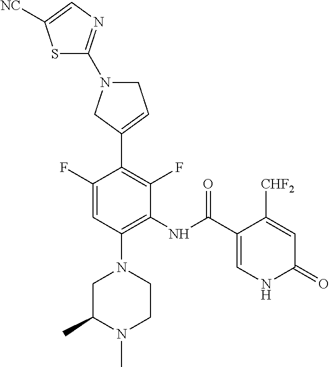 [SMILES: C[C@H]1CN(c2cc(F)c(C3=CCN(c4ncc(C#N)s4)C3)c(F)c2NC(=O)c2c[nH]c(=O)cc2C(F)F)CCN1C]

A mixture of intermediate (S)-4-(difluoromethyl)-N-(3-(2,5-dihydro-1H-pyrrol-3-yl)-6-(3,4-dimethylpiperazin-1-yl)-2,4-difluorophenyl)-6-oxo-1,6-dihydropyridine-3-carboxamide (25 mg, 0.052 mmol, preparation described in Example 36), 2-bromo-5-cyanothiazole (9.86 mg, 0.052 mmol) and triethylamine (0.029 ml, 0.209 mmol) in 2-propanol (2.0 ml) was heated in a microwave reactor at 150° C. for 1 h. The reaction mixture was cooled down to RT purified on reverse phase column, eluting with water (containing 0.1% HCOOH)/acetonitrile (containing 0.1% HCOOH) gradient. The desired product was isolated as an off white solid (21 mg, 65%) LCMS [M+1]⁺588.6.

### Example 96: (S)—N-(3-(1-(5-cyanothiazol-2-yl)-2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide

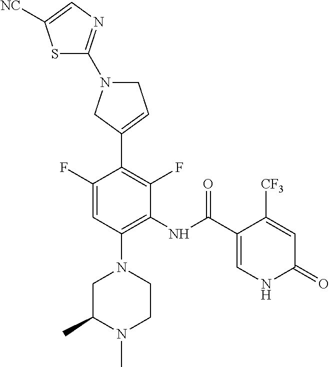 [SMILES: C[C@H]1CN(c2cc(F)c(C3=CCN(c4ncc(C#N)s4)C3)c(F)c2NC(=O)c2c[nH]c(=O)cc2C(F)(F)F)CCN1C]

The procedure followed was similar to Example 95 using (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (preparation described in Example 37). The title compound was isolated as an off white powder (22 mg, 69%). LCMS [M+1]⁺606.5.

### Example 97: (S)—N-(3-(1-(5-cyanothiazol-2-yl)-1,2,3,6-tetrahydropyridin-4-yl)-6-(3,4-dimethylpiperazin-1-yl)-2,4-difluorophenyl)-4-(difluoromethyl)-6-oxo-1,6-dihydropyridine-3-carboxamide

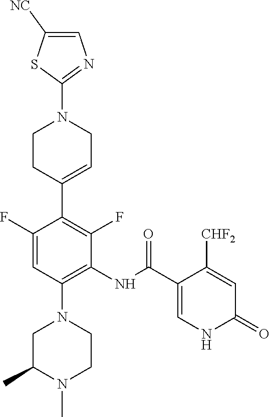 [SMILES: C[C@H]1CN(c2cc(F)c(C3=CCN(c4ncc(C#N)s4)CC3)c(F)c2NC(=O)c2c[nH]c(=O)cc2C(F)F)CCN1C]

The procedure followed was similar to Example 95 using (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (preparation described in Example 36). The title compound was isolated as an off white powder (25 mg, 78%). LCMS [M+1]⁺602.5.

### Example 98: (S)—N-(3-(1-(5-cyanothiazol-2-yl)-1,2,3,6-tetrahydropyridin-4-yl)-6-(3,4-dimethylpiperazin-1-yl)-2,4-difluorophenyl)-6-oxo-4-(trifluoromethyl)-1,6-dihydropyridine-3-carboxamide

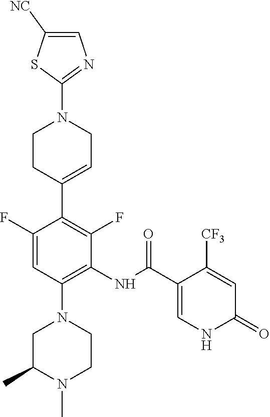 [SMILES: C[C@H]1CN(c2cc(F)c(C3=CCN(c4ncc(C#N)s4)CC3)c(F)c2NC(=O)c2c[nH]c(=O)cc2C(F)(F)F)CCN1C]

The procedure followed was similar to Example 95 using (S)—N-(6-(3,4-dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide (preparation described in Example 37). The title compound was isolated as an off white powder (21 mg, 66% yield). LCMS [M+1]⁺620.6.

### Example 99: (S)—N-(3-(1-(5-cyanothiazol-2-yl)-1,2,5,6-tetrahydropyridin-3-yl)-6-(3,4-dimethylpiperazin-1-yl)-2,4-difluorophenyl)-4-(difluoromethyl)-6-oxo-1,6-dihydropyridine-3-carboxamide

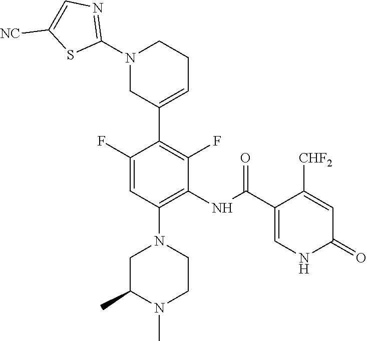 [SMILES: C[C@H]1CN(c2cc(F)c(C3=CCCN(c4ncc(C#N)s4)C3)c(F)c2NC(=O)c2c[nH]c(=O)cc2C(F)F)CCN1C]

The procedure followed was similar to Example 95 using (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide (preparation described in Example 36). The title compound was isolated as an off white powder (19.5 mg, 61%). LCMS [M+1]⁺602.6.

### Example 100: (S)—N-(3-(1-(5-cyanothiazol-2-yl)-1,2,5,6-tetrahydropyridin-3-yl)-6-(3,4-dimethylpiperazin-1-yl)-2,4-difluorophenyl)-6-oxo-4-(trifluoromethyl)-1,6-dihydropyridine-3-carboxamide

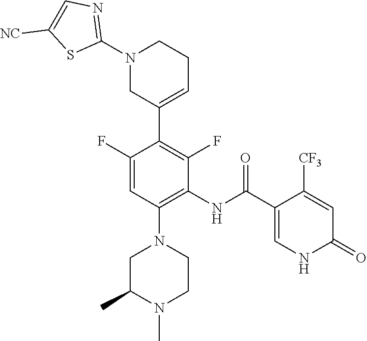 [SMILES: C[C@H]1CN(c2cc(F)c(C3=CCCN(c4ncc(C#N)s4)C3)c(F)c2NC(=O)c2c[nH]c(=O)cc2C(F)(F)F)CCN1C]

The procedure followed was similar to Example 95 using (S)—N-(6-(3,4-dimethylpiperazin-1-yl)-2,4-difluoro-3-(1,2,5,6-tetrahydropyridin-3-yl)phenyl)-6-oxo-4-(trifluoromethyl)-1,6-dihydropyridine-3-carboxamide (preparation described in Example 37). The title compound was isolated as an off white powder (21.5 mg, 67%). LCMS [M+1]⁺620.6.

### Example 101: N-(2,4-difluoro-3-(2-((S)-2-methylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide

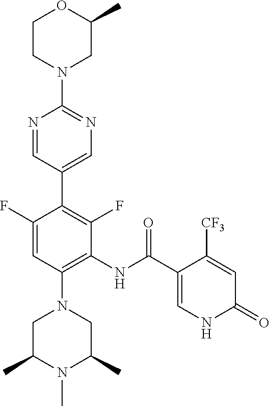 [SMILES: C[C@@H]1CN(c2cc(F)c(-c3cnc(N4CCO[C@@H](C)C4)nc3)c(F)c2NC(=O)c2c[nH]c(=O)cc2C(F)(F)F)C[C@H](C)N1C]

The title compound (beige solid, 31.0 mg, 48% yield) was prepared according to a procedure similar to Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (62 mg, 0.1 mmol preparation described in Example 13) and (S)-(2-(2-methylmorpholino)pyrimidin-5-yl)boronic acid (0.3 mmol, crude) followed by TFA deprotection (1 mL TFA/6 mL DCM). LCMS [M+H]⁺ 622.6.

### Example 102: N-(6-((S)-3,4-dimethylpiperazin-1-yl)-2,4-difluoro-3-(2-((S)-2-isopropylmorpholino)pyrimidin-5-yl)phenyl)-6-oxo-4-(trifluoromethyl)-1,6-dihydropyridine-3-carboxamide

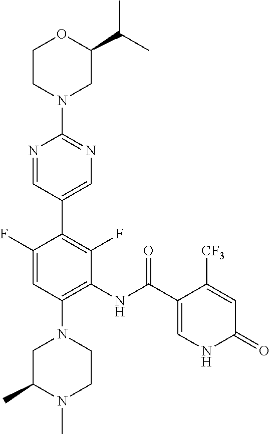 [SMILES: CC(C)[C@H]1CN(c2ncc(-c3c(F)cc(N4CCN(C)[C@@H](C)C4)c(NC(=O)c4c[nH]c(=O)cc4C(F)(F)F)c3F)cn2)CCO1]

To a mixture of 2-chloropyrimidine-5-boronic acid (158 mg, 1 mmol) and (S)-2-isopropylmorpholine (136 mg, 1.05 mmol) in EtOH (3 mL) was added triethylamine (0.35 mL, 2.5 mmol). The resulting mixture was stirred at 75° C. for 1.5 h. The solvents were removed and the residue was dried under high vacuum to give crude (S)-(2-(2-isopropylmorpholino)pyrimidin-5-yl)boronic acid as a light yellow solid (413 mg, 1 mmol, 61% assuming full conversion). LCMS [M+H]⁺ 252.3. The title compound (beige solid, 34.6 mg, 53%) was prepared according to a coupling procedure similar to that of Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol), (S)-(2-(2-isopropylmorpholino)pyrimidin-5-yl)boronic acid (0.25 mmol, crude) followed by TFA deprotection (1 mL TFA/6 mL DCM). LCMS [M+H]⁺ 636.5.

### Example 103: N-(2,4-difluoro-3-(2-((S)-2-isopropylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide

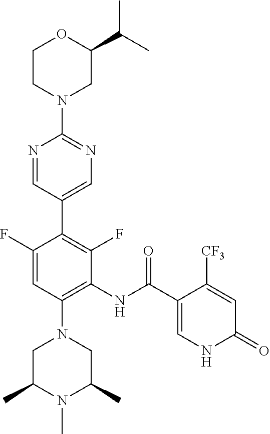 [SMILES: CC(C)[C@H]1CN(c2ncc(-c3c(F)cc(N4C[C@@H](C)N(C)[C@@H](C)C4)c(NC(=O)c4c[nH]c(=O)cc4C(F)(F)F)c3F)cn2)CCO1]

The title compound (beige solid, 41.4 mg, 63% yield) was prepared according to a procedure similar to Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (62 mg, 0.1 mmol), (S)-(2-(2-isopropylmorpholino)pyrimidin-5-yl)boronic acid (0.25 mmol, crude) followed by TFA deprotection (1 mL TFA/6 mL DCM). LCMS [M+H]⁺ 650.5.

### Example 104: N-(6-((S)-3,4-dimethylpiperazin-1-yl)-2,4-difluoro-3-(2-((R)-2-isopropylmorpholino)pyrimidin-5-yl)phenyl)-6-oxo-4-(trifluoromethyl)-1,6-dihydropyridine-3-carboxamide

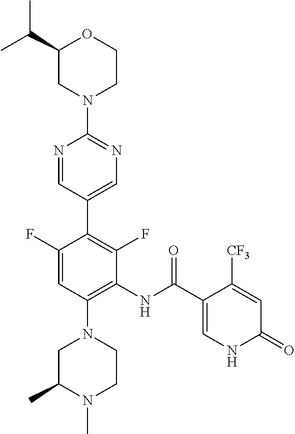 [SMILES: CC(C)[C@@H]1CN(c2ncc(-c3c(F)cc(N4CCN(C)[C@@H](C)C4)c(NC(=O)c4c[nH]c(=O)cc4C(F)(F)F)c3F)cn2)CCO1]

To a mixture of 2-chloropyrimidine-5-boronic acid (158 mg, 1 mmol) and (R)-2-isopropylmorpholine (136 mg, 1.05 mmol) in EtOH (3 mL) was added triethylamine (0.35 mL, 2.5 mmol). The resulting mixture was stirred at 75° C. for 1.5 h. Solvents were removed and the residue was dried under high vacuum to give crude (R)-(2-(2-isopropylmorpholino)pyrimidin-5-yl)boronic acid as a light yellow solid (409 mg, 1 mmol, 61% purity assuming full conversion). LCMS [M+H]⁺ 252.3. The title compound (beige solid, 31.1 mg, 49% yield) was prepared according to a coupling procedure similar to that described in Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (R)-(2-(2-isopropylmorpholino)pyrimidin-5-yl)boronic acid (0.25 mmol, crude) followed by TFA deprotection (1 mL TFA/6 mL DCM). LCMS [M+H]⁺ 636.5.

### Example 105: N-(2,4-difluoro-3-(2-((R)-2-isopropylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide

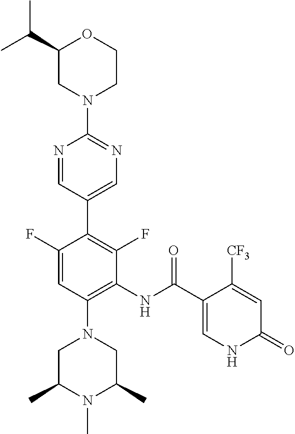 [SMILES: CC(C)[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)CCO1]

The title compound (beige solid, 40.9 mg, 62% yield) was prepared according to a coupling procedure similar to that in Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (62 mg, 0.1 mmol) and (R)-(2-(2-isopropylmorpholino)pyrimidin-5-yl)boronic acid (0.25 mmol, crude), followed by TFA deprotection (1 mL TFA/6 mL DCM). LCMS [M+H]⁺ 650.5.

### Example 106: (S)—N-(3-(1-(2-cyanopyrimidin-4-yl)-2,5-dihydro-1H-pyrrol-3-yl)-6-(3,4-dimethylpiperazin-1-yl)-2,4-difluorophenyl)-4-(difluoromethyl)-6-oxo-1,6-dihydropyridine-3-carboxamide

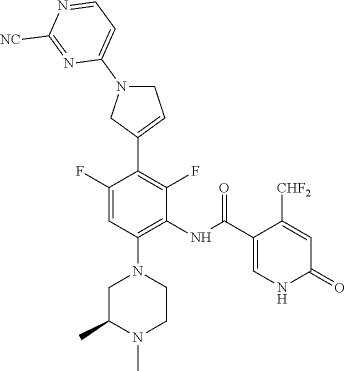 [SMILES: C[C@H]1CN(c2cc(F)c(C3=CCN(c4ccnc(C#N)n4)C3)c(F)c2NC(=O)c2c[nH]c(=O)cc2C(F)F)CCN1C]

A mixture of cesium carbonate (40.8 mg, 0.125 mmol), 4-bromopyrimidine-2-carbonitrile (12.66 mg, 0.069 mmol), and (S)-4-(difluoromethyl)-N-(3-(2,5-dihydro-1H-pyrrol-3-yl)-6-(3,4-dimethylpiperazin-1-yl)-2,4-difluorophenyl)-6-oxo-1,6-dihydropyridine-3-carboxamide (30 mg, 0.063 mmol, preparation described in Example 36) in N-methyl-2-pyrrolidone was heated in an oil bath at 85° C. for 30 min. The mixture was partitioned between DCM and water, the org. phase was separated, aq phase was extracted with dichloromethane (2×), the combined org phase was washed with brine, dried over Na₂SO₄ and concentrated to get the crude product which was purified by flash column chromatography (eluting with DCM containing 0-6% MeOH and 0-0.6% NH₄OH). The title compound was isolated as a beige white solid (16 mg, 40% yield). LCMS [M+1]⁺583.6.

### Example 107: (S)—N-(3-(1-(2-cyanopyrimidin-4-yl)-2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide

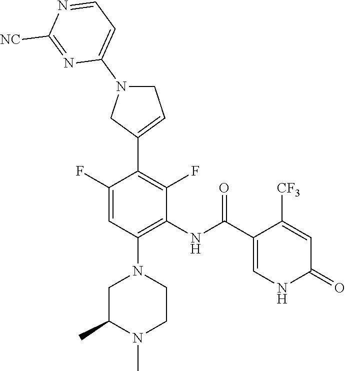 [SMILES: C[C@H]1CN(c2cc(F)c(C3=CCN(c4ccnc(C#N)n4)C3)c(F)c2NC(=O)c2c[nH]c(=O)cc2C(F)(F)F)CCN1C]

The procedure followed was similar to Example 95 using (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide. The title compound was isolated as a beige powder (6 mg, 28% yield). LCMS [M+1]⁺601.7.

### Example 108: (S)—N-(3-(1-(2-cyanopyrimidin-4-yl)-1,2,3,6-tetrahydropyridin-4-yl)-6-(3,4-dimethylpiperazin-1-yl)-2,4-difluorophenyl)-4-(difluoromethyl)-6-oxo-1,6-dihydropyridine-3-carboxamide

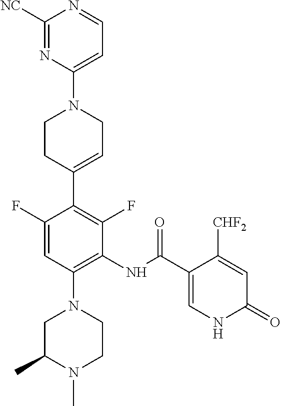 [SMILES: C[C@H]1CN(c2cc(F)c(C3=CCN(c4ccnc(C#N)n4)CC3)c(F)c2NC(=O)c2c[nH]c(=O)cc2C(F)F)CCN1C]

A mixture of cesium carbonate (20.96 mg, 0.064 mmol), 4-bromopyrimidine-2-carbonitrile (6.51 mg, 0.035 mmol), and (S)—N-(3-(2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide (16 mg, 0.032 mmol). The title compound was isolated as a beige powder (22 mg, 58%). LCMS [M+1]⁺597.7.

### Example 109: 2-(Difluoromethyl)-N-(3-(2-((2S,6R)-2,6-dimethylmorpholino)pyrimidin-5-yl)-2,4-difluoro-6-((3S,5R)-3,4,5-trimethylpiperazin-1-yl)phenyl)-4-fluorobenzamide Formic Acid

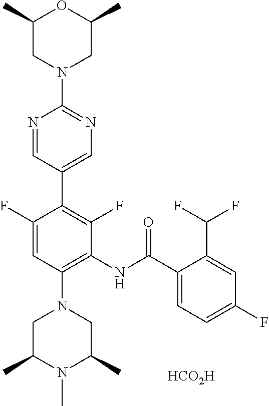 [SMILES: C[C@@H]1CN(c2ncc(-c3c(F)cc(N4C[C@@H](C)N(C)[C@@H](C)C4)c(NC(=O)c4ccc(F)cc4C(F)F)c3F)cn2)C[C@H](C)O1 ; "HCO2H"]

3-(2-((2S,6R)-2,6-dimethylmorpholino)pyrimidin-5-yl)-2,4-difluoro-6-((3S,5R)-3,4,5-trimethylpiperazin-1-yl)aniline (402 mg, 85% based on 93.88% purity) was prepared according to a coupling procedure similar to that described in Example 1, Step 6 using 3-bromo-2,4-difluoro-6-((3S,5R)-3,4,5-trimethylpiperazin-1-yl)aniline (334 mg, 1 mmol) and (2S,6R)-2,6-dimethyl-4-(5-(4,4,5,5-tetramethyl-1,3,2-dioxaborolan-2-yl)pyrimidin-2-yl)morpholine (479 mg, 1.5 mmol). LCMS [M+H]⁺ 447.5. The title compound (formic acid salt, light beige solid, 37.3 mg, 56%) was then prepared according to a procedure similar to that described in Example 13 using 3-(2-((2S,6R)-2,6-dimethylmorpholino)pyrimidin-5-yl)-2,4-difluoro-6-((3S,5R)-3,4,5-trimethylpiperazin-1-yl)aniline (48 mg, 93.88% purity, 0.1 mmol), 2-(difluoromethyl)-4-fluorobenzoic acid (34 mg, 0.18 mmol) and T3P (50% wt % in EtOAc, 0.12 mL, 0.2 mmol). LCMS [M+H]⁺ 619.5.

### Example 110: 2-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-4-fluorobenzamide formic acid

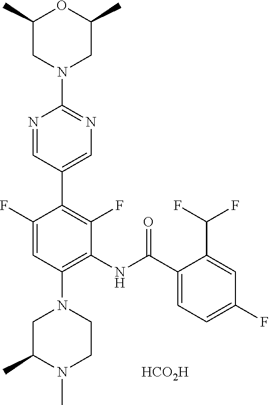 [SMILES: C[C@@H]1CN(c2ncc(-c3c(F)cc(N4CCN(C)[C@@H](C)C4)c(NC(=O)c4ccc(F)cc4C(F)F)c3F)cn2)C[C@H](C)O1 ; "HCO2H"]

3-(2-((2S,6R)-2,6-dimethylmorpholino)pyrimidin-5-yl)-6-((S)-3,4-dimethylpiperazin-1-yl)-2,4-difluoroaniline (browns solid, 473 mg, quantitative yield, 1 mmol, 91% purity assuming full conversion) was prepared according to a coupling procedure similar to that described in Example 1, Step 6 using (S)-3-bromo-6-(3,4-dimethylpiperazin-1-yl)-2,4-difluoroaniline (320 mg, 1 mmol) and (2S,6R)-2,6-dimethyl-4-(5-(4,4,5,5-tetramethyl-1,3,2-dioxaborolan-2-yl)pyrimidin-2-yl)morpholine (479 mg, 1.5 mmol). LCMS [M+H]⁺ 433.5. The title compound (formic acid salt, pale beige solid, 38.9 mg, 60%) was prepared according to the procedure in Example 13 using 3-(2-((2S,6R)-2,6-dimethylmorpholino)pyrimidin-5-yl)-6-((S)-3,4-dimethylpiperazin-1-yl)-2,4-difluoroaniline (0.1 mmol), 2-(difluoromethyl)-4-fluorobenzoic acid (34 mg, 0.18 mmol) and T3P (50% wt % in EtOAc, 0.12 mL, 0.2 mmol). LCMS [M+H]⁺ 605.5.

### Example 111: 4-(Difluoromethyl)-N-(3-(2-((2S,6R)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide

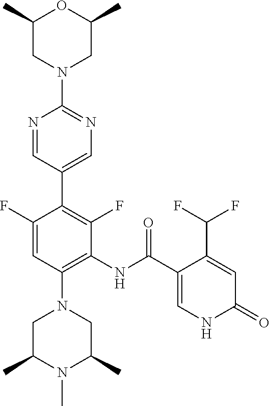 [SMILES: C[C@@H]1CN(c2ncc(-c3c(F)cc(N4C[C@@H](C)N(C)[C@@H](C)C4)c(NC(=O)c4c[nH]c(=O)cc4C(F)F)c3F)cn2)C[C@H](C)O1]

The title compound (beige solid, 25.8 mg, 41%) was prepared according to a procedure similar to Example 1, Step 6 using 3-(2-((2S,6R)-2,6-dimethylmorpholino)pyrimidin-5-yl)-2,4-difluoro-6-((3S,5R)-3,4,5-trimethylpiperazin-1-yl)aniline (48 mg, 93.88% purity, 0.1 mmol), 4-(difluoromethyl)-6-(2-(trimethylsilyl)ethoxy)nicotinic acid (52 mg, 0.18 mmol) and T3P (50% wt % in EtOAc, 0.12 mL, 0.2 mmol) followed by TFA deprotection (1 mL TFA/6 mL DCM). LCMS [M+H]⁺ 618.5.

### Example 112: (S)—N-(3-(1-(2-cyanopyrimidin-4-yl)-1,2,3,6-tetrahydropyridin-4-yl)-6-(3,4-dimethylpiperazin-1-yl)-2,4-difluorophenyl)-6-oxo-4-(trifluoromethyl)-1,6-dihydropyridine-3-carboxamide

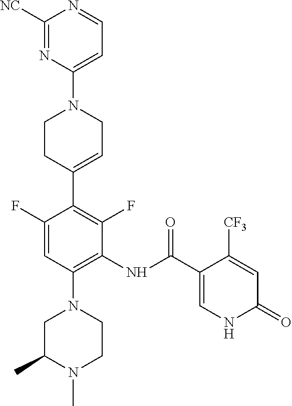 [SMILES: C[C@H]1CN(c2cc(F)c(C3=CCN(c4ccnc(C#N)n4)CC3)c(F)c2NC(=O)c2c[nH]c(=O)cc2C(F)(F)F)CCN1C]

The procedure was similar to Example 106 using (47.1 mg, 0.145 mmol),4-bromopyrimidine-2-carbonitrile (14.64 mg, 0.080 mmol) and (S)—N-(6-(3,4-dimethylpiperazin-1-yl)-2,4-difluoro-3-(1,2,3,6-tetrahydropyridin-4-yl)phenyl)-6-oxo-4-(trifluoromethyl)-1,6-dihydropyridine-3-carboxamide (37 mg, 0.072 mmol). The title compound was isolated as an off white powder (13 mg, 28%). LCMS [M+1]⁺=615.7.

### Example 113: (S)—N-(3-(1-(2-cyanopyrimidin-4-yl)-1,2,5,6-tetrahydropyridin-3-yl)-6-(3,4-dimethylpiperazin-1-yl)-2,4-difluorophenyl)-4-(difluoromethyl)-6-oxo-1,6-dihydropyridine-3-carboxamide

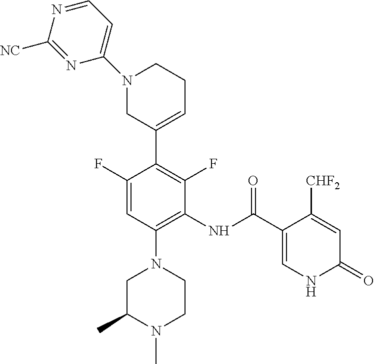 [SMILES: C[C@H]1CN(c2cc(F)c(C3=CCCN(c4ccnc(C#N)n4)C3)c(F)c2NC(=O)c2c[nH]c(=O)cc2C(F)F)CCN1C]

The procedure followed was similar to Example 106 using 4-bromopyrimidine-2-carbonitrile (12.30 mg, 0.067 mmol), and (S)-4-(difluoromethyl)-N-(6-(3,4-dimethylpiperazin-1-yl)-2,4-difluoro-3-(1,2,5,6-tetrahydropyridin-3-yl)phenyl)-6-oxo-1,6-dihydropyridine-3-carboxamide (30 mg, 0.061 mmol). The title compound was isolated as an off white powder (17 mg, 45%). LCMS [M+1]⁺=597.6.

### Example 114: (S)—N-(3-(1-(2-cyanopyrimidin-4-yl)-1,2,5,6-tetrahydropyridin-3-yl)-6-(3,4-dimethylpiperazin-1-yl)-2,4-difluorophenyl)-6-oxo-4-(trifluoromethyl)-1,6-dihydropyridine-3-carboxamide

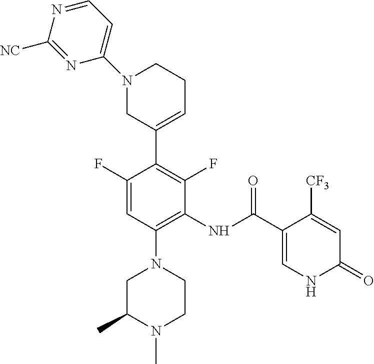 [SMILES: C[C@H]1CN(c2cc(F)c(C3=CCCN(c4ccnc(C#N)n4)C3)c(F)c2NC(=O)c2c[nH]c(=O)cc2C(F)(F)F)CCN1C]

A procedure similar to Example 106 using 4-bromopyrimidine-2-carbonitrile (11.87 mg, 0.065 mmol), and (S)—N-(6-(3,4-dimethylpiperazin-1-yl)-2,4-difluoro-3-(1,2,5,6-tetrahydropyridin-3-yl)phenyl)-6-oxo-4-(trifluoromethyl)-1,6-dihydropyridine-3-carboxamide (30 mg, 0.059 mmol, preparation described in Example 47) afforded the title compound as a beige powder (10.5 mg, 28%). LCMS [M+1]⁺615.7.

### Example 115:4-(Difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide

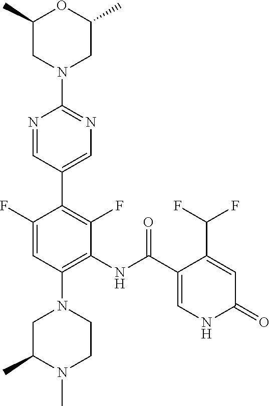 [SMILES: C[C@@H]1CN(c2ncc(-c3c(F)cc(N4CCN(C)[C@@H](C)C4)c(NC(=O)c4c[nH]c(=O)cc4C(F)F)c3F)cn2)C[C@@H](C)O1]

The title compound (beige solid, 23.4 mg, 38% yield) was prepared according to a procedure similar to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 85.5% purity, 0.1 mmol, preparation described in Example 33) and (2-((2R,6R)-2,6-dimethylmorpholino)pyrimidin-5-yl)boronic acid (0.2 mmol, crude) followed by TFA deprotection (1 mL TFA/6 mL DCM). LCMS [M+H]⁺ 604.5.

### Example 116:4-(Difluoromethyl)-N-(3-(2-((2R,6R)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide

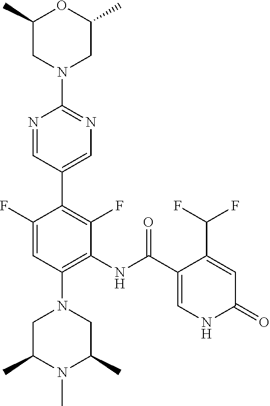 [SMILES: C[C@@H]1CN(c2ncc(-c3c(F)cc(N4C[C@@H](C)N(C)[C@@H](C)C4)c(NC(=O)c4c[nH]c(=O)cc4C(F)F)c3F)cn2)C[C@@H](C)O1]

Intermediate N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide was prepared as a dark brown foam (1.408 g, 99% based on 85.0% purity) according to by a procedure similar to that described in Example 13 using 3-bromo-2,4-difluoro-6-((3S,5R)-3,4,5-trimethylpiperazin-1-yl)aniline (668 mg, 2 mmol), 4-(difluoromethyl)-6-(2-(trimethylsilyl)ethoxy)nicotinic acid (752 mg, 2.6 mmol) and T3P (50% wt % in EtOAc, 3.57 mL, 6 mmol). LCMS [M+H]⁺ 605.3. The title compound (beige solid, 21.6 mg, 35%) was prepared according to a procedure similar to that described in Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (71 mg, 85.0% purity, 0.1 mmol) and (2-((2R,6R)-2,6-dimethylmorpholino)pyrimidin-5-yl)boronic acid (0.2 mmol+0.1 mmol, crude) followed by TFA deprotection (1 mL TFA/6 mL DCM). LCMS [M+H]⁺ 618.6.

### Example 117: 4-(Difluoromethyl)-N-(6-((S)-3,4-dimethylpiperazin-1-yl)-2,4-difluoro-3-(2-((R)-2-isopropylmorpholino)pyrimidin-5-yl)phenyl)-6-oxo-1,6-dihydropyridine-3-carboxamide

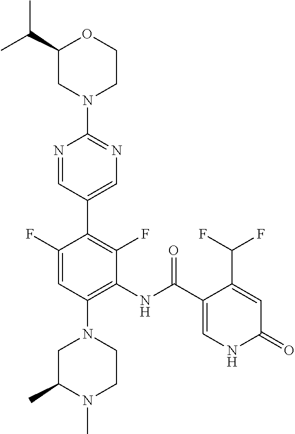 [SMILES: CC(C)[C@@H]1CN(c2ncc(-c3c(F)cc(N4CCN(C)[C@@H](C)C4)c(NC(=O)c4c[nH]c(=O)cc4C(F)F)c3F)cn2)CCO1]

The title compound (light beige solid, 28.0 mg, 46%) was prepared according to a procedure similar to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 85.5% purity, 0.1 mmol) and (R)-(2-(2-isopropylmorpholino)pyrimidin-5-yl)boronic acid (0.25 mmol, crude) followed by TFA deprotection (1 mL TFA/6 mL DCM). LCMS [M+H]⁺ 618.6.

### Example 118: N-(2,4-difluoro-3-(2-((R)-2-isopropylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide

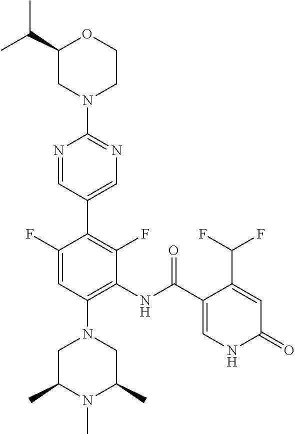 [SMILES: CC(C)[C@@H]1CN(c2ncc(-c3c(F)cc(N4C[C@@H](C)N(C)[C@@H](C)C4)c(NC(=O)c4c[nH]c(=O)cc4C(F)F)c3F)cn2)CCO1]

The title compound (light beige solid, 25.1 mg, 40%) was prepared according to a coupling procedure similar to that described in Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (71 mg, 85.0% purity, 0.1 mmol) and (R)-(2-(2-isopropylmorpholino)pyrimidin-5-yl)boronic acid (0.25 mmol, crude) followed by TFA deprotection (1 mL TFA/6 mL DCM). LCMS [M+H]⁺ 632.6.

### Example 119: 4-(Difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide

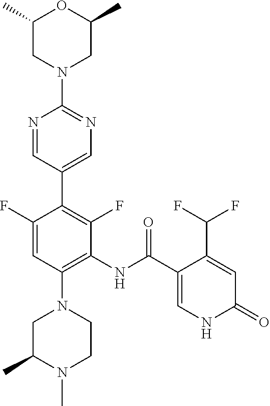 [SMILES: C[C@H]1CN(c2ncc(-c3c(F)cc(N4CCN(C)[C@@H](C)C4)c(NC(=O)c4c[nH]c(=O)cc4C(F)F)c3F)cn2)C[C@H](C)O1]

The title compound (light beige solid, 28.7 mg, 46% yield) was prepared according to a coupling procedure similar to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 85.5% purity, 0.1 mmol, preparation described in Example 33) and (2-((2S,6S)-2,6-dimethylmorpholino)pyrimidin-5-yl)boronic acid (0.2 mmol, crude) followed by TFA deprotection (1 mL TFA/6 mL DCM). LCMS [M+H]⁺ 604.6.

### Example 120: 4-(Difluoromethyl)-N-(3-(2-((2S,6S)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide

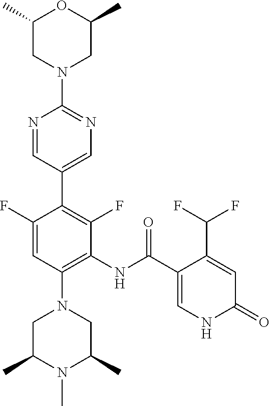 [SMILES: C[C@@H]1CN(c2cc(F)c(-c3cnc(N4C[C@H](C)O[C@@H](C)C4)nc3)c(F)c2NC(=O)c2c[nH]c(=O)cc2C(F)F)C[C@H](C)N1C]

The title compound (beige solid, 23.1 mg, 37% yield) was prepared according to a coupling procedure similar to Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (71 mg, 85.0% purity, 0.1 mmol, preparation described in Example 116) and (2-((2S,6S)-2,6-dimethylmorpholino)pyrimidin-5-yl)boronic acid (0.2 mmol, crude) followed by TFA deprotection (1 mL TFA/6 mL DCM). LCMS [M+H]⁺ 618.6.

### Example 121: 4-(Difluoromethyl)-N-(6-((S)-3,4-dimethylpiperazin-1-yl)-2,4-difluoro-3-(2-((S)-2-isopropylmorpholino)pyrimidin-5-yl)phenyl)-6-oxo-1,6-dihydropyridine-3-carboxamide

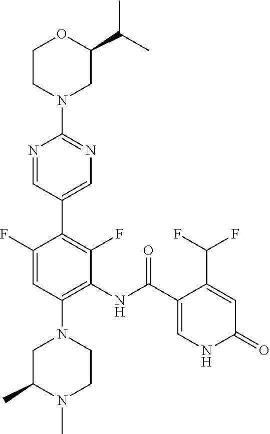 [SMILES: CC(C)[C@H]1CN(c2ncc(-c3c(F)cc(N4CCN(C)[C@@H](C)C4)c(NC(=O)c4c[nH]c(=O)cc4C(F)F)c3F)cn2)CCO1]

The title compound (beige solid, 23.6 mg, 38% yield) was prepared according to a procedure similar to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 85.5% purity, 0.1 mmol, from Example 33) and (S)-(2-(2-isopropylmorpholino)pyrimidin-5-yl)boronic acid (0.25 mmol, crude) followed by TFA deprotection (1 mL TFA/6 mL DCMLCMS [M+H]⁺ 618.6.

### Example 122: N-(2,4-Difluoro-3-(2-((S)-2-isopropylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide

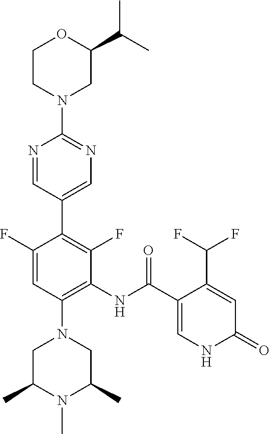 [SMILES: CC(C)[C@H]1CN(c2ncc(-c3c(F)cc(N4C[C@@H](C)N(C)[C@@H](C)C4)c(NC(=O)c4c[nH]c(=O)cc4C(F)F)c3F)cn2)CCO1]

The title compound (beige solid, 33.7 mg, 53% yield) was prepared according to a procedure similar to that described in Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (71 mg, 85.0% purity, 0.1 mmol) and (S)-(2-(2-isopropylmorpholino)pyrimidin-5-yl)boronic acid (0.25 mmol, crude) followed by TFA deprotection (1 mL TFA/6 mL DCM). LCMS [M+H]⁺ 632.6.

### Example 123: N-(6-((S)-3,4-dimethylpiperazin-1-yl)-2,4-difluoro-3-(2-((R)-2-isopropylmorpholino)pyrimidin-4-yl)phenyl)-6-oxo-4-(trifluoromethyl)-1,6-dihydropyridine-3-carboxamide

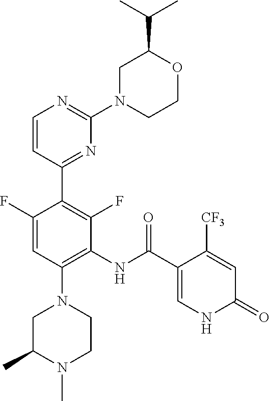 [SMILES: CC(C)[C@@H]1CN(c2nccc(-c3c(F)cc(N4CCN(C)[C@@H](C)C4)c(NC(=O)c4c[nH]c(=O)cc4C(F)(F)F)c3F)n2)CCO1]

6-((S)-3,4-dimethylpiperazin-1-yl)-2,4-difluoro-3-(2-((R)-2-isopropylmorpholino)pyrimidin-4-yl)aniline (292 mg, 64% yield) was prepared on 1 mmol scale according to the method used for the preparation of 3-(2-((2S,6R)-2,6-dimethylmorpholino)pyrimidin-4-yl)-6-((S)-3,4-dimethylpiperazin-1-yl)-2,4-difluoroaniline by replacing (2S,6R)-4-(4-bromopyrimidin-2-yl)-2,6-dimethylmorpholine with (R)-4-(4-bromopyrimidin-2-yl)-2-isopropylmorpholine (429 mg, 1.5 mmol). LCMS [M+H]⁺ 447.5. The title compound (off white solid, 7.6 mg, 6%) was prepared according to according to a procedure similar to that described in Example 13 using 6-((S)-3,4-dimethylpiperazin-1-yl)-2,4-difluoro-3-(2-((R)-2-isopropylmorpholino)pyrimidin-4-yl)aniline (89 mg, 0.2 mmol), 4-(trifluoromethyl)-6-(2-(trimethylsilyl)ethoxy)nicotinic acid (111 mg, 0.36 mmol) and T3P (50% wt % in EtOAc, 0.24 mL, 0.4 mmol) followed by TFA deprotection (1 mL TFA/6 mL DCM). LCMS [M+H]⁺ 636.6.

### Example 124: 4-(Difluoromethyl)-N-(6-((S)-3,4-dimethylpiperazin-1-yl)-2,4-difluoro-3-(2-((R)-2-isopropylmorpholino)pyrimidin-4-yl)phenyl)-6-oxo-1,6-dihydropyridine-3-carboxamide

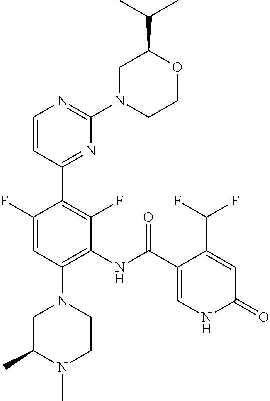 [SMILES: CC(C)[C@@H]1CN(c2nccc(-c3c(F)cc(N4CCN(C)[C@@H](C)C4)c(NC(=O)c4c[nH]c(=O)cc4C(F)F)c3F)n2)CCO1]

The title compound (pale beige solid, 21.4 mg, 17% yield) was prepared according to a procedure similar to that described in Example 13 using 6-((S)-3,4-dimethylpiperazin-1-yl)-2,4-difluoro-3-(2-((R)-2-isopropylmorpholino)pyrimidin-4-yl)aniline (89 mg, 0.2 mmol), 4-(difluoromethyl)-6-(2-(trimethylsilyl)ethoxy)nicotinic acid (104 mg, 0.36 mmol) and T3P (50% wt % in EtOAc, 0.24 mL, 0.4 mmol) followed by TFA deprotection (1 mL TFA/6 mL DCM). LCMS [M+H]⁺ 618.6.

### Example 125: N-(2,4-difluoro-3-(2-((S)-2-methylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide

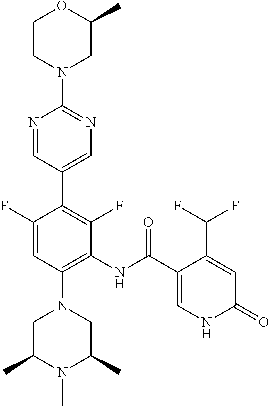 [SMILES: C[C@@H]1CN(c2cc(F)c(-c3cnc(N4CCO[C@@H](C)C4)nc3)c(F)c2NC(=O)c2c[nH]c(=O)cc2C(F)F)C[C@H](C)N1C]

The title compound (beige solid, 28.0 mg, 46% yield) was prepared according to a procedure similar to that described in Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (71 mg, 85.05% purity, 0.1 mmol) and (S)-(2-(2-methylmorpholino)pyrimidin-5-yl)boronic acid (0.3 mmol, crude) followed by TFA deprotection (1 mL TFA/6 mL DCM). LCMS [M+H]⁺ 604.6.

### Example 126: 4-(Difluoromethyl)-N-(6-((S)-3,4-dimethylpiperazin-1-yl)-2,4-difluoro-3-(2-((S)-2-methylmorpholino)pyrimidin-5-yl)phenyl)-6-oxo-1,6-dihydropyridine-3-carboxamide

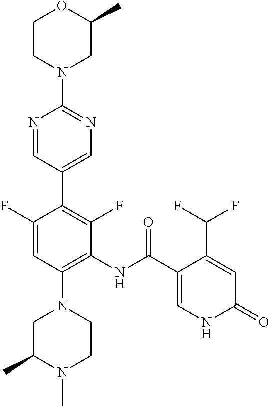 [SMILES: C[C@H]1CN(c2ncc(-c3c(F)cc(N4CCN(C)[C@@H](C)C4)c(NC(=O)c4c[nH]c(=O)cc4C(F)F)c3F)cn2)CCO1]

The title compound (beige solid, 22.4 mg, 37% yield) was prepared according to a procedure similar to that described in Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 85.5% purity, 0.1 mmol) and (S)-(2-(2-methylmorpholino)pyrimidin-5-yl)boronic acid (0.3 mmol, crude) followed by TFA deprotection (1 mL TFA/6 mL DCM); LCMS [M+H]⁺ 590.5.

### Example 127: N-(6-((S)-3,4-dimethylpiperazin-1-yl)-2,4-difluoro-3-(2-((R)-2-methylmorpholino)pyrimidin-5-yl)phenyl)-6-oxo-4-(trifluoromethyl)-1,6-dihydropyridine-3-carboxamide

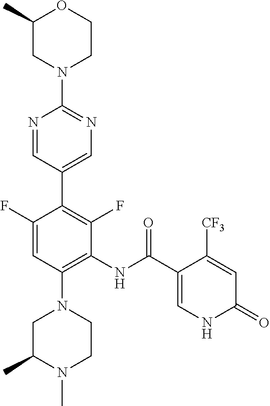 [SMILES: C[C@@H]1CN(c2ncc(-c3c(F)cc(N4CCN(C)[C@@H](C)C4)c(NC(=O)c4c[nH]c(=O)cc4C(F)(F)F)c3F)cn2)CCO1]

The title compound (pale beige solid, 76.4 mg, 62% yield) was prepared according to a coupling procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (122 mg, 0.2 mmol) and (R)-(2-(2-methylmorpholino)pyrimidin-5-yl)boronic acid (0.5 mmol, crude) followed by TFA deprotection (1 mL TFA/10 mL DCM). LCMS [M+H]⁺ 608.6.

### Example 128: N-(2,4-difluoro-3-(2-((R)-2-methylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide

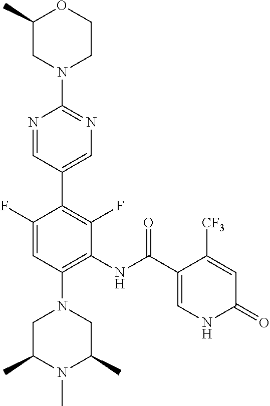 [SMILES: C[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)CCO1]

The title compound (light beige solid, 72.0 mg, 58% yield) was prepared according to a coupling procedure similar to that of Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (125 mg, 0.2 mmol) and (R)-(2-(2-methylmorpholino)pyrimidin-5-yl)boronic acid (0.5 mmol, crude) followed by TFA deprotection (1 mL TFA/10 mL DCM). LCMS [M+H]⁺ 622.5.

### Example 129: 4-(Difluoromethyl)-N-(6-((S)-3,4-dimethylpiperazin-1-yl)-2,4-difluoro-3-(2-((R)-2-methylmorpholino)pyrimidin-5-yl)phenyl)-6-oxo-1,6-dihydropyridine-3-carboxamide

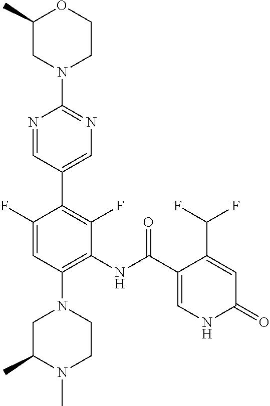 [SMILES: C[C@@H]1CN(c2ncc(-c3c(F)cc(N4CCN(C)[C@@H](C)C4)c(NC(=O)c4c[nH]c(=O)cc4C(F)F)c3F)cn2)CCO1]

The title compound (beige solid, 66.5 mg, 55% yield) was prepared according to a coupling procedure similar to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (138 mg, 85.5% purity, 0.2 mmol) and (R)-(2-(2-methylmorpholino)pyrimidin-5-yl)boronic acid (0.5 mmol, crude) followed by TFA deprotection (1 mL TFA/10 mL DCM). LCMS [M+H]⁺ 590.5.

### Example 130: N-(2,4-difluoro-3-(2-((R)-2-methylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide

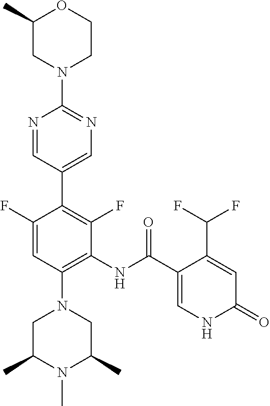 [SMILES: C[C@@H]1CN(c2ncc(-c3c(F)cc(N4C[C@@H](C)N(C)[C@@H](C)C4)c(NC(=O)c4c[nH]c(=O)cc4C(F)F)c3F)cn2)CCO1]

The title compound (beige solid, 63.0 mg, 52% yield) was prepared according to a coupling procedure similar to Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (142 mg, 85.02% purity, 0.2 mmol) and (R)-(2-(2-methylmorpholino)pyrimidin-5-yl)boronic acid (0.5 mmol, crude) followed by TFA deprotection (1 mL TFA/10 mL DCM). LCMS [M+H]⁺ 604.5.

### Example 131: N-(3-(2-((2S,6R)-2,6-dimethylmorpholino)thiazol-4-yl)-6-((S)-3,4-dimethylpiperazin-1-yl)-2,4-difluorophenyl)-6-oxo-4-(trifluoromethyl)-1,6-dihydropyridine-3-carboxamide

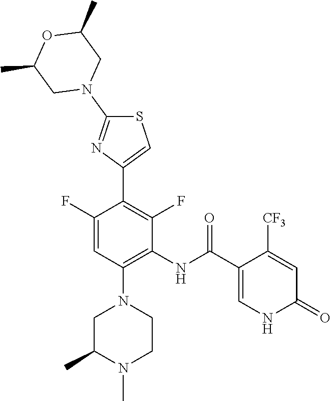 [SMILES: C[C@@H]1CN(c2nc(-c3c(F)cc(N4CCN(C)[C@@H](C)C4)c(NC(=O)c4c[nH]c(=O)cc4C(F)(F)F)c3F)cs2)C[C@H](C)O1]

To a 20 mL microwave via charge with (S)-3-bromo-6-(3,4-dimethylpiperazin-1-yl)-2,4-difluoroaniline (256 mg, 0.8 mmol), bis(pinacolato)diboron (406 mg, 1.6 mmol), Pd(dppf)Cl₂ (29 mg, 0.04 mmol) and KOAc (236 mg, 2.4 mmol) was added dioxane (6 mL) and the resulting mixture was heated at 120° C. in microwave for 16 h. To the crude product mixture was added (2S,6R)-4-(4-bromothiazol-2-yl)-2,6-dimethylmorpholine (266 mg, 0.96 mmol), bis(di-tert-butyl(4-dimethylaminophenyl)phosphine)dichloropalladium(II) (28 mg, 0.04 mmol) and 1 M K₃P04 (1.6 mL, 1.6 mmol). The resulting mixtures were heated in microwave at 110° C. for 2 h. After aqueous workup, it was purified by flash chromatography (gradient: EtOAc/hex 0-100% then MeOH/DCM 0-20%) to give 3-(2-((2S,6R)-2,6-dimethylmorpholino)thiazol-4-yl)-6-((S)-3,4-dimethylpiperazin-1-yl)-2,4-difluoroaniline as a dark brown (near black) oil (230 mg, 78.84% purity, 51.8%). LCMS [M+H]⁺ 438.43. The title compound (beige solid, 9.8 mg, 7.5%) was prepared according to a coupling procedure similar to Example 1, Step 6 using 4-(trifluoromethyl)-6-(2-(trimethylsilyl)ethoxy)nicotinic acid (115 mg, 0.37 mmol), T3P (50% wt % in EtOAc, 0.25 mL+0.12 mL, 0.41 mmol+0.21 mmol) and 3-(2-((2S,6R)-2,6-dimethylmorpholino)thiazol-4-yl)-6-((S)-3,4-dimethylpiperazin-1-yl)-2,4-difluoroaniline (115 mg, 78.84% purity, 0.21 mmol). LCMS [M+H]⁺ 627.5.

### Example 132: 4-(Difluoromethyl)-N-(3-(2-((2S,6R)-2,6-dimethylmorpholino)thiazol-4-yl)-6-((S)-3,4-dimethylpiperazin-1-yl)-2,4-difluorophenyl)-6-oxo-1,6-dihydropyridine-3-carboxamide

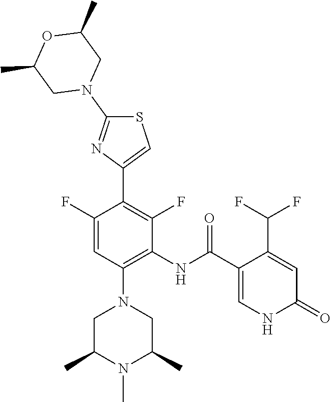 [SMILES: C[C@@H]1CN(c2nc(-c3c(F)cc(N4C[C@@H](C)N(C)[C@@H](C)C4)c(NC(=O)c4c[nH]c(=O)cc4C(F)F)c3F)cs2)C[C@H](C)O1]

The title compound (beige solid, 9.8 mg, 7.5% yield) was prepared according to a coupling procedure similar to that in Example 13, Step 7 using 4-(difluoromethyl)-6-(2-(trimethylsilyl)ethoxy)nicotinic acid (108 mg, 0.37 mmol), T3P (50% wt in EtOAc, 0.25 mL+0.12 mL, 0.41 mmol+0.21 mmol) and 3-(2-((2S,6R)-2,6-dimethylmorpholino)thiazol-4-yl)-6-((S)-3,4-dimethylpiperazin-1-yl)-2,4-difluoroaniline (115 mg, 78.84% purity, 0.21 mmol). LCMS [M+H]⁺ 609.5.

### Example 133: 4-(2,6-Difluoro-3-(6-oxo-4-(trifluoromethyl)-1,6-dihydropyridine-3-carboxamido)-4-((3S,5R)-3,4,5-trimethylpiperazin-1-yl)phenyl)-5,6-dihydropyridine-1(2H)-carboxylate

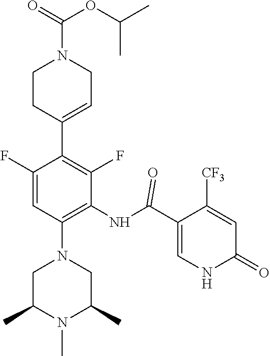 [SMILES: CC(C)OC(=O)N1CC=C(c2c(F)cc(N3C[C@@H](C)N(C)[C@@H](C)C3)c(NC(=O)c3c[nH]c(=O)cc3C(F)(F)F)c2F)CC1]

Using a procedure similar to Example 19, the title compound was synthesized as a beige solid (27.5 mg, 79% yield) from intermediate N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide.

### Example 134: 3-(2,6-Difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate

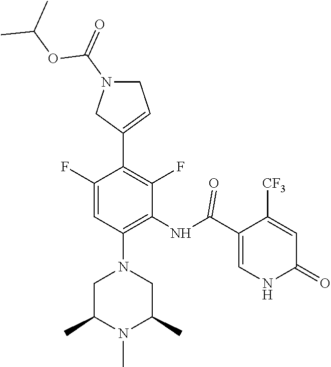 [SMILES: CC(C)OC(=O)N1CC=C(c2c(F)cc(N3C[C@@H](C)N(C)[C@@H](C)C3)c(NC(=O)c3c[nH]c(=O)cc3C(F)(F)F)c2F)C1]

Using a procedure similar to Example 19, the title compound was synthesized as an off white powder (26 mg, 76% yield) from intermediate N-(3-(2,5-dihydro-1H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide. LCMS [M+1]⁺=598.7.

### Example 135: 3,3-Difluorocyclobutyl 4-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-5,6-dihydropyridine-1(2H)-carboxylate

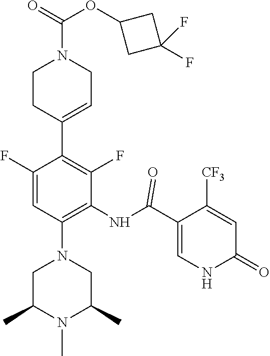 [SMILES: C[C@@H]1CN(c2cc(F)c(C3=CCN(C(=O)OC4CC(F)(F)C4)CC3)c(F)c2NC(=O)c2c[nH]c(=O)cc2C(F)(F)F)C[C@H](C)N1C]

Using a procedure similar to Example 42, the title compound was synthesized as an off white powder (19.5 mg, 67% yield) from intermediate N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide. LCMS [M+1]⁺660.7 g/mol.

### Example 136: 3,3-Difluorocyclobutyl 3-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate

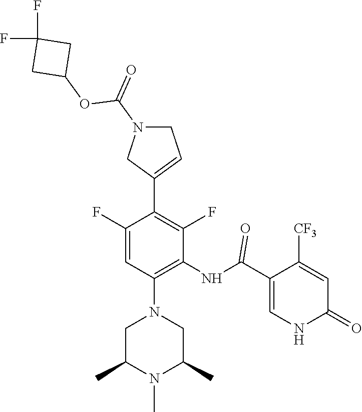 [SMILES: C[C@@H]1CN(c2cc(F)c(C3=CCN(C(=O)OC4CC(F)(F)C4)C3)c(F)c2NC(=O)c2c[nH]c(=O)cc2C(F)(F)F)C[C@H](C)N1C]

Using a procedure similar to Example 42, the title compound was prepared as a white powder (30.5 mg, 92% yield) from intermediate N-(3-(2,5-dihydro-1H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide. LCMS [M+1]⁺646.6

### Example 137: N-(2,4-difluoro-3-(1-(5-methoxypyrimidin-2-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide

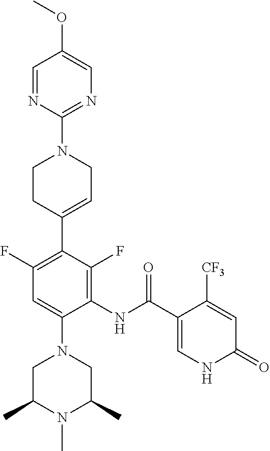 [SMILES: COc1cnc(N2CC=C(c3c(F)cc(N4C[C@@H](C)N(C)[C@@H](C)C4)c(NC(=O)c4c[nH]c(=O)cc4C(F)(F)F)c3F)CC2)nc1]

Using a procedure similar to Example 48, the title compound was synthesized as an yellow powder (13 mg, 41% yield) from intermediate N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide. LCMS [M+1]⁺=634.7.

### Example 138: N-(2,4-difluoro-3-(1-(5-methoxypyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide

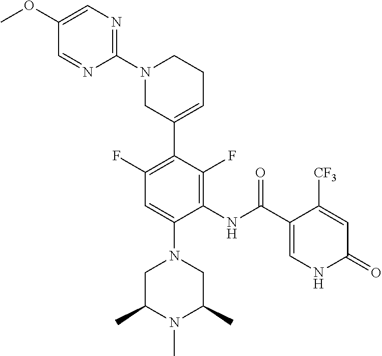 [SMILES: COc1cnc(N2CCC=C(c3c(F)cc(N4C[C@@H](C)N(C)[C@@H](C)C4)c(NC(=O)c4c[nH]c(=O)cc4C(F)(F)F)c3F)C2)nc1]

Using a procedure similar to Example 48, the title compound was prepared as an off white powder (13 mg, 41% yield) from intermediate N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide. LCMS [M+1]⁺634.7.

### Example 139: N-(2,4-difluoro-3-(1-(5-methoxypyrimidin-2-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide

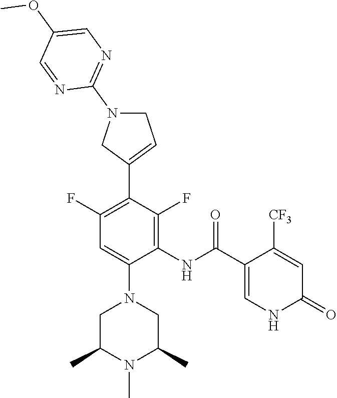 [SMILES: COc1cnc(N2CC=C(c3c(F)cc(N4C[C@@H](C)N(C)[C@@H](C)C4)c(NC(=O)c4c[nH]c(=O)cc4C(F)(F)F)c3F)C2)nc1]

The procedure followed was similar to Example 48 using N-(3-(2,5-dihydro-1H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide to give the title compound as a white powder (11 mg, 32% yield). LCMS [M+1]⁺620.8.

### Example 140: N-(2,4-difluoro-3-(1-(6-methoxypyrimidin-4-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide

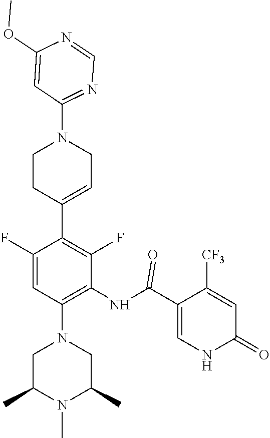 [SMILES: COc1cc(N2CC=C(c3c(F)cc(N4C[C@@H](C)N(C)[C@@H](C)C4)c(NC(=O)c4c[nH]c(=O)cc4C(F)(F)F)c3F)CC2)ncn1]

A procedure was similar to Example 48 using N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide and 4-iodo-6-methoxypyrimidine (15.72 mg, 0.067 mmol) to give the title compound as a white powder (21 mg, 62% yield). LCMS [M+1]⁺=634.7.

### Example 141: 1-Methylcyclobutyl 3-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate

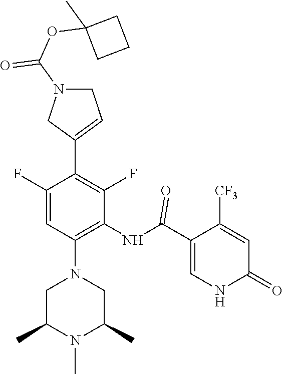 [SMILES: C[C@@H]1CN(c2cc(F)c(C3=CCN(C(=O)OC4(C)CCC4)C3)c(F)c2NC(=O)c2c[nH]c(=O)cc2C(F)(F)F)C[C@H](C)N1C]

The procedure was similar to that of Example 92 using N-(3-(2,5-dihydro-1H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide. The title compound was isolated as a white powder (23 mg, 72% yield). LCMS [M+1]⁺624.8.

### Example 142: N-(2,4-difluoro-3-(1-(6-methoxypyrimidin-4-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide

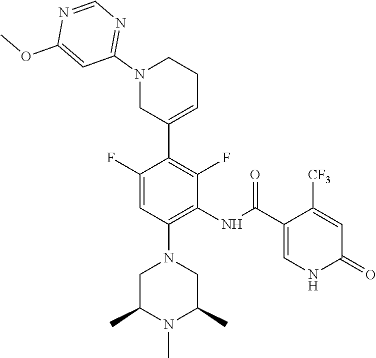 [SMILES: COc1cc(N2CCC=C(c3c(F)cc(N4C[C@@H](C)N(C)[C@@H](C)C4)c(NC(=O)c4c[nH]c(=O)cc4C(F)(F)F)c3F)C2)ncn1]

The procedure followed was similar to Example 48 using N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide and 4-iodo-6-methoxy pyrimidine to afford the title compound as an off white powder (18 mg, 57% yield. LCMS [M+1]⁺=634.7.

### Example 143: N-(2,4-difluoro-3-(1-(6-methoxypyrimidin-4-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide

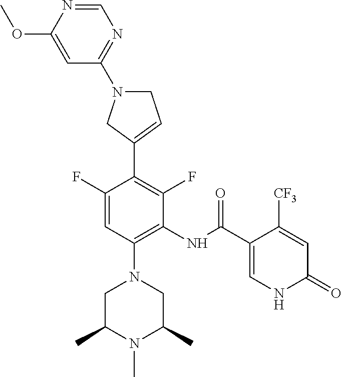 [SMILES: COc1cc(N2CC=C(c3c(F)cc(N4C[C@@H](C)N(C)[C@@H](C)C4)c(NC(=O)c4c[nH]c(=O)cc4C(F)(F)F)c3F)C2)ncn1]

The procedure followed was similar to Example 48 using N-(3-(2,5-dihydro-1H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide and 4-iodo-6-methoxy pyrimidine. The title compound was isolated as a white powder (21.5 mg, 67%). LCMS [M+1]⁺620.7.

### Example 144: 1-Methylcyclobutyl 4-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-5,6-dihydropyridine-1(2H)-carboxylate

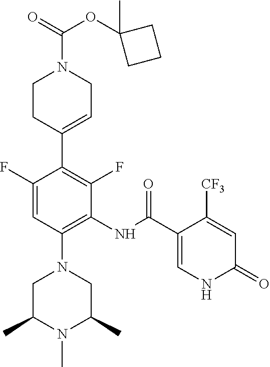 [SMILES: C[C@@H]1CN(c2cc(F)c(C3=CCN(C(=O)OC4(C)CCC4)CC3)c(F)c2NC(=O)c2c[nH]c(=O)cc2C(F)(F)F)C[C@H](C)N1C]

The procedure followed was similar to Example 42 using N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide to give the title compound was isolated as a white powder (18 mg, 50% yield). LCMS [M+1]⁺638.7.

### Example 145: N-(2,4-difluoro-3-(1-(5-fluoropyrimidin-2-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide

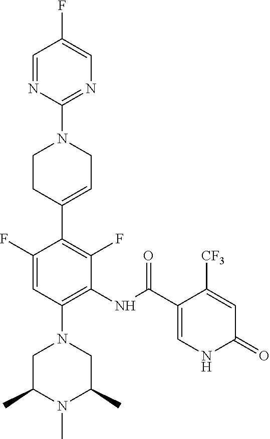 [SMILES: C[C@@H]1CN(c2cc(F)c(C3=CCN(c4ncc(F)cn4)CC3)c(F)c2NC(=O)c2c[nH]c(=O)cc2C(F)(F)F)C[C@H](C)N1C]

The procedure followed was similar to Example 48 using N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide and 2-bromo-5-fluoropyrimidine to afford the title compound as an off white powder (22.5 mg, 73% yield). LCMS [M+1]⁺622.7.

### Example 146: N-(2,4-difluoro-3-(1-(5-fluoropyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide

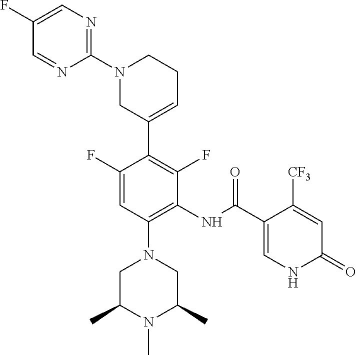 [SMILES: C[C@@H]1CN(c2cc(F)c(C3=CCCN(c4ncc(F)cn4)C3)c(F)c2NC(=O)c2c[nH]c(=O)cc2C(F)(F)F)C[C@H](C)N1C]

The procedure followed was similar to Example 48 using N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide and 2-bromo-5-fluoropyrimidine. The title compound was isolated as a white powder (23 mg, 76% yield). LCMS [M+1]⁺=622.7.

### Example 147: N-(2,4-difluoro-3-(1-(5-fluoropyrimidin-2-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide

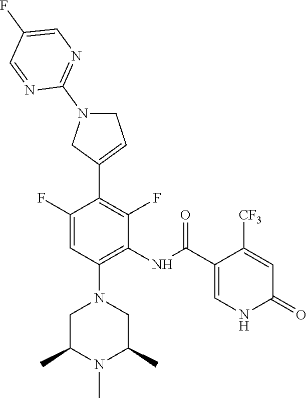 [SMILES: C[C@@H]1CN(c2cc(F)c(C3=CCN(c4ncc(F)cn4)C3)c(F)c2NC(=O)c2c[nH]c(=O)cc2C(F)(F)F)C[C@H](C)N1C]

The procedure followed was similar to Example 48 using N-(3-(2,5-dihydro-1H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide and 2-bromo-5-fluoropyrimidine. The title compound was isolated as a white powder (22.5 mg, 72% yield). LCMS [M+1]⁺608.7.

### Example 148: N-(3-(1-(5-cyanothiazol-2-yl)-1,2,3,6-tetrahydropyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

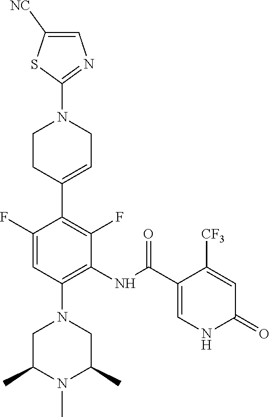 [SMILES: C[C@@H]1CN(c2cc(F)c(C3=CCN(c4ncc(C#N)s4)CC3)c(F)c2NC(=O)c2c[nH]c(=O)cc2C(F)(F)F)C[C@H](C)N1C]

A procedure similar to Example 48 using N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide and 2-bromo-5-cyanothiazole afforded the title compound as an off white powder (24 mg, 76% yield). LCMS [M+1]⁺634.7.

### Example 149: N-(3-(1-(5-cyanothiazol-2-yl)-1,2,5,6-tetrahydropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

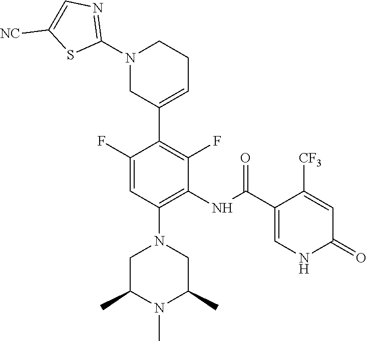 [SMILES: C[C@@H]1CN(c2cc(F)c(C3=CCCN(c4ncc(C#N)s4)C3)c(F)c2NC(=O)c2c[nH]c(=O)cc2C(F)(F)F)C[C@H](C)N1C]

A procedure similar to Example 48 using N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide and 2-bromo-5-cyanothiazole gave the title compound as an off white powder (23 mg, 73% yield). LCMS [M+1]⁺634.7.

### Example 150: N-(3-(1-(5-cyanothiazol-2-yl)-2,5-dihydro-1H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

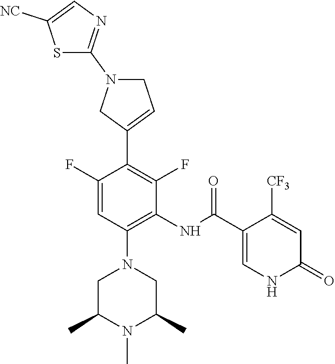 [SMILES: C[C@@H]1CN(c2cc(F)c(C3=CCN(c4ncc(C#N)s4)C3)c(F)c2NC(=O)c2c[nH]c(=O)cc2C(F)(F)F)C[C@H](C)N1C]

A procedure similar to Example 48 using N-(3-(2,5-dihydro-H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide and 2-bromo-5-cyanothiazole gave the title compound was isolated as an off white powder (22 mg, 69% yield). LCMS [M+1]⁺=620.7.

### Example 151: N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide

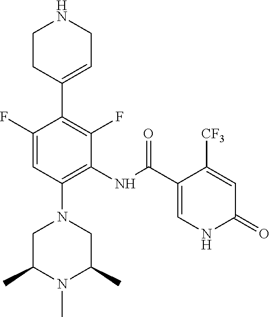 [SMILES: C[C@@H]1CN(c2cc(F)c(C3=CCNCC3)c(F)c2NC(=O)c2c[nH]c(=O)cc2C(F)(F)F)C[C@H](C)N1C]

N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (500 mg, 0.802 mmol), N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (500 mg, 0.802 mmol) and [1,1′-Bis(diphenylphosphino)ferrocene]dichloropalladium(II) (76 mg, 0.104 mmol) were mixed in 1,4-dioxane (12 mL). Potassium phosphate tribasic reagent grade, >=98% (4.01 ml, 4.01 mmol) was added and the vial was flushed with nitrogen. The mixture was heated in a microwave reactor to 100° C. for 1.25 h. Continued heating for a total of 2 h. The mixture was mixed with brine (10 mL) and EtOAc (10 mL), the organic phase was separated, aq phase was extracted with EA (8 mL×2). The combined extracts were dried over Na₂SO₄, concentrated and purified by sgc to afford the intermediate as an off white foam (348 mg). TFA (0.75 mL) was added to a solution of the SM in DCM (2.5 ml) at RT and the mixture was stirred at RT. LCMS after 10 min showed completion of the rxn. The title compound was isolated as an off white solid. (248 mg, 98% yield for last step). LCMS [M+H]⁺ 526.6.

### Example 152: N-(3-(1-(2-cyanopyrimidin-4-yl)-1,2,3,6-tetrahydropyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

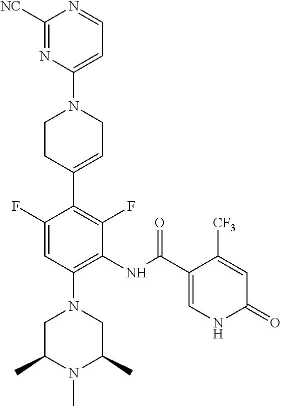 [SMILES: C[C@@H]1CN(c2cc(F)c(C3=CCN(c4ccnc(C#N)n4)CC3)c(F)c2NC(=O)c2c[nH]c(=O)cc2C(F)(F)F)C[C@H](C)N1C]

The procedure followed was similar to Example 48 using N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide and 4-bromopyrimidine-2-carbonitrile to give the title compound was isolated as an off white powder (17 mg, 41% yield). LCMS [M+1]⁺629.7.

### Example 153: (S)-2-(difluoromethyl)-N-(6-(3,4-dimethylpiperazin-1-yl)-2,4-difluoro-3-(5-methyl-4-(pyrrolidine-1-carbonyl)thiazol-2-yl)phenyl)-4-fluorobenzamide

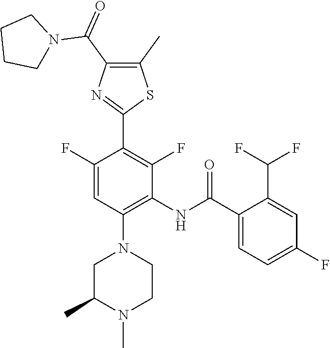 [SMILES: Cc1sc(-c2c(F)cc(N3CCN(C)[C@@H](C)C3)c(NC(=O)c3ccc(F)cc3C(F)F)c2F)nc1C(=O)N1CCCC1]

### Step 1: (S)-(2-(3-Amino-4-(3,4-dimethylpiperazin-1-yl)-2,6-difluorophenyl)-5-methylthiazol-4-yl)(pyrrolidin-1-yl)methanone

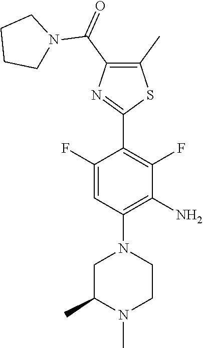 [SMILES: Cc1sc(-c2c(F)cc(N3CCN(C)[C@@H](C)C3)c(N)c2F)nc1C(=O)N1CCCC1]

To a 30 mL vial charged with (S)-3-bromo-6-(3,4-dimethylpiperazin-1-yl)-2,4-difluoroaniline (0.450 g, 1.405 mmol), bis(pinacolato)diboron (0.714 g, 2.81 mmol), [1,1′-bis(diphenylphosphino)ferrocene]dichloropalladium(II) (0.051 g, 0.070 mmol) and potassium acetate (0.414 g, 4.22 mmol) was added 1,4-dioxane (6 mL) and the resulting mixture was heated at 120° C. overnight. To this mixture was added (2-bromo-5-methylthiazol-4-yl)(pyrrolidin-1-yl)methanone (0.464 g, 1.687 mmol) (in 1 mL dioxane), bis(di-tert-butyl(4 dimethylaminophenyl)phosphine)dichloropalladium(II) (0.050 g, 0.070 mmol) and potassium phosphate (2.162 ml of a 1.3 M solution in water, 2.81 mmol). The resulting mixture was heated at 110° C. for 3 h. The reaction was partitioned between EtOAc and a saturated aqueous brine solution. The layers were separated and the aqueous layer was extracted with additional EtOAc. The combined organic layers were dried over MgSO₄ and the inorganics were removed by filtration. After concentration to dryness the residue was purified by flash chromatography [0.5-9.5% MeOH/DCM+0.5% NH₄OH] to afford the title compound (0.441 mmol, 31.4% yield) as a brown foam that was >95% pure by LCMS (@254 nm).

### Step 2: (S)-(2-(3-amino-4-(3,4-dimethylpiperazin-1-yl)-2,6-difluorophenyl)-5-methylthiazol-4-yl)(pyrrolidin-1-yl)methanone

A mixture of 2-(difluoromethyl)-4-fluorobenzoic acid (˜200 mg) and thionyl chloride (˜1 mL) was heated at 80° C. for 1 h and then concentrated to dryness and re-dissolved in DCM (5 mL). Approximately 1 mL of the acid chloride solution was added to a stirring solution of (S)-(2-(3-amino-4-(3,4-dimethylpiperazin-1-yl)-2,6-difluorophenyl)-5-methylthiazol-4-yl)(pyrrolidin-1-yl)methanone (0.064 g, 0.147 mmol) and triethylamine (0.061 ml, 0.441 mmol) in DCM (2 mL) at room temperature. After stirring for 4 h at room temperature the reaction mixture was heated to 50° C. for 1 h. The reaction mixture was concentrated onto celite and purified by reverse phase flash chromatography [10-60% MeCN/0.1% Formic Acid] to afford the title compound formate salt (0.072 mmol, 48.9% yield) as a near colorless solid after lyophilization. LCMS [M+H]⁺ 608.3.

### Example 154: (S)—N-(6-(3,4-dimethylpiperazin-1-yl)-2,4-difluoro-3-(5-methyl-4-(pyrrolidine-1-carbonyl)thiazol-2-yl)phenyl)-4-fluoro-2-(trifluoromethyl)benzamide

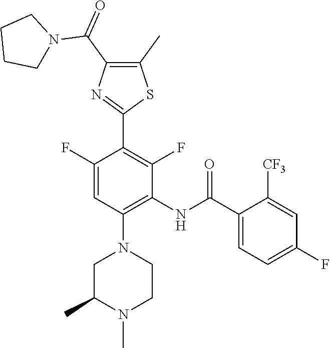 [SMILES: Cc1sc(-c2c(F)cc(N3CCN(C)[C@@H](C)C3)c(NC(=O)c3ccc(F)cc3C(F)(F)F)c2F)nc1C(=O)N1CCCC1]

4-Fluoro-2-(trifluoromethyl)benzoyl chloride (0.033 ml, 0.220 mmol, prepared in a manner similar to Step 1 of Example 155) was added to a stirring solution of (S)-(2-(3-amino-4-(3,4-dimethylpiperazin-1-yl)-2,6-difluorophenyl)-5-methylthiazol-4-yl)(pyrrolidin-1-yl)methanone (0.064 g, 0.147 mmol) and triethylamine (0.061 ml, 0.441 mmol) in DCM (2 mL) at RT. After stirring for 4 h the reaction mixture was heated to 50° C. for 1 h. An additional equivalent of acid chloride and triethylamine was added and the mixture at room temperature overnight. The process of addition of another equivalent of reagents was repeated, the mixture was heated to 45° C. for 2 h, and the addition of reagents was again repeated with more acid chloride and trimethylamine. After further heating the reaction mixture was concentrated onto celite and purified by reverse phase flash chromatography [10-60% MeCN/0.1% Formic Acid] to afford the title compound formate salt (0.045 mmol, 30.4% yield) as a near colorless solid after lyophilisation. LCMS [M+H]⁺ 626.3.

### Example 155: (S)—N-(3-(2-(8-oxa-3-azabicyclo[3.2.1]octan-3-yl)pyrimidin-5-yl)-6-(3,4-dimethylpiperazin-1-yl)-2,4-difluorophenyl)-4-(difluoromethyl)-6-oxo-1,6-dihydropyridine-3-carboxamide-

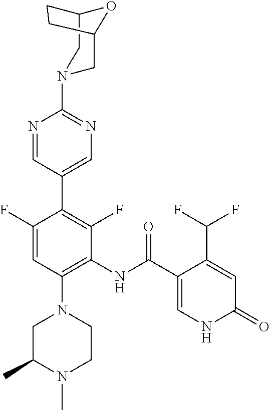 [SMILES: C[C@H]1CN(c2cc(F)c(-c3cnc(N4CC5CCC(C4)O5)nc3)c(F)c2NC(=O)c2c[nH]c(=O)cc2C(F)F)CCN1C]

The title compound (beige solid, 26.4 mg, 41%, 94.1% purity) was prepared according to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 0.1 mmol based on 85.5% purity), (2-((1R,5S)-8-oxa-3-azabicyclo[3.2.1]octan-3-yl)pyrimidin-5-yl)boronic acid (0.3 mmol, crude), followed by TFA deprotection (1 mL TFA/6 mL DCM, rt, 30 min). ¹H NMR (500 MHz, METHANOL-d4) δ=8.44 (s, 2H), 8.07 (s, 1H), 7.32 (t, J=55.0 Hz, 1H), 6.90 (br d, J=11.6 Hz, 1H), 6.82 (s, 1H), 4.51-4.44 (m, 2H), 4.35 (d, J=13.1 Hz, 2H), 3.26-3.15 (m, 4H), 3.01-2.86 (m, 2H), 2.64-2.49 (m, 2H), 2.46-2.35 (m, 4H), 2.01-1.93 (m, 2H), 1.86-1.78 (m, 2H), 1.13 (d, J=6.2 Hz, 3H); LCMS [M+H]⁺ 602.5.

### Example 156: N-(3-(2-(8-oxa-3-azabicyclo[3.2.1]octan-3-yl)pyrimidin-5-yl)-2,4-difluoro-6-((3S,5R)-3,4,5-trimethylpiperazin-1-yl)phenyl)-4-(difluoromethyl)-6-oxo-1,6-dihydropyridine-3-carboxamide

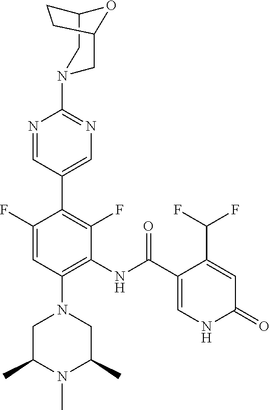 [SMILES: C[C@@H]1CN(c2cc(F)c(-c3cnc(N4CC5CCC(C4)O5)nc3)c(F)c2NC(=O)c2c[nH]c(=O)cc2C(F)F)C[C@H](C)N1C]

The title compound (light beige solid, 20.7 mg, 34%, 99.6% purity) was prepared according to a method similar to Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (71 mg, 0.1 mmol based on 85.0% purity) and (2-((1R,5S)-8-oxa-3-azabicyclo[3.2.1]octan-3-yl)pyrimidin-5-yl)boronic acid (0.3 mmol, crude), followed by TFA deprotection (1 mL TFA/6 mL DCM, rt, 30 min). ¹H NMR (500 MHz, METHANOL-d₄) δ=8.43 (s, 2H), 8.08 (s, 1H), 7.34 (t, J=55.0 Hz, 1H), 6.88 (br d, J=11.4 Hz, 1H), 6.82 (s, 1H), 4.51-4.43 (m, 2H), 4.35 (d, J=13.1 Hz, 2H), 3.24-3.16 (m, 4H), 2.70-2.63 (m, 2H), 2.62-2.53 (m, 2H), 2.41 (s, 3H), 1.99-1.92 (m, 2H), 1.85-1.79 (m, 2H), 1.18 (br d, J=6.0 Hz, 6H); LCMS [M+H]⁺ 616.5.

### Example 157: 2-(Difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-4-fluorobenzamide

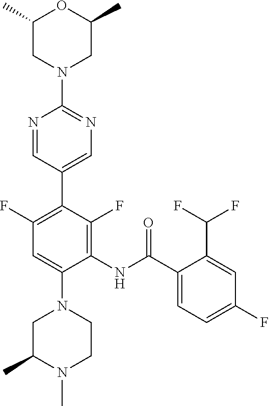 [SMILES: C[C@H]1CN(c2ncc(-c3c(F)cc(N4CCN(C)[C@@H](C)C4)c(NC(=O)c4ccc(F)cc4C(F)F)c3F)cn2)C[C@H](C)O1]

The title compound (white solid, 36.5 mg, 60%, 100% purity) was prepared according to Example 1, Step 6 using (S)—N-(3-bromo-6-(3,4-dimethylpiperazin-1-yl)-2,4-difluorophenyl)-2-(difluoromethyl)-4-fluorobenzamide (65 mg, 0.1 mmol based on 75.5% purity) and (2-((2S,6S)-2,6-dimethylmorpholino)pyrimidin-5-yl)boronic acid (0.25 mmol, crude). ¹H NMR (500 MHz, METHANOL-d₄) δ=9.98 (s, 2H), 9.44 (t, J=6.5 Hz, 1H), 9.10 (br d, J=9.2 Hz, 1H), 9.03-8.78 (m, 2H), 8.44 (br d, J=11.7 Hz, 1H), 5.69-5.58 (m, 2H), 5.55-5.46 (m, 2H), 5.14 (br dd, J=6.2, 13.2 Hz, 2H), 4.84-4.72 (m, 2H), 4.55-4.41 (m, 2H), 4.15 (br t, J=10.8 Hz, 1H), 4.04 (br t, J=10.6 Hz, 1H), 3.90 (s, 4H), 2.78 (d, J=6.4 Hz, 6H), 2.66 (br d, J=6.0 Hz, 3H); LCMS [M+H]⁺ 605.5.

### Example 158: 2-(Difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-4-fluorobenzamide

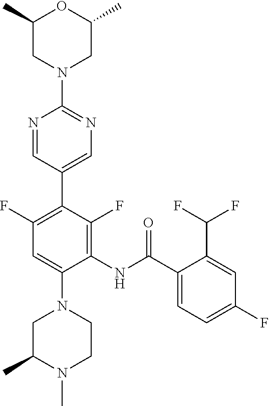 [SMILES: C[C@@H]1CN(c2ncc(-c3c(F)cc(N4CCN(C)[C@@H](C)C4)c(NC(=O)c4ccc(F)cc4C(F)F)c3F)cn2)C[C@@H](C)O1]

(S)—N-(3-bromo-6-(3,4-dimethylpiperazin-1-yl)-2,4-difluorophenyl)-2-(difluoromethyl)-4-fluorobenzamide (light greenish solid, 923 mg, 71% based on 75.5% purity) was prepared according to a method similar to Example 13, Step 7 using 2-(difluoromethyl)-4-fluorobenzoic acid (570 mg, 3 mmol), T3P (50% wt % in EtOAc, 2.38 mL, 4 mmol), pyridine (2 mL), ⁱPr₂NEt (1.4 mL, 4 mmol) and (S)-3-bromo-6-(3,4-dimethylpiperazin-1-yl)-2,4-difluoroaniline (640 mg, 2 mmol). LCMS [M+H]⁺ 492.3. The title compound (white solid, 25.2 mg, 42%, 99.7% purity) was prepared according to a method similar to Example 1, Step 6 using (S)—N-(3-bromo-6-(3,4-dimethylpiperazin-1-yl)-2,4-difluorophenyl)-2-(difluoromethyl)-4-fluorobenzamide (65 mg, 0.1 mmol based on 75.5% purity) and (2-((2R,6R)-2,6-dimethylmorpholino)pyrimidin-5-yl)boronic acid (0.25 mmol, crude). ¹H NMR (500 MHz, METHANOL-d₄) δ=8.45 (s, 2H), 7.91 (br dd, J=5.5, 7.9 Hz, 1H), 7.57 (dd, J=2.0, 9.2 Hz, 1H), 7.50-7.24 (m, 2H), 6.91 (br d, J=11.7 Hz, 1H), 4.11 (dt, J=3.5, 6.3 Hz, 2H), 3.98 (dd, J=3.3, 13.2 Hz, 2H), 3.61 (dd, J=6.2, 13.2 Hz, 2H), 3.30-3.19 (m, 2H), 3.03-2.88 (m, 2H), 2.62 (br t, J=10.9 Hz, 1H), 2.56-2.45 (m, 1H), 2.45-2.33 (m, 4H), 1.24 (d, J=6.5 Hz, 6H), 1.13 (d, J=6.2 Hz, 3H); LCMS [M+H]⁺ 605.5.

### Example 159: (S)-4-(difluoromethyl)-N-(6-(3,4-dimethylpiperazin-1-yl)-2,4-difluoro-3-(1-(2-methylthiazole-4-carbonyl)-1,2,3,6-tetrahydropyridin-4-yl)phenyl)-6-oxo-1,6-dihydropyridine-3-carboxamide

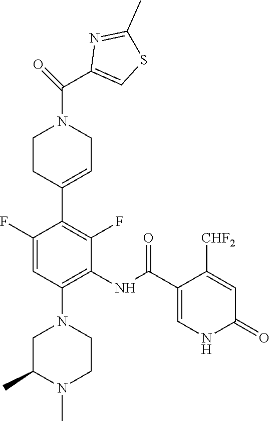 [SMILES: Cc1nc(C(=O)N2CC=C(c3c(F)cc(N4CCN(C)[C@@H](C)C4)c(NC(=O)c4c[nH]c(=O)cc4C(F)F)c3F)CC2)cs1]

A mixture of (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (25 mg, 0.051 mmol, prepared according to Example 34, Step 2), 2-methyl-1,3-thiazole-4-carboxylic acid hydrochloride (10.92 mg, 0.061 mmol), HATU (48.2 mg, 0.127 mmol) and N,N-Diisopropylethylamine (0.035 ml, 0.203 mmol) in N,N-Dimethylformamide (DMF) (3 ml) was stirred at room temperature. Complete disappearance of the starting material and formation of the desired product was observed after 1h. The reaction mixture was agitated with water (8 mL), brine (2 mL) and ethyl acetate (3 mL), the org phase was separated and the aqueous phase was extracted with ethyl acetate (2×3 mL). The combined organic phase was washed first with water (5 mL), then with brine (5 mL), dried over Na₂SO₄ and concentrated onto celite. The crude product was purified by silica gel chromatography, eluting with DCM containing 0-5% MeOH and 0-0.5% NH₄OH. The desired product was isolated as an off white powder (17 mg, 51.5%). ¹H NMR (500 MHz, METHANOL-d₄) δ=7.98-7.89 (m, 1H), 7.82-7.70 (m, 1H), 7.33-7.06 (m, 1H), 6.72-6.64 (m, 2H), 5.91-5.66 (m, 1H), 4.38-4.22 (m, 2H), 3.90-3.78 (m, 2H), 3.13-3.03 (m, 2H), 2.93-2.79 (m, 2H), 2.68-2.60 (m, 3H), 2.54-2.38 (m, 5H), 2.33 (s, 3H), 1.03 (d, J=6.1 Hz, 3H); LCMS [M+1]⁺=619.6 g/mol

### Example 160: 4-(Difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide

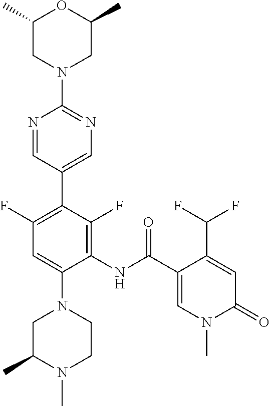 [SMILES: C[C@H]1CN(c2ncc(-c3c(F)cc(N4CCN(C)[C@@H](C)C4)c(NC(=O)c4cn(C)c(=O)cc4C(F)F)c3F)cn2)C[C@H](C)O1]

The title compound (white solid, 15.2 mg, 25%, 99.9% purity) was prepared according to a procedure similar to Example 1, Step 6 using (S)—N-(3-bromo-6-(3,4-dimethylpiperazin-1-yl)-2,4-difluorophenyl)-4-(difluoromethyl)-1-methyl-6-oxo-1,6-dihydropyridine-3-carboxamide (67 mg, 0.1 mmol based on 75.6% purity) and (2-((2S,6S)-2,6-dimethylmorpholino)pyrimidin-5-yl)boronic acid (0.2 mmol, crude). ¹H NMR (500 MHz, METHANOL-d₄) δ=8.44 (s, 2H), 8.34 (s, 1H), 7.32 (t, J=55.0 Hz, 1H), 6.90 (d, J=11.0 Hz, 1H), 6.84 (s, 1H), 4.16-4.07 (m, 2H), 3.98 (dd, J=3.3, 13.2 Hz, 2H), 3.66 (s, 3H), 3.64-3.58 (m, 2H), 3.26-3.15 (m, 2H), 2.99-2.86 (m, 2H), 2.57 (t, J=10.9 Hz, 1H), 2.52-2.44 (m, 1H), 2.41-2.32 (m, 4H), 1.24 (d, J=6.5 Hz, 6H), 1.11 (d, J=6.4 Hz, 3H); LCMS [M+H]⁺ 618.6.

### Example 161: 4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide

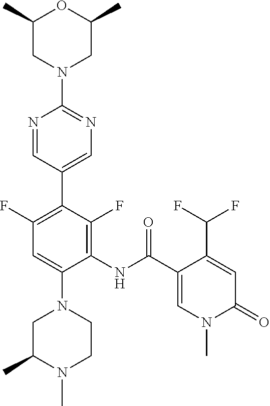 [SMILES: C[C@@H]1CN(c2ncc(-c3c(F)cc(N4CCN(C)[C@@H](C)C4)c(NC(=O)c4cn(C)c(=O)cc4C(F)F)c3F)cn2)C[C@H](C)O1]

The title compound (white solid, 15.8 mg, 26%, 99.8% purity) was prepared according to a procedure similar to Example 1, Step 6 using (S)—N-(3-bromo-6-(3,4-dimethylpiperazin-1-yl)-2,4-difluorophenyl)-4-(difluoromethyl)-1-methyl-6-oxo-1,6-dihydropyridine-3-carboxamide (67 mg, 0.1 mmol based on 75.6% purity) and (2-((2R,6R)-2,6-dimethylmorpholino)pyrimidin-5-yl)boronic acid (47 mg, 0.2 mmol). ¹H NMR (500 MHz, METHANOL-d₄) δ=8.44 (s, 2H), 8.34 (s, 1H), 7.32 (t, J=55.0 Hz, 1H), 6.90 (br d, J=11.7 Hz, 1H), 6.84 (s, 1H), 4.65 (br d, J=13.2 Hz, 2H), 3.71-3.63 (m, 5H), 3.27-3.13 (m, 2H), 3.00-2.88 (m, 2H), 2.67-2.48 (m, 4H), 2.38 (br s, 4H), 1.25 (d, J=6.2 Hz, 6H), 1.12 (br d, J=6.2 Hz, 3H); LCMS [M+H]⁺ 618.6.

### Example 162: 4-(Difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide

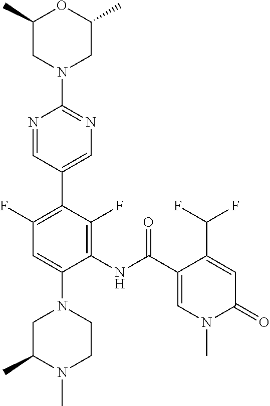 [SMILES: C[C@@H]1CN(c2ncc(-c3c(F)cc(N4CCN(C)[C@@H](C)C4)c(NC(=O)c4cn(C)c(=O)cc4C(F)F)c3F)cn2)C[C@@H](C)O1]

(S)—N-(3-bromo-6-(3,4-dimethylpiperazin-1-yl)-2,4-difluorophenyl)-4-(difluoromethyl)-1-methyl-6-oxo-1,6-dihydropyridine-3-carboxamide (lightbrown solid, 305 mg, 46%, 75.6% purity) was prepared according to a procedure similar to Example 13, Step 7 using 4-(difluoromethyl)-1-methyl-6-oxo-1,6-dihydropyridine-3-carboxylic acid (264 mg, 1.3 mmol), T₃P (50% wt % in EtOAc, 1.8 mL, 3 mmol), pyridine (3 mL) and (S)-3-bromo-6-(3,4-dimethylpiperazin-1-yl)-2,4-difluoroaniline (320 mg, 1 mmol). LCMS [M+H]⁺ 505.3. The title compound 13.5 mg, 21%, 97.1% purity) was prepared as an off-white solid according to a method similar to Example 1, Step 6 using (S)—N-(3-bromo-6-(3,4-dimethylpiperazin-1-yl)-2,4-difluorophenyl)-4-(difluoromethyl)-1-methyl-6-oxo-1,6-dihydropyridine-3-carboxamide (67 mg, 0.1 mmol based on 75.6% purity) and (2-((2R,6R)-2,6-dimethylmorpholino)pyrimidin-5-yl)boronic acid (0.2 mmol, crude). ¹H NMR (500 MHz, METHANOL-d₄) δ=8.43 (s, 2H), 8.34 (s, 1H), 7.32 (t, J=55.0 Hz, 1H), 6.90 (d, J=11.6 Hz, 1H), 6.83 (s, 1H), 4.11 (dt, J=3.5, 6.3 Hz, 2H), 3.97 (dd, J=3.2, 13.1 Hz, 2H), 3.66 (s, 3H), 3.63-3.56 (m, 2H), 3.26-3.14 (m, 2H), 2.99-2.87 (m, 2H), 2.58 (t, J=10.9 Hz, 1H), 2.53-2.46 (m, 1H), 2.40-2.33 (m, 4H), 1.24 (d, J=6.4 Hz, 6H), 1.11 (d, J=6.4 Hz, 3H); LCMS [M+H]⁺ 618.6.

### Example 163: N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide

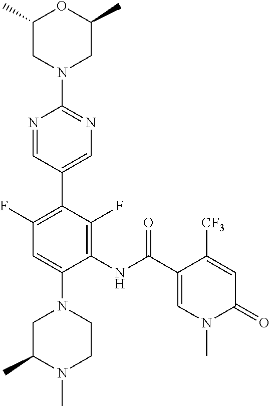 [SMILES: C[C@H]1CN(c2ncc(-c3c(F)cc(N4CCN(C)[C@@H](C)C4)c(NC(=O)c4cn(C)c(=O)cc4C(F)(F)F)c3F)cn2)C[C@H](C)O1]

The title compound (white solid, 18.4 mg, 23%, 99.7% purity) was prepared according to a method similar to Example 1, Step 6 using (S)—N-(3-bromo-6-(3,4-dimethylpiperazin-1-yl)-2,4-difluorophenyl)-1-methyl-6-oxo-4-(trifluoromethyl)-1,6-dihydropyridine-3-carboxamide (91 mg, 0.125 mmol based on 71.7% purity) and (2-((2S,6S)-2,6-dimethylmorpholino)pyrimidin-5-yl)boronic acid (0.2 mmol, crude). ¹H NMR (500 MHz, METHANOL-d₄) δ=8.44 (s, 2H), 8.26 (s, 1H), 6.96 (s, 1H), 6.92 (d, J=11.6 Hz, 1H), 4.11 (dt, J=3.5, 6.1 Hz, 2H), 3.98 (dd, J=2.8, 13.2 Hz, 2H), 3.67 (s, 3H), 3.61 (br dd, J=6.2, 13.2 Hz, 2H), 3.27-3.16 (m, 2H), 3.02-2.89 (m, 2H), 2.61 (br t, J=10.9 Hz, 1H), 2.54 (br s, 1H), 2.46-2.35 (m, 4H), 1.24 (d, J=6.5 Hz, 6H), 1.14 (br d, J=5.9 Hz, 3H); LCMS [M+H]⁺ 636.5.

### Example 164: N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide

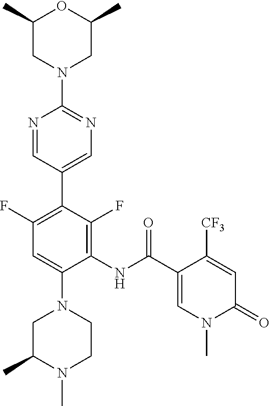 [SMILES: C[C@@H]1CN(c2ncc(-c3c(F)cc(N4CCN(C)[C@@H](C)C4)c(NC(=O)c4cn(C)c(=O)cc4C(F)(F)F)c3F)cn2)C[C@H](C)O1]

The title compound (white solid, 28.2 mg, 36%, 99.9% purity) was prepared according to a procedure similar to Example 1, Step 6 using (S)—N-(3-bromo-6-(3,4-dimethylpiperazin-1-yl)-2,4-difluorophenyl)-1-methyl-6-oxo-4-(trifluoromethyl)-1,6-dihydropyridine-3-carboxamide (91 mg, 0.125 mmol based on 71.7% purity) and (2-((2S,6R)-2,6-dimethylmorpholino)pyrimidin-5-yl)boronic acid (59 mg, 0.2 mmol). ¹H NMR (500 MHz, METHANOL-d4) δ=8.42 (s, 2H), 8.23 (s, 1H), 6.95-6.92 (m, 1H), 6.89 (br d, J=11.7 Hz, 1H), 4.62 (br d, J=13.2 Hz, 2H), 3.70-3.60 (m, 5H), 3.24-3.11 (m, 2H), 2.98-2.86 (m, 2H), 2.65-2.48 (m, 4H), 2.44-2.31 (m, 4H), 1.22 (br d, J=5.4 Hz, 6H), 1.11 (br d, J=5.7 Hz, 3H); LCMS [M+H]⁺ 636.5.

### Example 165: N-(3-(2-((2R,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide

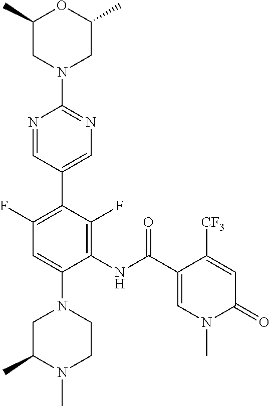 [SMILES: C[C@@H]1CN(c2ncc(-c3c(F)cc(N4CCN(C)[C@@H](C)C4)c(NC(=O)c4cn(C)c(=O)cc4C(F)(F)F)c3F)cn2)C[C@@H](C)O1]

(S)—N-(3-bromo-6-(3,4-dimethylpiperazin-1-yl)-2,4-difluorophenyl)-1-methyl-6-oxo-4-(trifluoromethyl)-1,6-dihydropyridine-3-carboxamide (beige foam, 278 mg, 38%, 71.7% purity) was prepared according to a procedure similar to Example 13, Step 7 using 1-methyl-6-oxo-4-(trifluoromethyl)-1,6-dihydropyridine-3-carboxylic acid (287 mg, 1.3 mmol), T3P (50% wt % in EtOAc, 1.8 mL, 3 mmol), pyridine (3 mL) and (S)-3-bromo-6-(3,4-dimethylpiperazin-1-yl)-2,4-difluoroaniline (320 mg, 1 mmol). LCMS [M+H]⁺ 523.4. The title compound (white solid, 23.2 mg, 29%, 99.9% purity) was prepared according to a procedure similar to Example 1, Step 6 using (S)—N-(3-bromo-6-(3,4-dimethylpiperazin-1-yl)-2,4-difluorophenyl)-1-methyl-6-oxo-4-(trifluoromethyl)-1,6-dihydropyridine-3-carboxamide (91 mg, 0.125 mmol based on 71.7% purity) and (2-((2R,6R)-2,6-dimethylmorpholino)pyrimidin-5-yl)boronic acid (0.2 mmol, crude). ¹H NMR (500 MHz, METHANOL-d₄) δ=8.44 (s, 2H), 8.26 (s, 1H), 6.96 (s, 1H), 6.92 (br d, J=11.9 Hz, 1H), 4.16-4.07 (m, 2H), 3.98 (br d, J=12.7 Hz, 2H), 3.67 (s, 3H), 3.61 (br dd, J=6.1, 13.1 Hz, 2H), 3.28-3.13 (m, 2H), 3.04-2.89 (m, 2H), 2.67-2.50 (m, 2H), 2.48-2.33 (m, 4H), 1.24 (br d, J=6.0 Hz, 6H), 1.14 (br d, J=5.5 Hz, 3H); LCMS [M+H]⁺ 636.5.

### Example 166: N-(3-(2-((2S,6S)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide Formic Acid Salt

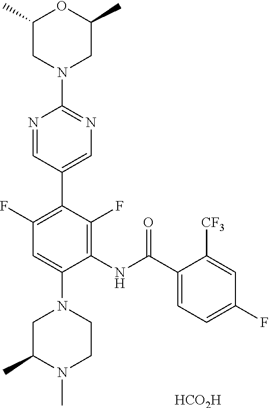 [SMILES: C[C@H]1CN(c2ncc(-c3c(F)cc(N4CCN(C)[C@@H](C)C4)c(NC(=O)c4ccc(F)cc4C(F)(F)F)c3F)cn2)C[C@H](C)O1 ; "HCO2H"]

The title compound (white solid, formic acid salt, 37.6 mg, 56%, 99.9% purity) was prepared according to a method similar to Example 1, Step 6 using (S)—N-(3-bromo-6-(3,4-dimethylpiperazin-1-yl)-2,4-difluorophenyl)-4-fluoro-2-(trifluoromethyl)benzamide (67 mg, 0.1 mmol based on 75.7%% purity) and (2-((2S,6S)-2,6-dimethylmorpholino)pyrimidin-5-yl)boronic acid (0.2 mmol, crude). ¹H NMR (500 MHz, METHANOL-d₄) δ=8.42 (s, 3H), 7.81 (br dd, J=5.3, 7.9 Hz, 1H), 7.65 (br d, J=8.8 Hz, 1H), 7.55 (br t, J=7.2 Hz, 1H), 6.95 (br d, J=11.5 Hz, 1H), 4.09 (dt, J=3.5, 6.0 Hz, 2H), 3.95 (br dd, J=2.9, 13.1 Hz, 2H), 3.59 (br dd, J=6.2, 13.1 Hz, 2H), 3.37-3.31 (m, 2H), 3.27-3.19 (m, 1H), 3.16-3.01 (m, 1H), 2.94-2.83 (m, 2H), 2.78 (br d, J=10.5 Hz, 1H), 2.68-2.60 (m, 3H), 1.26 (br d, J=5.7 Hz, 3H), 1.22 (br d, J=6.4 Hz, 6H); LCMS [M+H]⁺ 623.5.

### Example 167: N-(3-(2-((2R,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide Formic Acid Salt

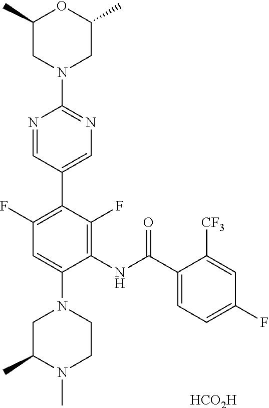 [SMILES: C[C@@H]1CN(c2ncc(-c3c(F)cc(N4CCN(C)[C@@H](C)C4)c(NC(=O)c4ccc(F)cc4C(F)(F)F)c3F)cn2)C[C@@H](C)O1 ; "HCO2H"]

(S)—N-(3-bromo-6-(3,4-dimethylpiperazin-1-yl)-2,4-difluorophenyl)-4-fluoro-2-(trifluoromethyl)benzamide (beige solid, 1.036 g, 77% based on 75.7% purity and two combined reactions) was prepared by two identical reactions using 4-Fluoro-2-(trifluoromethyl)benzoyl chloride (0.46 mL, 3 mmol) and (S)-3-bromo-6-(3,4-dimethylpiperazin-1-yl)-2,4-difluoroaniline (320 mg, 1 mmol) in DCM (25 mL), followed by hydrolysis of the combined two reactions using 1 M NaOH (15 mL) in MeOH/DCM (50 mL/10 mL, 45° C., 2 h). LCMS [M+H]⁺ 510.1. The title compound (white solid, formic acid salt, 39.2 mg, 59%, 99.9% purity) was prepared according to a procedure similar to Example 1, Step 6 using (S)—N-(3-bromo-6-(3,4-dimethylpiperazin-1-yl)-2,4-difluorophenyl)-4-fluoro-2-(trifluoromethyl)benzamide (67 mg, 0.1 mmol based on 75.7%% purity) and (2-((2R,6R)-2,6-dimethylmorpholino)pyrimidin-5-yl)boronic acid (0.2 mmol, crude). ¹H NNMR (500 MHz, METHANOL-d₄) δ=8.46 (s, 3H), 7.86-7.81 (m, 1H), 7.68 (br d, J=8.9 Hz, 1H), 7.58 (t, J=7.8 Hz, 1H), 6.97 (br d, J=11.6 Hz, 1H), 4.16-4.08 (m, 2H), 3.98 (br d, J=13.1 Hz, 2H), 3.62 (br dd, J=6.2, 13.1 Hz, 2H), 3.41-3.34 (m, 2H), 3.20 (br d, J=11.0 Hz, 1H), 3.11-3.02 (m, 1H), 2.90-2.72 (m, 3H), 2.60 (br s, 3H), 1.29-1.22 (m, J=6.2 Hz, 9H); LCMS [M+H]⁺ 623.5.

### Example 168: N-(2,4-difluoro-3-(6-((R)-2-methylmorpholino)pyridin-3-yl)-6-((3S,5R)-3,4,5-trimethylpiperazin-1-yl)phenyl)-6-oxo-4-(trifluoromethyl)-1,6-dihydropyridine-3-carboxamide

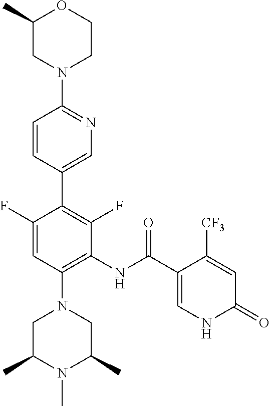 [SMILES: C[C@@H]1CN(c2ccc(-c3c(F)cc(N4C[C@@H](C)N(C)[C@@H](C)C4)c(NC(=O)c4c[nH]c(=O)cc4C(F)(F)F)c3F)cn2)CCO1]

The title compound (white solid, 32.9 mg, 53%, 99.8% purity) was prepared according to a method similar to Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (62 mg, 0.1 mmol) and (R)-2-methyl-4-(5-(4,4,5,5-tetramethyl-1,3,2-dioxaborolan-2-yl)pyridin-2-yl)morpholine (61 mg, 0.2 mmol), followed by TFA deprotection (1 mL TFA/10 mL DCM, rt, 1 h). ¹H NMR (500 MHz, METHANOL-d₄) δ=8.20 (s, 1H), 7.97 (s, 1H), 7.68 (br d, J=8.7 Hz, 1H), 6.95-6.93 (m, 1H), 6.92 (br d, J=9.2 Hz, 1H), 6.87 (br d, J=11.6 Hz, 1H), 4.17 (br d, J=12.6 Hz, 1H), 4.08 (br d, J=12.8 Hz, 1H), 4.00 (br dd, J=2.1, 11.5 Hz, 1H), 3.74-3.65 (m, 2H), 3.20 (br d, J=11.5 Hz, 2H), 2.96 (dt, J=3.3, 12.3 Hz, 1H), 2.69-2.52 (m, 5H), 2.40 (s, 3H), 1.25 (d, J=6.1 Hz, 3H), 1.18 (br d, J=6.1 Hz, 6H); LCMS [M+H]⁺ 621.5.

### Example 169: N-(6-((S)-3,4-dimethylpiperazin-1-yl)-2,4-difluoro-3-(6-((R)-2-methylmorpholino)pyridin-3-yl)phenyl)-6-oxo-4-(trifluoromethyl)-1,6-dihydropyridine-3-carboxamide

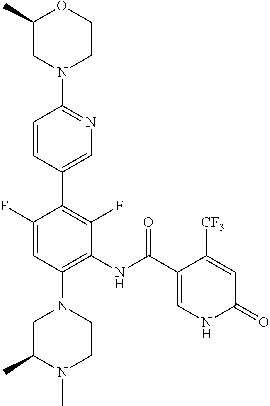 [SMILES: C[C@@H]1CN(c2ccc(-c3c(F)cc(N4CCN(C)[C@@H](C)C4)c(NC(=O)c4c[nH]c(=O)cc4C(F)(F)F)c3F)cn2)CCO1]

The title compound (white solid, 42.1 mg, 68%, 98.2% purity) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (R)-2-methyl-4-(5-(4,4,5,5-tetramethyl-1,3,2-dioxaborolan-2-yl)pyridin-2-yl)morpholine (61 mg, 0.2 mmol), followed by TFA deprotection (1 mL TFA/10 mL DCM, rt, 1 h). ¹H NMR (500 MHz, METHANOL-d₄) δ=8.16 (br s, 1H), 7.94 (s, 1H), 7.64 (br d, J=8.6 Hz, 1H), 6.92-6.89 (m, 1H), 6.88 (br d, J=9.0 Hz, 1H), 6.84 (br d, J=11.9 Hz, 1H), 4.13 (br d, J=12.6 Hz, 1H), 4.07-4.01 (m, 1H), 3.99-3.94 (m, 1H), 3.71-3.61 (m, 2H), 3.24-3.12 (m, 2H), 2.99-2.86 (m, 3H), 2.62-2.47 (m, 3H), 2.44-2.33 (m, 4H), 1.22 (d, J=6.1 Hz, 3H), 1.11 (br d, J=6.2 Hz, 3H); LCMS [M+H]⁺ 607.5.

### Example 170: N-(3-(6-((2S,6R)-2,6-dimethylmorpholino)pyridin-3-yl)-6-((S)-3,4-dimethylpiperazin-1-yl)-2,4-difluorophenyl)-6-oxo-4-(trifluoromethyl)-1,6-dihydropyridine-3-carboxamide

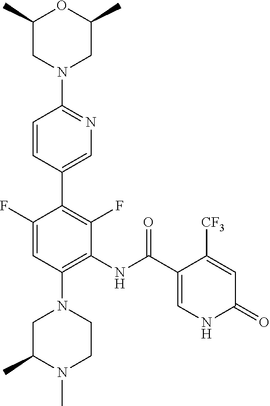 [SMILES: C[C@@H]1CN(c2ccc(-c3c(F)cc(N4CCN(C)[C@@H](C)C4)c(NC(=O)c4c[nH]c(=O)cc4C(F)(F)F)c3F)cn2)C[C@H](C)O1]

The title compound (white solid, 40.3 mg, 64%, 98.8% purity) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (2S,6R)-2,6-dimethyl-4-(5-(4,4,5,5-tetramethyl-1,3,2-dioxaborolan-2-yl)pyridin-2-yl)morpholine (64 mg, 0.2 mmol), followed by TFA deprotection (1 mL TFA/10 mL DCM, rt, 1 h). ¹H NMR (500 MHz, METHANOL-d₄) a=8.15 (br s, 1H), 7.94 (s, 1H), 7.63 (br d, J=8.7 Hz, 1H), 6.92-6.90 (m, 1H), 6.88 (br d, J=9.0 Hz, 1H), 6.84 (br d, J=11.7 Hz, 1H), 4.13 (br d, J=12.2 Hz, 2H), 3.74-3.66 (m, 2H), 3.24-3.11 (m, 2H), 2.99-2.84 (m, 2H), 2.59-2.45 (m, 4H), 2.43-2.32 (m, 4H), 1.23 (d, J=6.2 Hz, 6H), 1.11 (br d, J=6.1 Hz, 3H); LCMS [M+H]⁺ 621.6.

### Example 171: N-(6-((S)-3,4-dimethylpiperazin-1-yl)-2,4-difluoro-3-(6-((S)-2-methylmorpholino)pyridin-3-yl)phenyl)-6-oxo-4-(trifluoromethyl)-1,6-dihydropyridine-3-carboxamide

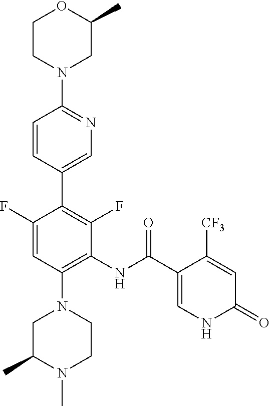 [SMILES: C[C@H]1CN(c2ccc(-c3c(F)cc(N4CCN(C)[C@@H](C)C4)c(NC(=O)c4c[nH]c(=O)cc4C(F)(F)F)c3F)cn2)CCO1]

The title compound (white solid, 29.6 mg, 48%, 99.1% purity) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and (S)-2-methyl-4-(5-(4,4,5,5-tetramethyl-1,3,2-dioxaborolan-2-yl)pyridin-2-yl)morpholine (61 mg, 0.2 mmol), followed by TFA deprotection (1 mL TFA/10 mL DCM, rt, 1 h). ¹H NMR (500 MHz, METHANOL-d₄) a=8.16 (br s, 1H), 7.95 (s, 1H), 7.64 (br d, J=8.7 Hz, 1H), 6.92-6.89 (m, 1H), 6.88 (br d, J=9.2 Hz, 1H), 6.84 (br d, J=11.7 Hz, 1H), 4.13 (br d, J=12.6 Hz, 1H), 4.04 (br d, J=12.6 Hz, 1H), 4.00-3.94 (m, 1H), 3.71-3.61 (m, 2H), 3.24-3.12 (m, 2H), 2.99-2.85 (m, 3H), 2.62-2.47 (m, 3H), 2.45-2.32 (m, 4H), 1.22 (d, J=6.2 Hz, 3H), 1.11 (br d, J=6.1 Hz, 3H); LCMS [M+H]⁺ 607.5.

### Example 172: N-(6-((S)-3,4-dimethylpiperazin-1-yl)-2,4-difluoro-3-(6-((R)-2-methylmorpholino)pyridin-3-yl)phenyl)-4-fluoro-2-(trifluoromethyl)benzamide

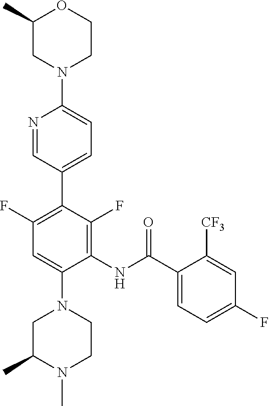 [SMILES: C[C@@H]1CN(c2ccc(-c3c(F)cc(N4CCN(C)[C@@H](C)C4)c(NC(=O)c4ccc(F)cc4C(F)(F)F)c3F)cn2)CCO1]

The title compound (white solid, 15.5 mg, 25%, 99.7% purity) was prepared according to a procedure similar to Example 1, Step 6 using (S)—N-(3-bromo-6-(3,4-dimethylpiperazin-1-yl)-2,4-difluorophenyl)-4-fluoro-2-(trifluoromethyl)benzamide (67 mg, 0.1 mmol based on 75.7%% purity) and (R)-2-methyl-4-(5-(4,4,5,5-tetramethyl-1,3,2-dioxaborolan-2-yl)pyridin-2-yl)morpholine (61 mg, 0.2 mmol). ¹H NMR (500 MHz, METHANOL-d₄) δ=8.21 (br s, 1H), 7.87-7.77 (m, 1H), 7.69 (br d, J=8.8 Hz, 1H), 7.66 (br d, J=9.3 Hz, 1H), 7.57 (br t, J=7.9 Hz, 1H), 6.99-6.84 (m, 2H), 4.18 (br d, J=12.6 Hz, 1H), 4.09 (br d, J=12.6 Hz, 1H), 4.01 (br d, J=11.2 Hz, 1H), 3.78-3.63 (m, 2H), 3.29 (br d, J=12.1 Hz, 1H), 3.24 (br d, J=11.6 Hz, 1H), 3.07-2.89 (m, 3H), 2.71-2.54 (m, 3H), 2.52-2.34 (m, 4H), 1.26 (br d, J=6.1 Hz, 3H), 1.17 (br d, J=5.9 Hz, 3H); LCMS [M+H]⁺ 608.4.

### Example 173: (S)—N-(6-(3,4-dimethylpiperazin-1-yl)-2,4-difluoro-3-(2-(piperazin-1-yl)pyrimidin-5-yl)phenyl)-4-fluoro-2-(trifluoromethyl)benzamide

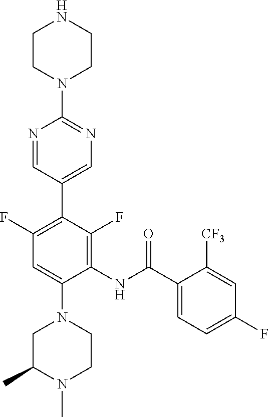 [SMILES: C[C@H]1CN(c2cc(F)c(-c3cnc(N4CCNCC4)nc3)c(F)c2NC(=O)c2ccc(F)cc2C(F)(F)F)CCN1C]

The title compound (brown solid, 36 mg, 31%, 99.3% purity) was prepared according to a procedure similar to Example 1, Step 6 using (S)—N-(3-bromo-6-(3,4-dimethylpiperazin-1-yl)-2,4-difluorophenyl)-4-fluoro-2-(trifluoromethyl)benzamide (135 mg, 0.2 mmol based on 75.7%% purity) and (2-(4-Boc-piperazino)pyrimidine-5-boronic acid pinacol ester (117 mg, 0.3 mmol), followed by TFA deprotection (1 mL TFA/10 mL DCM, rt, 1 h). ¹H NMR (500 MHz, METHANOL-d₄) δ=8.45 (s, 2H), 7.81 (t, J=7.3 Hz, 1H), 7.67 (br d, J=8.4 Hz, 1H), 7.57 (br t, J=7.5 Hz, 1H), 6.91 (br d, J=11.7 Hz, 1H), 3.92-3.84 (m, 4H), 3.30-3.19 (m, 2H), 3.00-2.88 (m, 6H), 2.60 (br t, J=10.9 Hz, 1H), 2.51 (br t, J=10.9 Hz, 1H), 2.37 (s, 4H), 1.14 (br d, J=6.0 Hz, 3H); LCMS [M+H]⁺ 594.5.

### Example 174: (S)—N-(6-(3,4-dimethylpiperazin-1-yl)-2,4-difluoro-3-(6-morpholinopyridin-3-yl)phenyl)-4-fluoro-2-(trifluoromethyl)benzamide

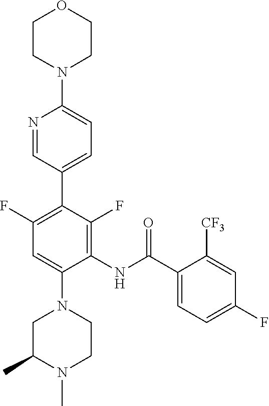

The title compound (white solid, 29.5 mg, 48%, 97.0% purity) was prepared according to a procedure similar to Example 1, Step 6 using (S)—N-(3-bromo-6-(3,4-dimethylpiperazin-1-yl)-2,4-difluorophenyl)-4-fluoro-2-(trifluoromethyl)benzamide (67 mg, 0.1 mmol based on 75.7%% purity) and 4-[5-(4,4,5,5-tetramethyl-[1,3,2]dioxaborolan-2-yl)-pyridin-2-yl]-morpholine (58 mg, 0.2 mmol). ¹H NMR (500 MHz, METHANOL-d₄) δ=8.22 (br s, 1H), 7.80 (br dd, J=5.4, 8.0 Hz, 1H), 7.71-7.64 (m, 2H), 7.57 (t, J=8.1 Hz, 1H), 6.95-6.85 (m, 2H), 3.82 (br t, J=4.7 Hz, 4H), 3.55 (br t, J=4.7 Hz, 4H), 3.31-3.19 (m, 2H), 3.00-2.90 (m, 2H), 2.65-2.49 (m, 2H), 2.44-2.36 (m, 4H), 1.15 (br d, J=6.2 Hz, 3H); LCMS [M+H]⁺ 594.4.

### Example 175: 4-(Difluoromethyl)-N-(3-(6-((2S,6R)-2,6-dimethylmorpholino)pyridin-3-yl)-6-((S)-3,4-dimethylpiperazin-1-yl)-2,4-difluorophenyl)-1-methyl-6-oxo-1,6-dihydropyridine-3-carboxamide

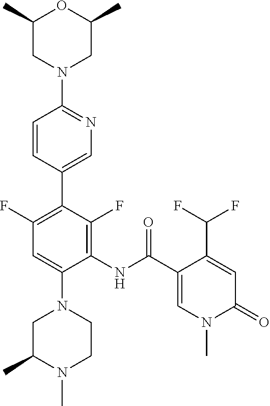 [SMILES: C[C@@H]1CN(c2ccc(-c3c(F)cc(N4CCN(C)[C@@H](C)C4)c(NC(=O)c4cn(C)c(=O)cc4C(F)F)c3F)cn2)C[C@H](C)O1]

The title compound (off white solid, 18.6 mg, 20%, 98.6% purity) was prepared according to a procedure similar to Example 1, Step 6 using (S)—N-(3-bromo-6-(3,4-dimethylpiperazin-1-yl)-2,4-difluorophenyl)-4-(difluoromethyl)-1-methyl-6-oxo-1,6-dihydropyridine-3-carboxamide (98 mg, 0.147 mmol based on 75.6% purity) and (2S,6R)-2,6-dimethyl-4-(5-(4,4,5,5-tetramethyl-1,3,2-dioxaborolan-2-yl)pyridin-2-yl)morpholine (93 mg, 0.29 mmol). ¹H NMR (500 MHz, METHANOL-d₄) δ=8.34 (s, 1H), 8.18 (s, 1H), 7.66 (br d, J=8.8 Hz, 1H), 7.32 (t, J=55.0 Hz, 1H), 6.91 (d, J=8.9 Hz, 1H), 6.87 (br d, J=11.6 Hz, 1H), 6.82 (br s, 1H), 4.16 (br d, J=12.1 Hz, 2H), 3.77-3.68 (m, 2H), 3.65 (s, 3H), 3.25-3.14 (m, 2H), 3.00-2.87 (m, 2H), 2.62-2.48 (m, 4H), 2.44-2.34 (m, 4H), 1.26 (d, J=6.2 Hz, 6H), 1.12 (br d, J=6.2 Hz, 3H); LCMS [M+H]⁺ 617.6.

### Example 176: N-(3-(6-((2S,6R)-2,6-dimethylmorpholino)pyridin-3-yl)-6-((S)-3,4-dimethylpiperazin-1-yl)-2,4-difluorophenyl)-4-fluoro-2-(trifluoromethyl)benzamide

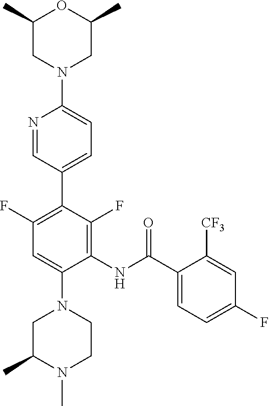 [SMILES: C[C@@H]1CN(c2ccc(-c3c(F)cc(N4CCN(C)[C@@H](C)C4)c(NC(=O)c4ccc(F)cc4C(F)(F)F)c3F)cn2)C[C@H](C)O1]

The title compound (white solid, 40.8 mg, 33%, 99.3% purity) was prepared according to a procedure similar to Example 1, Step 6 using (S)—N-(3-bromo-6-(3,4-dimethylpiperazin-1-yl)-2,4-difluorophenyl)-4-fluoro-2-(trifluoromethyl)benzamide (135 mg, 0.2 mmol based on 75. 7%% purity) and (2S,6R)-2,6-dimethyl-4-(5-(4,4,5,5-tetramethyl-1,3,2-dioxaborolan-2-yl)pyridin-2-yl)morpholine (191 mg, 0.6 mmol). ¹H NMR (500 MHz, METHANOL-d₄) δ=8.20 (br s, 1H), 7.81 (br dd, J=5.3, 8.1 Hz, 1H), 7.71-7.64 (m, 2H), 7.58 (t, J=8.1 Hz, 1H), 6.96-6.86 (m, 2H), 4.17 (br d, J=12.0 Hz, 2H), 3.79-3.69 (m, 2H), 3.30-3.19 (m, 2H), 3.03-2.90 (m, 2H), 2.65-2.48 (m, 4H), 2.47-2.35 (m, 4H), 1.26 (d, J=6.2 Hz, 6H), 1.16 (br d, J=6.1 Hz, 3H); LCMS [M+H]⁺ 622.6.

### Example 177: 2-(Di fluoromethyl)-N-(6-((S)-3,4-dimethylpiperazin-1-yl)-2,4-difluoro-3-(6-((R)-2-methylmorpholino)pyridin-3-yl)phenyl)-4-fluorobenzamide

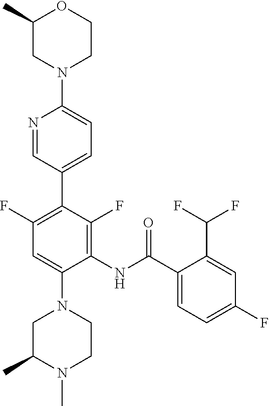 [SMILES: C[C@@H]1CN(c2ccc(-c3c(F)cc(N4CCN(C)[C@@H](C)C4)c(NC(=O)c4ccc(F)cc4C(F)F)c3F)cn2)CCO1]

(S)—N-(3-bromo-6-(3,4-dimethylpiperazin-1-yl)-2,4-difluorophenyl)-2-(difluoromethyl)-4-fluorobenzamide (light greenish solid, 923 mg, 71% based on 75.5% purity) was prepared according to a procedure similar to that described in Example 13, Step 7 using 2-(difluoromethyl)-4-fluorobenzoic acid (570 mg, 3 mmol), T3P (50% wt % in EtOAc, 2.38 mL, 4 mmol), pyridine (2 mL), ⁱPr₂NEt (1.4 mL, 4 mmol) and (S)-3-bromo-6-(3,4-dimethylpiperazin-1-yl)-2,4-difluoroaniline (640 mg, 2 mmol). The title compound (white solid, 30.9 mg, 52%, 98.7% purity) was prepared according to a procedure similar to that described in Example 1, Step 6 using (S)—N-(3-bromo-6-(3,4-dimethylpiperazin-1-yl)-2,4-difluorophenyl)-2-(difluoromethyl)-4-fluorobenzamide (65 mg, 0.1 mmol based on 75.5% purity) and (R)-2-methyl-4-(5-(4,4,5,5-tetramethyl-1,3,2-dioxaborolan-2-yl)pyridin-2-yl)morpholine (61 mg, 0.2 mmol). ¹H NMR (500 MHz, METHANOL-d₄) δ=8.20 (br s, 1H), 7.93-7.84 (m, 1H), 7.68 (br d, J=8.6 Hz, 1H), 7.56 (br d, J=9.0 Hz, 1H), 7.49-7.23 (m, 2H), 6.92 (d, J=8.6 Hz, 1H), 6.87 (d, J=11.7 Hz, 1H), 4.16 (br d, J=12.6 Hz, 1H), 4.07 (br d, J=13.0 Hz, 1H), 4.00 (br d, J=11.1 Hz, 1H), 3.75-3.64 (m, 2H), 3.29-3.16 (m, 2H), 3.03-2.85 (m, 3H), 2.67-2.53 (m, 2H), 2.48 (br t, J=10.6 Hz, 1H), 2.41-2.31 (m, 4H), 1.25 (d, J=6.2 Hz, 3H), 1.11 (br d, J=6.1 Hz, 3H); LCMS [M+H]⁺ 590.4.

### Example 178: 2-(Di fluoromethyl)-N-(3-(6-((2S,6R)-2,6-dimethylmorpholino)pyridin-3-yl)-6-((S)-3,4-dimethylpiperazin-1-yl)-2,4-difluorophenyl)-4-fluorobenzamide

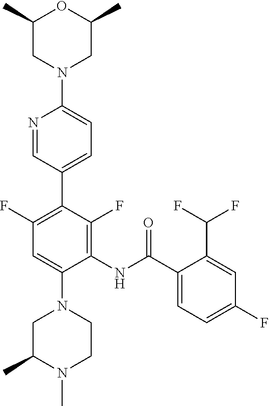 [SMILES: C[C@@H]1CN(c2ccc(-c3c(F)cc(N4CCN(C)[C@@H](C)C4)c(NC(=O)c4ccc(F)cc4C(F)F)c3F)cn2)C[C@H](C)O1]

The title compound (off white solid, 39.6 mg, 64%, 97.3% purity) was prepared according to a procedure similar to Example 1, Step 6 using (S)—N-(3-bromo-6-(3,4-dimethylpiperazin-1-yl)-2,4-difluorophenyl)-2-(difluoromethyl)-4-fluorobenzamide (65 mg, 0.1 mmol based on 75.5% purity) and (2S,6R)-2,6-dimethyl-4-(5-(4,4,5,5-tetramethyl-1,3,2-dioxaborolan-2-yl)pyridin-2-yl)morpholine (64 mg, 0.2 mmol). ¹H NMR (500 MHz, METHANOL-d4) δ=8.20 (br s, 1H), 7.94-7.86 (m, 1H), 7.68 (br d, J=8.7 Hz, 1H), 7.56 (br d, J=9.0 Hz, 1H), 7.50-7.24 (m, 2H), 6.92 (br d, J=8.9 Hz, 1H), 6.88 (br d, J=12.1 Hz, 1H), 4.17 (br d, J=12.6 Hz, 2H), 3.78-3.69 (m, 2H), 3.29-3.17 (m, 2H), 3.00-2.86 (m, 2H), 2.62-2.44 (m, 4H), 2.41-2.30 (m, 4H), 1.26 (d, J=6.1 Hz, 6H), 1.11 (br d, J=6.1 Hz, 3H); LCMS [M+H]⁺ 604.5.

### Example 179: 2-(Difluoromethyl)-N-(6-((S)-3,4-dimethylpiperazin-1-yl)-2,4-difluoro-3-(6-((S)-2-methylmorpholino)pyridin-3-yl)phenyl)-4-fluorobenzamide

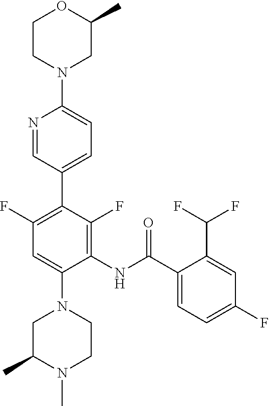 [SMILES: C[C@H]1CN(c2ccc(-c3c(F)cc(N4CCN(C)[C@@H](C)C4)c(NC(=O)c4ccc(F)cc4C(F)F)c3F)cn2)CCO1]

The title compound (pale beige solid, 34.3 mg, 57%, 98.4% purity) was prepared according to a procedure similar to Example 1, Step 6 using (S)—N-(3-bromo-6-(3,4-dimethylpiperazin-1-yl)-2,4-difluorophenyl)-2-(difluoromethyl)-4-fluorobenzamide (65 mg, 0.1 mmol based on 75.5% purity) and (S)-2-methyl-4-(5-(4,4,5,5-tetramethyl-1,3,2-dioxaborolan-2-yl)pyridin-2-yl)morpholine (62 mg, 0.2 mmol). ¹H NMR (500 MHz, METHANOL-d₄) δ=8.21 (br s, 1H), 7.93-7.85 (m, 1H), 7.69 (br d, J=8.6 Hz, 1H), 7.56 (br d, J=9.0 Hz, 1H), 7.50-7.24 (m, 2H), 6.92 (br d, J=8.9 Hz, 1H), 6.88 (br d, J=11.7 Hz, 1H), 4.17 (br d, J=12.6 Hz, 1H), 4.08 (br d, J=12.8 Hz, 1H), 4.01 (br d, J=11.2 Hz, 1H), 3.76-3.65 (m, 2H), 3.29-3.17 (m, 2H), 3.02-2.87 (m, 3H), 2.68-2.54 (m, 2H), 2.48 (br t, J=10.7 Hz, 1H), 2.35 (s, 4H), 1.26 (d, J=6.1 Hz, 3H), 1.12 (br d, J=6.1 Hz, 3H); LCMS [M+H]⁺ 590.4.

### Example 180: (S)-2-(Difluoromethyl)-N-(6-(3,4-dimethylpiperazin-1-yl)-2,4-difluoro-3-(6-morpholinopyridin-3-yl)phenyl)-4-fluorobenzamide

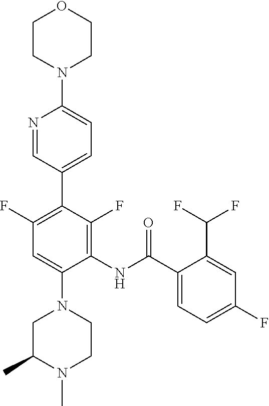

The title compound (white solid, 27.7 mg, 46%, 95.3% purity) was prepared according to a procedure similar to Example 1, Step 6 using (S)—N-(3-bromo-6-(3,4-dimethylpiperazin-1-yl)-2,4-difluorophenyl)-2-(difluoromethyl)-4-fluorobenzamide (65 mg, 0.1 mmol based on 75.5% purity) and 4-[5-(4,4,5,5-tetramethyl-[1,3,2]dioxaborolan-2-yl)-pyridin-2-yl]-morpholine (58 mg, 0.2 mmol). ¹H NMR (500 MHz, METHANOL-d₄) δ=8.22 (br s, 1H), 7.90 (t, J=7.2 Hz, 1H), 7.70 (br d, J=8.7 Hz, 1H), 7.56 (br d, J=8.8 Hz, 1H), 7.49-7.24 (m, 2H), 6.93 (d, J=8.7 Hz, 1H), 6.88 (d, J=11.6 Hz, 1H), 3.83 (br t, J=4.5 Hz, 4H), 3.56 (br t, J=4.5 Hz, 4H), 3.29-3.18 (m, 2H), 2.99-2.86 (m, 2H), 2.59 (br t, J=10.8 Hz, 1H), 2.46 (br t, J=10.5 Hz, 1H), 2.37-2.30 (m, 4H), 1.11 (br d, J=6.2 Hz, 3H); LCMS [M+H]⁺ 576.5.

### Example 181: 4-(Difluoromethyl)-N-(6-((S)-3,4-dimethylpiperazin-1-yl)-2,4-difluoro-3-(6-((R)-2-methylmorpholino)pyridin-3-yl)phenyl)-6-oxo-1,6-dihydropyridine-3-carboxamide

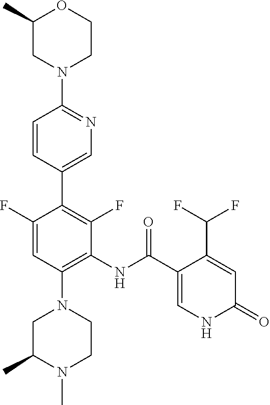 [SMILES: C[C@@H]1CN(c2ccc(-c3c(F)cc(N4CCN(C)[C@@H](C)C4)c(NC(=O)c4c[nH]c(=O)cc4C(F)F)c3F)cn2)CCO1]

The title compound (white solid, 20.5 mg, 34%, 98.1% purity) was prepared according to a procedure similar to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 0.1 mmol based on 85.5% purity) and (R)-2-methyl-4-(5-(4,4,5,5-tetramethyl-1,3,2-dioxaborolan-2-yl)pyridin-2-yl)morpholine (76 mg, 0.25 mmol), followed by TFA deprotection (1 mL TFA/10 mL DCM, rt, 1 h). ¹H NMR (500 MHz, METHANOL-d₄) δ=8.20 (s, 1H), 8.06 (s, 1H), 7.68 (br d, J=8.7 Hz, 1H), 7.32 (t, J=55.0 Hz, 1H), 6.92 (d, J=8.9 Hz, 1H), 6.87 (br d, J=11.6 Hz, 1H), 6.82 (s, 1H), 4.17 (br d, J=12.7 Hz, 1H), 4.08 (br d, J=12.8 Hz, 1H), 4.04-3.98 (m, 1H), 3.76-3.65 (m, 2H), 3.26-3.15 (m, 2H), 3.03-2.88 (m, 3H), 2.66-2.47 (m, 3H), 2.44-2.35 (m, 4H), 1.26 (d, J=6.1 Hz, 3H), 1.12 (br d, J=6.1 Hz, 3H); LCMS [M+H]⁺ 589.4.

### Example 182: 4-(Difluoromethyl)-N-(3-(6-((2S,6R)-2,6-dimethylmorpholino)pyridin-3-yl)-6-((S)-3,4-dimethylpiperazin-1-yl)-2,4-difluorophenyl)-6-oxo-1,6-dihydropyridine-3-carboxamide

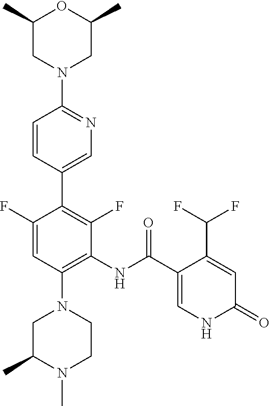 [SMILES: C[C@@H]1CN(c2ccc(-c3c(F)cc(N4CCN(C)[C@@H](C)C4)c(NC(=O)c4c[nH]c(=O)cc4C(F)F)c3F)cn2)C[C@H](C)O1]

The title compound (white solid, 19.1 mg, 31%, 98.3% purity) was prepared according to a procedure similar to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 0.1 mmol based on 85.5% purity) and (2S,6R)-2,6-dimethyl-4-(5-(4,4,5,5-tetramethyl-1,3,2-dioxaborolan-2-yl)pyridin-2-yl)morpholine (95 mg, 0.3 mmol), followed by TFA deprotection (1 mL TFA/10 mL DCM, rt, 1 h). ¹H NMR (500 MHz, METHANOL-d₄) δ=8.19 (s, 1H), 8.06 (s, 1H), 7.67 (br d, J=8.7 Hz, 1H), 7.32 (t, J=55.0 Hz, 1H), 6.92 (br d, J=9.0 Hz, 1H), 6.87 (br d, J=11.6 Hz, 1H), 6.83-6.81 (m, 1H), 4.17 (br d, J=12.6 Hz, 2H), 3.78-3.69 (m, 2H), 3.29-3.12 (m, 2H), 3.00-2.89 (m, 2H), 2.63-2.47 (m, 4H), 2.46-2.35 (m, 4H), 1.26 (d, J=6.2 Hz, 6H), 1.13 (br d, J=6.1 Hz, 3H); LCMS [M+H]⁺ 603.4.

### Example 183: 4-(Difluoromethyl)-N-(6-((S)-3,4-dimethylpiperazin-1-yl)-2,4-difluoro-3-(6-((S)-2-methylmorpholino)pyridin-3-yl)phenyl)-6-oxo-1,6-dihydropyridine-3-carboxamide

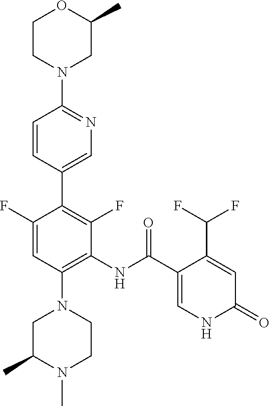 [SMILES: C[C@H]1CN(c2ccc(-c3c(F)cc(N4CCN(C)[C@@H](C)C4)c(NC(=O)c4c[nH]c(=O)cc4C(F)F)c3F)cn2)CCO1]

The title compound (white solid, 23.7 mg, 40%, 98.3% purity) was prepared according to Example 1, Step 6 using (S)—N-(3-bromo-6-(3,4-dimethylpiperazin-1-yl)-2,4-difluorophenyl)-4-(difluoromethyl)-6-(2-(trimethylsilyl)ethoxy)nicotinamide (69 mg, 0.1 mmol based on 85.5% purity) and (S)-2-methyl-4-(5-(4,4,5,5-tetramethyl-1,3,2-dioxaborolan-2-yl)pyridin-2-yl)morpholine (76 mg, 0.25 mmol), followed by TFA deprotection (1 mL TFA/10 mL DCM, 1 h). ¹H NMR (500 MHz, METHANOL-d₄) δ=8.19 (s, 1H), 8.06 (s, 1H), 7.67 (br d, J=8.8 Hz, 1H), 7.32 (t, J=55.0 Hz, 1H), 6.91 (br d, J=8.9 Hz, 1H), 6.86 (br d, J=11.6 Hz, 1H), 6.82 (br s, 1H), 4.17 (br d, J=12.7 Hz, 1H), 4.07 (br d, J=12.7 Hz, 1H), 4.00 (br d, J=11.1 Hz, 1H), 3.75-3.65 (m, 2H), 3.26-3.14 (m, 2H), 3.02-2.89 (m, 3H), 2.65-2.47 (m, 3H), 2.44-2.34 (m, 4H), 1.25 (d, J=6.1 Hz, 3H), 1.12 (br d, J=6.2 Hz, 3H); LCMS [M+H]⁺ 589.5.

### Example 184: (S)—N-(3-(benzo[d][1,3]dioxol-5-yl)-6-(3,4-dimethylpiperazin-1-yl)-2,4-difluorophenyl)-6-oxo-4-(trifluoromethyl)-1,6-dihydropyridine-3-carboxamide

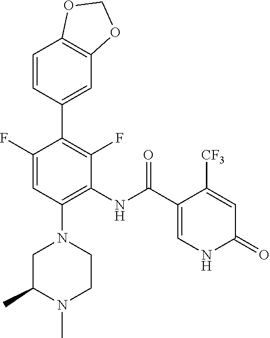 [SMILES: C[C@H]1CN(c2cc(F)c(-c3ccc4c(c3)OCO4)c(F)c2NC(=O)c2c[nH]c(=O)cc2C(F)(F)F)CCN1C]

The title compound (white solid, 42.6 mg, 77%, 99.9% purity) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and 3,4-methylenedioxyphenylboronic acid (33.2 mg, 0.2 mmol), followed by TFA deprotection (1 mL TFA/10 mL DCM, rt, 1 h). ¹H NMR (500 MHz, METHANOL-d₄) 6=7.97 (s, 1H), 6.95-6.89 (m, 4H), 6.84 (br d, J=11.6 Hz, 1H), 6.01 (s, 2H), 3.26-3.14 (m, 2H), 3.00-2.88 (m, 2H), 2.63-2.48 (m, 2H), 2.45-2.35 (m, 4H), 1.14 (br d, J=6.2 Hz, 3H); LCMS [M+H]⁺ 551.4.

### Example 185: (S)—N-(3-(2,3-dihydrobenzo[b][1,4]dioxin-6-yl)-6-(3,4-dimethylpiperazin-1-yl)-2,4-difluorophenyl)-6-oxo-4-(trifluoromethyl)-1,6-dihydropyridine-3-carboxamide

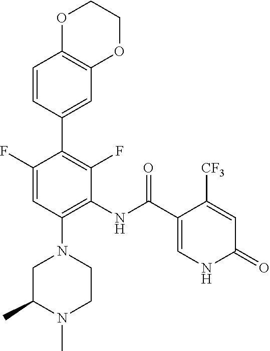 [SMILES: C[C@H]1CN(c2cc(F)c(-c3ccc4c(c3)OCCO4)c(F)c2NC(=O)c2c[nH]c(=O)cc2C(F)(F)F)CCN1C]

The title compound (white solid, 39.7 mg, 70%, 100% purity) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and 2,3-dihydro-1,4-benzodioxin-6-ylboronic acid (36 mg, 0.2 mmol), followed by TFA deprotection (1 mL TFA/10 mL DCM, rt, 1 h). ¹H NMR (500 MHz, METHANOL-d₄) δ=7.97 (s, 1H), 6.96-6.88 (m, 4H), 6.83 (br d, J=11.5 Hz, 1H), 4.29 (s, 4H), 3.26-3.15 (m, 2H), 3.00-2.88 (m, 2H), 2.63-2.49 (m, 2H), 2.45-2.35 (m, 4H), 1.14 (br d, J=6.1 Hz, 3H); LCMS [M+H]⁺ 565.4.

### Example 186: (S)—N-(6-(3,4-dimethylpiperazin-1-yl)-2,4-difluoro-3-(6-((tetrahydro-2H-pyran-4-yl)oxy)pyridin-3-yl)phenyl)-6-oxo-4-(trifluoromethyl)-1,6-dihydropyridine-3-carboxamide

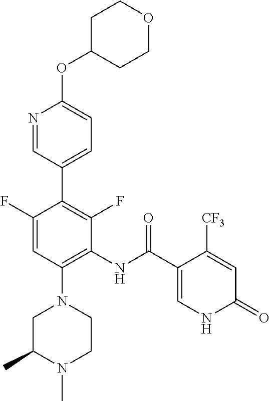 [SMILES: C[C@H]1CN(c2cc(F)c(-c3ccc(OC4CCOCC4)nc3)c(F)c2NC(=O)c2c[nH]c(=O)cc2C(F)(F)F)CCN1C]

The title compound (white solid, 36.8 mg, 60%, 99.9% purity) was prepared according to a procedure similar to Example 1, Step 6 using (S)—N-(3-bromo-6-(3,4-dimethylpiperazin-1-yl)-2,4-difluorophenyl)-4-(trifluoromethyl)-6-(2-(trimethylsilyl)ethoxy)nicotinamide (61 mg, 0.1 mmol) and 2-(tetrahydropyran-4-yloxy)-5-(4,4,5,5-tetramethyl-1,3,2-dioxaborolan-2-yl)pyridine (61 mg, 0.2 mmol), followed by TFA deprotection (1 mL TFA/10 mL DCM, rt, 1 h). ¹H NMR (500 MHz, METHANOL-d₄) δ=8.21 (br s, 1H), 7.97 (s, 1H), 7.78 (br d, J=8.6 Hz, 1H), 6.96-6.87 (m, 3H), 5.27 (td, J=4.2, 8.1 Hz, 1H), 4.03-3.96 (m, 2H), 3.68-3.60 (m, 2H), 3.28-3.16 (m, 2H), 3.04-2.90 (m, 2H), 2.64-2.47 (m, 2H), 2.47-2.36 (m, 4H), 2.15-2.07 (m, 2H), 1.84-1.75 (m, 2H), 1.15 (br d, J=6.1 Hz, 3H); LCMS [M+H]⁺ 608.4.

### Example 187: (S)-4-(difluoromethyl)-N-(3-(1-(2-((dimethylamino)methyl)thiazole-4-carbonyl)-1,2,3,6-tetrahydropyridin-4-yl)-6-(3,4-dimethylpiperazin-1-yl)-2,4-difluorophenyl)-6-oxo-1,6-dihydropyridine-3-carboxamide

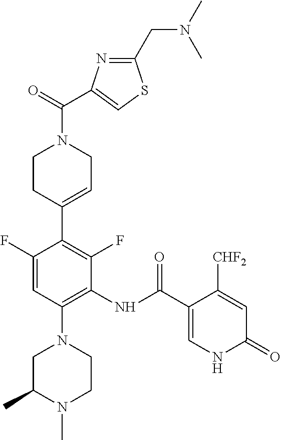 [SMILES: C[C@H]1CN(c2cc(F)c(C3=CCN(C(=O)c4csc(CN(C)C)n4)CC3)c(F)c2NC(=O)c2c[nH]c(=O)cc2C(F)F)CCN1C]

A mixture of (S)-4-(difluoromethyl)-N-(6-(3,4-dimethylpiperazin-1-yl)-2,4-difluoro-3-(1,2,3,6-tetrahydropyridin-4-yl)phenyl)-6-oxo-1,6-dihydropyridine-3-carboxamide (25 mg, 0.051 mmol), 2-[(dimethylamino)methyl]-1,3-thiazole-4-carboxylic acid (10.38 mg, 0.056 mmol), HATU (48.2 mg, 0.127 mmol) and N,N-diisopropylethylamine (0.035 mL, 0.203 mmol) in DMF (3 mL) was stirred at room temperature for 1 h. The mixture was quenched with water (8 mL), brine (2 mL) and EtOAc (3 mL), The org phase was separated and the aqueous phase was extracted with EtOAc (3×3 mL), the combined organic layers were washed with water (5 mL), brine (5 mL), dried over Na₂SO₄ and concentrated onto Celite. Purification by silica gel chromatography afforded the desired product which was isolated as white powder (14.5 mg, 41% yield). ¹H NMR (500 MHz, METHANOL-d4) δ=8.00-7.85 (m, 2H), 7.33-7.06 (m, 1H), 6.73-6.68 (m, 1H), 6.68-6.63 (m, 1H), 5.91-5.67 (m, 1H) 4.42-4.17 (m, 2H), 3.90-3.81 (m, 2H), 3.76 (s, 2H), 3.09-2.99 (m, 2H), 2.83-2.75 (m, 2H), 2.53-2.33 (m, 4H), 2.33-2.20 (m, 10H), 1.02-0.96 (m, 3H); LCMS [M+1]⁺=662.6 g/mol.

### Example 188: N-(2,4-difluoro-3-(1-(2-methylthiazole-4-carbonyl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide

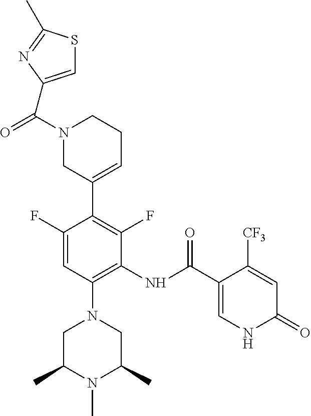 [SMILES: Cc1nc(C(=O)N2CCC=C(c3c(F)cc(N4C[C@@H](C)N(C)[C@@H](C)C4)c(NC(=O)c4c[nH]c(=O)cc4C(F)(F)F)c3F)C2)cs1]

The procedure was similar to Example 187 using N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide (20 mg, 0.038 mmol), 2-methyl-1,3-thiazole-4-carboxylic acid hydrochloride (8.20 mg, 0.046 mmol), HATU (36.2 mg, 0.095 mmol) and N,N-diisopropylethylamine (0.027 ml, 0.152 mmol) in DMF (3 mL) to give the desired product as a white powder (14 mg, 54% yield). ¹H NMR (500 MHz, METHANOL-d4) δ=8.01-7.90 (m, 1H), 7.90-7.83 (m, 1H), 6.97-6.91 (m, 1H), 6.86-6.72 (m, 1H), 6.17-5.98 (m, 1H), 4.51-4.34 (m, 2H), 3.99-3.84 (m, 2H), 3.26-3.12 (m, 2H), 2.84-2.68 (m, 3H), 2.67-2.52 (m, 4H), 2.52-2.45 (m, 2H), 2.42 (br s, 3H), 1.17 (brs, 6H); LCMS [M+1]⁺=651.6 g/mol

### Example 189: N-(3-(1-(2-((dimethylamino)methyl)thiazole-4-carbonyl)-1,2,5,6-tetrahydropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide

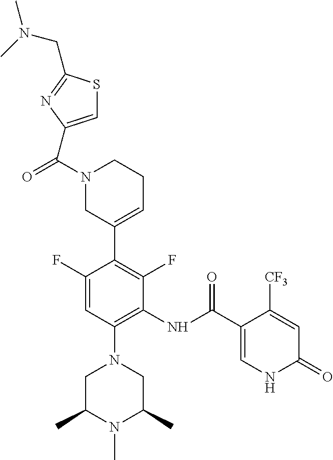 [SMILES: C[C@@H]1CN(c2cc(F)c(C3=CCCN(C(=O)c4csc(CN(C)C)n4)C3)c(F)c2NC(=O)c2c[nH]c(=O)cc2C(F)(F)F)C[C@H](C)N1C]

The procedure followed was similar to that of Example 187 using N-(2,4-difluoro-3-(1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide (20 mg, 0.038 mmol), 2-[(dimethylamino)methyl]-1,3-thiazole-4-carboxylic acid (8.51 mg, 0.046 mmol), HATU (36.2 mg, 0.095 mmol) and N,N-diisopropylethylamine (0.027 ml, 0.152 mmol) in DMF (3 mL) to give the desired product as a white powder (12 mg, 43% yield). ¹H NMR (500 MHz, METHANOL-d4) δ=7.97-7.88 (m, 1H), 7.86-7.77 (m, 1H), 6.87-6.80 (m, 1H), 6.76-6.61 (m, 1H), 6.01-5.88 (m, 1H), 4.43-4.25 (m, 2H), 3.85-3.71 (m, 4H), 3.14-3.01 (m, 2H), 2.61-2.49 (m, 4H), 2.41-2.33 (m, 5H), 2.31-2.26 (m, 3H), 2.26-2.18 (m, 3H), 1.12-1.05 (m, 6H); LCMS [M+1]⁺=694.6 g/mol.

### Example 190: N-(2,4-difluoro-3-(6-((R)-2-methylmorpholino)pyridin-3-yl)-6-((3S,5R)-3,4,5-trimethylpiperazin-1-yl)phenyl)-4-(difluoromethyl)-6-oxo-1,6-dihydropyridine-3-carboxamide

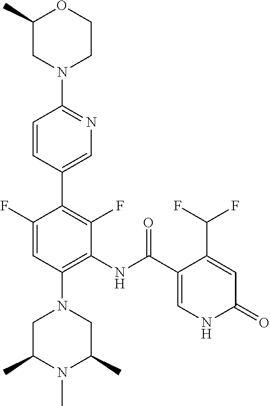 [SMILES: C[C@@H]1CN(c2ccc(-c3c(F)cc(N4C[C@@H](C)N(C)[C@@H](C)C4)c(NC(=O)c4c[nH]c(=O)cc4C(F)F)c3F)cn2)CCO1]

The title compound (light beige solid, 24.0 mg, 39%, 98.8% purity) was prepared according to a procedure similar to Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (71 mg, 0.1 mmol based on 85.0% purity) and (R)-2-methyl-4-(5-(4,4,5,5-tetramethyl-1,3,2-dioxaborolan-2-yl)pyridin-2-yl)morpholine (76 mg, 0.25 mmol), followed by TFA deprotection (1 mL TFA/10 mL DCM, rt, 1 h). ¹H NMR (500 MHz, METHANOL-d₄) δ=8.19 (br s, 1H), 8.06 (s, 1H), 7.67 (br d, J=8.7 Hz, 1H), 7.34 (t, J=55.0 Hz, 1H), 6.91 (br d, J=8.8 Hz, 1H), 6.85 (br d, J=11.7 Hz, 1H), 6.83-6.80 (m, 1H), 4.17 (br d, J=12.7 Hz, 1H), 4.08 (br d, J=12.7 Hz, 1H), 4.01 (br d, J=10.9 Hz, 1H), 3.75-3.64 (m, 2H), 3.19 (br d, J=11.5 Hz, 2H), 3.02-2.91 (m, 1H), 2.63 (br t, J=11.1 Hz, 3H), 2.56-2.46 (m, 2H), 2.37 (s, 3H), 1.25 (br d, J=6.0 Hz, 3H), 1.16 (br d, J=6.1 Hz, 6H); LCMS [M+H]⁺ 603.1.

### Example 191: 4-(Difluoromethyl)-N-(3-(6-((2S,6R)-2,6-dimethylmorpholino)pyridin-3-yl)-2,4-difluoro-6-((3S,5R)-3,4,5-trimethylpiperazin-1-yl)phenyl)-6-oxo-1,6-dihydropyridine-3-carboxamide

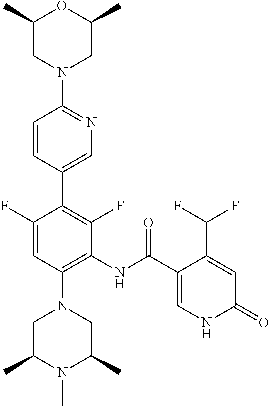 [SMILES: C[C@@H]1CN(c2ccc(-c3c(F)cc(N4C[C@@H](C)N(C)[C@@H](C)C4)c(NC(=O)c4c[nH]c(=O)cc4C(F)F)c3F)cn2)C[C@H](C)O1]

The title compound (pale beige solid, 36.0 mg, 58%, 98.5% purity) was prepared according to a procedure similar to Example 1, Step 6 using N-(3-bromo-2,4-difluoro-6-((3S,5R)-3,4,5-trimethylpiperazin-1-yl)phenyl)-4-(difluoromethyl)-6-(2-(trimethylsilyl)ethoxy)nicotinamide (71 mg, 0.1 mmol based on 85.0% purity) and (2S,6R)-2,6-dimethyl-4-(5-(4,4,5,5-tetramethyl-1,3,2-dioxaborolan-2-yl)pyridin-2-yl)morpholine (80 mg, 0.25 mmol), followed by TFA deprotection (1 mL TFA/10 mL DCM, rt, 1 h). ¹H NMR (500 MHz, METHANOL-d₄) 6=8.18 (br s, 1H), 8.06 (s, 1H), 7.66 (br d, J=8.2 Hz, 1H), 7.34 (t, J=55.0 Hz, 1H), 6.91 (br d, J=8.8 Hz, 1H), 6.87-6.80 (m, 2H), 4.16 (br d, J=12.6 Hz, 2H), 3.77-3.69 (m, 2H), 3.18 (br d, J=11.5 Hz, 2H), 2.65-2.46 (m, 6H), 2.36 (s, 3H), 1.26 (br d, J=6.1 Hz, 6H), 1.15 (br d, J=6.1 Hz, 6H); LCMS [M+H]⁺ 617.2.

### Example 192: N-(2,4-difluoro-3-(6-((S)-2-methylmorpholino)pyridin-3-yl)-6-((3S,5R)-3,4,5-trimethylpiperazin-1-yl)phenyl)-6-oxo-4-(trifluoromethyl)-1,6-dihydropyridine-3-carboxamide

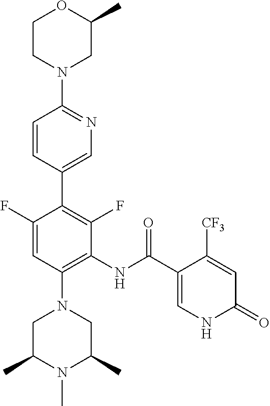 [SMILES: C[C@@H]1CN(c2cc(F)c(-c3ccc(N4CCO[C@@H](C)C4)nc3)c(F)c2NC(=O)c2c[nH]c(=O)cc2C(F)(F)F)C[C@H](C)N1C]

The title compound (white solid, 32.0 mg, 51%, 99.3% purity) was prepared according to a procedure similar to Example 1, Step 6 using N-(3-bromo-2,4-difluoro-6-((3S,5R)-3,4,5-trimethylpiperazin-1-yl)phenyl)-4-(trifluoromethyl)-6-(2-(trimethylsilyl)ethoxy)nicotinamide (62 mg, 0.1 mmol) and (S)-2-methyl-4-(5-(4,4,5,5-tetramethyl-1,3,2-dioxaborolan-2-yl)pyridin-2-yl)morpholine (61 mg, 0.2 mmol), followed by TFA deprotection (1 mL TFA/10 mL DCM, rt, 1 h). ¹H NMR (500 MHz, METHANOL-d4) δ=8.20 (br s, 1H), 7.97 (s, 1H), 7.68 (br d, J=8.7 Hz, 1H), 6.94 (s, 1H), 6.92 (br d, J=9.3 Hz, 1H), 6.87 (br d, J=11.6 Hz, 1H), 4.17 (br d, J=12.6 Hz, 1H), 4.08 (br d, J=12.8 Hz, 1H), 4.04-3.98 (m, 1H), 3.75-3.66 (m, 2H), 3.20 (br d, J=11.5 Hz, 2H), 2.97 (dt, J=3.3, 12.3 Hz, 1H), 2.69-2.52 (m, 5H), 2.40 (s, 3H), 1.26 (d, J=6.1 Hz, 3H), 1.18 (br d, J=6.1 Hz, 6H); LCMS [M+H]⁺ 621.5.

### C: Biological Assays

Compounds of the present application display inhibition of the interaction between WDR5 and its binding partners in the following assays:

### (i) Surface Plasmon Resonance (SPR) Assay

Exemplary compounds of the application were dissolved in 100% DMSO at 10 mM, assayed fresh, and then stored at −20° C. for repeat studies and other experiments. Full length WDR5 with an N-terminal His tag and C-terminal AviTag (Avidity Inc.) was expressed in E. coli with coexpression of BirA to biotin label the protein in vivo. Purification was via Ni-NTA. The purified WDR5 protein has a molecular weight of 41976 Da.

SPR studies were performed using a Biacore™ T200 instrument (GE Health Sciences Inc.). Biotinylated WDR5 protein (approximately 3000RU) was stably captured to streptavidin coupled SA chips according to the manufacture's protocol (GE Health Sciences Inc.). The running buffer used was HBS-EP (20 mM Hepes pH 7.4, 150 mM NaCl, 3 mM EDTA, 0.05% P-20) plus 5% DMSO with a flow rate of 40 μl/min. For SPR analysis, 5 different concentrations of each exemplary compound of the application were sprayed into 96 or 384 well plates using an HP D300 digital dispenser. The concentration ranged from about 195 nM to about 12 nM in a two-fold series. Concentration ranges were adjusted higher or lower for weaker or more potent compounds, respectively, when necessary. For the K_{D} determinations, single cycle kinetic analysis was performed with an on time of 60 seconds, and an off time of 300 or 600 seconds. Curve fitting and K_{D} calculations were performed with the Biacore T200 Evalutation software (GE Health Sciences Inc).

### Results

Table 1 shows the binding affinity values (K_{D}) of exemplary compounds of the application for the WDR5 protein. The exemplary compounds of the application have binding affinities ranging in the nanomolar concentrations.

### (ii) Fluorescence Polarization (FP) Binding Assays

H3 (1-15) (ARTKQTARKSTGGKA), and 9-Ala-FAM ((Ac)-ARAEVHLRK-(Ahx-Ahx)-K(5,6-FAM)) (where Ahx represents 6 amino hexanoic acid linkers) peptides for WDR5 were synthesized, N-terminal-labeled with isothiocyanate-fluorescein, and purified by Tufts University Core Services (Boston, Mass.). Compound binding assays were performed at a constant labeled peptide concentration of 30 or 20 nM for H3 (1-15) and 9-Ala-FAM respectively. WDR5 concentrations of 0.3 μM for H3 (1-15) and 0.05 μM for 9-Ala-FAM were used. For both the H3 (1-15) and 9-Ala-FAM peptides, 80 mM sodium phosphate buffer (pH 6.5), 20 mM KCl, and 0.008% Triton X-100 was used. For the H3 (1-15) peptide, FP assays were measured in 10 μl aliquots in 384-well Axygen plates using a Synergy 4 microplate reader (BioTek) with an excitation wavelength of 485 nm and emission wavelength of 528 nm. For the 9-Ala-FAM peptide, FP assays were measured in 125 μl aliquots in 96 well Microfluor plates using a ViewLux instrument (Perkin Elmer) with an excitation wavelength of 480 nm and emission wavelength of 540 nm. To determine Kdsp values, the data were fit to a hyperbolic function using Sigma Plot software (Systat Software). The Kdisₚ values represent the average of quadruplicate measurements.

### Results

Results of this assay are shown in Table 1 for exemplary compounds of the application.

(iii) Cell Proliferation Assay

MV4-11 cells were seeded into a 96-well plate at 1,000 cells/well in 150 μl medium (Alpha-MEM containing 10% FBS, 100 μg/ml Normocin, and 50 μg/ml Gentamycin, Invitrogen). A HP D300 digital dispenser was used to dose cells with DMSO or test compounds across a 10-point range of concentrations (high dose of 10 μM), and cultures were grown in a humidified 5% CO₂ incubator at 37° C. After five days, plates were removed from incubator and equilibrated to room temperature. An equal volume of ATPlite assay reagent was added to each well, and samples were processed according to manufacturer's instructions (Perkin Elmer). Luminescent signal was measured using an Envision plate reader equipped with a US-Luminescence detector.

### Results

Results of this assay are shown in Table 1 for exemplary compounds of the application.

### (iv) MLL1-WRAD2 Enzyme Assay

Compound potency is assessed through incorporation of 3H-SAM into oligonucleosomes purified from HeLa cells. Specifically, recombinant human MLL1 (aa 3745-3969, GenBank Accession No. NM_005933), WDR5 (aa 22-334, GenBank Accession No. NM_017588), RbBP5 (aa 1-538, GenBank Accession No. NM_005057), Ash2L (aa 2-534, GenBank Accession No. NM_001105214), and DPY-30 (aa 1-99, GenBank Accession No. NM_0325742), all with N-terminal His tag, are expressed in E. coli and mixed at a molar ratio of 1:1:1:1:2. 10 nM of the assembled MLL1-WRAD2 complex is mixed with 100 nM WRAD2 to enhance complex formation before incubation with 0.05 mg/ml nucleosome substrate and compounds (as 10 point duplicate dose response titrations) for 15 min in a buffer consisting of 50 mM Tris (pH 8.5), 5 mM MgCl₂, 50 mM NaCl, 1 mM DTT, 0.01% Brij-35, and 1% DMSO. Reaction is initiated with 1 μM 3H-SAM and incubated for 1 hour at 30° C. Reaction mixture is transferred to P81 filter-paper and washed with PBS before detection.

(v) Detection of in-Cell H3K4 Dimethylation

T24 cells are seeded into a 96-well plate at 400 cells/well in 150 μl medium (McCoy 5A containing 10% FBS, 100 μg/ml Normocin, and 50 μg/ml Gentamycin, Invitrogen). A HP D300 digital dispenser is used to dose cells with DMSO or test compounds across a 10-point range of concentrations (high dose of 10 μM), and cultures are grown in a humidified 5% CO₂ incubator at 37° C. After five days, plates are removed from incubator, media is aspirated, and the cells washed in PBS. Cell lysis, histone extraction, and detection of H3K4 dimethylation (H3K4me2) are performed using an AlphaLisa kit according to the manufacturer's instructions (Perkin Elmer). Signal is measured using an Envision plate reader.

Table 2 contains comparative data showing the effect of difluoro substitution on the biological activity of this class of compounds. The difluoro compounds of the present application consistently show excellent activity in all assays tested unlike other fluorinated and non-fluorinated analogs which may show better activity in one assay but not others.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1

| | | | | | GI |
|---|---|---|---|---|---|
| | | | FP IC₅₀ | SPR Kd | MV411 |
| No | Structure | Chemical Name | nM | uM | EC₅₀ uM |
| 1 | 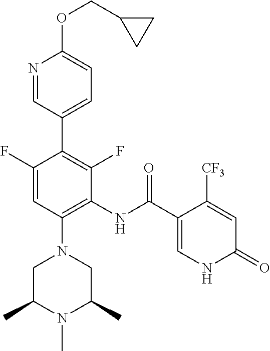 [SMILES: C[C@@H]1CN(c2cc(F)c(-c3ccc(OCC4CC4)nc3)c(F)c2NC(=O)c2c[nH]c(=O)cc2C(F)(F)F)C[C@H](C)N1C] | N-(3-(6- (cyclopropylmethoxy) pyridin-3-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | | 0.00059 | |
| 2 | 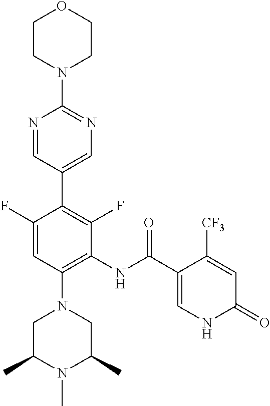 [SMILES: C[C@@H]1CN(c2cc(F)c(-c3cnc(N4CCOCC4)nc3)c(F)c2NC(=O)c2c[nH]c(=O)cc2C(F)(F)F)C[C@H](C)N1C] | N-(2,4-difluoro-3-(2- morpholinopyrimidin- 5-yl)-6-((3S,5R)- 3,4,5- trimethylpiperazin-1- yl)phenyl)-6-oxo-4- (trifluoromethyl)-1,6- dihydropyridine-3- carboxamide | 16.2546 | 0.00368 | 0.0301 |
| 3 | 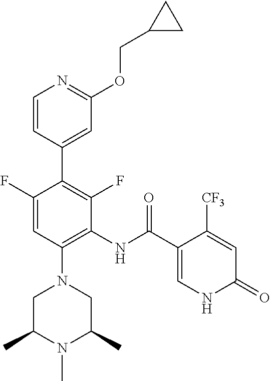 [SMILES: C[C@@H]1CN(c2cc(F)c(-c3ccnc(OCC4CC4)c3)c(F)c2NC(=O)c2c[nH]c(=O)cc2C(F)(F)F)C[C@H](C)N1C] | N-(3-(2- (cyclopropylmethoxy) pyridin-4-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 26.6100 | 0.00339 | |
| 4 | 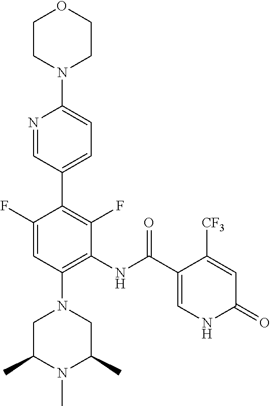 [SMILES: C[C@@H]1CN(c2cc(F)c(-c3ccc(N4CCOCC4)nc3)c(F)c2NC(=O)c2c[nH]c(=O)cc2C(F)(F)F)C[C@H](C)N1C] | N-(2,4-difluoro-3-(6- morpholinopyridin-3- yl)-6-((3S,5R)-3,4,5- trimethylpiperazin-1- yl)phenyl)-6-oxo-4- (trifluoromethyl)-1,6- dihydropyridine-3- carboxamide | 31.7760 | 0.00202 | |
| 5 | 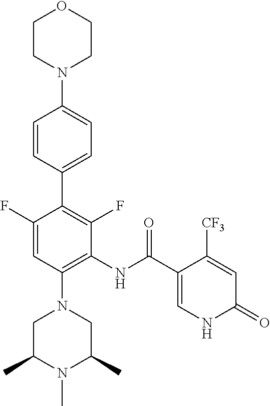 [SMILES: C[C@@H]1CN(c2cc(F)c(-c3ccc(N4CCOCC4)cc3)c(F)c2NC(=O)c2c[nH]c(=O)cc2C(F)(F)F)C[C@H](C)N1C] | N-(2,6-difluoro-4′- morpholino-4- ((3S,5R)-3,4,5- trimethylpiperazin-1- yl)-[1,1′-biphenyl]-3- yl)-6-oxo-4- (trifluoromethyl)-1,6- dihydropyridine-3- carboxamide | 32.1620 | 0.00355 | |
| 6 | 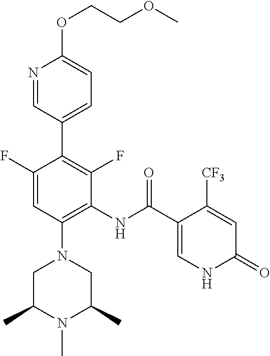 [SMILES: COCCOc1ccc(-c2c(F)cc(N3C[C@@H](C)N(C)[C@@H](C)C3)c(NC(=O)c3c[nH]c(=O)cc3C(F)(F)F)c2F)cn1] | N-(2,4-difluoro-3-(6- (2- methoxyethoxy) pyridin-3-yl)-6- ((3S,5R)-3,4,5- trimethylpiperazin-1- yl)phenyl)-6-oxo-4- (trifluoromethyl)-1,6- dihydropyridine-3- carboxamide | 32.9380 | 0.00283 | |
| 7 | 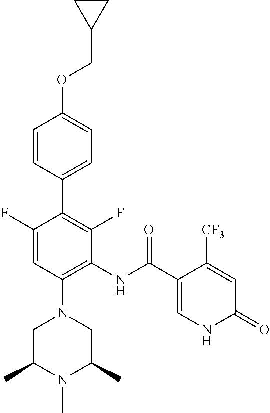 [SMILES: C[C@@H]1CN(c2cc(F)c(-c3ccc(OCC4CC4)cc3)c(F)c2NC(=O)c2c[nH]c(=O)cc2C(F)(F)F)C[C@H](C)N1C] | N-(4′- (cyclopropylmethoxy)- 2,6-difIuoro-4- ((3S,5R)-3,4,5- trimethylpiperazin-1- yl)-[1,1′-biphenyl]-3- yl)-6-oxo-4- (trifluoromethyl)-1,6- dihydropyridine-3- carboxamide | 17.9360 | | |
| 8 | 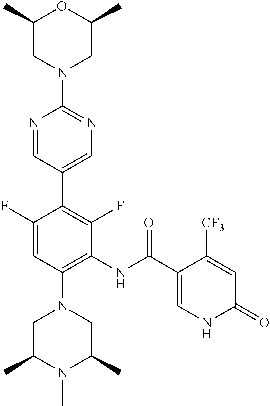 [SMILES: C[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)C[C@H](C)O1] | N-(3-(2-((2S,6R)-2,6- dimethylmorpholino) pyrimidin-5-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 21.2672 | 0.00082 | 0.031 |
| 9 | 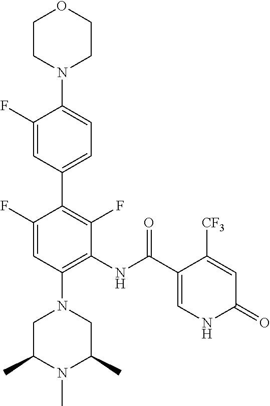 [SMILES: C[C@@H]1CN(c2cc(F)c(-c3ccc(N4CCOCC4)c(F)c3)c(F)c2NC(=O)c2c[nH]c(=O)cc2C(F)(F)F)C[C@H](C)N1C] | 6-Oxo-N-(2,3′,6- trifluoro-4′- morpholino-4- ((3S,5R)-3,4,5- trimethylpiperazin-1- yl)-[1,1′-biphenyl]-3- yl)-4-(trifluoromethyl)- 1,6-dihydropyridine-3- carboxamide | 15.3000 | 0.00133 | 0.123 |
| 10 | 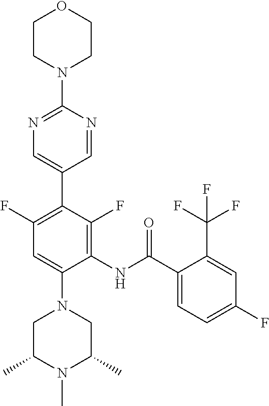 [SMILES: C[C@@H]1CN(c2cc(F)c(-c3cnc(N4CCOCC4)nc3)c(F)c2NC(=O)c2ccc(F)cc2C(F)(F)F)C[C@H](C)N1C] | N-[2,4-difluoro-3-(2- morpholin-4- ylpyrimidin-5-yl)-6- [(3R,5S)-3,4,5- trimethylpiperazin-1- yl]phenyl]-4-fluoro-2- (trifluoromethyl) benzamide | 10.0250 | 0.00033 | 0.0592 |
| 11 | 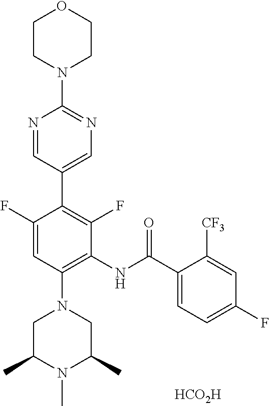 [SMILES: C[C@@H]1CN(c2cc(F)c(-c3cnc(N4CCOCC4)nc3)c(F)c2NC(=O)c2ccc(F)cc2C(F)(F)F)C[C@H](C)N1C ; "HCO2H"] | N-(2,4-difluoro-3-(2- morpholinopyrimidin- 5-yl)-6-((3S,5R)-3,4,5- trimethylpiperazin-1- yl)phenyl)-4-fluoro-2- (trifluoromethyl) benzamide formic acid | 9.8030 | 0.0008 | 0.414 |
| 12 | 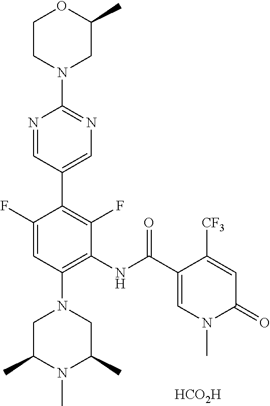 [SMILES: C[C@@H]1CN(c2cc(F)c(-c3cnc(N4CCO[C@@H](C)C4)nc3)c(F)c2NC(=O)c2cn(C)c(=O)cc2C(F)(F)F)C[C@H](C)N1C ; "HCO2H"] | N-(2,4-difluoro-3-(2- ((S)-2- methylmorpholino) pyrimidin-5-yl)-6- ((3S,5R)-3,4,5- trimethylpiperazin-1- yl)phenyl)-1-methyl- 6-oxo-4- (trifluoromethyl)-1,6- dihydropyridine-3- carboxamide formic acid | 24.5950 | 0.00142 | |
| 13 | 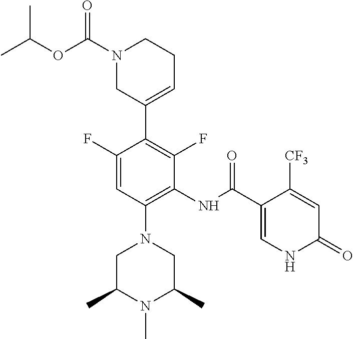 [SMILES: CC(C)OC(=O)N1CCC=C(c2c(F)cc(N3C[C@@H](C)N(C)[C@@H](C)C3)c(NC(=O)c3c[nH]c(=O)cc3C(F)(F)F)c2F)C1] | Isopropyl 5-(2,6- difluoro-3-(6-oxo-4- (trifluoromethyl)-1,6- dihydropyridine-3- carboxamido)-4- ((3S,5R)-3,4,5- trimethylpiperazin-1- yl)phenyl)-3,6- dihydropyridine- 1(2H)-carboxylate | 14.3520 | 0.00191 | 0.173 |
| 14 | 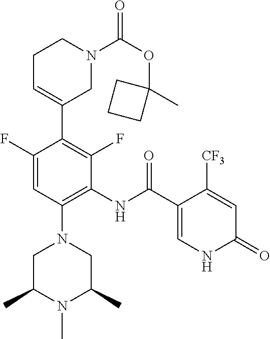 [SMILES: C[C@@H]1CN(c2cc(F)c(C3=CCCN(C(=O)OC4(C)CCC4)C3)c(F)c2NC(=O)c2c[nH]c(=O)cc2C(F)(F)F)C[C@H](C)N1C] | 1-Methylcyclobutyl 5- (2,6-difluoro-3-(6-oxo- 4-(trifluoromethyl)-1,6- dihydropyridine-3- carboxamido)-4- ((3S,5R)-3,4,5- trimethylpiperazin-1- yl)phenyl)-3,6- dihydropyridine- 1(2H)-carboxylate | 10.1530 | 0.00188 | |
| 15 | 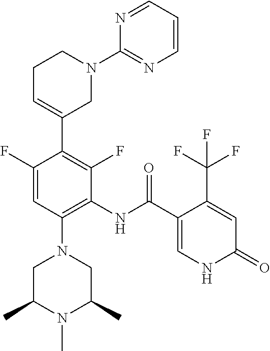 [SMILES: C[C@@H]1CN(c2cc(F)c(C3=CCCN(c4ncccn4)C3)c(F)c2NC(=O)c2c[nH]c(=O)cc2C(F)(F)F)C[C@H](C)N1C] | N-(2,4-difluoro-3-(1- (pyrimidin-2-yl)- 1,2,5,6- tetrahydropyridin-3- yl)-6-((3S,5R)-3,4,5- trimethylpiperazin-1- yl)phenyl)-6-oxo-4- (trifluoromethyl)-1,6- dihydropyridine-3- carboxamide | 21.1210 | 0.00169 | |
| 16 | 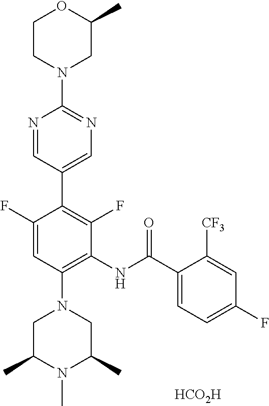 [SMILES: C[C@@H]1CN(c2cc(F)c(-c3cnc(N4CCO[C@@H](C)C4)nc3)c(F)c2NC(=O)c2ccc(F)cc2C(F)(F)F)C[C@H](C)N1C ; "HCO2H"] | N-(2,4-difluoro-3- (2-((S)-2- methylmorpholino) pyrimidin-5-yl)-6- ((3S,5R)-3,4,5- trimethylpiperazin-1- yl)phenyl)-4-fluoro-2- (trifluoromethyl) benzamide formic acid | 6.9890 | 0.00122 | |
| 17 | 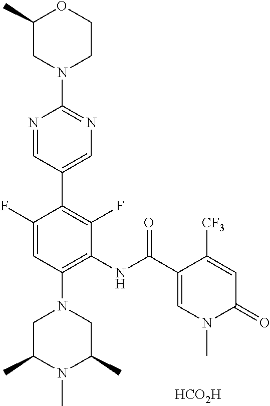 [SMILES: C[C@@H]1CN(c2ncc(-c3c(F)cc(N4C[C@@H](C)N(C)[C@@H](C)C4)c(NC(=O)c4cn(C)c(=O)cc4C(F)(F)F)c3F)cn2)CCO1 ; "HCO2H"] | N-(2,4-difluoro-3-(2- ((R)-2- methylmorpholino) pyrimidin-5-yl)-6- ((3S,5R)-3,4,5- trimethylpiperazin-1- yl)phenyl)-1-methyl- 6-oxo-4- (trifluoromethyl)-1,6- dihydropyridine-3- carboxamide formic acid | 10.0680 | 0.00175 | |
| 18 | 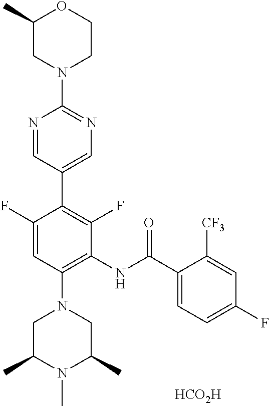 [SMILES: C[C@@H]1CN(c2ncc(-c3c(F)cc(N4C[C@@H](C)N(C)[C@@H](C)C4)c(NC(=O)c4ccc(F)cc4C(F)(F)F)c3F)cn2)CCO1 ; "HCO2H"] | N-(2,4-difluoro-3-(2- ((R)-2- methylmorpholino) pyrimidin-5-yl)-6- ((3S,5R)-3,4,5- trimethylpiperazin-1- yl)phenyl)-4-fluoro-2- (trifluoromethyl) benzamide formic acid | 10.9790 | 0.00171 | |
| 19 | 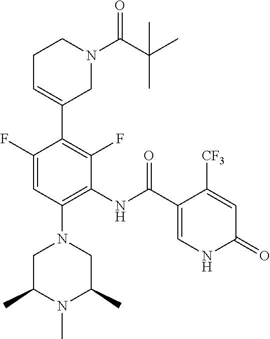 [SMILES: C[C@@H]1CN(c2cc(F)c(C3=CCCN(C(=O)C(C)(C)C)C3)c(F)c2NC(=O)c2c[nH]c(=O)cc2C(F)(F)F)C[C@H](C)N1C] | N-(2,4-difluoro-3-(1- pivaloyl-1,2,5,6- tetrahydropyridin-3- yl)-6-((3S,5R)-3,4,5- trimethylpiperazin-1- yl)phenyl)-6-oxo-4- (trifluoromethyl)-1,6- dihydropyridine-3- carboxamide | 31.6470 | 0.00531 | |
| 20 | 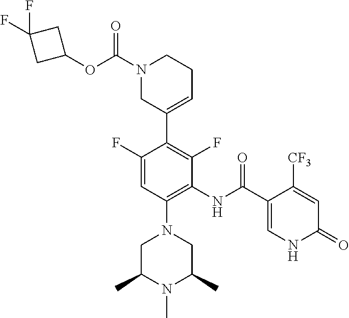 [SMILES: C[C@@H]1CN(c2cc(F)c(C3=CCCN(C(=O)OC4CC(F)(F)C4)C3)c(F)c2NC(=O)c2c[nH]c(=O)cc2C(F)(F)F)C[C@H](C)N1C] | 3,3-Difluorocyclo- butyl 5-(2,6- difluoro-3-(6- oxo-4- (trifluoromethyl)-1,6- dihydropyridine-3- carboxamido)-4- ((3S,5R)-3,4,5- trimethylpiperazin-1- yl)phenyl)-3,6- dihydropyridine- 1(2H)-carboxylate | 14.1380 | 0.00206 | 0.153 |
| 21 | 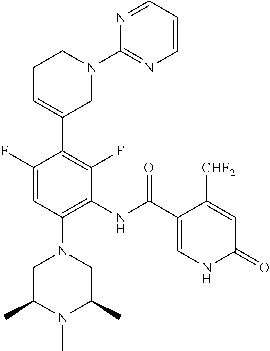 [SMILES: C[C@@H]1CN(c2cc(F)c(C3=CCCN(c4ncccn4)C3)c(F)c2NC(=O)c2c[nH]c(=O)cc2C(F)F)C[C@H](C)N1C] | N-(2,4-difluoro-3-(1- (pyrimidin-2-yl)- 1,2,5,6- tetrahydropyridin-3- yl)-6-((3S,5R)-3,4,5- trimethylpiperazin-1- yl)phenyl)-4- (difluoromethyl)-6- oxo-1,6- dihydropyridine-3- carboxamide | 37.1180 | 0.0045 | |
| 22 | 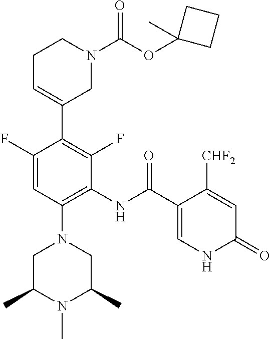 [SMILES: C[C@@H]1CN(c2cc(F)c(C3=CCCN(C(=O)OC4(C)CCC4)C3)c(F)c2NC(=O)c2c[nH]c(=O)cc2C(F)F)C[C@H](C)N1C] | 1-Methylcyclobutyl 5-(3-(4- (difluoromethyl)- 6-oxo-1,6- dihydropyridine-3- carboxamido)-2,6- difluoro-4-((3S,5R)- 3,4,5- trimethylpiperazin-1- yl)phenyl)-3,6- dihydropyridine- 1(2H)-carboxylate | 23.0370 | 0.00174 | |
| 23 | 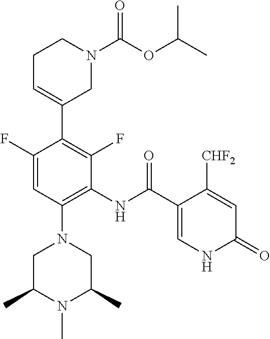 [SMILES: CC(C)OC(=O)N1CCC=C(c2c(F)cc(N3C[C@@H](C)N(C)[C@@H](C)C3)c(NC(=O)c3c[nH]c(=O)cc3C(F)F)c2F)C1] | Isopropyl 5-(3-(4- (difluoromethyl)-6- oxo-1,6- dihydropyridine-3- carboxamido)-2,6- difluoro-4-((3S,5R)- 3,4,5- trimethylpiperazin-1- yl)phenyl)-3,6- dihydropyridine- 1(2H)-carboxylate | 31.4420 | 0.00283 | |
| 24 | 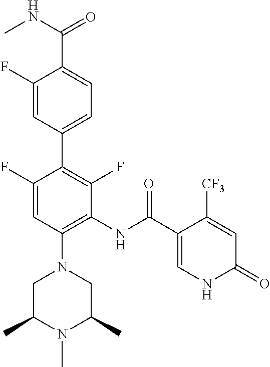 [SMILES: CNC(=O)c1ccc(-c2c(F)cc(N3C[C@@H](C)N(C)[C@@H](C)C3)c(NC(=O)c3c[nH]c(=O)cc3C(F)(F)F)c2F)cc1F] | 6-Oxo-N-(2,3′,6- trifluoro-4′- (methylcarbamoyl)-4- ((3S,5R)-3,4,5- trimethylpiperazin-1- yl)-[1,1′-biphenyl]-3- yl)-4-(trifluoromethyl)- 1,6-dihydropyridine-3- carboxamide | 24.8780 | 0.003 | 0.101 |
| 25 | 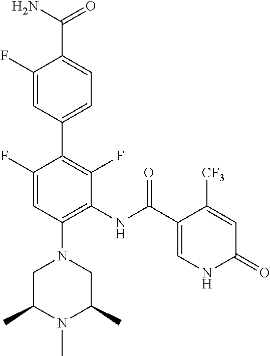 [SMILES: C[C@@H]1CN(c2cc(F)c(-c3ccc(C(N)=O)c(F)c3)c(F)c2NC(=O)c2c[nH]c(=O)cc2C(F)(F)F)C[C@H](C)N1C] | N-(4′-carbamoyl-2,3′,6- trifluoro-4-((3S,5R)- 3,4,5- trimethylpiperazin-1- yl)-[1,1′-biphenyl]-3- yl)-6-oxo-4- (trifluoromethyl)-1,6- dihydropyridine-3- carboxamide | 19.5090 | 0.00205 | 0.148 |
| 26 | 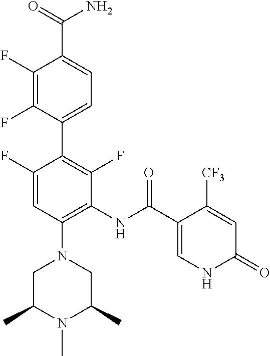 [SMILES: C[C@@H]1CN(c2cc(F)c(-c3ccc(C(N)=O)c(F)c3F)c(F)c2NC(=O)c2c[nH]c(=O)cc2C(F)(F)F)C[C@H](C)N1C] | N-(4′-carbamoyl- 2,2′,3′,6-tetrafluoro-4- ((3S,5R)-3,4,5- trimethylpiperazin-1- yl)-[1,1′-biphenyl]-3- yl)-6-oxo-4- (trifluoromethyl)-1,6- dihydropyridine-3- carboxamide | 22.2150 | 0.00449 | 0.316 |
| 27 | 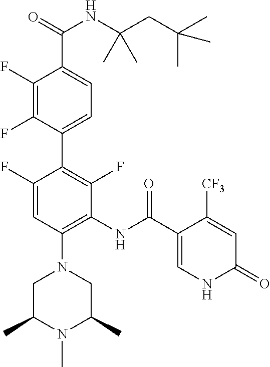 [SMILES: C[C@@H]1CN(c2cc(F)c(-c3ccc(C(=O)NC(C)(C)CC(C)(C)C)c(F)c3F)c(F)c2NC(=O)c2c[nH]c(=O)cc2C(F)(F)F)C[C@H](C)N1C] | 6-Oxo-N-(2,2′,3′,6- tetrafluoro-4′-((2,4,4- trimethylpentan-2- yl)carbamoyl)-4- ((3S,5R)-3,4,5- trimethylpiperazin-1- yl)-[1,1′-biphenyl]-3- yl)-4-(trifluoromethyl)- 1,6-dihydropyridine-3- carboxamide | 34.8470 | 0.00113 | 0.221 |
| 28 | 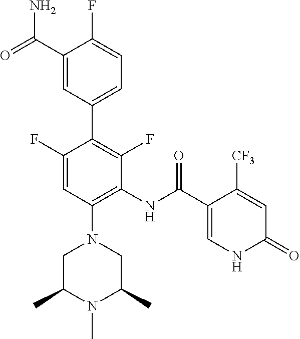 [SMILES: C[C@@H]1CN(c2cc(F)c(-c3ccc(F)c(C(N)=O)c3)c(F)c2NC(=O)c2c[nH]c(=O)cc2C(F)(F)F)C[C@H](C)N1C] | N-(3′-carbamoyl-2,4′,6- trifluoro-4-((3S,5R)- 3,4,5- trimethylpiperazin-1- yl)-[1,1′-biphenyl]-3- yl)-6-oxo-4- (trifluoromethyl)-1,6- dihydropyridine-3- carboxamide | 36.2280 | 0.00501 | 2.87 |
| 29 | 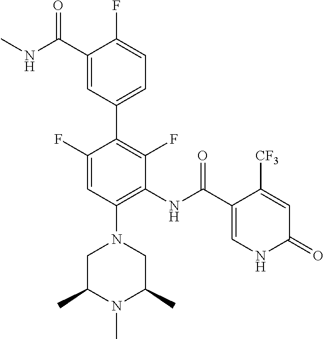 [SMILES: CNC(=O)c1cc(-c2c(F)cc(N3C[C@@H](C)N(C)[C@@H](C)C3)c(NC(=O)c3c[nH]c(=O)cc3C(F)(F)F)c2F)ccc1F] | 6-Oxo-N-(2,4′,6- trifluoro-3′- (methylcarbamoyl)-4- ((3S,5R)-3,4,5- trimethylpiperazin-1- yl)-[1,1′-biphenyl]-3- yl)-4-(trifluoromethyl)- 1,6-dihydropyridine-3- carboxamide | 22.3660 | 0.0039 | 1.11 |
| 30 | 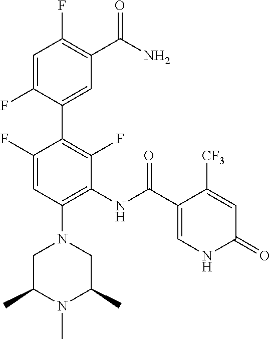 [SMILES: C[C@@H]1CN(c2cc(F)c(-c3cc(C(N)=O)c(F)cc3F)c(F)c2NC(=O)c2c[nH]c(=O)cc2C(F)(F)F)C[C@H](C)N1C] | N-(5′-carbamoyl- 2,2′,4′,6-tetrafluoro-4- ((3S,5R)-3,4,5- trimethylpiperazin-1- yl)-[1,1′-biphenyl]-3- yl)-6-oxo-4- (trifluoromethyl)-1,6- dihydropyridine-3- carboxamide | 85.8650 | 0.0236 | 18.5 |
| 31 | 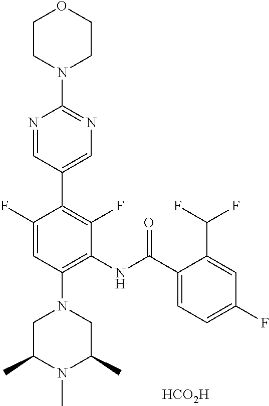 [SMILES: C[C@@H]1CN(c2cc(F)c(-c3cnc(N4CCOCC4)nc3)c(F)c2NC(=O)c2ccc(F)cc2C(F)F)C[C@H](C)N1C ; "HCO2H"] | N-(2,4-difluoro-3-(2- morpholinopyrimidin- 5-yl)-6-((3S,5R)-3,4,5- trimethylpiperazin-1- yl)phenyl)-2- (difluoromethyl)-4- fluorobenzamide formic acid | 19.5950 | 0.00104 | 0.0966 |
| 32 | 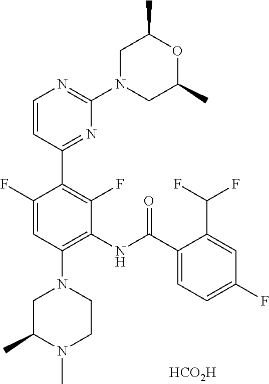 [SMILES: C[C@@H]1CN(c2nccc(-c3c(F)cc(N4CCN(C)[C@@H](C)C4)c(NC(=O)c4ccc(F)cc4C(F)F)c3F)n2)C[C@H](C)O1 ; "HCO2H"] | 2-(Difluoromethyl)-N- (3-(2-((2S,6R)-2,6- dimethylmorpholino) pyrimidin-4-yl)-6-((S)- 3,4-dimethylpiperazin- 2-yl)-2,4- difluorophenyl)4- fluorobenzamide formic acid | 32.9590 | 0.00507 | 0.286 |
| 33 | 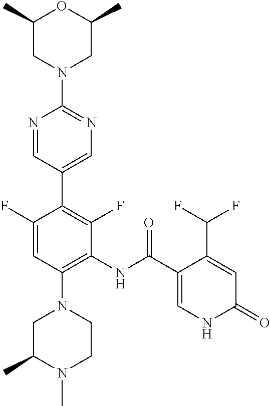 [SMILES: C[C@@H]1CN(c2ncc(-c3c(F)cc(N4CCN(C)[C@@H](C)C4)c(NC(=O)c4c[nH]c(=O)cc4C(F)F)c3F)cn2)C[C@H](C)O1] | 4-(Difluoromethyl)-N- (3-(2-((2S,6R)-2,6- dimethylmorpholino) pyrimidin-5-yl)-6-((S)- 3,4-dimethylpiperazin- 1-yl)-2,4- difluorophenyl)-6-oxo- 1,6-dihydropyridine-3- carboxamide | 14.9230 | 0.00061 | 0.0145 |
| 34 | 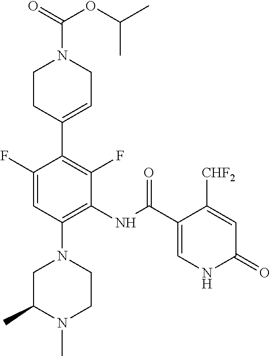 [SMILES: CC(C)OC(=O)N1CC=C(c2c(F)cc(N3CCN(C)[C@@H](C)C3)c(NC(=O)c3c[nH]c(=O)cc3C(F)F)c2F)CC1] | Isopropyl (S)-4-(3-(4- (difluoromethyl)-6- oxo-1,6- dihydropyridine-3- carboxamido)-4-(3,4- dimethylpiperazin-1- yl)-2,6- difluorophenyl)-3,6- dihydropyridine- 1(2H)-carboxylate | 22.0470 | 0.00202 | 0.0444 |
| 35 | 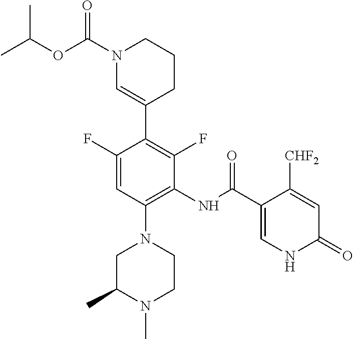 [SMILES: CC(C)OC(=O)N1C=C(c2c(F)cc(N3CCN(C)[C@@H](C)C3)c(NC(=O)c3c[nH]c(=O)cc3C(F)F)c2F)CCC1] | Isopropyl (S)-5-(3-(4- (difluoromethyl)-6- oxo-1,6- dihydropyridine-3- carboxamido)-4-(3,4- dimethylpiperazin-1- yl)-2,6- difluorophenyl)-3,6- dihydropyridine- 1(2H)-carboxylate | 29.4120 | 0.00629 | 0.409 |
| 36 | 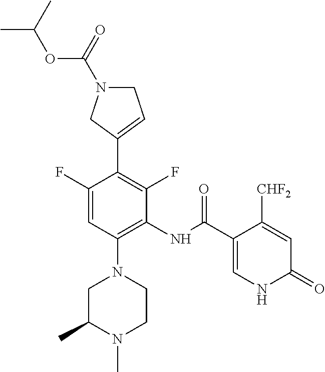 [SMILES: CC(C)OC(=O)N1CC=C(c2c(F)cc(N3CCN(C)[C@@H](C)C3)c(NC(=O)c3c[nH]c(=O)cc3C(F)F)c2F)C1] | Isopropyl (S)-3-(3-(4- (difluoromethyl)-6- oxo-1,6- dihydropyridine-3- carboxamido)-4-(3,4- dimethylpiperazin-1- yl)-2,6- difluorophenyl)-2,5- dihydro-1H-pyrrole-1- carboxylate | 13.1576 | 0.00104 | 0.0545 |
| 37 | 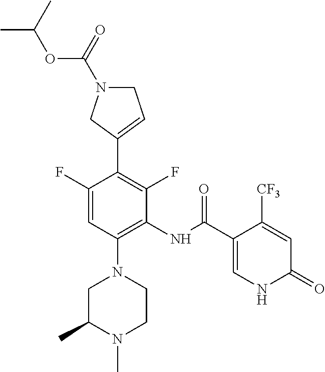 [SMILES: CC(C)OC(=O)N1CC=C(c2c(F)cc(N3CCN(C)[C@@H](C)C3)c(NC(=O)c3c[nH]c(=O)cc3C(F)(F)F)c2F)C1] | Isopropyl (S)-3-(4- (3,4- dimethylpiperazin-1- yl)-2,6-difluoro-3-(6- oxo-4- (trifluoromethyl)-1,6- dihydropyridine-3- carboxamido)phenyl)- 2,5-dihydro-1H- pyrrole-1-carboxylate | 16.1970 | 0.00224 | 0.108 |
| 38 | 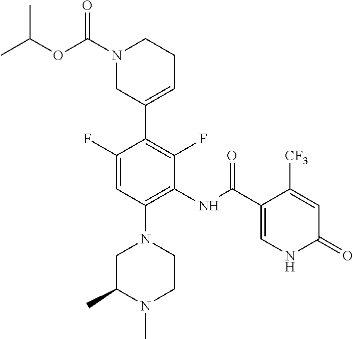 [SMILES: CC(C)OC(=O)N1CCC=C(c2c(F)cc(N3CCN(C)[C@@H](C)C3)c(NC(=O)c3c[nH]c(=O)cc3C(F)(F)F)c2F)C1] | Isopropyl (S)-5-(4- (3,4- dimethylpiperazin-1- yl)-2,6-difluoro-3-(6- oxo-4- (trifluoromethyl)-1,6- dihydropyridine-3- carboxamido)phenyl)- 3,6-dihydropyridine- 1(2H)-carboxylate | 17.4140 | 0.0164 | 0.185 |
| 39 | 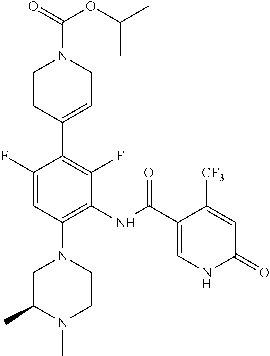 [SMILES: CC(C)OC(=O)N1CC=C(c2c(F)cc(N3CCN(C)[C@@H](C)C3)c(NC(=O)c3c[nH]c(=O)cc3C(F)(F)F)c2F)CC1] | Isopropyl (S)-4-(4- (3,4- dimethylpiperazin-1- yl)-2,6-difluoro-3-(6- oxo-4- (trifluoromethyl)-1,6- dihydropyridine-3- carboxamido)phenyl)- 3,6-dihydropyridine- 1(2H)-carboxylate | 15.2352 | 0.00158 | 0.0354 |
| 40 | 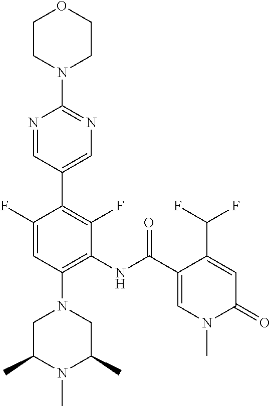 [SMILES: C[C@@H]1CN(c2cc(F)c(-c3cnc(N4CCOCC4)nc3)c(F)c2NC(=O)c2cn(C)c(=O)cc2C(F)F)C[C@H](C)N1C] | N-(2,4-difluoro-3-(2- morpholinopyrimidin- 5-yl)-6-((3S,5R)-3,4,5- trimethylpiperazin-1- yl)phenyl)-4- (difluoromethyl)-1- methyl-6-oxo-1,6- dihydropyridine-3- carboxamide | 20.0920 | 0.00285 | 0.286 |
| 41 | 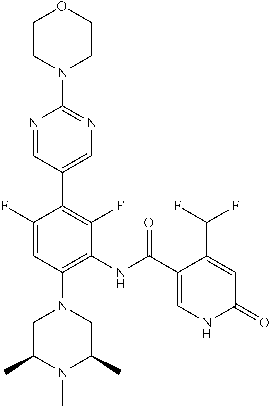 [SMILES: C[C@@H]1CN(c2cc(F)c(-c3cnc(N4CCOCC4)nc3)c(F)c2NC(=O)c2c[nH]c(=O)cc2C(F)F)C[C@H](C)N1C] | N-(2,4-difluoro-3-(2- morpholinopyrimidin- 5-yl)-6-((3S,5R)-3,4,5- trimethylpiperazin-1- yl)phenyl)-4- (difluoromethyl)-6- oxo-1,6- dihydropyridine-3- carboxamide | 7.0960 | 0.00084 | 0.0488 |
| 42 | 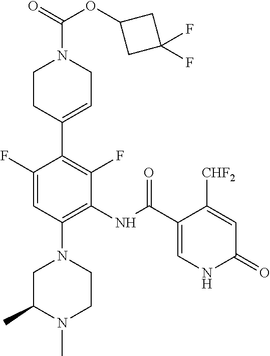 [SMILES: C[C@H]1CN(c2cc(F)c(C3=CCN(C(=O)OC4CC(F)(F)C4)CC3)c(F)c2NC(=O)c2c[nH]c(=O)cc2C(F)F)CCN1C] | (3,3- Difluorocyclobutyl (S)-4-(3-(4- (difluoromethyl)-6- oxo-1,6- dihydropyridine-3- carboxamido)-4-(3,4- dimethylpiperazin-1- yl)-2,6- difluorophenyl)-3,6- dihydropyridine- 1(2H)-carboxylate | 11.2050 | 0.00238 | 0.281 |
| 43 | 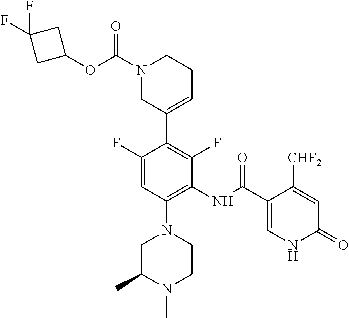 [SMILES: C[C@H]1CN(c2cc(F)c(C3=CCCN(C(=O)OC4CC(F)(F)C4)C3)c(F)c2NC(=O)c2c[nH]c(=O)cc2C(F)F)CCN1C] | 3,3-Difluorocyclo- butyl (S)-5-(3-(4- (difluoromethyl)-6- oxo-1,6- dihydropyridine-3- carboxamido)-4-(3,4- dimethylpiperazin-1- yl)-2,6- difluorophenyl)-3,6- dihydropyridine- 1(2H)-carboxylate | 15.9370 | 0.0017 | 0.653 |
| 44 | 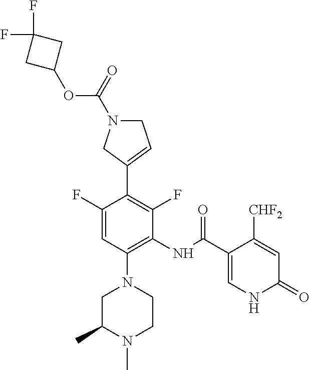 [SMILES: C[C@H]1CN(c2cc(F)c(C3=CCN(C(=O)OC4CC(F)(F)C4)C3)c(F)c2NC(=O)c2c[nH]c(=O)cc2C(F)F)CCN1C] | 3,3-Difluorocyclo- butyl (S)-5-(3-(4- (difluoromethyl)-6- oxo-1,6- dihydropyridine-3- carboxamido)-4-(3,4- dimethylpiperazin-1- yl)-2,6- difluorophenyl)-3,6- dihydropyridine- 1(2H)-carboxylate | 14.3700 | 0.00232 | 0.18 |
| 45 | 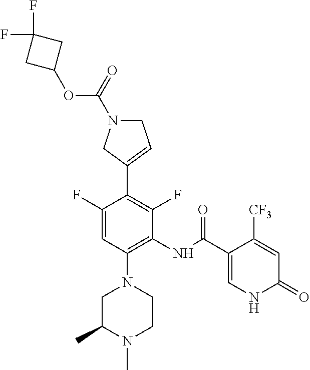 [SMILES: C[C@H]1CN(c2cc(F)c(C3=CCN(C(=O)OC4CC(F)(F)C4)C3)c(F)c2NC(=O)c2c[nH]c(=O)cc2C(F)(F)F)CCN1C] | 3,3-Difluorocyclo- butyl (S)-3-(4-(3,4- dimethylpiperazin-1- yl)-2,6-difluoro-3-(6- oxo-4- (trifluoromethyl)-1,6- dihydropyridine-3- carboxamido)phenyl)- 2,5-dihydro-1H- pyrrole-1-carboxylate | 12.0663 | 0.00132 | 0.0767 |
| 46 | 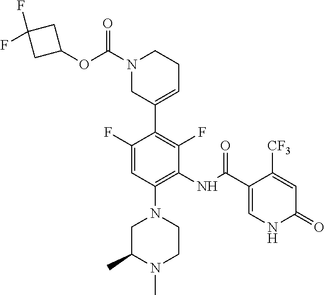 [SMILES: C[C@H]1CN(c2cc(F)c(C3=CCCN(C(=O)OC4CC(F)(F)C4)C3)c(F)c2NC(=O)c2c[nH]c(=O)cc2C(F)(F)F)CCN1C] | 3,3-Difluorocyclo- butyl (S)-5-(4-(3,4- dimethylpiperazin-1- yl)-2,6-difluoro-3-(6- oxo-4- (trifluoromethyl)-1,6- dihydropyridine-3- carboxamido)phenyl)- 3,6-dihydropyridine- 1(2H)-carboxylate | 14.9200 | 0.00297 | 0.607 |
| 47 | 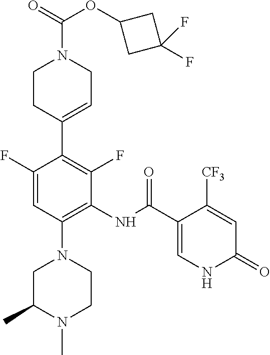 [SMILES: C[C@H]1CN(c2cc(F)c(C3=CCN(C(=O)OC4CC(F)(F)C4)CC3)c(F)c2NC(=O)c2c[nH]c(=O)cc2C(F)(F)F)CCN1C] | 3,3-Difluorocyclo- butyl (S)-4-(4-(3,4- dimethylpiperazin-1- yl)-2,6-difluoro-3-(6- oxo-4- (trifluoromethyl)-1,6- dihydropyridine-3- carboxamido)phenyl)- 3,6-dihydropyridine- 1(2H)-carboxylate | 17.4826 | 0.00135 | 0.063 |
| 48 | 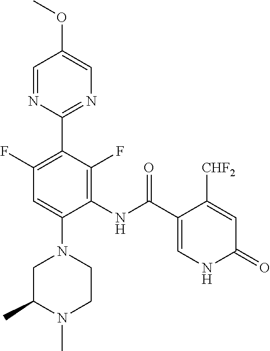 [SMILES: COc1cnc(-c2c(F)cc(N3CCN(C)[C@@H](C)C3)c(NC(=O)c3c[nH]c(=O)cc3C(F)F)c2F)nc1] | (S)-4-(difluoro- methyl)-N-(6-(3,4- dimethylpiperazin-1- yl)-2,4-difluoro-3-(1- (5-methoxypyrimidin- 2-yl)-1,2,3,6- tetrahydropyridin-4- yl)phenyl)-6-oxo-1,6- dihydropyridine-3- carboxamide | 22.4970 | 0.00149 | 0.27 |
| 49 | 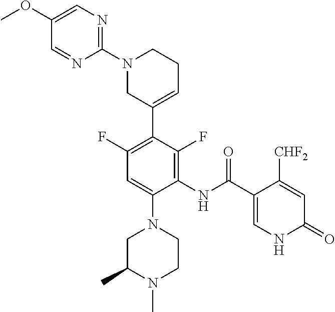 [SMILES: COc1cnc(N2CCC=C(c3c(F)cc(N4CCN(C)[C@@H](C)C4)c(NC(=O)c4c[nH]c(=O)cc4C(F)F)c3F)C2)nc1] | (S)-4-(difluoromethyl)- N-(6-(3,4- dimethylpiperazin-1- yl)-2,4-difluoro-3-(1- (5-methoxypyrimidin- 2-yl)-1,2,5,6- tetrahydropyridin-3- yl)phenyl)-6-oxo-1,6- dihydropyridine-3- carboxamide | 21.6550 | 0.00268 | 0.332 |
| 50 | 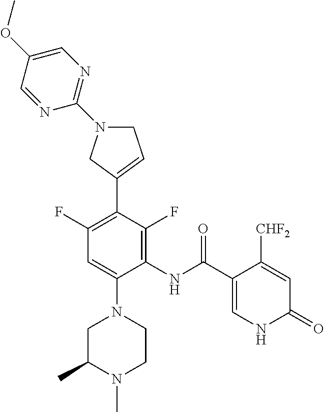 [SMILES: COc1cnc(N2CC=C(c3c(F)cc(N4CCN(C)[C@@H](C)C4)c(NC(=O)c4c[nH]c(=O)cc4C(F)F)c3F)C2)nc1] | (S)-4-(difluoromethyl)- N-(6-(3,4- dimethylpiperazin-1- yl)-2,4-difluoro-3-(1- (5-methoxypyrimidin- 2-yl)-2,5-dihydro-1H- pyrrol-3-yl)phenyl)-6- oxo-1,6- dihydropyridine-3- carboxamide | 11.8100 | 0.0008 | 0.0832 |
| 51 | 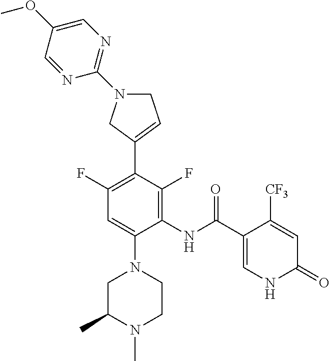 [SMILES: COc1cnc(N2CC=C(c3c(F)cc(N4CCN(C)[C@@H](C)C4)c(NC(=O)c4c[nH]c(=O)cc4C(F)(F)F)c3F)C2)nc1] | (S)-N-(6-(3,4- dimethylpiperazin-1- yl)-2,4-difluoro-3-(1- (5-methoxypyrimidin- 2-yl)-2,5-dihydro-1H- pyrrol-3-yl)phenyl)-6- oxo-4- (trifluoromethyl)-1,6- dihydropyridine-3- carboxamide | 16.9800 | 0.0011 | 0.111 |
| 52 | 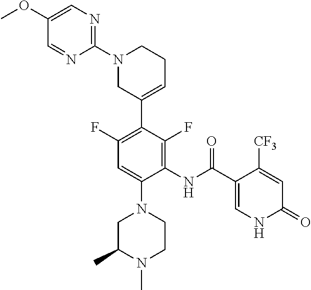 [SMILES: COc1cnc(N2CCC=C(c3c(F)cc(N4CCN(C)[C@@H](C)C4)c(NC(=O)c4c[nH]c(=O)cc4C(F)(F)F)c3F)C2)nc1] | (S)-N-(6-(3,4- dimethylpiperazin-1- yl)-2,4-difluoro-3-(1- (5-methoxypyrimidin- 2-yl)-1,2,5,6- tetrahydropyridin-3- yl)phenyl)-6-oxo-4- (trifluoromethyl)-1,6- dihydropyridine-3- carboxamide | 20.0970 | 0.00046 | 0.285 |
| 53 | 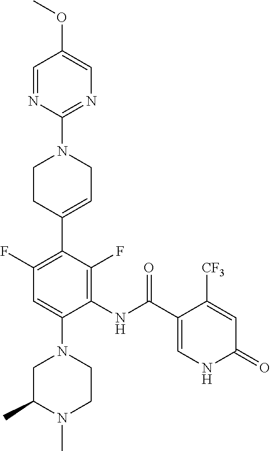 [SMILES: COc1cnc(N2CC=C(c3c(F)cc(N4CCN(C)[C@@H](C)C4)c(NC(=O)c4c[nH]c(=O)cc4C(F)(F)F)c3F)CC2)nc1] | (S)-N-(6-(3,4- dimethylpiperazin-1- yl)-2,4-difluoro-3-(1- (5-methoxypyrimidin- 2-yl)-1,2,3,6- tetrahydropyridin-4- yl)phenyl)-6-oxo-4- (trifluoromethyl)-1,6- dihydropyridine-3- carboxamide | 19.4810 | 0.00102 | 0.0758 |
| 54 | 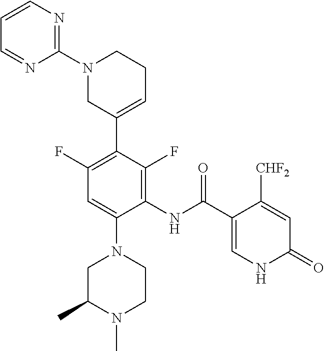 [SMILES: C[C@H]1CN(c2cc(F)c(C3=CCCN(c4ncccn4)C3)c(F)c2NC(=O)c2c[nH]c(=O)cc2C(F)F)CCN1C] | (S)-4-(difluoromethyl)- N-(6-(3,4- dimethylpiperazin-1- yl)-2,4-difluoro-3-(1- (pyrimidin-2-yl)- 1,2,5,6- tetrahydropyridin-3- yl)phenyl)-6-oxo-1,6- dihydropyridine-3- carboxamide | 28.8210 | 0.00383 | 0.693 |
| 55 | 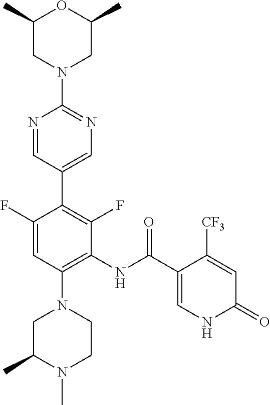 [SMILES: C[C@@H]1CN(c2ncc(-c3c(F)cc(N4CCN(C)[C@@H](C)C4)c(NC(=O)c4c[nH]c(=O)cc4C(F)(F)F)c3F)cn2)C[C@H](C)O1] | N-(3-(2-((2S,6R)-2,6- dimethylmorpholino) pyrimidin-5-yl)-6-((S)- 3,4-dimethylpiperazin- 1-yl)-2,4- difluorophenyl)-6-oxo- 4-(trifluoromethyl)-1,6- dihydropyridine-3- carboxamide | 14.7860 | 0.00078 | 0.0238 |
| 56 | 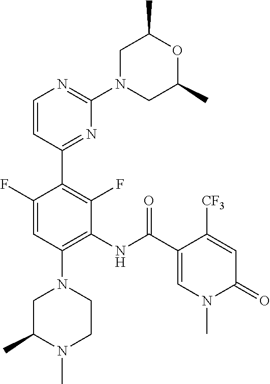 [SMILES: C[C@@H]1CN(c2nccc(-c3c(F)cc(N4CCN(C)[C@@H](C)C4)c(NC(=O)c4cn(C)c(=O)cc4C(F)(F)F)c3F)n2)C[C@H](C)O1] | N-(3-(2-((2S,6R)-2,6- dimethylmorpholino) pyrimidin-4-yl)-6-((S)- 3.4-dimethylpiperazin- 1-yl)-2,4- difluorophenyl)-1- methyl-6-oxo-4- (trifluoromethyl)-1,6- dihydropyridine-3- carboxamide | 36.6540 | 0.00979 | 1.32 |
| 57 | 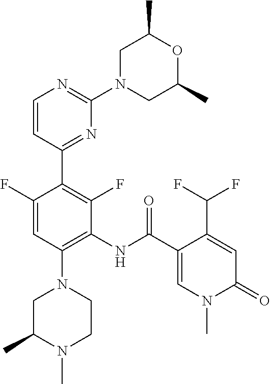 [SMILES: C[C@@H]1CN(c2nccc(-c3c(F)cc(N4CCN(C)[C@@H](C)C4)c(NC(=O)c4cn(C)c(=O)cc4C(F)F)c3F)n2)C[C@H](C)O1] | 4-(Difluoromethyl)-N- (3-(2-((2S,6R)-2,6- dimethylmorpholino) pyrimidin-4-yl)-6-((S)- 3,4-dimethylpiperazin- 1-yl)-2,4- difluorophenyl)-1- methyl-6-oxo-1,6- dihydropyridine-3- carboxamide | 26.8210 | 0.00763 | 0.367 |
| 58 | 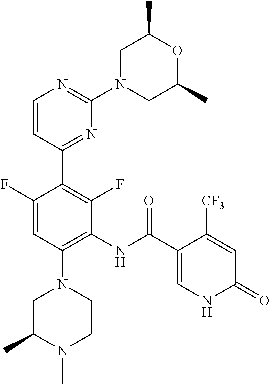 [SMILES: C[C@@H]1CN(c2nccc(-c3c(F)cc(N4CCN(C)[C@@H](C)C4)c(NC(=O)c4c[nH]c(=O)cc4C(F)(F)F)c3F)n2)C[C@H](C)O1] | N-(3-(2-((2S,6R)-2,6- dimethylmorpholino) pyrimidin-4-yl)-6-((S)- 3,4-dimethylpiperazin- 1-yl)-2,4- difluorophenyl)-6-oxo- 4-(trifluoromethyl)-1,6- dihydropyridine-3- carboxamide | 15.1360 | 0.00125 | 0.0991 |
| 59 | 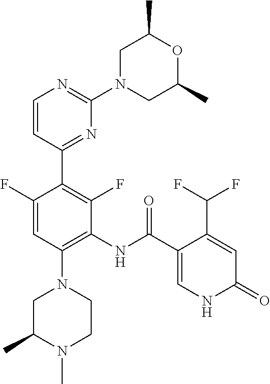 [SMILES: C[C@@H]1CN(c2nccc(-c3c(F)cc(N4CCN(C)[C@@H](C)C4)c(NC(=O)c4c[nH]c(=O)cc4C(F)F)c3F)n2)C[C@H](C)O1] | 4-(Difluoromethyl)-N- (3-(2-((2S,6R)-2,6- dimethylmorpholino) pyrimidin-4-yl)-6-((S)- 3,4-dimethylpiperazin- 1-yl)-2,4- difluorophenyl)-6-oxo- 1,6-dihydropyridine-3- carboxamide | 14.3860 | 0.00114 | 0.0566 |
| 60 | 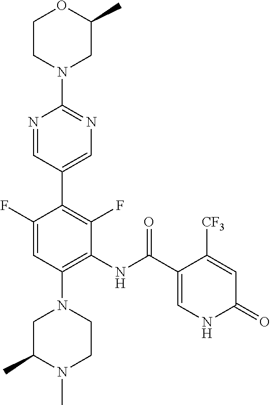 [SMILES: C[C@H]1CN(c2ncc(-c3c(F)cc(N4CCN(C)[C@@H](C)C4)c(NC(=O)c4c[nH]c(=O)cc4C(F)(F)F)c3F)cn2)CCO1] | N-(6-((S)-3,4- dimethylpiperazin-1- yl)-2,4-difluoro-3-(2- ((S)-2- methylmorpholino) pyrimidin-5-yl)phenyl)- 6-oxo-4- (trifluoromethyl)-1,6- dihydropyridine-3- carboxamide | 10.9610 | 0.00126 | 0.0115 |
| 61 | 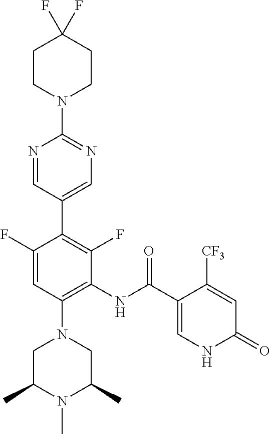 [SMILES: C[C@@H]1CN(c2cc(F)c(-c3cnc(N4CCC(F)(F)CC4)nc3)c(F)c2NC(=O)c2c[nH]c(=O)cc2C(F)(F)F)C[C@H](C)N1C] | N-(3-(2-(4,4- difluoropiperidin-1- yl)pyrimidin-5-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 11.6800 | 0.00126 | 0.0229 |
| 62 | 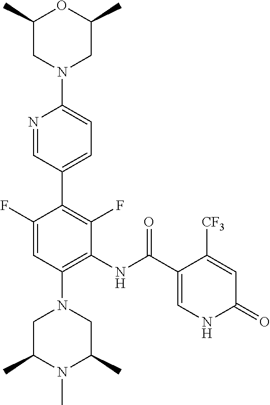 [SMILES: C[C@@H]1CN(c2ccc(-c3c(F)cc(N4C[C@@H](C)N(C)[C@@H](C)C4)c(NC(=O)c4c[nH]c(=O)cc4C(F)(F)F)c3F)cn2)C[C@H](C)O1] | N-(3-(6-((2S,6R)-2,6- dimethylmorpholino) pyridin-3-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 14.4110 | 0.00214 | 0.0249 |
| 63 | 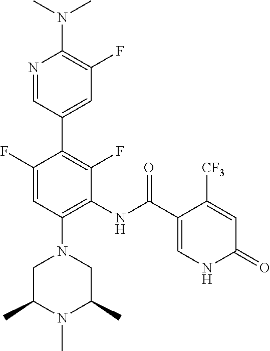 [SMILES: C[C@@H]1CN(c2cc(F)c(-c3cnc(N(C)C)c(F)c3)c(F)c2NC(=O)c2c[nH]c(=O)cc2C(F)(F)F)C[C@H](C)N1C] | N-(3-(6- (dimethylamino)-5- fluoropyridin-3-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 12.9590 | 0.00181 | 0.0618 |
| 64 | 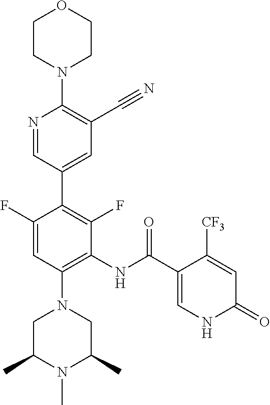 [SMILES: C[C@@H]1CN(c2cc(F)c(-c3cnc(N4CCOCC4)c(C#N)c3)c(F)c2NC(=O)c2c[nH]c(=O)cc2C(F)(F)F)C[C@H](C)N1C] | N-(3-(5-cyano-6- morpholinopyridin-3- yl)-2,4-difluoro-6- ((3S,5R)-3,4,5- trimethylpiperazin-1- yl)phenyl)-6-oxo-4- (trifluoromethyl)-1,6- dihydropyridine-3- carboxamide | 14.9200 | 0.00248 | 0.0327 |
| 65 | 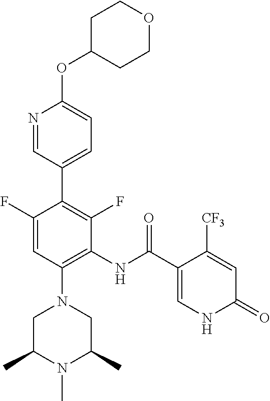 [SMILES: C[C@@H]1CN(c2cc(F)c(-c3ccc(OC4CCOCC4)nc3)c(F)c2NC(=O)c2c[nH]c(=O)cc2C(F)(F)F)C[C@H](C)N1C] | N-(2,4-difluoro-3-(6- ((tetrahydro-2H-pyran- 4-yl)oxy)pyridin-3-yl)- 6-((3S,5R)-3,4,5- trimethylpiperazin-1- yl)phenyl)-6-oxo-4- (trifluoromethyl)-1,6- dihydropyridine-3- carboxamide | 10.2110 | 0.00151 | 0.0338 |
| 66 | 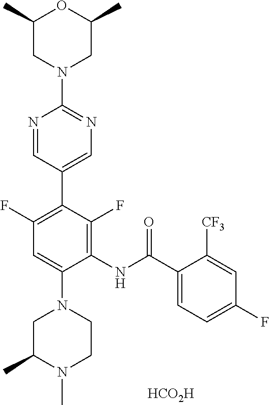 [SMILES: C[C@@H]1CN(c2ncc(-c3c(F)cc(N4CCN(C)[C@@H](C)C4)c(NC(=O)c4ccc(F)cc4C(F)(F)F)c3F)cn2)C[C@H](C)O1 ; "HCO2H"] | N-(3-(2-((2S,6R)-2,6- dimethylmorpholino) pyrimidin-5-yl)-6-((S)- 3,4-dimethylpiperazin- 1-yl)-2,4- difluorophenyl)-4- fluoro-2- (trifluoromethyl) benzamide formic acid | 14.0820 | 0.00202 | 0.0458 |
| 67 | 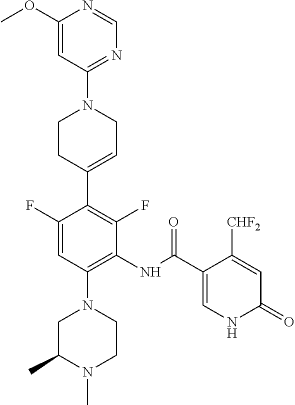 [SMILES: COc1cc(N2CC=C(c3c(F)cc(N4CCN(C)[C@@H](C)C4)c(NC(=O)c4c[nH]c(=O)cc4C(F)F)c3F)CC2)ncn1] | (S)-4-(difluoromethyl)- N-(6-(3,4- dimethylpiperazin-1- yl)-2,4-difluoro-3-(1- (6-methoxypyrimidin- 4-yl)-1,2,3,6- tetrahydropyridin-4- yl)phenyl)-6-oxo-1,6- dihydropyridine-3- carboxamide | 19.7260 | 0.00097 | 0.101 |
| 68 | 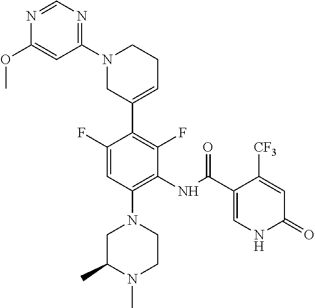 [SMILES: COc1cc(N2CCC=C(c3c(F)cc(N4CCN(C)[C@@H](C)C4)c(NC(=O)c4c[nH]c(=O)cc4C(F)(F)F)c3F)C2)ncn1] | (S)-N-(6-(3,4- dimethylpiperazin-1- yl)-2,4-difluoro-3-(1- (6-methoxypyrimidin- 4-yl)-1,2,5,6- tetrahydropyridin-3- yl)phenyl)-6-oxo-4- (trifluoromethyl)-1,6- dihydropyridine-3- carboxamide | 19.4520 | 0.00137 | 0.358 |
| 69 | 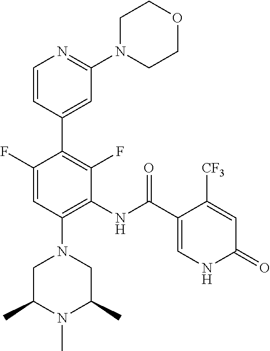 [SMILES: C[C@@H]1CN(c2cc(F)c(-c3ccnc(N4CCOCC4)c3)c(F)c2NC(=O)c2c[nH]c(=O)cc2C(F)(F)F)C[C@H](C)N1C] | N-(2,4-difluoro-3-(2- morpholinopyridin-4- yl)-6-((3S,5R)-3,4,5- trimethylpiperazin-1- yl)phenyl)-6-oxo-4- (trifluoromethyl)-1,6- dihydropyridine-3- carboxamide | 23.1520 | 0.00301 | 0.417 |
| 70 | 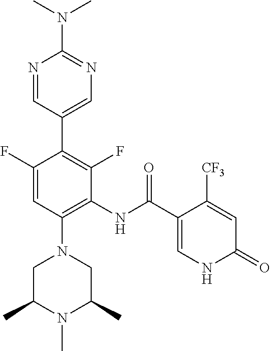 [SMILES: C[C@@H]1CN(c2cc(F)c(-c3cnc(N(C)C)nc3)c(F)c2NC(=O)c2c[nH]c(=O)cc2C(F)(F)F)C[C@H](C)N1C] | N-(3-(2- (dimethylamino) pyrimidin-5-yl)-2,4- difluoro- 6-((3S,5R)-3,4,5- trimethylpiperazin-1- yl)phenyl)-6-oxo-4- (trifluoromethyl)-1,6- dihydropyridine-3- carboxamide | 16.5330 | 0.00091 | 0.0267 |
| 71 | 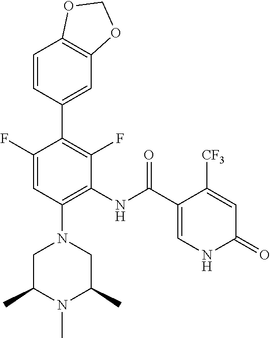 [SMILES: C[C@@H]1CN(c2cc(F)c(-c3ccc4c(c3)OCO4)c(F)c2NC(=O)c2c[nH]c(=O)cc2C(F)(F)F)C[C@H](C)N1C] | N-(3- (benzo[d][1,3]dioxol- 5-yl)-2,4-difluoro-6- ((3S,5R)-3,4,5- trimethylpiperazin-1- yl)phenyl)-6-oxo-4- (trifluoromethyl)-1,6- dihydropyridine-3- carboxamide | 15.1290 | 0.00165 | 0.0644 |
| 72 | 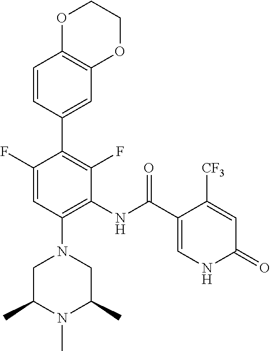 [SMILES: C[C@@H]1CN(c2cc(F)c(-c3ccc4c(c3)OCCO4)c(F)c2NC(=O)c2c[nH]c(=O)cc2C(F)(F)F)C[C@H](C)N1C] | N-(3-(2,3- dihydrobenzo[b][1,4] dioxin-6-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 18.1850 | 0.0007 | 0.0991 |
| 73 | 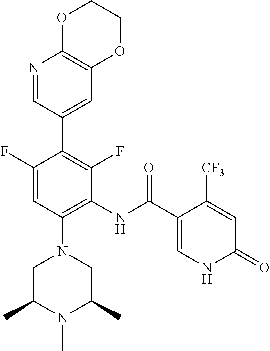 [SMILES: C[C@@H]1CN(c2cc(F)c(-c3cnc4c(c3)OCCO4)c(F)c2NC(=O)c2c[nH]c(=O)cc2C(F)(F)F)C[C@H](C)N1C] | N-(3-(2,3-dihydro- [1,4]dioxino[2,3- b]pyridin-7-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 16.7730 | 0.00126 | 0.11 |
| 74 | 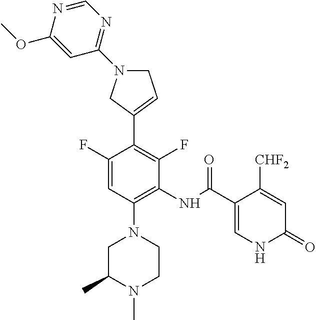 [SMILES: COc1cc(N2CC=C(c3c(F)cc(N4CCN(C)[C@@H](C)C4)c(NC(=O)c4c[nH]c(=O)cc4C(F)F)c3F)C2)ncn1] | (S)-4-(difluoromethyl)- N-(6-(3,4- dimethylpiperazin-1- yl)-2,4-difluoro-3-(1- (6-methoxypyrimidin- 4-yl)-2,5-dihydro-1H- pyrrol-3-yl)phenyl)-6- oxo-1,6- dihydropyridine-3- carboxamide | 16.4450 | 0.00212 | 0.0367 |
| 75 | 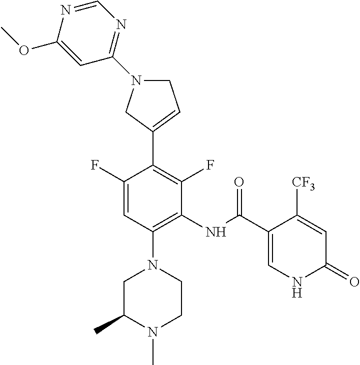 [SMILES: COc1cc(N2CC=C(c3c(F)cc(N4CCN(C)[C@@H](C)C4)c(NC(=O)c4c[nH]c(=O)cc4C(F)(F)F)c3F)C2)ncn1] | (S)-N-(6-(3,4- dimethylpiperazin-1- yl)-2,4-difluoro-3-(1- (6-methoxypyrimidin- 4-yl)-2,5-dihydro-1H- pyrrol-3-yl)phenyl)-6- oxo-4- (trifluoromethyl)-1,6- dihydropyridine-3- carboxamide | 18.4610 | 0.0018 | 0.0351 |
| 76 | 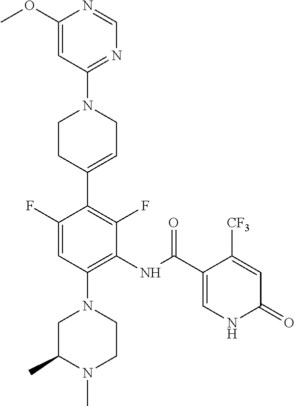 [SMILES: COc1cc(N2CC=C(c3c(F)cc(N4CCN(C)[C@@H](C)C4)c(NC(=O)c4c[nH]c(=O)cc4C(F)(F)F)c3F)CC2)ncn1] | (S)-N-(6-(3,4- dimethylpiperazin-1- yl)-2,4-difluoro-3-(1- (6-methoxypyrimidin- 4-yl)-1,2,3,6- tetrahydropyridin-4- yl)phenyl)-6-oxo-4- (trifluoromethyl)-1,6- dihydropyridine-3- carboxamide | 21.6470 | 0.00142 | 0.042 |
| 77 | 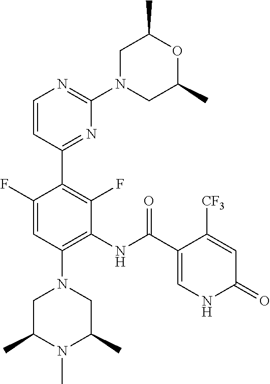 [SMILES: C[C@@H]1CN(c2nccc(-c3c(F)cc(N4C[C@@H](C)N(C)[C@@H](C)C4)c(NC(=O)c4c[nH]c(=O)cc4C(F)(F)F)c3F)n2)C[C@H](C)O1] | N-(3-(2-((2S,6R)-2,6- dimethylmorpholino) pyrimidin-4-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 10.0980 | 0.00112 | 0.108 |
| 78 | 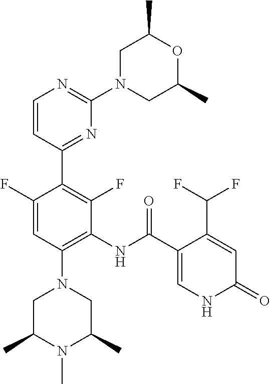 [SMILES: C[C@@H]1CN(c2nccc(-c3c(F)cc(N4C[C@@H](C)N(C)[C@@H](C)C4)c(NC(=O)c4c[nH]c(=O)cc4C(F)F)c3F)n2)C[C@H](C)O1] | 4-(Difluoromethyl)-N- (3-(2-((2S,6R)-2,6- dimethylmorpholino) pyrimidin-4-yl)-2,4- difluoro-6-((3S,5R)- 3,4,5- trimethylpiperazin-1- yl)phenyl)-6-oxo-1,6- dihydropyridine-3- carboxamide | 14.6950 | 0.00116 | 0.0641 |
| 79 | 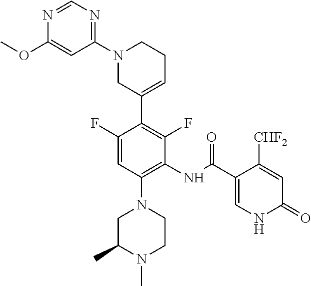 [SMILES: COc1cc(N2CCC=C(c3c(F)cc(N4CCN(C)[C@@H](C)C4)c(NC(=O)c4c[nH]c(=O)cc4C(F)F)c3F)C2)ncn1] | (S)-4-(difluoromethyl)- N-(6-(3,4- dimethylpiperazin-1- yl)-2,4-difluoro-3-(1- (6-methoxypyrimidin- 4-yl)-1,2,5,6- tetrahydropyridin-3- yl)phenyl)-6-oxo-1,6- dihydropyridine-3- carboxamide | 13.5530 | 0.00167 | 0.223 |
| 80 | 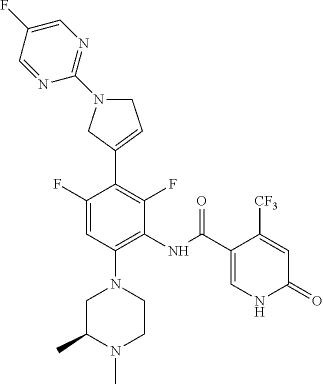 [SMILES: C[C@H]1CN(c2cc(F)c(C3=CCN(c4ncc(F)cn4)C3)c(F)c2NC(=O)c2c[nH]c(=O)cc2C(F)(F)F)CCN1C] | (S)-N-(6-(3,4- dimethylpiperazin-1- yl)-2,4-difluoro-3-(1- (5-fluoropyrimidin-2- yl)-2,5-dihydro-1H- pyrrol-3-yl)phenyl)-6- oxo-4- (trifluoromethyl)-1,6- dihydropyridine-3- carboxamide | 23.0140 | 0.00127 | 0.0583 |
| 81 | 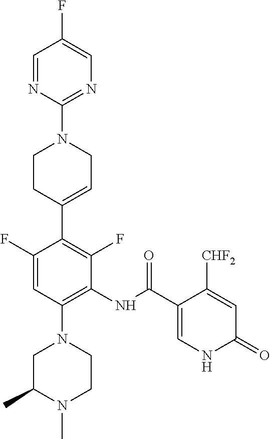 [SMILES: C[C@H]1CN(c2cc(F)c(C3=CCN(c4ncc(F)cn4)CC3)c(F)c2NC(=O)c2c[nH]c(=O)cc2C(F)F)CCN1C] | (S)-4-(difluoromethyl)- N-(6-(3,4- dimethylpiperazin-1- yl)-2,4-difluoro-3-(1- (5-fluoropyrimidin-2- yl)-1,2,3,6- tetrahydropyridin-4- yl)phenyl)-6-oxo-1,6- dihydropyridine-3- carboxamide | 12.5430 | 0.00137 | 0.044 |
| 82 | 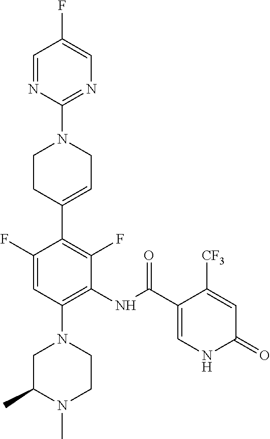 [SMILES: C[C@H]1CN(c2cc(F)c(C3=CCN(c4ncc(F)cn4)CC3)c(F)c2NC(=O)c2c[nH]c(=O)cc2C(F)(F)F)CCN1C] | (S)-N-(6-(3,4- dimethylpiperazin-1- yl)-2,4-difluoro-3-(1- (5-fluoropyrimidin-2- yl)-1,2,3,6- tetrahydropyridin-4- yl)phenyl)-6-oxo-4- (trifluoromethyl)-1,6- dihydropyridine-3- carboxamide | 16.0270 | 0.00122 | 0.0528 |
| 83 | 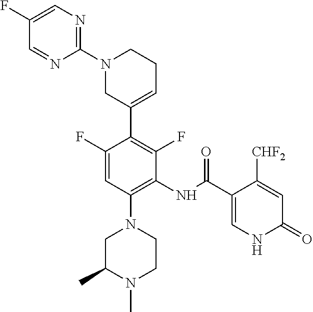 [SMILES: C[C@H]1CN(c2cc(F)c(C3=CCCN(c4ncc(F)cn4)C3)c(F)c2NC(=O)c2c[nH]c(=O)cc2C(F)F)CCN1C] | (S)-4-(difluoromethyl)- N-(6-(3,4- dimethylpiperazin-1- yl)-2,4-difluoro-3-(1- (5-fluoropyrimidin-2- yl)-1,2,5,6- tetrahydropyridin-3- yl)phenyl)-6-oxo-1,6- dihydropyridine-3- carboxamide | 15.5030 | 0.00115 | 0.382 |
| 84 | 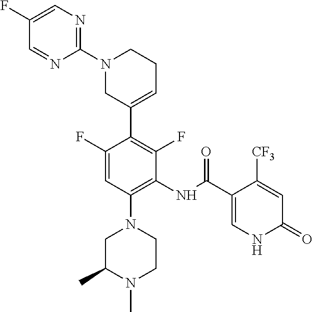 [SMILES: C[C@H]1CN(c2cc(F)c(C3=CCCN(c4ncc(F)cn4)C3)c(F)c2NC(=O)c2c[nH]c(=O)cc2C(F)(F)F)CCN1C] | (S)-N-(6-(3,4- dimethylpiperazin-1- yl)-2,4-difluoro-3-(1- (5-fluoropyrimidin-2- yl)-1,2,5,6- tetrahydropyridin-3- yl)phenyl)-6-oxo-4- (trifluoromethyl)-1,6- dihydropyridine-3- carboxamide | 16.4250 | 0.00143 | 0.162 |
| 85 | 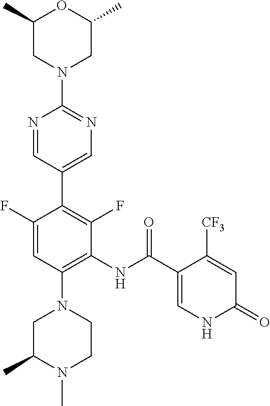 [SMILES: C[C@@H]1CN(c2ncc(-c3c(F)cc(N4CCN(C)[C@@H](C)C4)c(NC(=O)c4c[nH]c(=O)cc4C(F)(F)F)c3F)cn2)C[C@@H](C)O1] | N-(3-(2-((2R,6R)-2,6- dimethylmorpholino) pyrimidin-5-yl)-6-((S)- 3,4-dimethylpiperazin- 1-yl)-2,4- difluorophenyl)-6-oxo- 4-(trifluoromethyl)-1,6- dihydropyridine-3- carboxamide | 13.7870 | 0.00124 | 0.0103 |
| 86 | 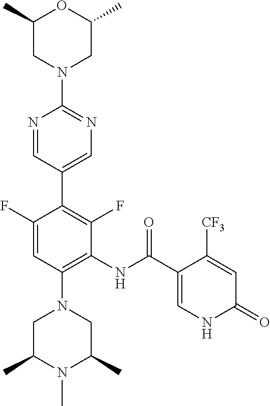 [SMILES: C[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)C[C@@H](C)O1] | N-(3-(2-((2R,6R)-2,6- dimethylmorpholino) pyrimidin-5-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 13.0660 | 0.00088 | 0.0105 |
| 87 | 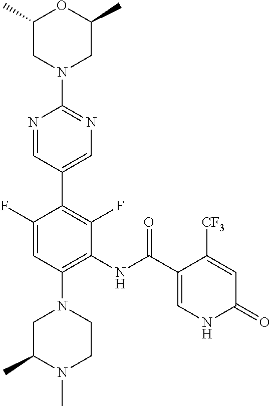 [SMILES: C[C@H]1CN(c2ncc(-c3c(F)cc(N4CCN(C)[C@@H](C)C4)c(NC(=O)c4c[nH]c(=O)cc4C(F)(F)F)c3F)cn2)C[C@H](C)O1] | N-(3-(2-((2S,6S)-2,6- dimethylmorpholino) pyrimidin-5-yl)-6-((S)- 3,4-dimethylpiperazin- 1-yl)-2,4- difluorophenyl)-6-oxo- 4-(trifluoromethyl)-1,6- dihydropyridine-3- carboxamide | 9.8480 | 0.00147 | 0.0194 |
| 88 | 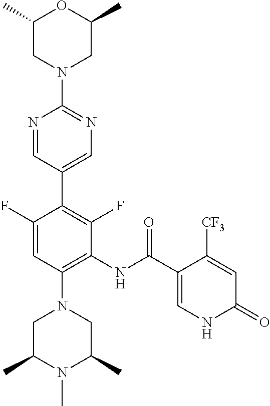 [SMILES: C[C@@H]1CN(c2cc(F)c(-c3cnc(N4C[C@H](C)O[C@@H](C)C4)nc3)c(F)c2NC(=O)c2c[nH]c(=O)cc2C(F)(F)F)C[C@H](C)N1C] | N-(3-(2-((2S,6S)-2,6- dimethylmorpholino) pyrimidin-5-yl)-2,4- difluoro-6-((3S,5R)- 3,4,5- trimethylpiperazin-1- yl)phenyl)-6-oxo-4- (trifluoromethyl)-1,6- dihydropyridine-3- carboxamide | 13.7810 | 0.00249 | 0.00955 |
| 89 | 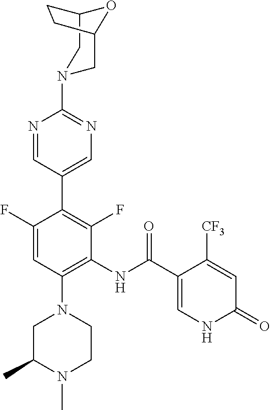 [SMILES: C[C@H]1CN(c2cc(F)c(-c3cnc(N4CC5CCC(C4)O5)nc3)c(F)c2NC(=O)c2c[nH]c(=O)cc2C(F)(F)F)CCN1C] | N-(3-(2-((1R,5S)-8- oxa-3- azabicyclo[3.2.1]octan- 3-yl)pyrimidin-5-yl)-6- ((S)-3,4- dimethylpiperazin-1- yl)-2,4- difluorophenyl)-6-oxo- 4-(trifluoromethyl)-1,6- dihydropyridine-3- carboxamide | 12.5110 | 0.00083 | 0.0308 |
| 90 | 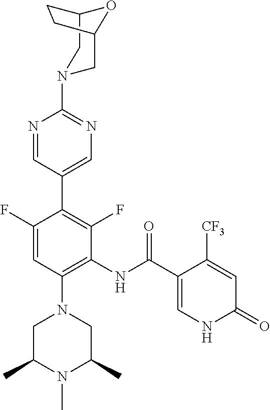 [SMILES: C[C@@H]1CN(c2cc(F)c(-c3cnc(N4CC5CCC(C4)O5)nc3)c(F)c2NC(=O)c2c[nH]c(=O)cc2C(F)(F)F)C[C@H](C)N1C] | N-(3-(2-((1R,5S)-8- oxa-3- azabicyclo[3.2.1]octan- 3-yl)pyrimidin-5-yl)- 2,4-difluoro-6- ((3S,5R)-3,4,5- trimethylpiperazin-1- yl)phenyl)-6-oxo-4- (trifluoromethyl)-1,6- dihydropyridine-3- carboxamide | 15.0250 | 0.00089 | 0.0299 |
| 91 | 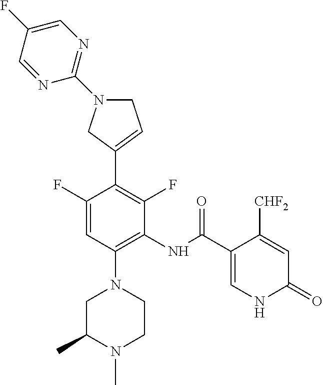 [SMILES: C[C@H]1CN(c2cc(F)c(C3=CCN(c4ncc(F)cn4)C3)c(F)c2NC(=O)c2c[nH]c(=O)cc2C(F)F)CCN1C] | (S)-4-(difluoromethyl)- N-(6-(3,4- dimethylpiperazin-1- yl)-2,4-difluoro-3-(1- (5-fluoropyrimidin-2- yl)-2,5-dihydro-1H- pyrrol-3-yl)phenyl)-6- oxo-1,6- dihydropyridine-3- carboxamide | 16.0170 | 0.00155 | 0.0383 |
| 92 | 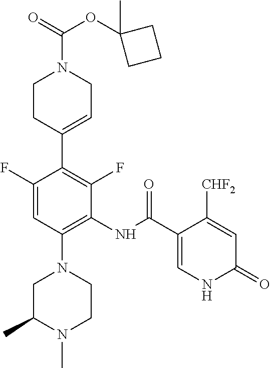 [SMILES: C[C@H]1CN(c2cc(F)c(C3=CCN(C(=O)OC4(C)CCC4)CC3)c(F)c2NC(=O)c2c[nH]c(=O)cc2C(F)F)CCN1C] | 1-Methylcyclobutyl (S)-4-(3-(4- (difluoromethyl)-6- oxo-1,6- dihydropyridine-3- carboxamido)-4-(3,4- dimethylpiperazin-1- yl)-2,6- difluorophenyl)-3,6- dihydropyridine- 1(2H)-carboxylate | 15.6170 | 0.00279 | 0.0228 |
| 93 | 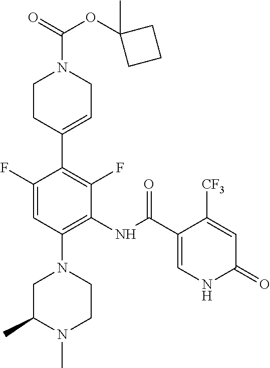 [SMILES: C[C@H]1CN(c2cc(F)c(C3=CCN(C(=O)OC4(C)CCC4)CC3)c(F)c2NC(=O)c2c[nH]c(=O)cc2C(F)(F)F)CCN1C] | 1-Methylcyclobutyl (S)-4-(4-(3,4- dimethylpiperazin-1- yl)-2,6-difluoro-3-(6- oxo-4- (trifluoromethyl)-1,6- dihydropyridine-3- carboxamido)phenyl)- 3,6-dihydropyridine- 1(2H)-carboxylate | 15.4380 | 0.00215 | 0.0194 |
| 94 | 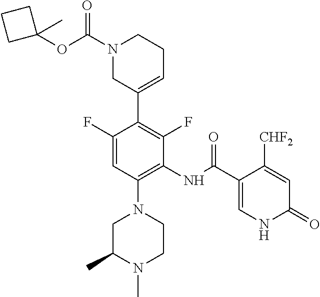 [SMILES: C[C@H]1CN(c2cc(F)c(C3=CCCN(C(=O)OC4(C)CCC4)C3)c(F)c2NC(=O)c2c[nH]c(=O)cc2C(F)F)CCN1C] | 1-Methylcyclobutyl (S)-5-(3-(4- (difluoromethyl)-6- oxo-1,6- dihydropyridine-3- carboxamido)-4-(3,4- dimethylpiperazin-1- yl)-2,6- difluorophenyl)-3,6- dihydropyridine- 1(2H)-carboxylate | 17.4250 | 0.00217 | 0.0939 |
| 95 | 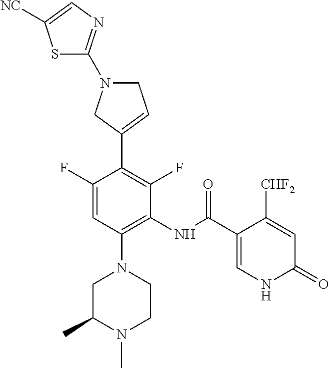 [SMILES: C[C@H]1CN(c2cc(F)c(C3=CCN(c4ncc(C#N)s4)C3)c(F)c2NC(=O)c2c[nH]c(=O)cc2C(F)F)CCN1C] | (S)-N-(3-(1-(5- cyanothiazol-2-yl)-2,5- dihydro-1H-pyrrol-3- yl)-6-(3,4- dimethylpiperazin-1- yl)-2,4- difluorophenyl)-4- (difluoromethyl)-6- oxo-1,6- dihydropyridine-3- carboxamide | 13.8270 | 0.00079 | 0.0964 |
| 96 | 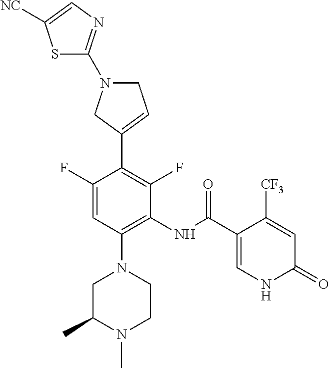 [SMILES: C[C@H]1CN(c2cc(F)c(C3=CCN(c4ncc(C#N)s4)C3)c(F)c2NC(=O)c2c[nH]c(=O)cc2C(F)(F)F)CCN1C] | (S)-N-(3-(1-(5- cyanothiazol-2-yl)-2,5- dihydro-1H-pyrrol-3- yl)-6-(3,4- dimethylpiperazin-1- yl)-2,4- difluorophenyl)-6-oxo- 4-(trifluoromethyl)-1,6- dihydropyridine-3- carboxamide | 11.9240 | 0.00051 | 0.0701 |
| 97 | 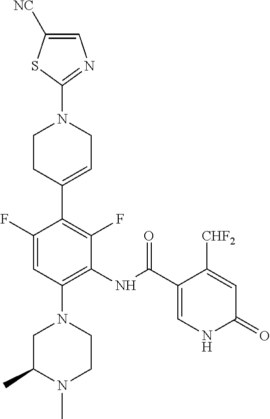 [SMILES: C[C@H]1CN(c2cc(F)c(C3=CCN(c4ncc(C#N)s4)CC3)c(F)c2NC(=O)c2c[nH]c(=O)cc2C(F)F)CCN1C] | (S)-N-(3-(1-(5- cyanothiazol-2-yl)- 1,2,3,6- tetrahydropyridin-4- yl)-6-(3,4- dimethylpiperazin-1- yl)-2,4- difluorophenyl)-4- (difluoromethyl)-6- oxo-1,6- dihydropyridine-3- carboxamide | 13.6430 | 0.00078 | 0.138 |
| 98 | 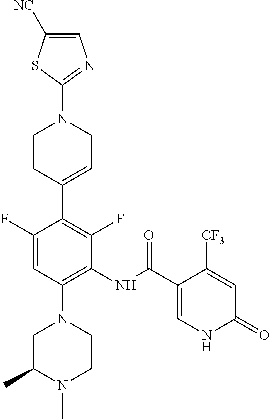 [SMILES: C[C@H]1CN(c2cc(F)c(C3=CCN(c4ncc(C#N)s4)CC3)c(F)c2NC(=O)c2c[nH]c(=O)cc2C(F)(F)F)CCN1C] | (S)-N-(3-(1-(5- cyanothiazol-2-yl)- 1,2,3,6- tetrahydropyridin-4- yl)-6-(3,4- dimethylpiperazin-1- yl)-2,4- difluorophenyl)-6-oxo- 4-(trifluoromethyl)-1,6- dihydropyridine-3- carboxamide | 13.1400 | 0.00077 | 0.0977 |
| 99 | 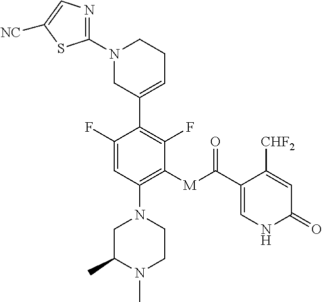 | (S)-N-(3-(1-(5- cyanothiazol-2-yl)- 1,2,5,6- tetrahydropyridin-3- yl)-6-(3,4- dimethylpiperazin-1- yl)-2,4- difluorophenyl)4- (difluoromethyl)-6- oxo-1,6- dihydropyridine-3- carboxamide | 14.6530 | 0.00162 | 0.169 |
| 100 | 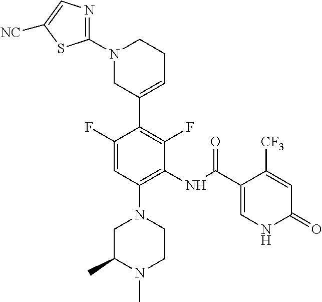 [SMILES: C[C@H]1CN(c2cc(F)c(C3=CCCN(c4ncc(C#N)s4)C3)c(F)c2NC(=O)c2c[nH]c(=O)cc2C(F)(F)F)CCN1C] | (S)-N-(3-(1-(5- cyanothiazol-2-yl)- 1,2,5,6- tetrahydropyridin-3- yl)-6-(3,4- dimethylpiperazin-1- yl)-2,4- difluorophenyl)-6-oxo- 4-(trifluoromethyl)-1,6- dihydropyridine-3- carboxamide | 11.8600 | 0.00145 | 0.181 |
| 101 | 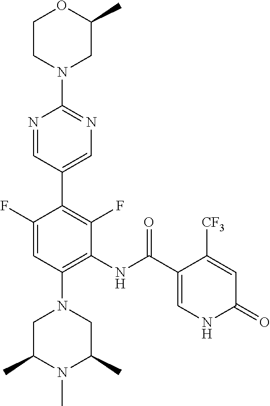 [SMILES: C[C@@H]1CN(c2cc(F)c(-c3cnc(N4CCO[C@@H](C)C4)nc3)c(F)c2NC(=O)c2c[nH]c(=O)cc2C(F)(F)F)C[C@H](C)N1C] | N-(2,4-difluoro-3-(2- ((S)-2- methylmorpholino) pyrimidin-5-yl)-6- ((3S,5R)-3,4,5- trimethylpiperazin-1- yl)phenyl)-6-oxo-4- (trifluoromethyl)-1,6- dihydropyridine-3- carboxamide | 21.0200 | 0.00617 | 0.0118 |
| 102 | 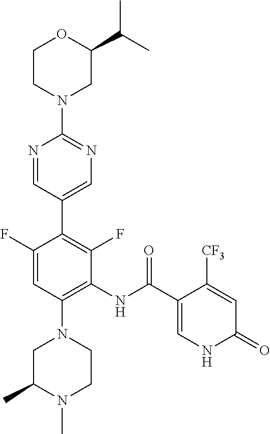 [SMILES: CC(C)[C@H]1CN(c2ncc(-c3c(F)cc(N4CCN(C)[C@@H](C)C4)c(NC(=O)c4c[nH]c(=O)cc4C(F)(F)F)c3F)cn2)CCO1] | N-(6-((S)-3,4- dimethylpiperazin-1- yl)-2,4-difluoro-3-(2- ((S)-2- isopropylmorpholino) pyrimidin-5-yl)phenyl)- 6-oxo-4- (trifluoromethyl)-1,6- dihydropyridine-3- carboxamide | 11.6890 | 0.00044 | 0.0143 |
| 103 | 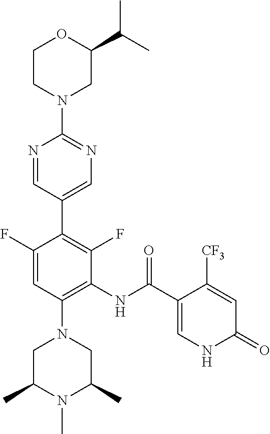 [SMILES: CC(C)[C@H]1CN(c2ncc(-c3c(F)cc(N4C[C@@H](C)N(C)[C@@H](C)C4)c(NC(=O)c4c[nH]c(=O)cc4C(F)(F)F)c3F)cn2)CCO1] | N-(2,4-difluoro-3-(2- ((S)-2- isopropylmorpholino) pyrimidin-5-yl)-6- ((3S,5R)-3,4,5- trimethylpiperazin-1- yl)phenyl)-6-oxo-4- (trifluoromethyl)-1,6- dihydropyridine-3- carboxamide | 17.7220 | 0.00501 | 0.0147 |
| 104 | 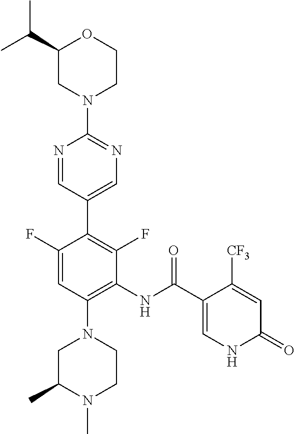 [SMILES: CC(C)[C@@H]1CN(c2ncc(-c3c(F)cc(N4CCN(C)[C@@H](C)C4)c(NC(=O)c4c[nH]c(=O)cc4C(F)(F)F)c3F)cn2)CCO1] | N-(6-((S)-3,4- dimethylpiperazin-1- yl)-2,4-difluoro-3-(2- ((R)-2- isopropylmorpholino) pyrimidin-5-yl)phenyl)- 6-oxo-4- (trifluoromethyl)-1,6- dihydropyridine-3- carboxamide | 16.5120 | 0.00225 | 0.0203 |
| 105 | 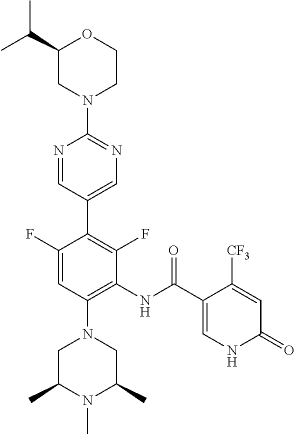 [SMILES: CC(C)[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)CCO1] | N-(2,4-difluoro-3-(2- ((R)-2- isopropylmorpholino) pyrimidin-5-yl)-6- ((3S,5R)-3,4,5- trimethylpiperazin-1- yl)phenyl)-6-oxo-4- (trifluoromethyl)-1,6- dihydropyridine-3- carboxamide | 14.6620 | 0.00389 | 0.0143 |
| 106 | 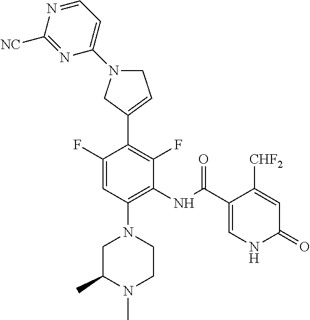 [SMILES: C[C@H]1CN(c2cc(F)c(C3=CCN(c4ccnc(C#N)n4)C3)c(F)c2NC(=O)c2c[nH]c(=O)cc2C(F)F)CCN1C] | (S)-N-(3-(1-(2- cyanopyrimidin-4-yl)- 2,5-dihydro-1H-pyrrol- 3-yl)-6-(3,4- dimethylpiperazin-1- yl)-2,4- difluorophenyl)-4- (difluoromethyl)-6- oxo-1,6- dihydropyridine-3- carboxamide | 15.0440 | 0.00093 | 0.0276 |
| 107 | 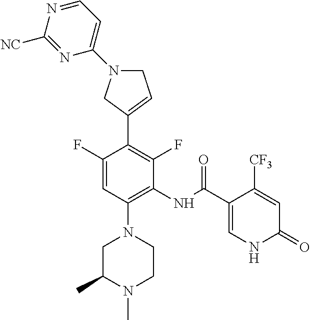 [SMILES: C[C@H]1CN(c2cc(F)c(C3=CCN(c4ccnc(C#N)n4)C3)c(F)c2NC(=O)c2c[nH]c(=O)cc2C(F)(F)F)CCN1C] | (S)-N-(3-(1-(2- cyanopyrimidin-4-yl)- 2,5-dihydro-1H-pyrrol- 3-yl)-6-(3,4- dimethylpiperazin-1- yl)-2,4- difluorophenyl)-6-oxo- 4-(trifluoromethyl)-1,6- dihydropyridine-3- carboxamide | 14.3390 | 0.00262 | 0.0601 |
| 108 | 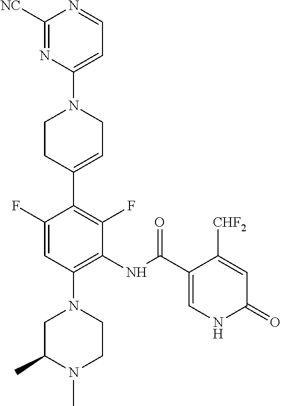 [SMILES: C[C@H]1CN(c2cc(F)c(C3=CCN(c4ccnc(C#N)n4)CC3)c(F)c2NC(=O)c2c[nH]c(=O)cc2C(F)F)CCN1C] | (S)-N-(3-(1-(2- cyanopyrimidin-4-yl)- 1,2,3,6- tetrahydropyridin-4- yl)-6-(3,4- dimethylpiperazin-1- yl)-2,4- difluorophenyl)-4- (difluoromethyl)-6- oxo-1,6- dihydropyridine-3- carboxamide | 14.2250 | 0.00165 | 0.0299 |
| 109 | 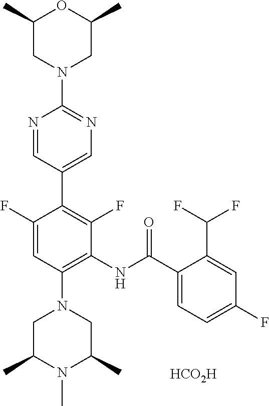 [SMILES: C[C@@H]1CN(c2ncc(-c3c(F)cc(N4C[C@@H](C)N(C)[C@@H](C)C4)c(NC(=O)c4ccc(F)cc4C(F)F)c3F)cn2)C[C@H](C)O1 ; "HCO2H"] | : 2-(Difluoromethyl)- N-(3-(2-((2S,6R)-2,6- dimethylmorpholino) pyrimidin-5-yl)-2,4- difluoro-6-((3S,5R)- 3,4,5- trimethylpiperazin-1- yl)phenyl)-4- fluorobenzamide formic acid | 14.3930 | 0.00259 | 0.00734 |
| 110 | 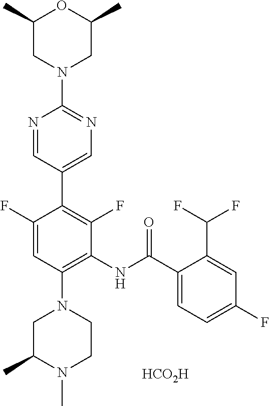 [SMILES: C[C@@H]1CN(c2ncc(-c3c(F)cc(N4CCN(C)[C@@H](C)C4)c(NC(=O)c4ccc(F)cc4C(F)F)c3F)cn2)C[C@H](C)O1 ; "HCO2H"] | 2-(Difluoromethyl)-N- (3-(2-((2S,6R)-2,6- dimethylmorpholino) pyrimidin-5-yl)-6-((S)- 3,4-dimethylpiperazin- 1-yl)-2,4- difluorophenyl)-4- fluorobenzamide formic acid | 10.8550 | 0.00194 | 0.0332 |
| 111 | 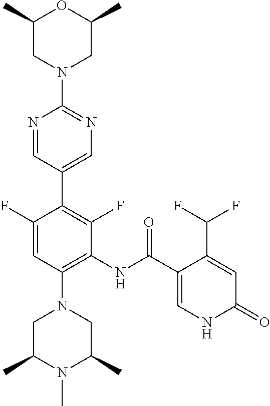 [SMILES: C[C@@H]1CN(c2ncc(-c3c(F)cc(N4C[C@@H](C)N(C)[C@@H](C)C4)c(NC(=O)c4c[nH]c(=O)cc4C(F)F)c3F)cn2)C[C@H](C)O1] | 4-(Difluoromethyl)-N- (3-(2-((2S,6R)-2,6- dimethylmorpholino) pyrimidin-5-yl)-2,4- difluoro-6-((3S,5R)- 3,4,5- trimethylpiperazin-1- yl)phenyl)-6-oxo-1,6- dihydropyridine-3- carboxamide | 14.1380 | 0.00129 | 0.00948 |
| 112 | 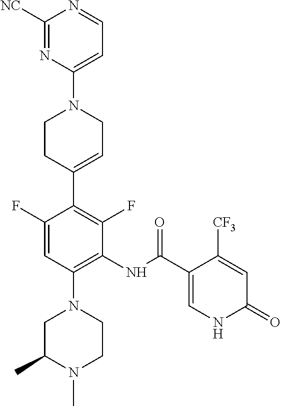 [SMILES: C[C@H]1CN(c2cc(F)c(C3=CCN(c4ccnc(C#N)n4)CC3)c(F)c2NC(=O)c2c[nH]c(=O)cc2C(F)(F)F)CCN1C] | (S)-N-(3-(1-(2- cyanopyrimidin-4-yl)- 1,2,3,6- tetrahydropyridin-4- yl)-6-(3,4- dimethylpiperazin-1- yl)-2,4- difluorophenyl)-6-oxo- 4-(trifluoromethyl)-1,6- dihydropyridine-3- carboxamide | 15.0190 | 0.0024 | 0.035 |
| 113 | 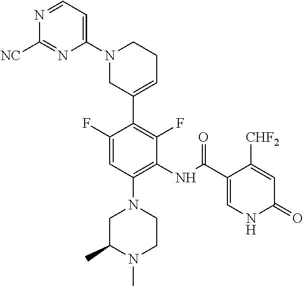 [SMILES: C[C@H]1CN(c2cc(F)c(C3=CCCN(c4ccnc(C#N)n4)C3)c(F)c2NC(=O)c2c[nH]c(=O)cc2C(F)F)CCN1C] | (S)-N-(3-(2-(2- cyanopyrimidin-4-yl)- 1,2,5,6- tetrahydropyridin-3- yl)-6-(3,4- dimethylpiperazin-1- yl)-2,4- difluorophenyl)-4- (difluoromethyl)-6- oxo-1,6- dihydropyridine-3- carboxamide | 14.8070 | 0.00199 | 0.0859 |
| 114 | 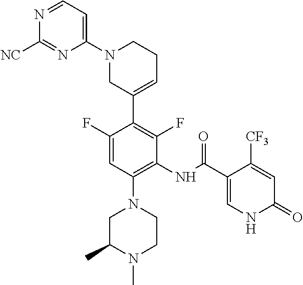 [SMILES: C[C@H]1CN(c2cc(F)c(C3=CCCN(c4ccnc(C#N)n4)C3)c(F)c2NC(=O)c2c[nH]c(=O)cc2C(F)(F)F)CCN1C] | (S)-N-(3-(1-(2- cyanopyrimidin-4-yl)- 1,2,5,6- tetrahydropyridin-3- yl)-6-(3,4- dimethylpiperazin-1- yl)-2,4- difluorophenyl)-6-oxo- 4-(trifluoromethyl)-1,6- dihydropyridine-3- carboxamide | 15.9420 | 0.00242 | 0.0366 |
| 115 | 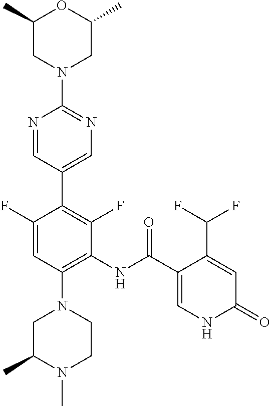 [SMILES: C[C@@H]1CN(c2ncc(-c3c(F)cc(N4CCN(C)[C@@H](C)C4)c(NC(=O)c4c[nH]c(=O)cc4C(F)F)c3F)cn2)C[C@@H](C)O1] | 4-(Difluoromethyl)-N- (3-(2-((2R,6R)-2,6- dimethylmorpholino) pyrimidin-5-yl)-6-((S)- 3,4-dimethylpiperazin- 1-yl)-2,4- difluorophenyl)-6-oxo- 1,6-dihydropyridine-3- carboxamide | 13.7530 | 0.0007 | 0.00938 |
| 116 | 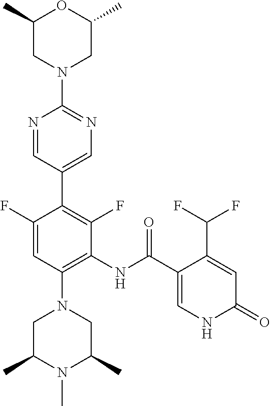 [SMILES: C[C@@H]1CN(c2ncc(-c3c(F)cc(N4C[C@@H](C)N(C)[C@@H](C)C4)c(NC(=O)c4c[nH]c(=O)cc4C(F)F)c3F)cn2)C[C@@H](C)O1] | 4-(Difluoromethyl)-N- (3-(2-((2R,6R)-2,6- dimethylmorpholino) pyrimidin-5-yl)-2,4- difluoro-6-((3S,5R)- 3,4,5- trimethylpiperazin-1- yl)phenyl)-6-oxo-1,6- dihydropyridine-3- carboxamide | 15.2390 | 0.00112 | 0.0365 |
| 117 | 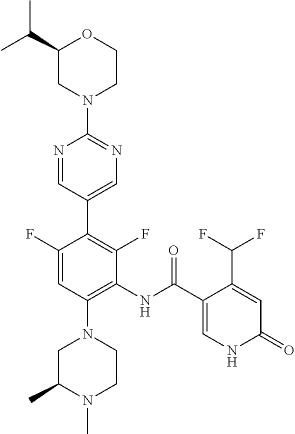 [SMILES: CC(C)[C@@H]1CN(c2ncc(-c3c(F)cc(N4CCN(C)[C@@H](C)C4)c(NC(=O)c4c[nH]c(=O)cc4C(F)F)c3F)cn2)CCO1] | 4-(Difluoromethyl)-N- (6-((S)-3,4- dimethylpiperazin-1- yl)-2,4-difluoro-3-(2- ((R)-2- isopropylmorpholino) pyrimidin-5-yl) phenyl)-6-oxo-1,6- dihydropyridine-3- carboxamide | 13.6090 | 0.00084 | 0.0282 |
| 118 | 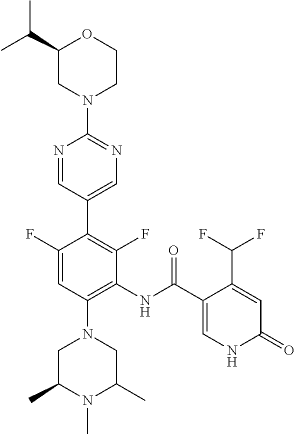 [SMILES: CC(C)[C@@H]1CN(c2ncc(-c3c(F)cc(N4CC(C)N(C)[C@@H](C)C4)c(NC(=O)c4c[nH]c(=O)cc4C(F)F)c3F)cn2)CCO1] | N-(2,4-Difluoro-3-(2- ((R)-2- isopropylmorpholino) pyrimidin-5-yl)-6- ((3S,5R)-3,4,5- trimethylpiperazin-1- yl)phenyl)-4- (difluoromethyl)-6- oxo-1,6- dihydropyridine-3- carboxamide | 12.1530 | 0.00095 | 0.0122 |
| 119 | 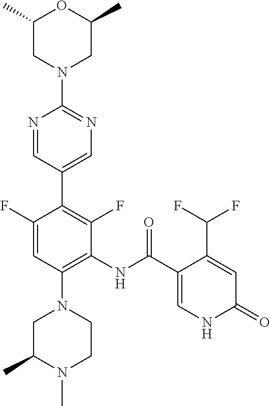 [SMILES: C[C@H]1CN(c2ncc(-c3c(F)cc(N4CCN(C)[C@@H](C)C4)c(NC(=O)c4c[nH]c(=O)cc4C(F)F)c3F)cn2)C[C@H](C)O1] | 4-(Difluoromethyl)-N- (3-(2-((2S,6S)-2,6- dimethylmorpholino) pyrimidin-5-yl)-6-((S)- 3,4-dimethylpiperazin- 1-yl)-2,4- difluorophenyl)-6-oxo- 1,6-dihydropyridine-3- carboxamide | 11.7140 | 0.0009 | 0.0204 |
| 120 | 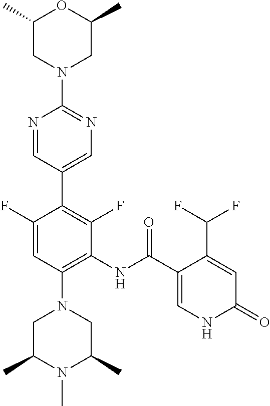 [SMILES: C[C@@H]1CN(c2cc(F)c(-c3cnc(N4C[C@H](C)O[C@@H](C)C4)nc3)c(F)c2NC(=O)c2c[nH]c(=O)cc2C(F)F)C[C@H](C)N1C] | 4-(Difluoromethyl)-N- (3-(2-((2S,6S)-2,6- dimethylmorpholino) pyrimidin-5-yl)-2,4- difluoro-6-((3S,5R)- 3,4,5- trimethylpiperazin-1- yl)phenyl)-6-oxo-1,6- dihydropyridine-3- carboxamide | 11.9990 | 0.00135 | 0.00207 |
| 121 | 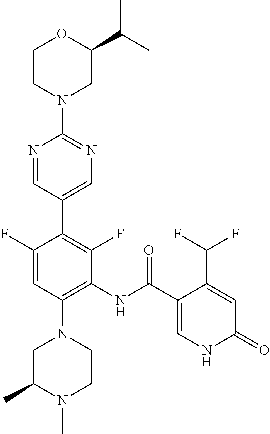 [SMILES: CC(C)[C@H]1CN(c2ncc(-c3c(F)cc(N4CCN(C)[C@@H](C)C4)c(NC(=O)c4c[nH]c(=O)cc4C(F)F)c3F)cn2)CCO1] | 4-(Difluoromethyl)-N- (6-((S)-3 dimethylpiperazin-1- yl)-2,4-difluoro-3-(2- ((S)-2- isopropylmorpholino) pyrimidin-5-yl)phenyl)- 6-oxo-1,6- dihydropyridine-3- carboxamide | 11.9430 | 0.00107 | 0.0157 |
| 122 | 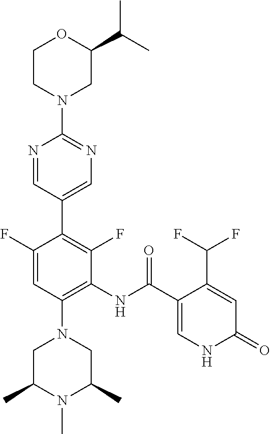 [SMILES: CC(C)[C@H]1CN(c2ncc(-c3c(F)cc(N4C[C@@H](C)N(C)[C@@H](C)C4)c(NC(=O)c4c[nH]c(=O)cc4C(F)F)c3F)cn2)CCO1] | N-(2,4-difluoro-3-(2- ((S)-2- isopropylmorpholino) pyrimidin-5-yl)-6- ((3S,5R)-3,4,5- trimethylpiperazin-1- yl)phenyl)-4- (difluoromethyl)-6- oxo-1,6- dihydropyridine-3- carboxamide | 12.4810 | 0.00198 | 0.0221 |
| 123 | 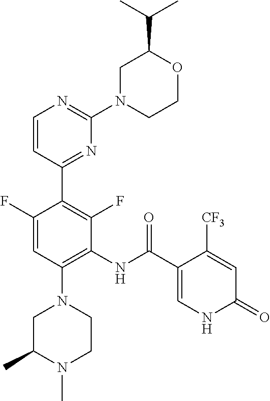 [SMILES: CC(C)[C@@H]1CN(c2nccc(-c3c(F)cc(N4CCN(C)[C@@H](C)C4)c(NC(=O)c4c[nH]c(=O)cc4C(F)(F)F)c3F)n2)CCO1] | N-(6-((S)-3,4- dimethylpiperazin-1- yl)-2,4-difluoro-3-(2- ((R)-2- isopropylmorpholino) pyrimidin-4-yl)phenyl)- 6-oxo-4- (trifluoromethyl)-1,6- dihydropyridine-3- carboxamide | 14.8530 | 0.00168 | 0.0618 |
| 124 | 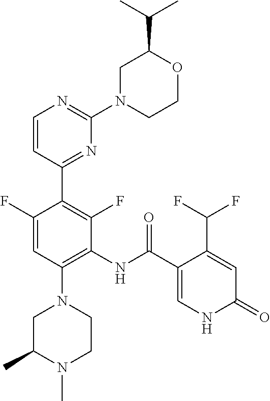 [SMILES: CC(C)[C@@H]1CN(c2nccc(-c3c(F)cc(N4CCN(C)[C@@H](C)C4)c(NC(=O)c4c[nH]c(=O)cc4C(F)F)c3F)n2)CCO1] | 4-(Difluoromethyl)-N- (6-((S)-3,4- dimethylpiperazin-1- yl)-2,4-difluoro-3-(2- ((R)-2- isopropylmorpholino) pyrimidin-4-yl) phenyl)-6-oxo-1,6- dihydropyridine-3- carboxamide | 12.4230 | 0.0008 | 0.0559 |
| 125 | 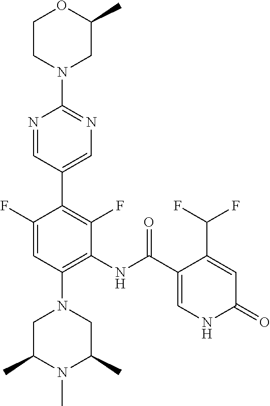 [SMILES: C[C@@H]1CN(c2cc(F)c(-c3cnc(N4CCO[C@@H](C)C4)nc3)c(F)c2NC(=O)c2c[nH]c(=O)cc2C(F)F)C[C@H](C)N1C] | N-(2,4-difluoro-3-(2- ((S)-2- methylmorpholino) pyrimidin-5-yl)-6- ((3S,5R)-3,4,5- trimethylpiperazin-1- yl)phenyl)-4- (difluoromethyl)-6- oxo-1,6- dihydropyridine-3- carboxamide | 11.8850 | 0.00069 | 0.0194 |
| 126 | 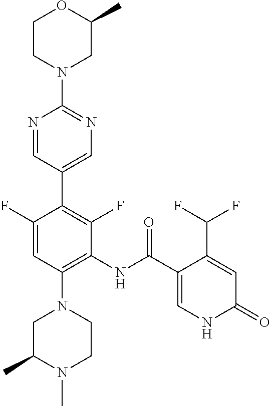 [SMILES: C[C@H]1CN(c2ncc(-c3c(F)cc(N4CCN(C)[C@@H](C)C4)c(NC(=O)c4c[nH]c(=O)cc4C(F)F)c3F)cn2)CCO1] | 4-(Difluoromethyl)-N- (6-((S)-3,4- dimethylpiperazin-1- yl)-2,4-difluoro-3-(2- ((S)-2- methylmorpholino) pyrimidin-5-y l) phenyl)-6-oxo-1,6- dihydropyridine-3- carboxamide | 11.7690 | 0.00056 | 0.0118 |
| 127 | 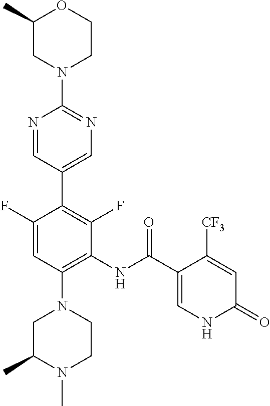 [SMILES: C[C@@H]1CN(c2ncc(-c3c(F)cc(N4CCN(C)[C@@H](C)C4)c(NC(=O)c4c[nH]c(=O)cc4C(F)(F)F)c3F)cn2)CCO1] | N-(6-((S)-3,4- dimethylpiperazin-1- yl)-2,4-difluoro-3-(2- ((R)-2- methylmorpholino) pyrimidin-5-yl) phenyl)-6-oxo-4- (trifluoromethyl)-1,6- dihydropyridine-3- carboxamide | 11.6790 | 0.00069 | 0.0102 |
| 128 | 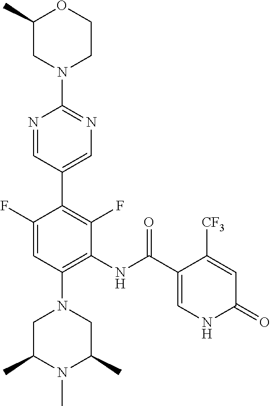 [SMILES: C[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)CCO1] | N-(2,4-difluoro-3-(2- ((R)-2- methylmorpholino) pyrimidin-5-yl)-6- ((3S,5R)-3,4,5- trimethylpiperazin-1- yl)phenyl)-6-oxo-4- (trifluoromethyl)-1,6- dihydropyridine-3- carboxamide | 12.7590 | 0.00069 | 0.00585 |
| 129 | 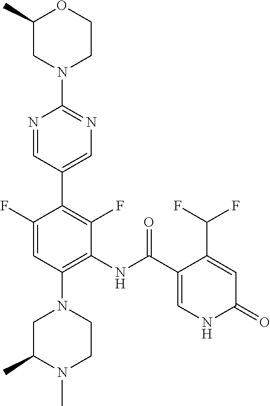 [SMILES: C[C@@H]1CN(c2ncc(-c3c(F)cc(N4CCN(C)[C@@H](C)C4)c(NC(=O)c4c[nH]c(=O)cc4C(F)F)c3F)cn2)CCO1] | 4-(Difluoromethyl)-N- (6-((S)-3,4- dimethylpiperazin-1- yl)-2,4-difluoro-3-(2- ((R)-2- methylmorpholino) pyrimidin-5-yl) phenyl)-6-oxo-1,6- dihydropyridine-3- carboxamide | 8.7950 | 0.00034 | 0.0135 |
| 130 | 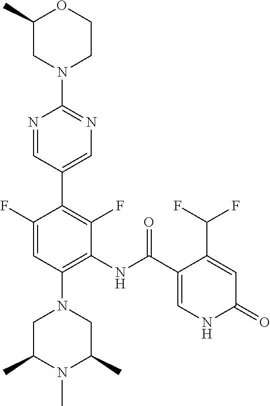 [SMILES: C[C@@H]1CN(c2ncc(-c3c(F)cc(N4C[C@@H](C)N(C)[C@@H](C)C4)c(NC(=O)c4c[nH]c(=O)cc4C(F)F)c3F)cn2)CCO1] | N-(2,4-difluoro-3-(2- ((R)-2- methylmorpholino) pyrimidin-5-yl)-6- ((3S,5R)-3,4,5- trimethylpiperazin-1- yl)phenyl)-4- (difluoromethyl)-6- oxo-1,6- dihydropyridine-3- carboxamide | 8.9170 | 0.00055 | 0.0102 |
| 131 | 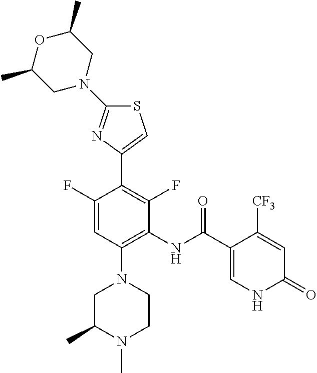 [SMILES: C[C@@H]1CN(c2nc(-c3c(F)cc(N4CCN(C)[C@@H](C)C4)c(NC(=O)c4c[nH]c(=O)cc4C(F)(F)F)c3F)cs2)C[C@H](C)O1] | N-(3-(2-((2S,6R)-2,6- dimethylmorpholino) thiazol-4-yl)-6-((S)-3,4- dimethylpiperazin-1- yl)-2,4- difluorophenyl)-6-oxo- 4-(trifluoromethyl)-1,6- dihydropyridine-3- carboxamide | 16.6310 | 0.00512 | 0.208 |
| 132 | 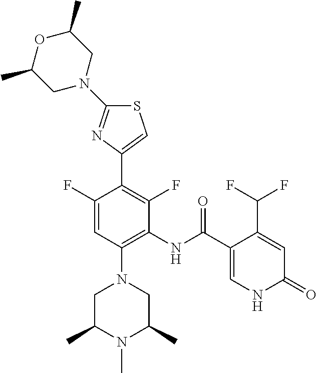 [SMILES: C[C@@H]1CN(c2nc(-c3c(F)cc(N4C[C@@H](C)N(C)[C@@H](C)C4)c(NC(=O)c4c[nH]c(=O)cc4C(F)F)c3F)cs2)C[C@H](C)O1] | 4-(Difluoromethyl)-N- (3-(2-((2S,6R)-2,6- dimethylmorpholino) thiazol-4-yl)-6-((S)- 3,4-dimethylpiperazin- 1-yl)-2,4- difluorophenyl)-6-oxo- 1,6-dihydropyridine-3- carboxamide | 12.3570 | 0.00201 | 0.132 |
| 133 | 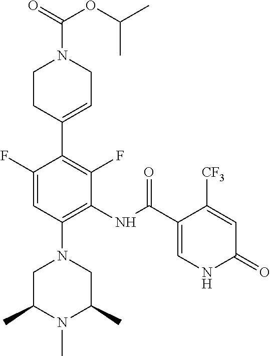 [SMILES: CC(C)OC(=O)N1CC=C(c2c(F)cc(N3C[C@@H](C)N(C)[C@@H](C)C3)c(NC(=O)c3c[nH]c(=O)cc3C(F)(F)F)c2F)CC1] | 4-(2,6-Difluoro-3-(6- oxo-4- (trifluoromethyl)-1,6- dihydropyridine-3- carboxamido)-4- ((3S,5R)-3,4,5- trimethylpiperazin-1- yl)phenyl)-5,6- dihydropyridine- 1(2H)-carboxylate | 12.2940 | 0.00262 | 0.0585 |
| 134 | 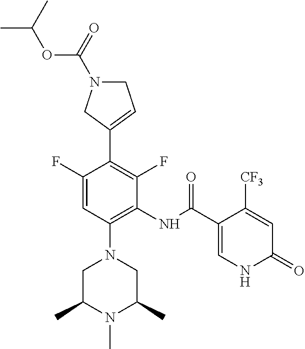 [SMILES: CC(C)OC(=O)N1CC=C(c2c(F)cc(N3C[C@@H](C)N(C)[C@@H](C)C3)c(NC(=O)c3c[nH]c(=O)cc3C(F)(F)F)c2F)C1] | 3-(2,6-Difluoro-3-(6- oxo-4- (trifluoromethyl)-1,6- dihydropyridine-3- carboxamido)-4- ((3S,5R)-3,4,5- trimethylpiperazin-1- yl)phenyl)-2,5- dihydro-1H-pyrrole-1- carboxylate | 11.3870 | 0.00087 | 0.0558 |
| 135 | 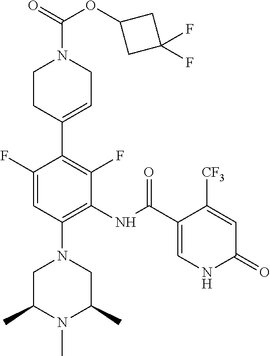 [SMILES: C[C@@H]1CN(c2cc(F)c(C3=CCN(C(=O)OC4CC(F)(F)C4)CC3)c(F)c2NC(=O)c2c[nH]c(=O)cc2C(F)(F)F)C[C@H](C)N1C] | 3,3-Difluorocyclobutyl 4-(2,6-difluoro-3-(6- oxo-4- (trifluoromethyl)-1,6- dihydropyridine-3- carboxamido)-4- ((3S,5R)-3,4,5- trimethylpiperazin-1- yl)phenyl)-5,6- dihydropyridine- 1(2H)-carboxylate | 12.5120 | 0.00231 | 0.0674 |
| 136 | 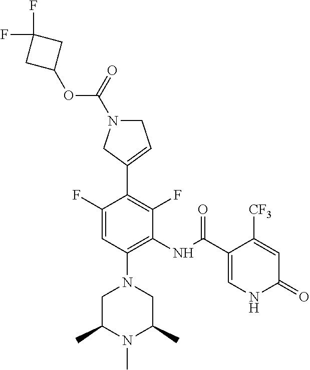 [SMILES: C[C@@H]1CN(c2cc(F)c(C3=CCN(C(=O)OC4CC(F)(F)C4)C3)c(F)c2NC(=O)c2c[nH]c(=O)cc2C(F)(F)F)C[C@H](C)N1C] | 3,3-Difluorocyclobutyl 3-(2,6-difluoro-3-(6- oxo-4- (trifluoromethyl)-1,6- dihydropyridine-3- carboxamido)-4- ((3S,5R)-3,4,5- trimethylpiperazin-1- yl)phenyl)-2,5- dihydro-1H-pyrrole-1- carboxylate | 14.9290 | 0.00116 | 0.042 |
| 137 | 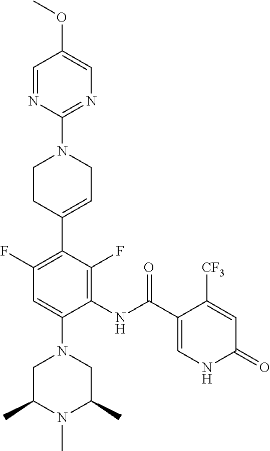 [SMILES: COc1cnc(N2CC=C(c3c(F)cc(N4C[C@@H](C)N(C)[C@@H](C)C4)c(NC(=O)c4c[nH]c(=O)cc4C(F)(F)F)c3F)CC2)nc1] | N-(2,4-difluoro-3-(1- (5-methoxypyrimidin- 2-yl)-1,2,3,6- tetrahydropyridin-4- yl)-6-((3S,5R)-3,4,5- trimethylpiperazin-1- yl)phenyl)-6-oxo-4- (trifluoromethyl)-1,6- dihydropyridine-3- carboxamide | 17.0180 | 0.00257 | 0.0751 |
| 138 | 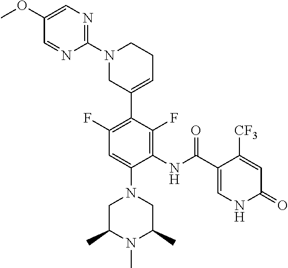 [SMILES: COc1cnc(N2CCC=C(c3c(F)cc(N4C[C@@H](C)N(C)[C@@H](C)C4)c(NC(=O)c4c[nH]c(=O)cc4C(F)(F)F)c3F)C2)nc1] | N-(2,4-difluoro-3-(1- (5-methoxypyrimidin- 2-yl)-1,2,5,6- tetrahydropyridin-3- yl)-6-((3S,5R)-3,4,5- trimethylpiperazin-1- yl)phenyl)-6-oxo-4- (trifluoromethyl)-1,6- dihydropyridine-3- carboxamide | 19.5450 | 0.00193 | 0.109 |
| 139 | 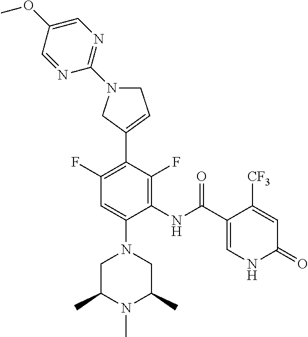 [SMILES: COc1cnc(N2CC=C(c3c(F)cc(N4C[C@@H](C)N(C)[C@@H](C)C4)c(NC(=O)c4c[nH]c(=O)cc4C(F)(F)F)c3F)C2)nc1] | N-(2,4-difluoro-3-(1- (5-methoxypyrimidin- 2-yl)-2,5-dihydro-1H- pyrrol-3-yl)-6- ((3S,5R)-3,4,5- trimethylpiperazin-1- yl)phenyl)-6-oxo-4- (trifluoromethyl)-1,6- dihydropyridine-3- carboxamide | 11.9760 | 0.00165 | 0.0185 |
| 140 | 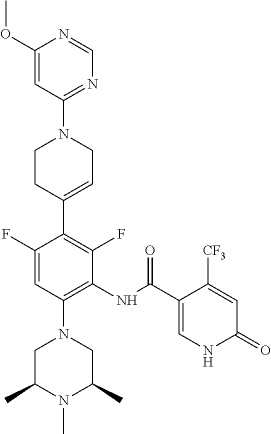 [SMILES: COc1cc(N2CC=C(c3c(F)cc(N4C[C@@H](C)N(C)[C@@H](C)C4)c(NC(=O)c4c[nH]c(=O)cc4C(F)(F)F)c3F)CC2)ncn1] | N-(2,4-difluoro-3-(1- (6-methoxypyrimidin- 4-yl)-1,2,3,6- tetrahydropyridin-4- yl)-6-((3S,5R)-3,4,5- trimethylpiperazin-1- yl)phenyl)-6-oxo-4- (trifluoromethyl)-1,6- dihydropyridine-3- carboxamide | 14.5030 | 0.0014 | 0.0309 |
| 141 | 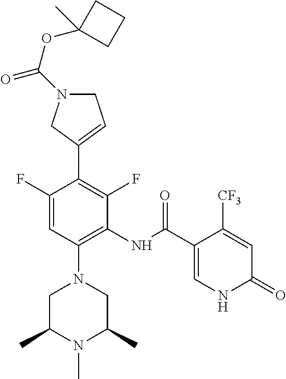 [SMILES: C[C@@H]1CN(c2cc(F)c(C3=CCN(C(=O)OC4(C)CCC4)C3)c(F)c2NC(=O)c2c[nH]c(=O)cc2C(F)(F)F)C[C@H](C)N1C] | 1-Methylcyclobutyl 3- (2,6-difluoro-3-(6-oxo- 4-(trifluoromethyl)-1,6- dihydropyridine-3- carboxamido)-4- ((3S,5R)-3,4,5- trimethylpiperazin-1- yl)phenyl)-2,5- dihydro-1H-pyrrole-1- carboxylate | 10.0980 | 0.00096 | 0.0312 |
| 142 | 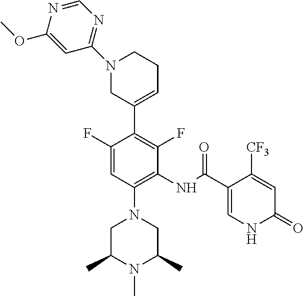 [SMILES: COc1cc(N2CCC=C(c3c(F)cc(N4C[C@@H](C)N(C)[C@@H](C)C4)c(NC(=O)c4c[nH]c(=O)cc4C(F)(F)F)c3F)C2)ncn1] | N-(2,4-difluoro-3-(1- (6-methoxypyrimidin- 4-yl)-1,2,5,6- tetrahydropyridin-3- yl)-6-((3S,5R)-3,4,5- trimethylpiperazin-1- yl)phenyl)-6-oxo-4- (trifluoromethyl)-1,6- dihydropyridine-3- carboxamide | 13.1800 | 0.00176 | 0.0383 |
| 143 | 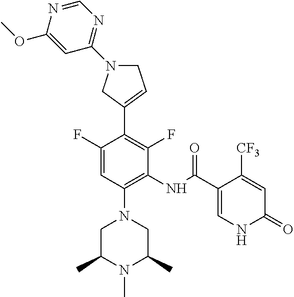 [SMILES: COc1cc(N2CC=C(c3c(F)cc(N4C[C@@H](C)N(C)[C@@H](C)C4)c(NC(=O)c4c[nH]c(=O)cc4C(F)(F)F)c3F)C2)ncn1] | N-(2,4-difluoro-3-(1- (6-methoxypyrimidin- 4-yl)-2,5-dihydro-1H- pyrrol-3-yl)-6- ((3S,5R)-3,4,5- trimethylpiperazin-1- yl)phenyl)-6-oxo-4- (trifluoromethyl)-1,6- dihydropyridine-3- carboxamide | 14.3790 | 0.00092 | 0.0177 |
| 144 | 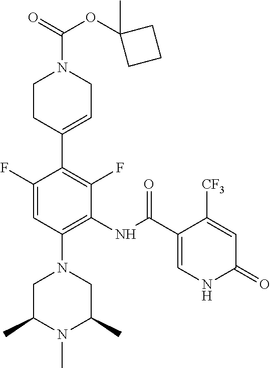 [SMILES: C[C@@H]1CN(c2cc(F)c(C3=CCN(C(=O)OC4(C)CCC4)CC3)c(F)c2NC(=O)c2c[nH]c(=O)cc2C(F)(F)F)C[C@H](C)N1C] | 1-Methylcyclobutyl 4- (2,6-difluoro-3-(6-oxo- 4-(trifluoromethyl)-1,6- dihydropyridine-3- carboxamido)-4- ((3S,5R)-3,4,5- trimethylpiperazin-1- yl)phenyl)-5,6- dihydropyridine- 1(2H)-carboxylate | 12.6640 | 0.00153 | 0.0114 |
| 145 | 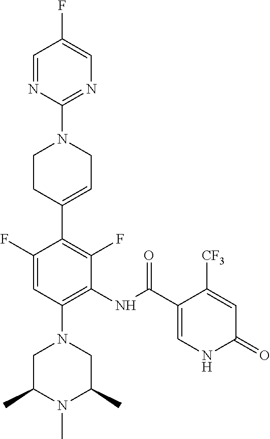 [SMILES: C[C@@H]1CN(c2cc(F)c(C3=CCN(c4ncc(F)cn4)CC3)c(F)c2NC(=O)c2c[nH]c(=O)cc2C(F)(F)F)C[C@H](C)N1C] | N-(2,4-difluoro-3-(1- (5-fluoropyrimidin-2- yl)-1,2,3,6- tetrahydropyridin-4- yl)-6-((3S,5R)-3,4,5- trimethylpiperazin-1- yl)phenyl)-6-oxo-4- (trifluoromethyl)-1,6- dihydropyridine-3- carboxamide | 13.9010 | 0.00084 | 0.0589 |
| 146 | 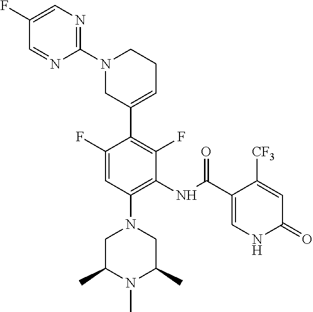 [SMILES: C[C@@H]1CN(c2cc(F)c(C3=CCCN(c4ncc(F)cn4)C3)c(F)c2NC(=O)c2c[nH]c(=O)cc2C(F)(F)F)C[C@H](C)N1C] | N-(2,4-difluoro-3-(1- (5-fluoropyrimidin-2- yl)-1,2,5,6- tetrahydropyridin-3- yl)-6-((3S,5R)-3,4,5- trimethylpiperazin-1- yl)phenyl)-6-oxo-4- (trifluoromethyl)-1,6- dihydropyridine-3- carboxamide | 20.9570 | 0.00165 | 0.239 |
| 147 | 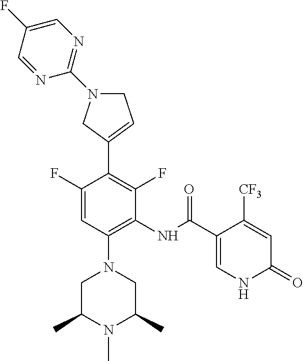 [SMILES: C[C@@H]1CN(c2cc(F)c(C3=CCN(c4ncc(F)cn4)C3)c(F)c2NC(=O)c2c[nH]c(=O)cc2C(F)(F)F)C[C@H](C)N1C] | N-(2,4-difluoro-3-(1- (5-fluoropyrimidin-2- yl)-2,5-dihydro-1H- pyrrol-3-yl)-6- ((3S,5R)-3,4,5- trimethylpiperazin-1- yl)phenyl)-6-oxo-4- (trifluoromethyl)-1,6- dihydropyridine-3- carboxamide | 19.4720 | 0.00071 | 0.0376 |
| 148 | 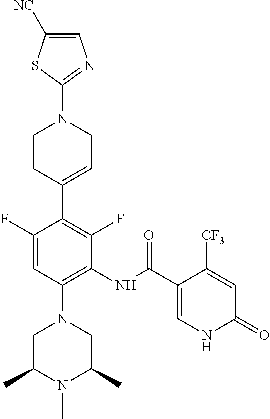 [SMILES: C[C@@H]1CN(c2cc(F)c(C3=CCN(c4ncc(C#N)s4)CC3)c(F)c2NC(=O)c2c[nH]c(=O)cc2C(F)(F)F)C[C@H](C)N1C] | N-(3-(1-(5- cyanothiazol-2-yl)- 1,2,3,6- tetrahydropyridin-4- yl)-2,4-difluoro-6- ((3S,5R)-3,4,5- trimethylpiperazin-1- yl)phenyl)-6-oxo-4- (trifluoromethyl)-1,6- dihydropyridine-3- carboxamide | 12.8700 | 0.00209 | 0.0749 |
| 149 | 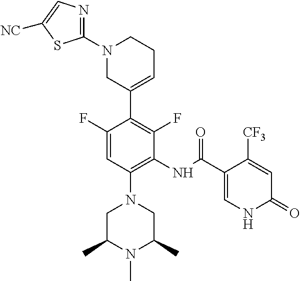 [SMILES: C[C@@H]1CN(c2cc(F)c(C3=CCCN(c4ncc(C#N)s4)C3)c(F)c2NC(=O)c2c[nH]c(=O)cc2C(F)(F)F)C[C@H](C)N1C] | N-(3-(1-(5- cyanothiazol-2-yl)- 1,2,5,6- tetrahydropyridin-3- yl)-2,4-difluoro-6- ((3S,5R)-3,4,5- trimethylpiperazin-1- yl)phenyl)-6-oxo-4- (trifluoromethyl)-1,6- dihydropyridine-3- carboxamide | 14.5820 | 0.0018 | 0.106 |
| 150 | 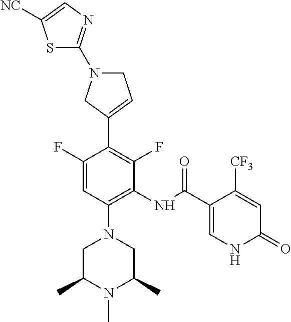 [SMILES: C[C@@H]1CN(c2cc(F)c(C3=CCN(c4ncc(C#N)s4)C3)c(F)c2NC(=O)c2c[nH]c(=O)cc2C(F)(F)F)C[C@H](C)N1C] | N-(3-(1-(5- cyanothiazol-2-yl)-2,5- dihydro-1H-pyrrol-3- yl)-2,4-difluoro-6- ((3S,5R)-3,4,5- trimethylpiperazin-1- yl)phenyl)-6-oxo-4- (trifluoromethyl)-1,6- dihydropyridine-3- carboxamide | 12.9150 | 0.00053 | 0.0484 |
| 151 | 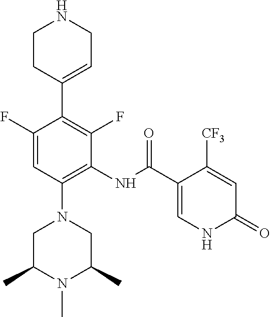 [SMILES: C[C@@H]1CN(c2cc(F)c(C3=CCNCC3)c(F)c2NC(=O)c2c[nH]c(=O)cc2C(F)(F)F)C[C@H](C)N1C] | N-(2,4-difluoro-3- (1,2,3,6- tetrahydropyridin-4- yl)-6-((3S,5R)-3,4,5- trimethylpiperazin-1- yl)phenyl)-6-oxo-4- (trifluoromethyl)-1,6- dihydropyridine-3- carboxamide | ND | ND | ND |
| 152 | 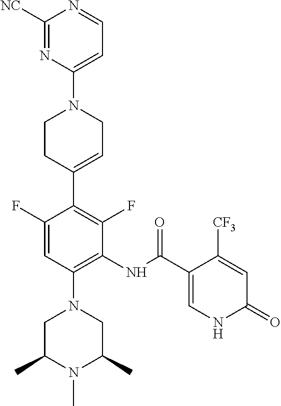 [SMILES: C[C@@H]1CN(c2cc(F)c(C3=CCN(c4ccnc(C#N)n4)CC3)c(F)c2NC(=O)c2c[nH]c(=O)cc2C(F)(F)F)C[C@H](C)N1C] | N-(3-(1-(2- cyanopyrimidin-4-yl)- 1,2,3,6- tetrahydropyridin-4- yl)-2,4-difluoro-6- ((3S,5R)-3,4,5- trimethylpiperazin-1- yl)phenyl)-6-oxo-4- (trifluoromethyl)-1,6- dihydropyridine-3- carboxamide | 15.2550 | 0.00068 | 0.024 |
| 153 | 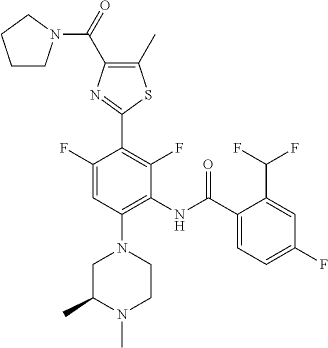 [SMILES: Cc1sc(-c2c(F)cc(N3CCN(C)[C@@H](C)C3)c(NC(=O)c3ccc(F)cc3C(F)F)c2F)nc1C(=O)N1CCCC1] | (S)-2-(difluoromethyl)- N-(6-(3,4- dimethylpiperazin-1- yl)-2,4-difluoro-3-(5- methyl-4-(pyrrolidine- 1-carbonyl)thiazol-2- yl)phenyl)-4- fluorobenzamide | 5.0000 | 0.00118 | 0.0701 |
| 154 | 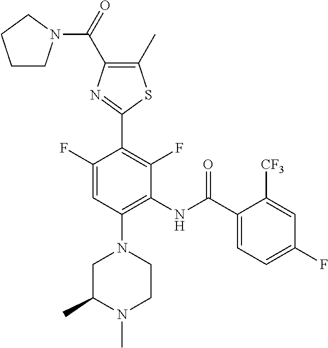 [SMILES: Cc1sc(-c2c(F)cc(N3CCN(C)[C@@H](C)C3)c(NC(=O)c3ccc(F)cc3C(F)(F)F)c2F)nc1C(=O)N1CCCC1] | (S)-N-(6-(3,4- dimethylpiperazin-1- yl)-2,4-difluoro-3-(5- methyl-4-(pyrrolidine- 1-carbonyl)thiazol-2- yl)phenyl)-4-fluoro-2- (trifluoromethyl) benzamide | 11.3610 | 0.00064 | 0.092 |
| 155 | 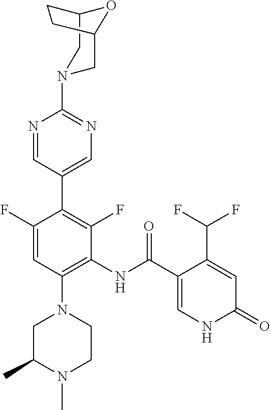 [SMILES: C[C@H]1CN(c2cc(F)c(-c3cnc(N4CC5CCC(C4)O5)nc3)c(F)c2NC(=O)c2c[nH]c(=O)cc2C(F)F)CCN1C] | (S)-N-(3-(2-(8-oxa-3- azabicyclo[3.2.1] octan-3-yl)pyrimidin- 5-yl)-6-(3,4- dimethylpiperazin-1- yl)-2,4- difluorophenyl)-4- (difluoromethyl)-6- oxo-1,6- dihydropyridine-3- carboxamide | 11.2070 | 0.00121 | 0.074 |
| 156 | 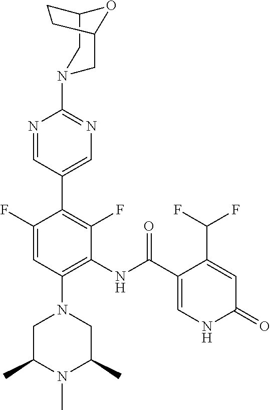 [SMILES: C[C@@H]1CN(c2cc(F)c(-c3cnc(N4CC5CCC(C4)O5)nc3)c(F)c2NC(=O)c2c[nH]c(=O)cc2C(F)F)C[C@H](C)N1C] | N-(3-(2-(8-oxa-3- azabicyclo[3.2.1] octan-3-yl)pyrimidin- 5-yl)-2,4-difluoro-6- ((3S,5R)-3,4,5- trimethylpiperazin-1- yl)phenyl)-4- (difluoromethyl)-6- oxo-1,6- dihydropyridine-3- carboxamide | 15.4210 | 0.00196 | 0.0812 |
| 157 | 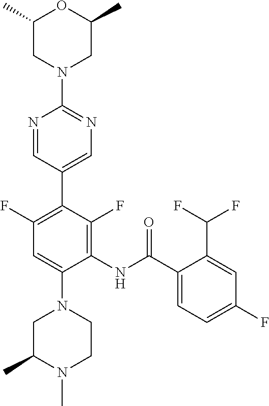 [SMILES: C[C@H]1CN(c2ncc(-c3c(F)cc(N4CCN(C)[C@@H](C)C4)c(NC(=O)c4ccc(F)cc4C(F)F)c3F)cn2)C[C@H](C)O1] | 2-(Difluoromethyl)- N-(3-(2-((2S,6S)-2,6- dimethylmorpholino) pyrimidin-5-yl)-6- ((S)-3,4- dimethylpiperazin-1- yl)-2,4- difluorophenyl)-4- fluorobenzamide | 16.0820 | 0.00022 | 0.0656 |
| 158 | 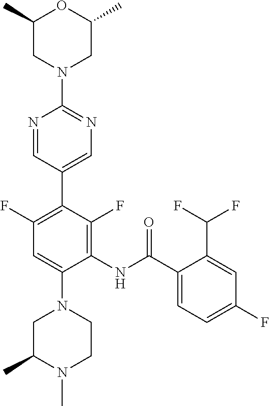 [SMILES: C[C@@H]1CN(c2ncc(-c3c(F)cc(N4CCN(C)[C@@H](C)C4)c(NC(=O)c4ccc(F)cc4C(F)F)c3F)cn2)C[C@@H](C)O1] | 2-(Difluoromethyl)- N-(3-(2-((2R,6R)-2,6- dimethylmorpholino) pyrimidin-5-yl)-6- ((S)-3,4- dimethylpiperazin-1- yl)-2,4- difluorophenyl)-4- fluorobenzamide | 13.9570 | 0.00043 | 0.0883 |
| 159 | 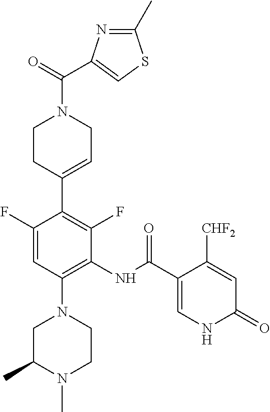 [SMILES: Cc1nc(C(=O)N2CC=C(c3c(F)cc(N4CCN(C)[C@@H](C)C4)c(NC(=O)c4c[nH]c(=O)cc4C(F)F)c3F)CC2)cs1] | (S)-4- (difluoromethyl)-N- (6-(3,4- dimethylpiperazin-1- yl)-2,4-difluoro-3-(1- (2-methylthiazole-4- carbonyl)-1,2,3,6- tetrahydropyridin-4- yl)phenyl)-6-oxo-1,6- dihydropyridine-3- carboxamide | 13.8940 | 0.0022 | 0.44 |
| 160 | 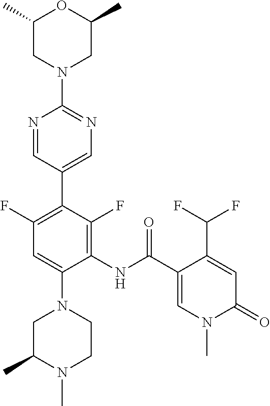 [SMILES: C[C@H]1CN(c2ncc(-c3c(F)cc(N4CCN(C)[C@@H](C)C4)c(NC(=O)c4cn(C)c(=O)cc4C(F)F)c3F)cn2)C[C@H](C)O1] | 4-(Difluoromethyl)- N-(3-(2-((2S,6S)-2,6- dimethylmorpholino) pyrimidin-5-yl)-6- ((S)-3,4- dimethylpiperazin-1- yl)-2,4- difluorophenyl)-1- methyl-6-oxo-1,6- dihydropyridine-3- carboxamide | 14.0310 | 0.00136 | 0.254 |
| 161 | 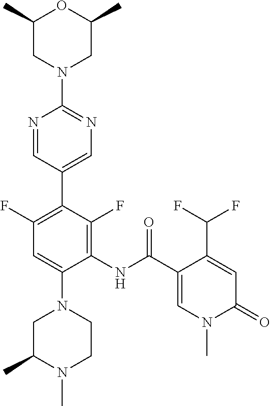 [SMILES: C[C@@H]1CN(c2ncc(-c3c(F)cc(N4CCN(C)[C@@H](C)C4)c(NC(=O)c4cn(C)c(=O)cc4C(F)F)c3F)cn2)C[C@H](C)O1] | 4-(Difluoromethyl)- N-(3-(2-((2S,6R)-2,6- dimethylmorpholino) pyrimidin-5-yl)-6- ((S)-3,4- dimethylpiperazin-1- yl)-2,4- difluorophenyl)-1- methyl-6-oxo-1,6- dihydropyridine-3- carboxamide | 11.8840 | 0.0011 | 0.0793 |
| 162 | 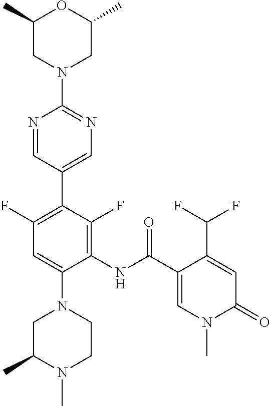 [SMILES: C[C@@H]1CN(c2ncc(-c3c(F)cc(N4CCN(C)[C@@H](C)C4)c(NC(=O)c4cn(C)c(=O)cc4C(F)F)c3F)cn2)C[C@@H](C)O1] | 4-(Difluoromethyl)- N-(3-(2-((2R,6R)-2,6- dimethylmorpholino) pyrimidin-5-yl)-6- ((S)-3,4- dimethylpiperazin-1- yl)-2,4- difluorophenyl)-1- methyl-6-oxo-1,6- dihydropyridine-3- carboxamide | 14.0650 | 0.00125 | 0.21 |
| 163 | 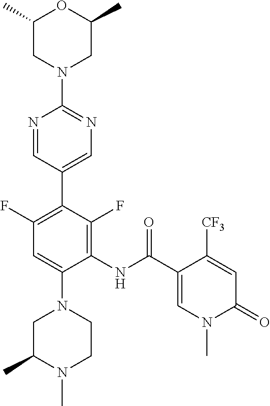 [SMILES: C[C@H]1CN(c2ncc(-c3c(F)cc(N4CCN(C)[C@@H](C)C4)c(NC(=O)c4cn(C)c(=O)cc4C(F)(F)F)c3F)cn2)C[C@H](C)O1] | N-(3-(2-((2S,6S)-2,6- dimethylmorpholino) pyrimidin-5-yl)-6- ((S)-3,4- dimethylpiperazin-1- yl)-2,4- difluorophenyl)-1- methyl-6-oxo-4- (trifluoromethyl)-1,6- dihydropyridine-3- carboxamide | 15.2730 | 0.0011 | 0.0834 |
| 164 | 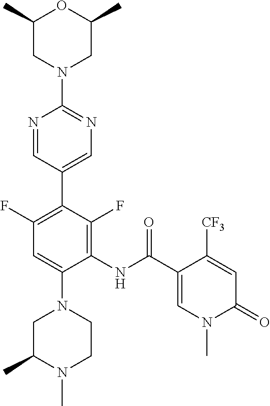 [SMILES: C[C@@H]1CN(c2ncc(-c3c(F)cc(N4CCN(C)[C@@H](C)C4)c(NC(=O)c4cn(C)c(=O)cc4C(F)(F)F)c3F)cn2)C[C@H](C)O1] | N-(3-(2-((2S,6R)-2,6- dimethylmorpholino) pyrimidin-5-yl)-6- ((S)-3,4- dimethylpiperazin-1- yl)-2,4- difluorophenyl)-1- methyl-6-oxo-4- (trifluoromethyl)-1,6- dihydropyridine-3- carboxamide | 13.2550 | 0.00116 | 0.0719 |
| 165 | 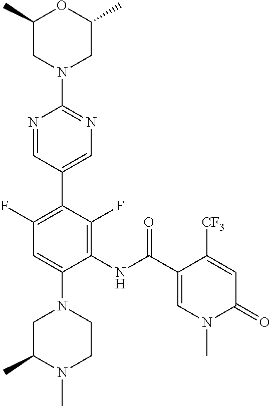 [SMILES: C[C@@H]1CN(c2ncc(-c3c(F)cc(N4CCN(C)[C@@H](C)C4)c(NC(=O)c4cn(C)c(=O)cc4C(F)(F)F)c3F)cn2)C[C@@H](C)O1] | N-(3-(2-((2R,6R)-2,6- dimethylmorpholino) pyrimidin-5-yl)-6- ((S)-3,4- dimethylpiperazin-1- yl)-2,4- difluorophenyl)-1- methyl-6-oxo-4- (trifluoromethyl)-1,6- dihydropyridine-3- carboxamide | 13.6080 | 0.00082 | 0.169 |
| 166 | 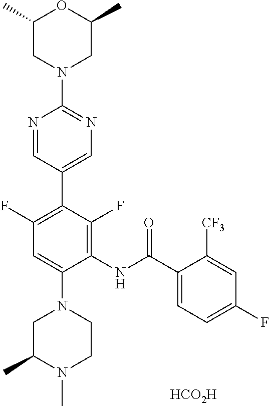 [SMILES: C[C@H]1CN(c2ncc(-c3c(F)cc(N4CCN(C)[C@@H](C)C4)c(NC(=O)c4ccc(F)cc4C(F)(F)F)c3F)cn2)C[C@H](C)O1 ; "HCO2H"] | N-(3-(2-((2S,6S)-2,6- dimethylmorpholino) pyrimidin-5-yl)-6- ((S)-3,4- dimethylpiperazin-1- yl)-2,4- difluorophenyl)-4- fluoro-2- (trifluoromethyl) benzamide formic acid salt | 11.6970 | 0.00073 | 0.0636 |
| 167 | 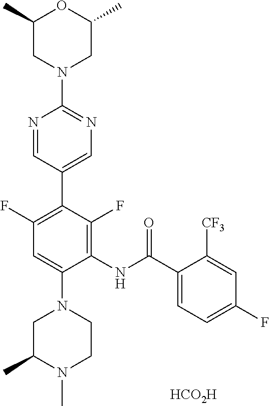 [SMILES: C[C@@H]1CN(c2ncc(-c3c(F)cc(N4CCN(C)[C@@H](C)C4)c(NC(=O)c4ccc(F)cc4C(F)(F)F)c3F)cn2)C[C@@H](C)O1 ; "HCO2H"] | N-(3-(2-((2R,6R)-2,6- dimethylmorpholino) pyrimidin-5-yl)-6- ((S)-3,4- dimethylpiperazin-1- yl)-2,4- difluorophenyl)-4- fluoro-2- (trifluoromethyl) benzamide formic acid salt | 11.4960 | 0.00066 | 0.0755 |
| 168 | 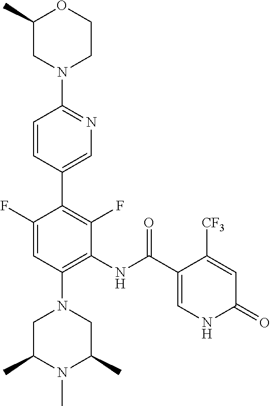 [SMILES: C[C@@H]1CN(c2ccc(-c3c(F)cc(N4C[C@@H](C)N(C)[C@@H](C)C4)c(NC(=O)c4c[nH]c(=O)cc4C(F)(F)F)c3F)cn2)CCO1] | N-(2,4-difluoro-3-(6- ((R)-2- methylmorpholino) pyridin-3-yl)-6- ((3S,5R)-3,4,5- trimethylpiperazin-1- yl)phenyl)-6-oxo-4- (trifluoromethyl)-1,6- dihydropyridine-3- carboxamide | 16.6140 | 0.00087 | 0.0368 |
| 169 | 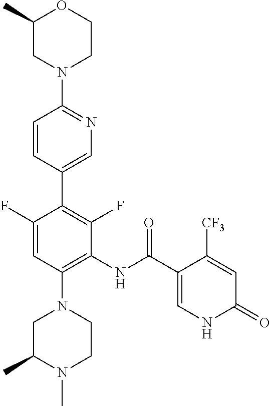 [SMILES: C[C@@H]1CN(c2ccc(-c3c(F)cc(N4CCN(C)[C@@H](C)C4)c(NC(=O)c4c[nH]c(=O)cc4C(F)(F)F)c3F)cn2)CCO1] | N-(6-((S)-3,4- dimethylpiperazin-1- yl)-2,4-difluoro-3-(6- ((R)-2- methylmorpholino) pyridin-3-yl)phenyl)- 6-oxo-4- (trifluoromethyl)-1,6- dihydropyridine-3- carboxamide | 16.7370 | 0.00099 | 0.0514 |
| 170 | 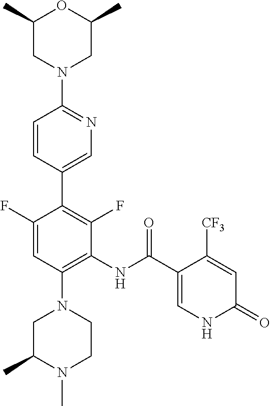 [SMILES: C[C@@H]1CN(c2ccc(-c3c(F)cc(N4CCN(C)[C@@H](C)C4)c(NC(=O)c4c[nH]c(=O)cc4C(F)(F)F)c3F)cn2)C[C@H](C)O1] | N-(3-(6-((2S,6R)-2,6- dimethylmorpholino) pyridin-3-yl)-6-((S)- 3,4- dimethylpiperazin-1- yl)-2,4- difluorophenyl)-6- oxo-4- (trifluoromethyl)-1,6- dihydropyridine-3- carboxamide | 15.6260 | 0.00083 | 0.0323 |
| 171 | 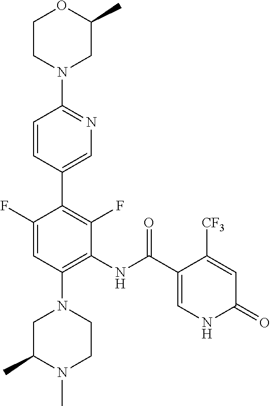 [SMILES: C[C@H]1CN(c2ccc(-c3c(F)cc(N4CCN(C)[C@@H](C)C4)c(NC(=O)c4c[nH]c(=O)cc4C(F)(F)F)c3F)cn2)CCO1] | N-(6-((S)-3,4- dimethylpiperazin-1- yl)-2,4-difluoro-3-(6- ((S)-2- methylmorpholino) pyridin-3-yl)phenyl)- 6-oxo-4- (trifluoromethyl)-1,6- dihydropyridine-3- carboxamide | 16.1330 | 0.00049 | 0.0519 |
| 172 | 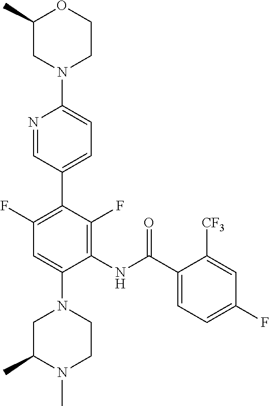 [SMILES: C[C@@H]1CN(c2ccc(-c3c(F)cc(N4CCN(C)[C@@H](C)C4)c(NC(=O)c4ccc(F)cc4C(F)(F)F)c3F)cn2)CCO1] | N-(6-((S)-3,4- dimethylpiperazin-1- yl)-2,4-difluoro-3-(6- ((R)-2- methylmorpholino) pyridin-3-yl)phenyl)- 4-fluoro-2- (trifluoromethyl) benzamide | 18.8560 | 0.00102 | 0.124 |
| 173 | 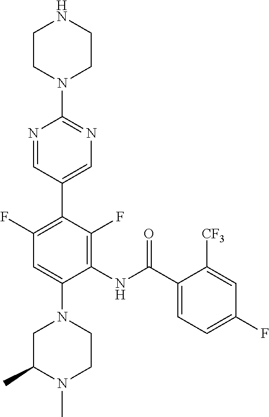 [SMILES: C[C@H]1CN(c2cc(F)c(-c3cnc(N4CCNCC4)nc3)c(F)c2NC(=O)c2ccc(F)cc2C(F)(F)F)CCN1C] | (S)-N-(6-(3,4- dimethylpiperazin-1- yl)-2,4-difluoro-3-(2- (piperazin-1- yl)pyrimidin-5- yl)phenyl)-4-fluoro-2- (trifluoromethyl) benzamide | 16.0240 | 0.00135 | 0.0244 |
| 174 | 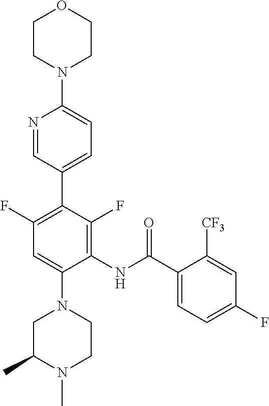 [SMILES: C[C@H]1CN(c2cc(F)c(-c3ccc(N4CCOCC4)nc3)c(F)c2NC(=O)c2ccc(F)cc2C(F)(F)F)CCN1C] | (S)-N-(6-(3,4- dimethylpiperazin-1- yl)-2,4-difluoro-3-(6- morpholinopyridin-3- yl)phenyk)-4-fluoro-2- (trifluoromethyl) benzamide | 19.5370 | 0.00171 | 0.191 |
| 175 | 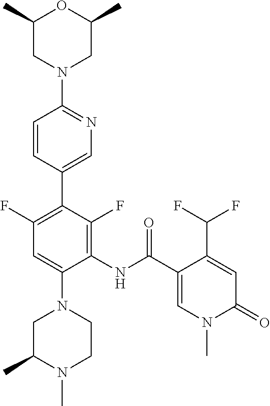 [SMILES: C[C@@H]1CN(c2ccc(-c3c(F)cc(N4CCN(C)[C@@H](C)C4)c(NC(=O)c4cn(C)c(=O)cc4C(F)F)c3F)cn2)C[C@H](C)O1] | 4-(Difluoromethyl)- N-(3-(6-((2S,6R)-2,6- dimethylmorpholino) pyridin-3-yl)-6-((S)- 3,4- dimethylpiperazin-1- yl)-2,4- difluorophenyl)-1- methyl-6-oxo-1,6- dihydropyridine-3- carboxamide | 24.6180 | 0.00157 | 0.255 |
| 176 | 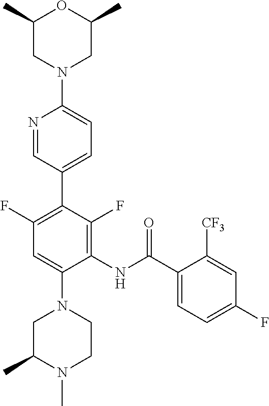 [SMILES: C[C@@H]1CN(c2ccc(-c3c(F)cc(N4CCN(C)[C@@H](C)C4)c(NC(=O)c4ccc(F)cc4C(F)(F)F)c3F)cn2)C[C@H](C)O1] | N-(3-(6-((2S,6R)-2,6- dimethylmorpholino) pyridin-3-yl)-6-((S)- 3,4- dimethylpiperazin-1- yl)-2,4- difluorophenyl)-4- fluoro-2- (trifluoromethyl) benzamide | 19.8210 | 0.00146 | 0.139 |
| 177 | 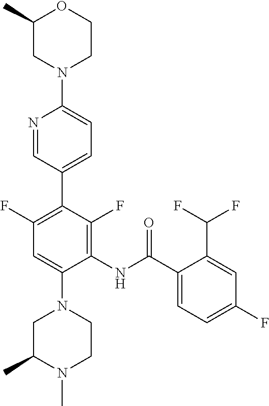 [SMILES: C[C@@H]1CN(c2ccc(-c3c(F)cc(N4CCN(C)[C@@H](C)C4)c(NC(=O)c4ccc(F)cc4C(F)F)c3F)cn2)CCO1] | 2-(Difluoromethyl)- N-(6-((S)-3,4- dimethylpiperazin-1- yl)-2,4-difluoro-3-(6- ((R)-2- methylmorpholino) pyridin-3-yl)phenyl)-4- fluorobenzamide | 22.9720 | 0.00172 | 0.101 |
| 178 | 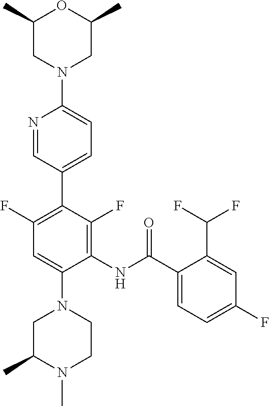 [SMILES: C[C@@H]1CN(c2ccc(-c3c(F)cc(N4CCN(C)[C@@H](C)C4)c(NC(=O)c4ccc(F)cc4C(F)F)c3F)cn2)C[C@H](C)O1] | 2-(Difluoromethyl)- N-(3-(6-((2S,6R)-2,6- dimethylmorpholino) pyridin-3-yl)-6-((S)- 3,4- dimethylpiperazin-1- yl)-2,4- difluorophenyl)-4- fluorobenzamide | 19.7120 | 0.00142 | 0.189 |
| 179 | 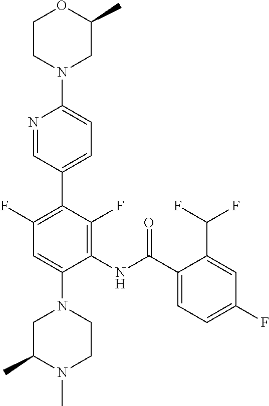 [SMILES: C[C@H]1CN(c2ccc(-c3c(F)cc(N4CCN(C)[C@@H](C)C4)c(NC(=O)c4ccc(F)cc4C(F)F)c3F)cn2)CCO1] | 2-(Difluoromethyl)- N-(6-((S)-3,4- dimethylpiperazin-1- yl)-2,4-difluoro-3-(6- ((S)-2- methylmorpholino) pyridin-3-yl)phenyl)-4- fluorobenzamide | 20.0820 | 0.00159 | 0.141 |
| 180 | 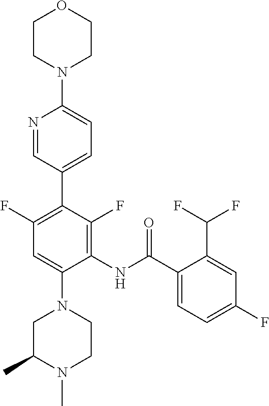 [SMILES: C[C@H]1CN(c2cc(F)c(-c3ccc(N4CCOCC4)nc3)c(F)c2NC(=O)c2ccc(F)cc2C(F)F)CCN1C] | (S)-2- (Difluoromethyl)-N- (6-(3,4- dimethylpiperazin-1- yl)-2,4-difluoro-3-(6- morpholinopyridin-3- yl)phenyl)-4- fluorobenzamide | 22.3770 | 0.00251 | 0.198 |
| 181 | 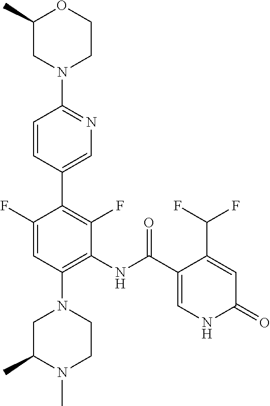 [SMILES: C[C@@H]1CN(c2ccc(-c3c(F)cc(N4CCN(C)[C@@H](C)C4)c(NC(=O)c4c[nH]c(=O)cc4C(F)F)c3F)cn2)CCO1] | 4-(Difluoromethyl)- N-(6-((S)-3,4- dimethylpiperazin-1- yl)-2,4-difluoro-3-(6- ((R)-2- methylmorpholino) pyridin-3-yl)phenyl)- 6-oxo-1,6- dihydropyridine-3- carboxamide | 24.4610 | 0.00064 | 0.0299 |
| 182 | 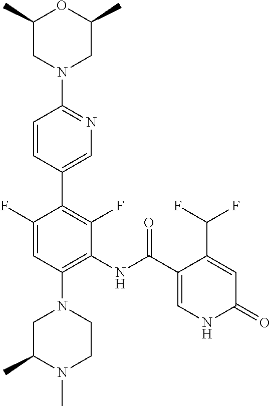 [SMILES: C[C@@H]1CN(c2ccc(-c3c(F)cc(N4CCN(C)[C@@H](C)C4)c(NC(=O)c4c[nH]c(=O)cc4C(F)F)c3F)cn2)C[C@H](C)O1] | 4-(Difluoromethyl)- N-(3-(6-((2S,6R)-2,6- dimethylmorpholino) pyridin-3-yl)-6-((S)- 3,4- dimethylpiperazin-1- yl)-2,4- difluorophenyl)-6- oxo-1,6- dihydropyridine-3- carboxamide | 25.4350 | 0.00115 | 0.031 |
| 183 | 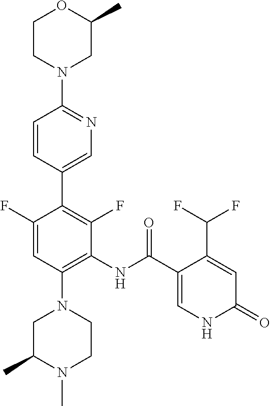 [SMILES: C[C@H]1CN(c2ccc(-c3c(F)cc(N4CCN(C)[C@@H](C)C4)c(NC(=O)c4c[nH]c(=O)cc4C(F)F)c3F)cn2)CCO1] | 4-(Difluoromethyl)- N-(6-((S)-3,4- dimethylpiperazin-1- yl)-2,4-difluoro-3-(6- ((S)-2- methylmorpholino) pyridin-3-yl)phenyl)- 6-oxo-1,6- dihydropyridine-3- carboxamide | 22.7370 | 0.00134 | 0.0244 |
| 184 | 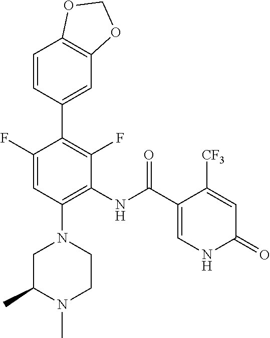 [SMILES: C[C@H]1CN(c2cc(F)c(-c3ccc4c(c3)OCO4)c(F)c2NC(=O)c2c[nH]c(=O)cc2C(F)(F)F)CCN1C] | (S)-N-(3- (benzo[d][1,3]dioxol- 5-yl)-6-(3,4- dimethylpiperazin-1- yl)-2,4- difluorophenyl)-6- oxo-4- (trifluoromethyl)-1,6- dihydropyridine-3- carboxamide | 25.5030 | 0.00117 | 0.0903 |
| 185 | 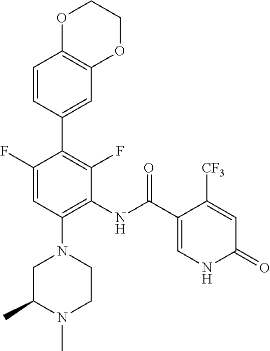 [SMILES: C[C@H]1CN(c2cc(F)c(-c3ccc4c(c3)OCCO4)c(F)c2NC(=O)c2c[nH]c(=O)cc2C(F)(F)F)CCN1C] | (S)-N-(3-(2,3- dihydrobenzo[b][1,4] dioxin-6-yl)-6-(3,4- dimethylpiperazin-1- yl)-2,4- difluorophenyl)-6- oxo-4- (trifluoromethyl)-1,6- dihydropyridine-3- carboxamide | 26.4590 | 0.00129 | 0.17 |
| 186 | 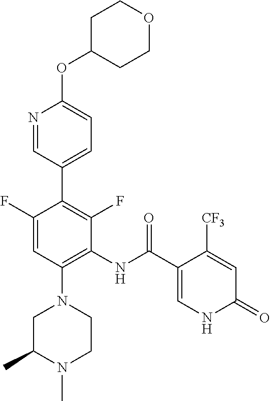 [SMILES: C[C@H]1CN(c2cc(F)c(-c3ccc(OC4CCOCC4)nc3)c(F)c2NC(=O)c2c[nH]c(=O)cc2C(F)(F)F)CCN1C] | (S)-N-(6-(3,4- dimethylpiperazin-1- yl)-2,4-difluoro-3-(6- ((tetrahydro-2H- pyran-4- yl)oxy)pyridin-3- yl)phenyl)-6-oxo-4- (trifluoromethyl)-1,6- dihydropyridine-3- carboxamide | 26.8930 | 0.0008 | 0.0399 |
| 187 | 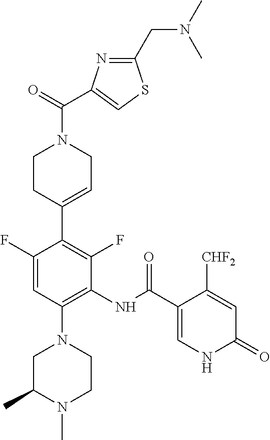 [SMILES: C[C@H]1CN(c2cc(F)c(C3=CCN(C(=O)c4csc(CN(C)C)n4)CC3)c(F)c2NC(=O)c2c[nH]c(=O)cc2C(F)F)CCN1C] | (S)-4- (difluoromethyl)-N- (3-(1-(2- ((dimethylamino) methyl)thiazole-4- carbonyl)-1,2,3,6- tetrahydropyridin-4- yl)-6-(3,4- dimethylpiperazin-1- yl)-2,4- difluorophenyl)-6- oxo-1,6- dihydropyridine-3- carboxamide | 14.6890 | 0.00295 | 0.913 |
| 188 | 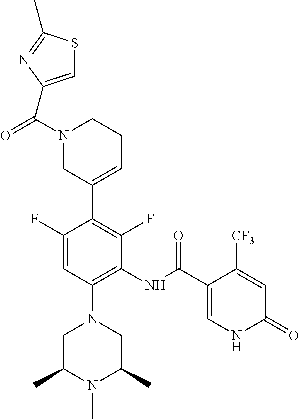 [SMILES: Cc1nc(C(=O)N2CCC=C(c3c(F)cc(N4C[C@@H](C)N(C)[C@@H](C)C4)c(NC(=O)c4c[nH]c(=O)cc4C(F)(F)F)c3F)C2)cs1] | N-(2,4-difluoro-3-(1- (2-methylthiazole-4- carbonyl)-1,2,5,6- tetrahydropyridin-3- yl)-6-((3S,5R)-3,4,5- trimethylpiperazin-1- yl)phenyl)-6-oxo-4- (trifluoromethyl)-1,6- dihydropyridine-3- carboxamide | 13.0960 | 0.00254 | 0.468 |
| 189 | 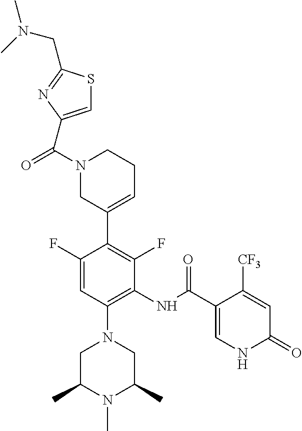 [SMILES: C[C@@H]1CN(c2cc(F)c(C3=CCCN(C(=O)c4csc(CN(C)C)n4)C3)c(F)c2NC(=O)c2c[nH]c(=O)cc2C(F)(F)F)C[C@H](C)N1C] | N-(3-(1-(2- ((dimethylamino) methyl)thiazole-4- carbonyl)-1,2,5,6- tetrahydropyridin-3- yl)-2,4-difluoro-6- ((3S,5R)-3,4,5- trimethylpiperazin-1- yl)phenyl)-6-oxo-4- (trifluoromethyl)-1,6- dihydropyridine-3- carboxamide | 12.6560 | 0.00151 | 0.487 |
| 190 | 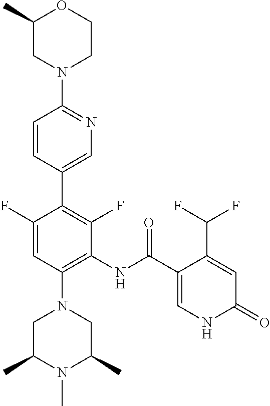 [SMILES: C[C@@H]1CN(c2ccc(-c3c(F)cc(N4C[C@@H](C)N(C)[C@@H](C)C4)c(NC(=O)c4c[nH]c(=O)cc4C(F)F)c3F)cn2)CCO1] | N-(2,4-difluoro-3-(6- ((R)-2- methylmorpholino) pyridin-3-yl)-6- ((3S,5R)-3,4,5- trimethylpiperazin-1- yl)phenyl)-4- (difluoromethyl)-6- oxo-1,6- dihydropyridine-3- carboxamide | 12.8070 | 0.00096 | 0.0486 |
| 191 | 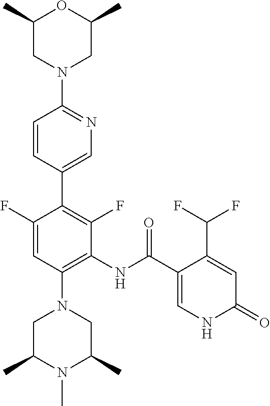 [SMILES: C[C@@H]1CN(c2ccc(-c3c(F)cc(N4C[C@@H](C)N(C)[C@@H](C)C4)c(NC(=O)c4c[nH]c(=O)cc4C(F)F)c3F)cn2)C[C@H](C)O1] | 4-(Difluoromethyl)- N-(3-(6-((2S,6R)-2,6- dimethylmorpholino) pyridin-3-yl)-2,4- difluoro-6-((3S,5R)- 3,4,5- trimethylpiperazin-1- yl)phenyl)-6-oxo-1,6- dihydropyridine-3- carboxamide | 10.6850 | 0.00107 | 0.0408 |
| 192 | 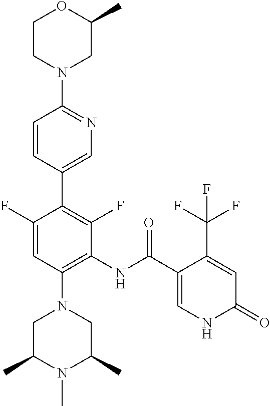 [SMILES: C[C@@H]1CN(c2cc(F)c(-c3ccc(N4CCO[C@@H](C)C4)nc3)c(F)c2NC(=O)c2c[nH]c(=O)cc2C(F)(F)F)C[C@H](C)N1C] | N-(2,4-difluoro-3-(6- ((S)-2- methylmorpholino) pyridin-3-yl)-6- ((3S,5R)-3,4,5- trimethylpiperazin-1- yl)phenyl)-6-oxo-4- (trifluoromethyl)-1,6- dihydropyridine-3- carboxamide | 16.416 | 0.00097 | 0.0701 |

TABLE 2
Representative examples of the effect of DiFluoro-substitution

| | Assay | | |
|---|---|---|---|
| Structure | Results | Compound | Assay Results |
| 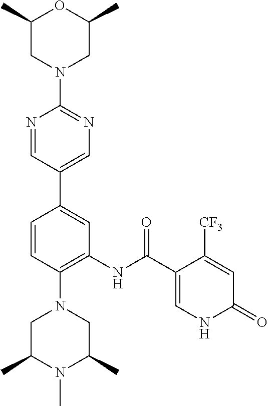 [SMILES: C[C@@H]1CN(c2ncc(-c3ccc(N4C[C@@H](C)N(C)[C@@H](C)C4)c(NC(=O)c4c[nH]c(=O)cc4C(F)(F)F)c3)cn2)C[C@H](C)O1] | K_{D} (SPR) = 0.0029 μM IC₅₀ (HMT) = NT | 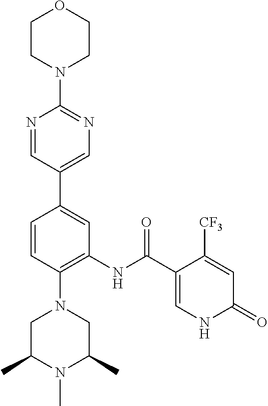 [SMILES: C[C@@H]1CN(c2ccc(-c3cnc(N4CCOCC4)nc3)cc2NC(=O)c2c[nH]c(=O)cc2C(F)(F)F)C[C@H](C)N1C] | K_{D} (SPR) = 0.035 μM IC₅₀ (HMT) = 1.573 μM IC₅₀ (MV4-11) = NT |
| 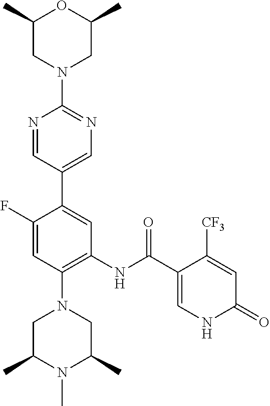 [SMILES: C[C@@H]1CN(c2ncc(-c3cc(NC(=O)c4c[nH]c(=O)cc4C(F)(F)F)c(N4C[C@@H](C)N(C)[C@@H](C)C4)cc3F)cn2)C[C@H](C)O1] | K_{D} (SPR) = 0.0004 μM IC₅₀ (HMT) = 0.150 | 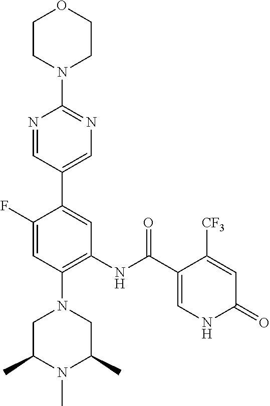 [SMILES: C[C@@H]1CN(c2cc(F)c(-c3cnc(N4CCOCC4)nc3)cc2NC(=O)c2c[nH]c(=O)cc2C(F)(F)F)C[C@H](C)N1C] | K_{D} (SPR) = 0.0003 μM IC₅₀ (HMT) = 0.036 μM IC₅₀ (MV4-11) = 0.025 μM |
| 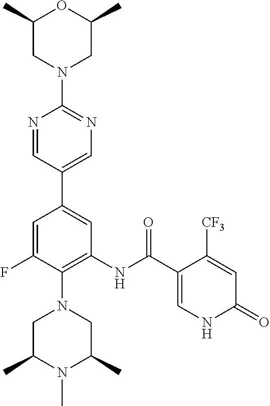 [SMILES: C[C@@H]1CN(c2ncc(-c3cc(F)c(N4C[C@@H](C)N(C)[C@@H](C)C4)c(NC(=O)c4c[nH]c(=O)cc4C(F)(F)F)c3)cn2)C[C@H](C)O1] | K_{D} (SPR) = 0.048 μM IC₅₀ (HMT) = NT μM | 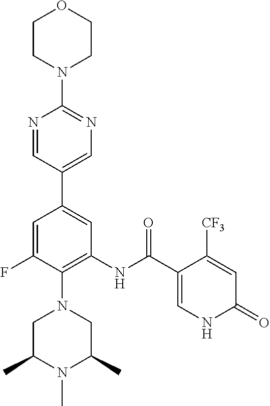 [SMILES: C[C@@H]1CN(c2c(F)cc(-c3cnc(N4CCOCC4)nc3)cc2NC(=O)c2c[nH]c(=O)cc2C(F)(F)F)C[C@H](C)N1C] | K_{D} (SPR) = 0.077 μM IC₅₀ (HMT) = NT IC₅₀ (MV4-11) = 3.539 μM |
| 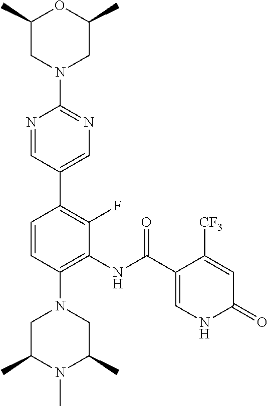 [SMILES: C[C@@H]1CN(c2ncc(-c3ccc(N4C[C@@H](C)N(C)[C@@H](C)C4)c(NC(=O)c4c[nH]c(=O)cc4C(F)(F)F)c3F)cn2)C[C@H](C)O1] | K_{D} (SPR) = 0.0018 μM IC₅₀ (HMT) = NT μM | 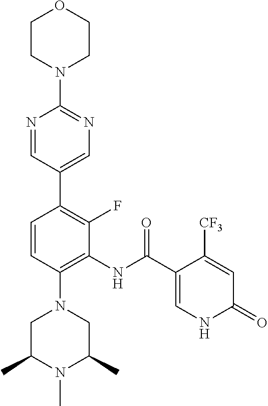 [SMILES: C[C@@H]1CN(c2ccc(-c3cnc(N4CCOCC4)nc3)c(F)c2NC(=O)c2c[nH]c(=O)cc2C(F)(F)F)C[C@H](C)N1C] | K_{D} (SPR) = 0.0011 μM IC₅₀ (HMT) = 0.154 μM IC₅₀ (MV4-11) = NT |
| 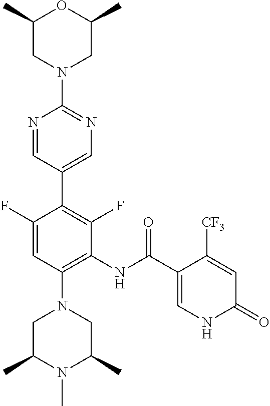 [SMILES: C[C@@H]1CN(c2ncc(-c3c(F)cc(N4C[C@@H](C)N(C)[C@@H](C)C4)c(NC(=O)c4c[nH]c(=O)cc4C(F)(F)F)c3F)cn2)C[C@H](C)O1] | K_{D} (SPR) = 0.0018 μM IC₅₀ (HMT) = 0.041 μM IC₅₀ (MV4-11) = 0.031 μM | 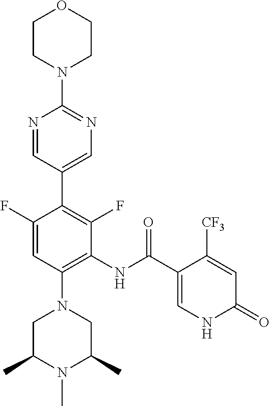 [SMILES: C[C@@H]1CN(c2cc(F)c(-c3cnc(N4CCOCC4)nc3)c(F)c2NC(=O)c2c[nH]c(=O)cc2C(F)(F)F)C[C@H](C)N1C] | K_{D} (SPR) = 0.0037 μM IC₅₀ (HMT) = 0.063 μM IC₅₀ (MV4-11) = 0.030 μM |

NT: Not tested

1. A compound of Formula (I) or a pharmaceutically acceptable salt and/or solvate thereof: 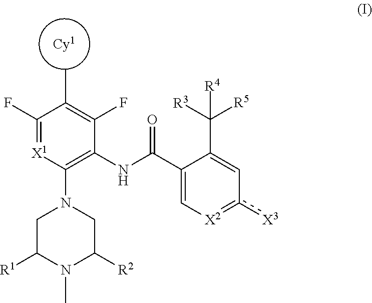
wherein:
R¹ and R² are independently selected from H and CH₃;
R³, R⁴ and R⁵ are independently selected from H and F, provided that at least two of R³, R⁴ and R⁵ are F; [symbol]
is a single or double bond, provided that one [symbol] is a single bond and the other [symbol] is a double bond;
X¹ is selected from CH and N;
X² is NH or NCH₃ when the adjacent [symbol] is a single bond or X² is CH when the adjacent [symbol] is a double bond;
X³ is F when the adjacent [symbol] is a single bond or X³ is O when [symbol] is a double bond;
Cy¹ is a substituted phenyl, substituted 5- or 6-membered heteroaromatic monocyclic ring, substituted 5- or 6-membered heterocycloalkyl monocyclic ring, an optionally substituted 9- or 10-membered aromatic bicyclic ring, optionally substituted 9- or 10-membered heteroaromatic bicyclic ring or optionally substituted 9- or 10-membered heterocycloalkyl bicyclic ring;
when Cy¹ is a monocyclic ring Cy¹ is substituted with at least one Cy² and optionally one or two F, CN or C₁₋₄alkyl; or Cy¹ is substituted with N(C₁₋₁₀alkyl)(C₁₋₁₀alkyl), OCH₂C₃₋₆cycloalkyl, OC₃₋₆cycloalkyl, OC₄₋₆hetereocycloalkyl, OC₅₋₆hetereoaryl, Ophenyl, OCH₂C₄₋₆hetereocycloalkyl, OCH₂C₅₋₆hetereoaryl, C₃₋₆cycloalkyl, phenyl, C₅₋₆hetereoaryl, C₄₋₆heterocycloalkyl, O—CH₂CH₂OC₁₋₄alkyl, OCH₂OC₁₋₄alkyl, C(O)NH₂, C(O)NHC₁₋₁₀alkyl, C(O)N(C₁₋₁₀alkyl)(C₁₋₁₀alkyl), C(O)OC₁₋₁₀alkyl, C(O)OC₁₋₁₀fluoroalkyl, C(O)C₁₋₁₀alkyl, C(O)OH, C(O)C₄₋₆cycloalkyl, C(O)C₄₋₆heterocycloalkyl, C(O)C₅₋₆heteroaryl, C(O)phenyl, C(O)OC₄₋₆cycloalkyl, C(O)OC₅₋₆heteroaryl, C(O)Ophenyl or C(O)OC₄₋₆heterocycloalkyl and optionally one or two F, CN or C₁₋₄alkyl, wherein each cycloalkyl, phenyl, heterocycloalkyl and heteroaryl in the Cy¹ substituents is optionally substituted with one to four substituents independently selected from F, C₁₋₄alkyl, C₀₋₄alkyleneNHC₁₋₄alkyl and C₀₋₄alkyleneN(C₁₋₄alkyl)(C₁₋₄alkyl);
when Cy¹ is a bicyclic ring, Cy¹ is optionally substituted with Cy² and/or one or two F, CN or C₁₋₄alkyl;
Cy² is an optionally substituted phenyl, optionally substituted 5- or 6-membered heteroaromatic monocyclic ring, optionally substituted 5- or 6-membered heterocycloalkyl monocyclic ring, optionally substituted 9- or 10-membered aromatic bicyclic ring, optionally substituted 8-, 9- or 10-membered heteroaromatic bicyclic ring or optionally substituted 8-, 9- or 10-membered heterocycloalkyl bicyclic ring; and
the optional substituents on Cy² are independently selected from one or two of F, C₁₋₄alkyl, C₁₋₄fluoroalkyl, OC₁₋₄alkyl, OC₁₋₄fluoroalkyl and CN.

2. The compound of claim 1, wherein at least one of R¹ and R² is CH₃.

3.-4. (canceled)

5. The compound of claim 1, wherein R¹ and R² are selected to provide one of the following groups in the compounds of Formula I:

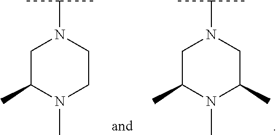

6. The compound of claim 1, wherein R³, R⁴, R⁵, X² and X³ are selected to provide one of the following groups in the compounds of Formula I: 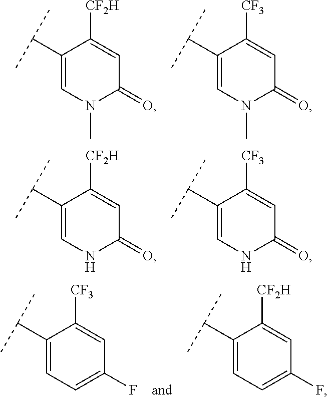
and tautomers thereof.

7. The compound of claim 1, wherein R³, R⁴, R⁵, X² and X³ are selected to provide following groups in the compound of Formula I 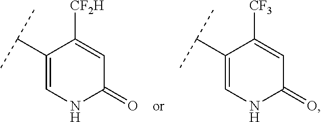
and the corresponding tautomers are

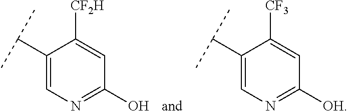

8. The compound of claim 1, wherein when Cy¹ is a monocyclic ring Cy¹ is substituted with at least one Cy² and optionally one or two F or C₁₋₄alkyl; or Cy¹ is substituted with N(CH₃)₂.

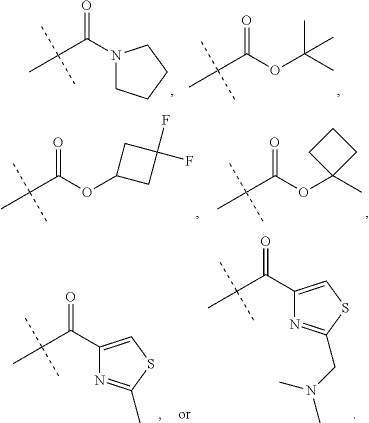

9. The compound of claim 1, wherein Cy¹ is a monocyclic 5- or 6-membered heterocyclic ring substituted with Cy² or a 5- or 6-membered heteroaromatic ring substituted with Cy².

10. The compound of claim 1, wherein Cy¹ is a 6-membered heterocyclic ring substituted with Cy² at the para or meta position from the point of attachment of Cy¹ to the remainder of the compound of Formula I or a 6-membered heteroaromatic ring substituted with Cy² at the para or meta position from the point of attachment of Cy¹ to the remainder of the compound of Formula I.

11. The compound of claim 1, wherein Cy¹ is a 5-membered heterocyclic ring substituted with Cy² at the beta or gamma position from the point of attachment of Cy¹ to the remainder of the compound of Formula I or a 5-membered heteroaromatic ring substituted with Cy² at the beta or gamma position from the point of attachment of Cy¹ to the remainder of the compound of Formula I.

12.-13. (canceled)

14. The compound of claim 1, wherein Cy¹ is selected from:

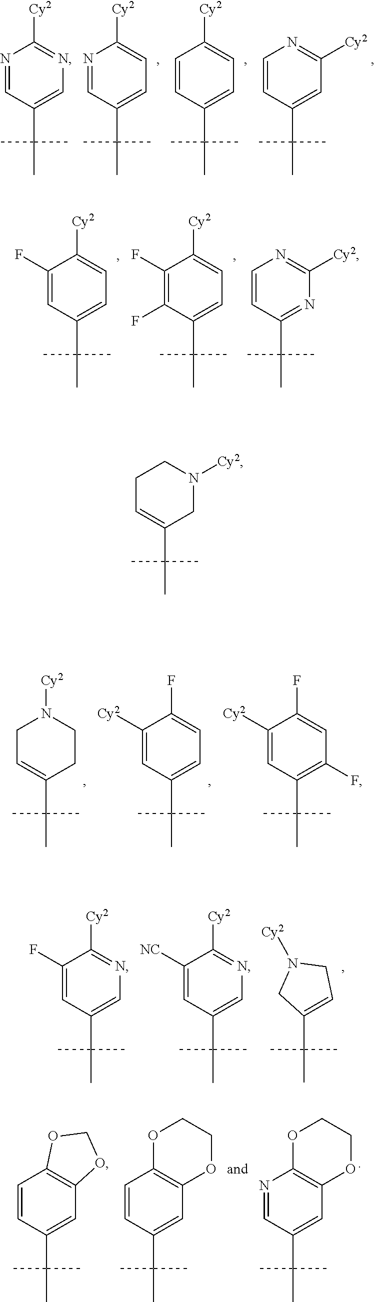

15. The compound of claim 1, wherein Cy² is an optionally substituted phenyl, an optionally substituted 5 or 6 membered heteroaromatic monocyclic ring, or an optionally substituted 5 or 6 membered heterocycloalkyl monocyclic ring.

16.-17. (canceled)

18. The compound of claim 1, wherein Cy² is selected from optionally substituted morpholinyl, optionally substituted piperidinyl, optionally substituted pyrimidinyl and optionally substituted thiazolyl.

19.-20. (canceled)

21. The compound of claim 1, wherein the optional substituents on Cy² are selected from one or two of F, CH₃, CF₃, OCH₃, OCF₃ and CN.

22. (canceled)

23. The compound of claim 1, wherein Cy² is selected from:

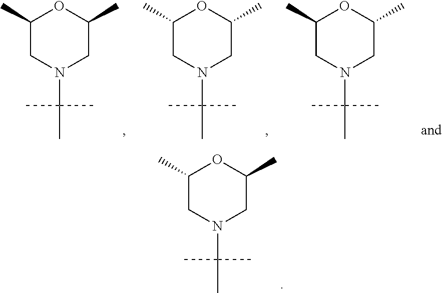

24. The compound of claim 1, wherein the compound of Formula I has the following structure: 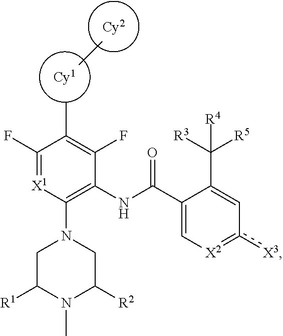
wherein:
R¹ and R² are independently selected from H and CH₃;
R³, R⁴ and R⁵ are independently selected from H and F, provided that at least two of R³, R⁴ and R⁵ are F; [symbol]
is a single or double bond, provided that one [symbol] is a single bond and the other [symbol] is a double bond;
X¹ is selected from CH and N;
X² is NH or NCH₃ when the adjacent [symbol] is a single bond and X² is CH when the adjacent [symbol] is a double bond;
X³ is F when the adjacent [symbol] is a single bond and X³ is O when [symbol] is a double bond;
Cy¹ is phenyl, 5- or 6-membered heteroaromatic monocyclic ring, or 5- or 6-membered heterocycloalkyl monocyclic ring further optionally substituted with one or two F, CN or C₁₋₄alkyl;
Cy² is an optionally substituted phenyl, optionally substituted 5- or 6-membered heteroaromatic monocyclic ring, or optionally substituted 5- or 6-membered heterocycloalkyl monocyclic ring or an optionally substituted 9- or 10-membered aromatic bicyclic ring, optionally substituted 9- or 10-membered heteroaromatic bicyclic ring or optionally substituted 9- or 10-membered heterocycloalkyl bicyclic ring; and
the optional substituents on Cy² are selected from one or two of F, C₁₋₆alkyl, C₁₋₆fluoroalkyl, OC₁₋₆alkyl, OC₁₋₆fluoroalkyl and CN, and
pharmaceutically acceptable salts and/or solvates thereof.

25. (canceled)

26. The compound of claim 1, wherein the compound of Formula (I) is selected from:
N-(3-(6-(cyclopropylmethoxy)pyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(2-morpholinopyrimidin-5-yl)-6-((3S,5R)-3,4,5-trimethylpiperazin-1-yl)phenyl)-6-oxo-4-(trifluoromethyl)-1,6-dihydropyridine-3-carboxamide
N-(3-(2-(cyclopropylmethoxy)pyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(6-morpholinopyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,6-difluoro-4′-morpholino-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
N-(2,4-difluoro-3-(6-(2-methoxyethoxy)pyridin-3-yl)-6-((3S,5R)-3,4,5-trimethylpiperazin-1-yl)phenyl)-6-oxo-4-(trifluoromethyl)-1,6-dihydropyridine-3-carboxamide
N-(4′-(cyclopropylmethoxy)-2,6-difluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
N-(3-(2-((2S,6R)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
6-Oxo-N-(2,3′,6-trifluoro-4′-morpholino-4-((3S,5R)-3,4,5-trimethylpiperazin-1-yl)-[1,1′-biphenyl]-3-yl)-4-(trifluoromethyl)-1,6-dihydropyridine-3-carboxamide
N-[2,4-difluoro-3-(2-morpholin-4-ylpyrimidin-5-yl)-6-[(3R,5S)-3,4,5-trimethylpiperazin-1-yl]phenyl]-4-fluoro-2-(trifluoromethyl)benzamide
N-(2,4-difluoro-3-(2-morpholinopyrimidin-5-yl)-6-((3S,5R)-3,4,5-trimethylpiperazin-1-yl)phenyl)-4-fluoro-2-(trifluoromethyl)benzamide formic acid
N-(2,4-difluoro-3-(2-((S)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide formic acid
Isopropyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate
1-Methylcyclobutyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate
N-(2,4-difluoro-3-(1-(pyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(2-((S)-2-methylmorpholino)pyrimidin-5-yl)-6-((3S,5R)-3,4,5-trimethylpiperazin-1-yl)phenyl)-4-fluoro-2-(trifluoromethyl)benzamide formic acid
N-(2,4-difluoro-3-(2-((R)-2-methylmorpholino)pyrimidin-5-yl)-6-((3S,5R)-3,4,5-trimethylpiperazin-1-yl)phenyl)-1-methyl-6-oxo-4-(trifluoromethyl)-1,6-dihydropyridine-3-carboxamide formic acid
N-(2,4-difluoro-3-(2-((R)-2-methylmorpholino)pyrimidin-5-yl)-6-((3S,5R)-3,4,5-trimethylpiperazin-1-yl)phenyl)-4-fluoro-2-(trifluoromethyl)benzamide formic acid
N-(2,4-difluoro-3-(1-pivaloyl-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
3,3-Difluorocyclobutyl 5-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate
N-(2,4-difluoro-3-(1-(pyrimidin-2-yl)-1,2,5,6-tetrahydropyridin-3-yl)-6-((3S,5R)-3,4,5-trimethylpiperazin-1-yl)phenyl)-4-(difluoromethyl)-6-oxo-1,6-dihydropyridine-3-carboxamide
1-Methylcyclobutyl 5-(3-(4-(difluoromethyl)-6-oxo-1,6-dihydropyridine-3-carboxamido)-2,6-difluoro-4-((3S,5R)-3,4,5-trimethylpiperazin-1-yl)phenyl)-3,6-dihydropyridine-1(2H)-carboxylate
Isopropyl 5-(3-(4-(difluoromethyl)-6-oxo-1,6-dihydropyridine-3-carboxamido)-2,6-difluoro-4-((3S,5R)-3,4,5-trimethylpiperazin-1-yl)phenyl)-3,6-dihydropyridine-1(2H)-carboxylate
6-Oxo-N-(2,3′,6-trifluoro-4′-(methylcarbamoyl)-4-((3S,5R)-3,4,5-trimethylpiperazin-1-yl)-[1,1′-biphenyl]-3-yl)-4-(trifluoromethyl)-1,6-dihydropyridine-3-carboxamide
N-(4′-carbamoyl-2,3′,6-trifluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
N-(4′-carbamoyl-2,2′,3′,6-tetrafluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
6-Oxo-N-(2,2′,3′,6-tetrafluoro-4′-((2,4,4-trimethylpentan-2-yl)carbamoyl)-4-((3S,5R)-3,4,5-trimethylpiperazin-1-yl)-[1,1′-biphenyl]-3-yl)-4-(trifluoromethyl)-1,6-dihydropyridine-3-carboxamide
N-(3′-carbamoyl-2,4′,6-trifluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
6-Oxo-N-(2,4′,6-trifluoro-3′-(methylcarbamoyl)-4-((3S,5R)-3,4,5-trimethylpiperazin-1-yl)-[1,1′-biphenyl]-3-yl)-4-(trifluoromethyl)-1,6-dihydropyridine-3-carboxamide
N-(5′-carbamoyl-2,2′,4′,6-tetrafluoro-4-((3S,5R)-3,4,5-trimethylpiperazin-1-yl)-[1,1′-biphenyl]-3-yl)-6-oxo-4-(trifluoromethyl)-1,6-dihydropyridine-3-carboxamide
N-(2,4-difluoro-3-(2-morpholinopyrimidin-5-yl)-6-((3S,5R)-3,4,5-trimethylpiperazin-1-yl)phenyl)-2-(difluoromethyl)-4-fluorobenzamide formic acid
2-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-4-fluorobenzamide formic acid
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide
Isopropyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
Isopropyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
Isopropyl (S)-3-(3-(4-(difluoromethyl)-6-oxo-1,6-dihydropyridine-3-carboxamido)-4-(3,4-dimethylpiperazin-1-yl)-2,6-difluorophenyl)-2,5-dihydro-1H-pyrrole-1-carboxylate
Isopropyl (S)-3-(4-(3,4-dimethylpiperazin-1-yl)-2,6-difluoro-3-(6-oxo-4-(trifluoromethyl)-1,6-dihydropyridine-3-carboxamido)phenyl)-2,5-dihydro-1H-pyrrole-1-carboxylate
Isopropyl (S)-5-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
Isopropyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
N-(2,4-difluoro-3-(2-morpholinopyrimidin-5-yl)-6-((3S,5R)-3,4,5-trimethylpiperazin-1-yl)phenyl)-4-(difluoromethyl)-1-methyl-6-oxo-1,6-dihydropyridine-3-carboxamide
N-(2,4-difluoro-3-(2-morpholinopyrimidin-5-yl)-6-((3S,5R)-3,4,5-trimethylpiperazin-1-yl)phenyl)-4-(difluoromethyl)-6-oxo-1,6-dihydropyridine-3-carboxamide
(3,3-Difluorocyclobutyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
3,3-Difluorocyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
3,3-Difluorocyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
3,3-Difluorocyclobutyl (S)-3-(4-(3,4-dimethylpiperazin-1-yl)-2,6-difluoro-3-(6-oxo-4-(trifluoromethyl)-1,6-dihydropyridine-3-carboxamido)phenyl)-2,5-dihydro-1H-pyrrole-1-carboxylate
3,3-Difluorocyclobutyl (S)-5-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
3,3-Difluorocyclobutyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
(S)-4-(difluoromethyl)-N-(6-(3,4-dimethylpiperazin-1-yl)-2,4-difluoro-3-(1-(5-methoxypyrimidin-2-yl)-1,2,3,6-tetrahydropyridin-4-yl)phenyl)-6-oxo-1,6-dihydropyridine-3-carboxamide
(S)-4-(difluoromethyl)-N-(6-(3,4-dimethylpiperazin-1-yl)-2,4-difluoro-3-(1-(5-methoxypyrimidin-2-yl)-1,2,5,6-tetrahydropyridin-3-yl)phenyl)-6-oxo-1,6-dihydropyridine-3-carboxamide
(S)-4-(difluoromethyl)-N-(6-(3,4-dimethylpiperazin-1-yl)-2,4-difluoro-3-(1-(5-methoxypyrimidin-2-yl)-2,5-dihydro-1H-pyrrol-3-yl)phenyl)-6-oxo-1,6-dihydropyridine-3-carboxamide
(S)—N-(6-(3,4-dimethylpiperazin-1-yl)-2,4-difluoro-3-(1-(5-methoxypyrimidin-2-yl)-2,5-dihydro-1H-pyrrol-3-yl)phenyl)-6-oxo-4-(trifluoromethyl)-1,6-dihydropyridine-3-carboxamide
(S)—N-(6-(3,4-dimethylpiperazin-1-yl)-2,4-difluoro-3-(1-(5-methoxypyrimidin-2-yl)-1,2,5,6-tetrahydropyridin-3-yl)phenyl)-6-oxo-4-(trifluoromethyl)-1,6-dihydropyridine-3-carboxamide
(S)—N-(6-(3,4-dimethylpiperazin-1-yl)-2,4-difluoro-3-(1-(5-methoxypyrimidin-2-yl)-1,2,3,6-tetrahydropyridin-4-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(pyrimidin-2-yl)-1,2,5,6-tetrahydropyridin-3-yl)phenyl)-6-oxo-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
N-(3-(2-((2S,6R)-2,6-dimethylmorpholino)pyrimidin-4-yl)-6-((S)-3,4-dimethylpiperazin-1-yl)-2,4-difluorophenyl)-1-methyl-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-1-methyl-6-oxo-1,6-dihydropyridine-3-carboxamide
N-(3-(2-((2S,6R)-2,6-dimethylmorpholino)pyrimidin-4-yl)-6-((S)-3,4-dimethylpiperazin-1-yl)-2,4-difluorophenyl)-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-4-yl)-6-((S)-3,4-dimethylpiperazin-1-yl)-2,4-difluorophenyl)-6-oxo-1,6-dihydropyridine-3-carboxamide
N-(6-((S)-3,4-dimethylpiperazin-1-yl)-2,4-difluoro-3-(2-((S)-2-methylmorpholino)pyrimidin-5-yl)phenyl)-6-oxo-4-(trifluoromethyl)-1,6-dihydropyridine-3-carboxamide
N-(3-(2-(4,4-difluoropiperidin-1-yl)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(6-((2S,6R)-2,6-dimethylmorpholino)pyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(6-(dimethylamino)-5-fluoropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(5-cyano-6-morpholinopyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(6-((tetrahydro-2H-pyran-4-yl)oxy)pyridin-3-yl)-6-((3S,5R)-3,4,5-trimethylpiperazin-1-yl)phenyl)-6-oxo-4-(trifluoromethyl)-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide formic acid
(S)-4-(difluoromethyl)-N-(6-(3,4-dimethylpiperazin-1-yl)-2,4-difluoro-3-(1-(6-methoxypyrimidin-4-yl)-1,2,3,6-tetrahydropyridin-4-yl)phenyl)-6-oxo-1,6-dihydropyridine-3-carboxamide
(S)—N-(6-(3,4-dimethylpiperazin-1-yl)-2,4-difluoro-3-(1-(6-methoxypyrimidin-4-yl)-1,2,5,6-tetrahydropyridin-3-yl)phenyl)-6-oxo-4-(trifluoromethyl)-1,6-dihydropyridine-3-carboxamide
N-(2,4-difluoro-3-(2-morpholinopyridin-4-yl)-6-((3S,5R)-3,4,5-trimethylpiperazin-1-yl)phenyl)-6-oxo-4-(trifluoromethyl)-1,6-dihydropyridine-3-carboxamide
N-(3-(2-(dimethylamino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(Benzo[d][1,3]dioxol-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(2,3-dihydrobenzo[b][1,4]dioxin-6-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(2,3-dihydro-[1,4]dioxino[2,3-b]pyridin-7-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(6-methoxypyrimidin-4-yl)-2,5-dihydro-1H-pyrrol-3-yl)phenyl)-6-oxo-1,6-dihydropyridine-3-carboxamide
(S)—N-(6-(3,4-dimethylpiperazin-1-yl)-2,4-difluoro-3-(1-(6-methoxypyrimidin-4-yl)-2,5-dihydro-1H-pyrrol-3-yl)phenyl)-6-oxo-4-(trifluoromethyl)-1,6-dihydropyridine-3-carboxamide
(S)—N-(6-(3,4-dimethylpiperazin-1-yl)-2,4-difluoro-3-(1-(6-methoxypyrimidin-4-yl)-1,2,3,6-tetrahydropyridin-4-yl)phenyl)-6-oxo-4-(trifluoromethyl)-1,6-dihydropyridine-3-carboxamide
N-(3-(2-((2S,6R)-2,6-dimethylmorpholino)pyrimidin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-4-yl)-2,4-difluoro-6-((3S,5R)-3,4,5-trimethylpiperazin-1-yl)phenyl)-6-oxo-1,6-dihydropyridine-3-carboxamide
(S)-4-(Difluoromethyl)-N-(6-(3,4-dimethylpiperazin-1-yl)-2,4-difluoro-3-(1-(6-methoxypyrimidin-4-yl)-1,2,5,6-tetrahydropyridin-3-yl)phenyl)-6-oxo-1,6-dihydropyridine-3-carboxamide
(S)—N-(6-(3,4-dimethylpiperazin-1-yl)-2,4-difluoro-3-(1-(5-fluoropyrimidin-2-yl)-2,5-dihydro-1H-pyrrol-3-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(5-fluoropyrimidin-2-yl)-1,2,3,6-tetrahydropyridin-4-yl)phenyl)-6-oxo-1,6-dihydropyridine-3-carboxamide
(S)—N-(6-(3,4-dimethylpiperazin-1-yl)-2,4-difluoro-3-(1-(5-fluoropyrimidin-2-yl)-1,2,3,6-tetrahydropyridin-4-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(5-fluoropyrimidin-2-yl)-1,2,5,6-tetrahydropyridin-3-yl)phenyl)-6-oxo-1,6-dihydropyridine-3-carboxamide
(S)—N-(6-(3,4-dimethylpiperazin-1-yl)-2,4-difluoro-3-(1-(5-fluoropyrimidin-2-yl)-1,2,5,6-tetrahydropyridin-3-yl)phenyl)-6-oxo-4-(trifluoromethyl)-1,6-dihydropyridine-3-carboxamide
N-(3-(2-((2R,6R)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
N-(3-(2-((2R,6R)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(2-((2S,6S)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
N-(3-(2-((2S,6S)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(2-((1R,5S)-8-oxa-3-azabicyclo[3.2.1]octan-3-yl)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
N-(3-(2-((1R,5S)-8-oxa-3-azabicyclo[3.2.1]octan-3-yl)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
(S)-4-(difluoromethyl)-N-(6-(3,4-dimethylpiperazin-1-yl)-2,4-difluoro-3-(1-(5-fluoropyrimidin-2-yl)-2,5-dihydro-1H-pyrrol-3-yl)phenyl)-6-oxo-1,6-dihydropyridine-3-carboxamide
1-Methylcyclobutyl (S)-4-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
1-Methylcyclobutyl (S)-4-(4-(3,4-dimethylpiperazin-1-yl)-2,6-difluoro-3-(6-oxo-4-(trifluoromethyl)-1,6-dihydropyridine-3-carboxamido)phenyl)-3,6-dihydropyridine-1(2H)-carboxylate
1-Methylcyclobutyl (S)-5-(3-(4-(difluoromethyl)-6-oxo-1,6-dihydropyridine-3-carboxamido)-4-(3,4-dimethylpiperazin-1-yl)-2,6-difluorophenyl)-3,6-dihydropyridine-1(2H)-carboxylate
(S)—N-(3-(1-(5-cyanothiazol-2-yl)-2,5-dihydro-1H-pyrrol-3-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(5-cyanothiazol-2-yl)-2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(1-(5-cyanothiazol-2-yl)-1,2,3,6-tetrahydropyridin-4-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(5-cyanothiazol-2-yl)-1,2,3,6-tetrahydropyridin-4-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(1-(5-cyanothiazol-2-yl)-1,2,5,6-tetrahydropyridin-3-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(5-cyanothiazol-2-yl)-1,2,5,6-tetrahydropyridin-3-yl)-6-(3,4-dimethylpiperazin-1-yl)-2,4-difluorophenyl)-6-oxo-4-(trifluoromethyl)-1,6-dihydropyridine-3-carboxamide
N-(2,4-difluoro-3-(2-((S)-2-methylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide
N-(6-((S)-3,4-dimethylpiperazin-1-yl)-2,4-difluoro-3-(2-((S)-2-isopropylmorpholino)pyrimidin-5-yl)phenyl)-6-oxo-4-(trifluoromethyl)-1,6-dihydropyridine-3-carboxamide
N-(2,4-difluoro-3-(2-((S)-2-isopropylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide
N-(6-((S)-3,4-dimethylpiperazin-1-yl)-2,4-difluoro-3-(2-((R)-2-isopropylmorpholino)pyrimidin-5-yl)phenyl)-6-oxo-4-(trifluoromethyl)-1,6-dihydropyridine-3-carboxamide
N-(2,4-difluoro-3-(2-((R)-2-isopropylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide
(S)—N-(3-(1-(2-cyanopyrimidin-4-yl)-2,5-dihydro-1H-pyrrol-3-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(2-cyanopyrimidin-4-yl)-2,5-dihydro-1H-pyrrol-3-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(1-(2-cyanopyrimidin-4-yl)-1,2,3,6-tetrahydropyridin-4-yl)-6-(3,4-dimethylpiperazin-1-yl)-2,4-difluorophenyl)-4-(difluoromethyl)-6-oxo-1,6-dihydropyridine-3-carboxamide
2-(Difluoromethyl)-N-(3-(2-((2S,6R)-2,6-dimethylmorpholino)pyrimidin-5-yl)-2,4-difluoro-6-((3S,5R)-3,4,5-trimethylpiperazin-1-yl)phenyl)-4-fluorobenzamide formic acid
2-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-4-fluorobenzamide formic acid
4-(Difluoromethyl)-N-(3-(2-((2S,6R)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide
(S)—N-(3-(1-(2-cyanopyrimidin-4-yl)-1,2,3,6-tetrahydropyridin-4-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(1-(2-cyanopyrimidin-4-yl)-1,2,5,6-tetrahydropyridin-3-yl)-6-(3,4-dimethylpiperazin-1-yl)-2,4-difluorophenyl)-4-(difluoromethyl)-6-oxo-1,6-dihydropyridine-3-carboxamide
(S)—N-(3-(1-(2-cyanopyrimidin-4-yl)-1,2,5,6-tetrahydropyridin-3-yl)-6-(3,4-dimethylpiperazin-1-yl)-2,4-difluorophenyl)-6-oxo-4-(trifluoromethyl)-1,6-dihydropyridine-3-carboxamide
4-(Difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide
4-(Difluoromethyl)-N-(3-(2-((2R,6R)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide
4-(Difluoromethyl)-N-(6-((S)-3,4-dimethylpiperazin-1-yl)-2,4-difluoro-3-(2-((R)-2-isopropylmorpholino)pyrimidin-5-yl)phenyl)-6-oxo-1,6-dihydropyridine-3-carboxamide
N-(2,4-Difluoro-3-(2-((R)-2-isopropylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide
4-(Difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide
4-(Difluoromethyl)-N-(3-(2-((2S,6S)-2,6-dimethylmorpholino)pyrimidin-5-yl)-2,4-difluoro-6-((3S,5R)-3,4,5-trimethylpiperazin-1-yl)phenyl)-6-oxo-1,6-dihydropyridine-3-carboxamide
4-(Difluoromethyl)-N-(6-((S)-3,4-dimethylpiperazin-1-yl)-2,4-difluoro-3-(2-((S)-2-isopropylmorpholino)pyrimidin-5-yl)phenyl)-6-oxo-1,6-dihydropyridine-3-carboxamide
N-(2,4-difluoro-3-(2-((S)-2-isopropylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide
N-(6-((S)-3,4-dimethylpiperazin-1-yl)-2,4-difluoro-3-(2-((R)-2-isopropylmorpholino)pyrimidin-4-yl)phenyl)-6-oxo-4-(trifluoromethyl)-1,6-dihydropyridine-3-carboxamide
4-(Difluoromethyl)-N-(6-((S)-3,4-dimethylpiperazin-1-yl)-2,4-difluoro-3-(2-((R)-2-isopropylmorpholino)pyrimidin-4-yl)phenyl)-6-oxo-1,6-dihydropyridine-3-carboxamide
N-(2,4-difluoro-3-(2-((S)-2-methylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide
4-(Difluoromethyl)-N-(6-((S)-3,4-dimethylpiperazin-1-yl)-2,4-difluoro-3-(2-((S)-2-methylmorpholino)pyrimidin-5-yl)phenyl)-6-oxo-1,6-dihydropyridine-3-carboxamide
N-(6-((S)-3,4-dimethylpiperazin-1-yl)-2,4-difluoro-3-(2-((R)-2-methylmorpholino)pyrimidin-5-yl)phenyl)-6-oxo-4-(trifluoromethyl)-1,6-dihydropyridine-3-carboxamide
N-(2,4-difluoro-3-(2-((R)-2-methylmorpholino)pyrimidin-5-yl)-6-((3S,5R)-3,4,5-trimethylpiperazin-1-yl)phenyl)-6-oxo-4-(trifluoromethyl)-1,6-dihydropyridine-3-carboxamide
4-(Difluoromethyl)-N-(6-((S)-3,4-dimethylpiperazin-1-yl)-2,4-difluoro-3-(2-((R)-2-methylmorpholino)pyrimidin-5-yl)phenyl)-6-oxo-1,6-dihydropyridine-3-carboxamide
N-(2,4-difluoro-3-(2-((R)-2-methylmorpholino)pyrimidin-5-yl)-6-((3S,5R)-3,4,5-trimethylpiperazin-1-yl)phenyl)-4-(difluoromethyl)-6-oxo-1,6-dihydropyridine-3-carboxamide
N-(3-(2-((2S,6R)-2,6-dimethylmorpholino)thiazol-4-yl)-6-((S)-3,4-dimethylpiperazin-1-yl)-2,4-difluorophenyl)-6-oxo-4-(trifluoromethyl)-1,6-dihydropyridine-3-carboxamide
4-(Difluoromethyl)-N-(3-(2-((2S,6R)-2,6-dimethylmorpholino)thiazol-4-yl)-6-((S)-3,4-dimethylpiperazin-1-yl)-2,4-difluorophenyl)-6-oxo-1,6-dihydropyridine-3-carboxamide
4-(2,6-Difluoro-3-(6-oxo-4-(trifluoromethyl)-1,6-dihydropyridine-3-carboxamido)-4-((3S,5R)-3,4,5-trimethylpiperazin-1-yl)phenyl)-5,6-dihydropyridine-1(2H)-carboxylate
3-(2,6-Difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate
3,3-Difluorocyclobutyl 4-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-5,6-dihydropyridine-1(2H)-carboxylate
3,3-Difluorocyclobutyl 3-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate
N-(2,4-difluoro-3-(1-(5-methoxypyrimidin-2-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(5-methoxypyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(5-methoxypyrimidin-2-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(6-methoxypyrimidin-4-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide
1-Methylcyclobutyl 3-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-2,5-dihydro-1H-pyrrole-1-carboxylate
N-(2,4-difluoro-3-(1-(6-methoxypyrimidin-4-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(6-methoxypyrimidin-4-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide
1-Methylcyclobutyl 4-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-5,6-dihydropyridine-1(2H)-carboxylate
N-(2,4-difluoro-3-(1-(5-fluoropyrimidin-2-yl)-1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(5-fluoropyrimidin-2-yl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1-(5-fluoropyrimidin-2-yl)-2,5-dihydro-1H-pyrrol-3-yl)-6-((3S,5R)-3,4,5-trimethylpiperazin-1-yl)phenyl)-6-oxo-4-(trifluoromethyl)-1,6-dihydropyridine-3-carboxamide
N-(3-(1-(5-cyanothiazol-2-yl)-1,2,3,6-tetrahydropyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(1-(5-cyanothiazol-2-yl)-1,2,5,6-tetrahydropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(3-(1-(5-cyanothiazol-2-yl)-2,5-dihydro-1H-pyrrol-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(1,2,3,6-tetrahydropyridin-4-yl)-6-((3S,5R)-3,4,5-trimethylpiperazin-1-yl)phenyl)-6-oxo-4-(trifluoromethyl)-1,6-dihydropyridine-3-carboxamide
N-(3-(1-(2-cyanopyrimidin-4-yl)-1,2,3,6-tetrahydropyridin-4-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
(S)-2-(difluoromethyl)-N-(6-(3,4-dimethylpiperazin-1-yl)-2,4-difluoro-3-(5-methyl-4-(pyrrolidine-1-carbonyl)thiazol-2-yl)phenyl)-4-fluorobenzamide
(S)—N-(6-(3,4-dimethylpiperazin-1-yl)-2,4-difluoro-3-(5-methyl-4-(pyrrolidine-1-carbonyl)thiazol-2-yl)phenyl)-4-fluoro-2-(trifluoromethyl)benzamide
(S)—N-(3-(2-(8-oxa-3-azabicyclo[3.2.1]octan-3-yl)pyrimidin-5-yl)-6-(3,4-dimethylpiperazin-1-yl)-2,4-difluorophenyl)-4-(difluoromethyl)-6-oxo-1,6-dihydropyridine-3-carboxamide
N-(3-(2-(8-oxa-3-azabicyclo[3.2.1]octan-3-yl)pyrimidin-5-yl)-2,4-difluoro-6-((3S,5R)-3,4,5-trimethylpiperazin-1-yl)phenyl)-4-(difluoromethyl)-6-oxo-1,6-dihydropyridine-3-carboxamide
2-(Difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-4-fluorobenzamide 2-(Difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-4-fluorobenzamide
(S)-4-(difluoromethyl)-N-(6-(3,4-dimethylpiperazin-1-yl)-2,4-difluoro-3-(1-(2-methylthiazole-4-carbonyl)-1,2,3,6-tetrahydropyridin-4-yl)phenyl)-6-oxo-1,6-dihydropyridine-3-carboxamide
4-(Difluoromethyl)-N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide
4-(Difluoromethyl)-N-(3-(2-((2R,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide
N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2R,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2S,6S)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide formic acid salt
N-(3-(2-((2R,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide formic acid salt
N-(2,4-difluoro-3-(6-((R)-2-methylmorpholino)pyridin-3-yl)-6-((3S,5R)-3,4,5-trimethylpiperazin-1-yl)phenyl)-6-oxo-4-(trifluoromethyl)-1,6-dihydropyridine-3-carboxamide
N-(6-((S)-3,4-dimethylpiperazin-1-yl)-2,4-difluoro-3-(6-((R)-2-methylmorpholino)pyridin-3-yl)phenyl)-6-oxo-4-(trifluoromethyl)-1,6-dihydropyridine-3-carboxamide
N-(3-(6-((2S,6R)-2,6-dimethylmorpholino)pyridin-3-yl)-6-((S)-3,4-dimethylpiperazin-1-yl)-2,4-difluorophenyl)-6-oxo-4-(trifluoromethyl)-1,6-dihydropyridine-3-carboxamide
N-(6-((S)-3,4-dimethylpiperazin-1-yl)-2,4-difluoro-3-(6-((S)-2-methylmorpholino)pyridin-3-yl)phenyl)-6-oxo-4-(trifluoromethyl)-1,6-dihydropyridine-3-carboxamide
N-(6-((S)-3,4-dimethylpiperazin-1-yl)-2,4-difluoro-3-(6-((R)-2-methylmorpholino)pyridin-3-yl)phenyl)-4-fluoro-2-(trifluoromethyl)benzamide
(S)—N-(6-(3,4-dimethylpiperazin-1-yl)-2,4-difluoro-3-(2-(piperazin-1-yl)pyrimidin-5-yl)phenyl)-4-fluoro-2-(trifluoromethyl)benzamide
(S)—N-(6-(3,4-dimethylpiperazin-1-yl)-2,4-difluoro-3-(6-morpholinopyridin-3-yl)phenyl)-4-fluoro-2-(trifluoromethyl)benzamide
4-(Difluoromethyl)-N-(3-(6-((2S,6R)-2,6-dimethylmorpholino)pyridin-3-yl)-6-((S)-3,4-dimethylpiperazin-1-yl)-2,4-difluorophenyl)-1-methyl-6-oxo-1,6-dihydropyridine-3-carboxamide
N-(3-(6-((2S,6R)-2,6-dimethylmorpholino)pyridin-3-yl)-6-((S)-3,4-dimethylpiperazin-1-yl)-2,4-difluorophenyl)-4-fluoro-2-(trifluoromethyl)benzamide
2-(Difluoromethyl)-N-(6-((S)-3,4-dimethylpiperazin-1-yl)-2,4-difluoro-3-(6-((R)-2-methylmorpholino)pyridin-3-yl)phenyl)-4-fluorobenzamide
2-(Difluoromethyl)-N-(3-(6-((2S,6R)-2,6-dimethylmorpholino)pyridin-3-yl)-6-((S)-3,4-dimethylpiperazin-1-yl)-2,4-difluorophenyl)-4-fluorobenzamide
2-(Difluoromethyl)-N-(6-((S)-3,4-dimethylpiperazin-1-yl)-2,4-difluoro-3-(6-((S)-2-methylmorpholino)pyridin-3-yl)phenyl)-4-fluorobenzamide
(S)-2-(difluoromethyl)-N-(6-(3,4-dimethylpiperazin-1-yl)-2,4-difluoro-3-(6-morpholinopyridin-3-yl)phenyl)-4-fluorobenzamide
4-(Difluoromethyl)-N-(6-((S)-3,4-dimethylpiperazin-1-yl)-2,4-difluoro-3-(6-((R)-2-methylmorpholino)pyridin-3-yl)phenyl)-6-oxo-1,6-dihydropyridine-3-carboxamide
4-(Difluoromethyl)-N-(3-(6-((2S,6R)-2,6-dimethylmorpholino)pyridin-3-yl)-6-((S)-3,4-dimethylpiperazin-1-yl)-2,4-difluorophenyl)-6-oxo-1,6-dihydropyridine-3-carboxamide
4-(Difluoromethyl)-N-(6-((S)-3,4-dimethylpiperazin-1-yl)-2,4-difluoro-3-(6-((S)-2-methylmorpholino)pyridin-3-yl)phenyl)-6-oxo-1,6-dihydropyridine-3-carboxamide
(S)—N-(3-(benzo[d][1,3]dioxol-5-yl)-6-(3,4-dimethylpiperazin-1-yl)-2,4-difluorophenyl)-6-oxo-4-(trifluoromethyl)-1,6-dihydropyridine-3-carboxamide
(S)—N-(3-(2,3-dihydrobenzo[b][1,4]dioxin-6-yl)-6-(3,4-dimethylpiperazin-1-yl)-2,4-difluorophenyl)-6-oxo-4-(trifluoromethyl)-1,6-dihydropyridine-3-carboxamide
(S)—N-(6-(3,4-dimethylpiperazin-1-yl)-2,4-difluoro-3-(6-((tetrahydro-2H-pyran-4-yl)oxy)pyridin-3-yl)phenyl)-6-oxo-4-(trifluoromethyl)-1,6-dihydropyridine-3-carboxamide
(S)-4-(difluoromethyl)-N-(3-(1-(2-((dimethylamino)methyl)thiazole-4-carbonyl)-1,2,3,6-tetrahydropyridin-4-yl)-6-(3,4-dimethylpiperazin-1-yl)-2,4-difluorophenyl)-6-oxo-1,6-dihydropyridine-3-carboxamide
N-(2,4-difluoro-3-(1-(2-methylthiazole-4-carbonyl)-1,2,5,6-tetrahydropyridin-3-yl)-6-((3S,5R)-3,4,5-trimethylpiperazin-1-yl)phenyl)-6-oxo-4-(trifluoromethyl)-1,6-dihydropyridine-3-carboxamide
N-(3-(1-(2-((dimethylamino)methyl)thiazole-4-carbonyl)-1,2,5,6-tetrahydropyridin-3-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
N-(2,4-difluoro-3-(6-((R)-2-methylmorpholino)pyridin-3-yl)-6-((3S,5R)-3,4,5-trimethylpiperazin-1-yl)phenyl)-4-(difluoromethyl)-6-oxo-1,6-dihydropyridine-3-carboxamide and
4-(Difluoromethyl)-N-(3-(6-((2S,6R)-2,6-dimethylmorpholino)pyridin-3-yl)-2,4-difluoro-6-((3S,5R)-3,4,5-trimethylpiperazin-1-yl)phenyl)-6-oxo-1,6-dihydropyridine-3-carboxamide
and pharmaceutically acceptable salts and/or solvates thereof.

27. The compound of claim 1, wherein the compound of Formula I is selected from:
N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-4-fluoro-2-(trifluoromethyl)benzamide
N-(3-(2-((2S,6S)-2,6-dimethylmorpholino)pyrimidin-5-yl)-6-((S)-3,4-dimethylpiperazin-1-yl)-2,4-difluorophenyl)-1-methyl-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-1-methyl-6-oxo-1,6-dihydropyridine-3-carboxamide
N-(2,4-difluoro-3-(6-((R)-2-methylmorpholino)pyridin-3-yl)-6-((3S,5R)-3,4,5-trimethylpiperazin-1-yl)phenyl)-4-(difluoromethyl)-6-oxo-1,6-dihydropyridine-3-carboxamide
N-(3-(2-((2S,6R)-2,6-dimethylmorpholino)pyrimidin-5-yl)-2,4-difluoro-6-((3S,5R)-3,4,5-trimethylpiperazin-1-yl)phenyl)-6-oxo-4-(trifluoromethyl)-1,6-dihydropyridine-3-carboxamide
4-(Difluoromethyl)-N-(3-(2-((2S,6R)-2,6-dimethylmorpholino)pyrimidin-5-yl)-6-((S)-3,4-dimethylpiperazin-1-yl)-2,4-difluorophenyl)-6-oxo-1,6-dihydropyridine-3-carboxamide
N-(3-(2-((2S,6R)-2,6-dimethylmorpholino)pyrimidin-5-yl)-6-((S)-3,4-dimethylpiperazin-1-yl)-2,4-difluorophenyl)-6-oxo-4-(trifluoromethyl)-1,6-dihydropyridine-3-carboxamide
1-Methylcyclobutyl 4-(2,6-difluoro-3-(6-oxo-4-(trifluoromethyl)-1,6-dihydropyridine-3-carboxamido)-4-((3S,5R)-3,4,5-trimethylpiperazin-1-yl)phenyl)-3,6-dihydropyridine-1(2H)-carboxylate
N-(2,4-difluoro-3-(6-((R)-2-methylmorpholino)pyridin-3-yl)-6-((3S,5R)-3,4,5-trimethylpiperazin-1-yl)phenyl)-6-oxo-4-(trifluoromethyl)-1,6-dihydropyridine-3-carboxamide and
N-(2,4-difluoro-3-(6-((S)-2-methylmorpholino)pyridin-3-yl)-6-((3S,5R)-3,4,5-trimethylpiperazin-1-yl)phenyl)-6-oxo-4-(trifluoromethyl)-1,6-dihydropyridine-3-carboxamide
and pharmaceutically acceptable salts and/or solvates thereof.
